(19) United States
(12) Patent Application Publication
(10) Pub. No.: US 2026/0136827 A1
(43) Pub. Date: May 14, 2026

(54) LIGHT-EMITTING DEVICE INCLUDING ORGANIC COMPOUND, ELECTRONIC APPARATUS AND ELECTRONIC DEVICE INCLUDING THE LIGHT-EMITTING DEVICE, AND THE ORGANIC COMPOUND

(71) Applicant: Samsung Display Co., Ltd., Yongin-si (KR)

(72) Inventors: Jihye Kim, Yongin-si (KR); Gyeongheon Kim, Yongin-si (KR); Hoilim Kim, Yongin-si (KR); Junyoung Lee, Yongin-si (KR)

(21) Appl. No.: 19/385,387

(22) Filed: Nov. 11, 2025

(30) Foreign Application Priority Data
Nov. 13, 2024 (KR) 10-2024-0160985

(51) Int. Cl. H10K 85/60; C07B 59/00; C07C 255/47; C07C 255/52; C07D 209/88; C07D 233/64; C07D 233/84; C07D 235/02; C07D 235/20; C07D 251/20; C07D 251/22; C07D 251/24; C07D 317/46; C07D 339/06; C07D 403/14; C07D 519/00; H10K 50/17

(52) U.S. Cl. CPC H10K 85/626; C07B 59/001; C07B 59/002; C07C 255/47; C07C 255/52; C07D 209/88; C07D 233/64; C07D 233/84; C07D 235/02; C07D 235/20; C07D 251/20; C07D 251/22; C07D 251/24; C07D 317/46; C07D 339/06; C07D 403/14; C07D 519/00; H10K 85/654; H10K 85/657; H10K 85/6572; C07B 2200/05; C07C 2602/08; C07C 2602/22; H10K 50/17

(57) ABSTRACT

A light-emitting device including an organic compound represented by Formula 1, and an electronic apparatus and an electronic device, each including the light-emitting device are provided. The organic compound is represented by Formula 1:

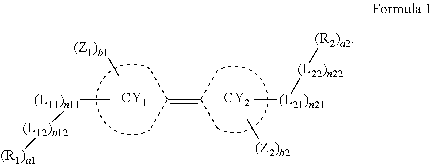

### CROSS-REFERENCE TO RELATED APPLICATION

This application claims priority to and the benefit of Korean Patent Application No. 10-2024-0160985, filed on Nov. 13, 2024, in the Korean Intellectual Property Office, the entire disclosure of which is incorporated herein by reference.

### BACKGROUND

### 1. Field

One or more aspects of embodiments of the present disclosure relate to a light-emitting device including an organic compound, an electronic apparatus including the light-emitting device, an electronic device including the light-emitting device, and the organic compound.

### 2. Description of the Related Art

From among light-emitting devices, self-emissive devices have relatively wide viewing angles, high contrast ratios, short response times, and excellent or suitable characteristics in terms of luminance, driving voltage, and response speed.

In a light-emitting device, a first electrode is on a substrate, and a hole transport region, an emission layer, an electron transport region, and a second electrode are sequentially on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. The excitons may transition (e.g., relax) from an excited state to a ground state, thereby generating light.

### SUMMARY

One or more aspects of embodiments of the present disclosure are directed toward a light-emitting device including an organic compound, an electronic device including the light-emitting device, an electronic apparatus including the light-emitting device, and the organic compound.

However, it should be noted that the preceding are merely examples, and the scope of the disclosure is not limited to the herein-mentioned aspects. Additional aspects will be set forth in part in the description which follows and, in part, will be apparent from the description, or may be learned by practice of the presented embodiments of the disclosure.

According to one or more embodiments, a light-emitting device includes
a first electrode,
a second electrode opposite to (e.g., facing) the first electrode, and
an interlayer between the first electrode and the second electrode and including an emission layer, wherein
the light-emitting device includes an organic compound represented by Formula 1:

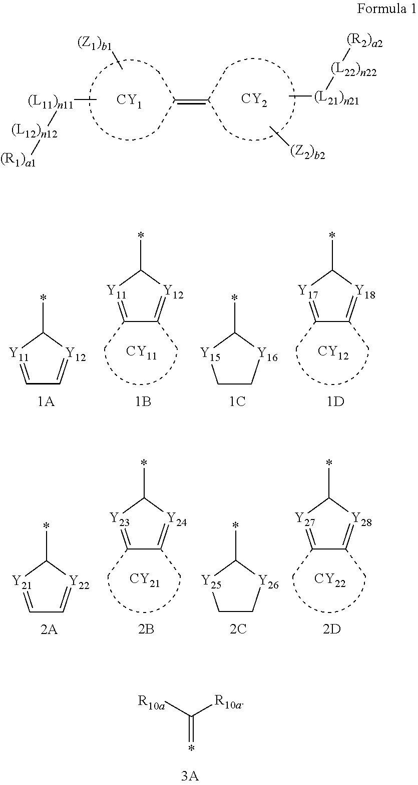

In Formulae 1, 1A to 1D, 2A to 2D, and 3A,
ring CY₁ may be represented by any one of Formulae 1A to 1D,
ring CY₂ may be represented by any one of Formulae 2A to 2D,
ring CY₁₁, ring CY₁₂, ring CY₂₁, and ring CY₂₂ may each independently be a
C₅-C₆₀ carbocyclic group or a C₁-C₆₀ heterocyclic group,
Y₁₁ may be C(R₁₁), Si(R₁₁), or N,
Y₁₂ may be C(R₁₂), Si(R₁₂), or N,
Y₁₃ may be C(R₁₃), Si(R₁₃), or N,
Y₁₄ may be C(R₁₄), Si(R₁₄), or N,
Y₁₅ may be C(R₁₅)(R₁₅′), Si(R₁₅)(R₁₅′), N(R₁₅), O, S, or Se,
Y₁₆ may be C(R₁₆)(R₁₆′), Si(R₁₆)(R₁₆′), N(R₁₆), O, S, or Se,
Y₁₇ may be C(R₁₇)(R₁₇′), Si(R₁₇)(R₁₇′), N(R₁₇), O, S, or Se,
Y₁₈ may be C(R₁₈)(R₁₈′), Si(R₁₈)(R₁₈′), N(R₁₈), O, S, or Se,
Y₂₁ may be C(R₂₁), Si(R₂₁), or N,
Y₂₂ may be C(R₂₂), Si(R₂₂), or N,
Y₂₃ may be C(R₂₃), Si(R₂₃), or N,
Y₂₄ may be C(R₂₄), Si(R₂₄), or N,
Y₂₅ may be C(R₂₅)(R₂₅′), Si(R₂₅)(R₂₅′), N(R₂₅), O, S, or Se,
Y₂₆ may be C(R₂₆)(R₂₆′), Si(R₂₆)(R₂₆′), N(R₂₆), O, S, or Se,
Y₂₇ may be C(R₂₇)(R₂₇′), Si(R₂₇)(R₂₇′), N(R₂₇), O, S, or Se,
Y₂₈ may be C(R₂₈)(R₂₈′), Si(R₂₈)(R₂₈′), N(R₂₈), O, S, or Se,
L₁₁, L₁₂, L₂₁, and L₂₂ may each independently be a single bond (e.g., a single covalent bond), *—C(R₃)(R₄)—*′, *—Si(R₃)(R₄)—*′, *—Ge(R₃)(R₄)—*′, *—S—*, *—Se—*′, *—O—*′, *—C(═O)—*′, *—S(═O)—*′, *—S(═O)₂—*′, *═C(R₃)—*′, *—C(R₃)═C(R₄)—*′, *—C(═S)—*′, *—C≡C—*′, a C₅-C₆₀ carbocyclic group unsubstituted or substituted with at least one R₁₀ₐ, or a C₁-C₆₀ heterocyclic group unsubstituted or substituted with at least one R₁₀ₐ,
n11, n12, n21, and n22 may each independently be an integer from 1 to 10,
R₁ to R₄, R₁₁ to R₁₈, R₁₅′ to R₁₈′, R₂₁ to R₂₈, R₂₅′ to R₂₈′, Z₁, and Z₂ may each independently be a group represented by Formula 3A, hydrogen, deuterium, —F, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group, a C₆-C₆₀ arylthio group unsubstituted or substituted with at least one R₁₀ₐ, a C₆-C₆₀ arylseleno group unsubstituted or substituted with at least one R₁₀ₐ, a C₇-C₆₀ arylalkyl group unsubstituted or substituted with at least one R₁₀ₐ, a C₂-C₆₀ heteroaryl alkyl group unsubstituted or substituted with at least one R₁₀ₐ, —Si(Q₁)(Q₂)(Q₃), —Ge(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more adjacent substituents selected from among R₁ to R₄, R₁₁ to R₁₈, R₁₅′ to R₁₈′, R₂₁ to R₂₈, R₂₅′ to R₂₈′, Z₁, and Z₂ may be optionally bonded to each other to form (provide) a C₅-C₃₀ carbocyclic group unsubstituted or substituted with at least one R₁₀ₐ, or a C₂-C₃₀ heterocyclic group unsubstituted or substituted with at least one R₁₀ₐ,
a1 may be an integer from 0 to 30,
a2 may be an integer from 0 to 30,
b1 may be an integer from 0 to 10,
b2 may be an integer from 0 to 10, and
* indicates a binding site to the double bond (e.g., a double covalent bond) of Formula 1,
the organic compound represented by Formula 1 may include at least one cyano group,
R₁₀ₐ may be
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

According to one or more embodiments, an electronic apparatus includes the light-emitting device.

According to one or more embodiments, an electronic device includes the light-emitting device.

According to one or more embodiments, provided is an organic compound represented by Formula 1.

### BRIEF DESCRIPTION OF THE DRAWINGS

The accompanying drawings are included to provide a further understanding of the preceding and other aspects, features, and advantages of one or more embodiments of the disclosure and are incorporated in and constitute a part of this specification. The drawings illustrate example embodiments that will be more apparent from the following description taken in conjunction with the accompanying drawings. In the drawings:

FIG. 1 is a schematic view of the structure of a light-emitting device according to one or more embodiments;

FIG. 2 is a schematic view of the structure of an electronic apparatus according to one or more embodiments;

FIG. 3 is a schematic view of the structure of an electronic apparatus according to another embodiment;

FIG. 4 is a schematic view of an electronic device including a light-emitting device according to one or more embodiments;

FIG. 5 is a schematic view of the exterior of a vehicle as an electronic device including a light-emitting device according to one or more embodiments; and

FIGS. 6A-6C are each a schematic view of the interior of a vehicle according to one or more embodiments.

### DETAILED DESCRIPTION

Reference will now be made in detail to embodiments, examples of which are illustrated in the accompanying drawings, wherein like reference numerals refer to like elements throughout the specification. In this regard, the present embodiments may have different forms and should not be construed as being limited to the descriptions set forth herein. It should be noted that in the following description, only portions useful for understanding an operation according to the disclosure are described, and descriptions of other portions are not included in order not to obscure the subject matter of the disclosure. Accordingly, the embodiments are merely described as follows, by referring to the figures, to explain aspects of embodiments of the present description in enough detail so that those skilled in the art may easily implement the technical spirit of the disclosure to which the disclosure belongs. As used herein, the term “and/or” includes any and all combinations of one or more of the associated listed items. Throughout the disclosure, the expressions “at least one of a, b or c,” “at least any of a, b, and c,” and “at least any selected from a group consisting of a, b, and c,” and/or the like indicate only a, only b, only c, both (e.g., simultaneously) a and b, both (e.g., simultaneously) a and c, both (e.g., simultaneously) b and c, all of a, b, and c, or variations thereof, (for example, abc, ab, bc, and cc).

Because the disclosure may have diverse modified embodiments, embodiments are illustrated in the drawings and are described in the detailed description. An effect and a characteristic of the disclosure, and a method of accomplishing these will be apparent when referring to embodiments described with reference to the drawings. The disclosure may, however, be embodied in many different forms and should not be construed as limited to the embodiments set forth herein.

Hereinafter, embodiments of the disclosure will be described in detail with reference to the accompanying drawings. The same or corresponding components will be denoted by the same reference numerals, and thus redundant description thereof will not be provided.

It will be understood that although the terms “first,” “second,” and/or the like may be used herein to describe one or more suitable components, these components should not be limited by these terms. These components are only used to distinguish one component from another. Therefore, a first component may refer to a second component within a range without departing from the scope disclosed herein.

An expression used in the singular such as “a,” “an” and “the” encompasses the expression of the plural, unless it has a clearly different meaning in the context.

It will be further understood that the terms “includes”, “include,” “including,” “having,” “have,” “has,” “comprise,” “comprises,” and/or “comprising,” as used herein specify the presence of stated features or elements, but do not preclude the presence or addition of one or more other features or elements.

In the following embodiments, when one or more suitable components such as layers, films, regions, plates, and/or the like are said to be “on” or “coupled” to another component, this may include not only a case in which other components are “immediately on” or “coupled” to the layers, films, regions, or plates, but also a case in which other components may be placed therebetween. Sizes of elements in the drawings may be exaggerated for convenience of explanation. In embodiments, because sizes and thicknesses of components in the drawings are arbitrarily illustrated for convenience of explanation, the following embodiments are not limited thereto.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

Spatially relative terms such as “below”, “lower”, “above”, “on top”, “on the top”, “under”, “on”, and/or the like may be used for descriptive purposes, thereby describing a relationship between one element or feature and another element(s) or feature(s) as shown in the drawings. Spatially relative terms are intended to include other directions in use, in operation, and/or in manufacturing, in addition to the direction depicted in the drawings. For example, when a device shown in the drawing is turned upside down, elements depicted as being positioned “under” other elements or features are positioned in a direction “on” the other elements or features. Therefore, in one or more embodiments, the term “under” may include both directions of on and under. In some embodiments, the device may face in other directions (for example, rotated 90 degrees or in other directions) and thus the spatially relative terms used herein are interpreted according thereto.

The term “interlayer” as used herein refers to a single layer and/or all of multiple layers between the first electrode and the second electrode of the light-emitting device.

Unless otherwise defined, all terms (including chemical, technical and scientific terms) used herein have a same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

When a certain embodiment may be implemented differently, a specific process order may be performed differently from the described order. For example, two consecutively described processes may be performed substantially at the same time or performed in an order opposite to the described order.

In this context, “consisting essentially of” indicates that any additional components will not materially affect the chemical, physical, optical or electrical properties of the semiconductor film.

Hereinafter, one or more embodiments of the disclosure are described in more detail with reference to the attached drawings.

### Light-Emitting Device

According to one or more embodiments, the light-emitting device may include: a first electrode; a second electrode opposite to (e.g., facing) the first electrode; and an interlayer between the first electrode and the second electrode and including an emission layer, and
the light-emitting device may include an organic compound represented by Formula 1 (that is, a first compound):

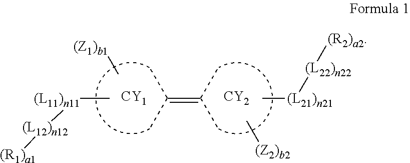

For a description of Formula 1, reference may be made to the present specification.

In one or more embodiments,
the first electrode may be an anode,
the second electrode may be a cathode,
the interlayer may further include a hole transport region located between the first electrode and the emission layer and an electron transport region located between the emission layer and the second electrode,
the hole transport region may include at least one selected from among a hole injection layer, a hole transport layer, a buffer layer, an emission auxiliary layer, and an electron-blocking layer, and
the electron transport region may include at least one selected from among a hole-blocking layer, an electron transport layer, and an electron injection layer.

In one or more embodiments, the organic compound may be included between the first electrode and the second electrode of the light-emitting device. For example, the hole transport region may include the organic compound represented by Formula 1. For example, the hole injection layer may include the organic compound represented by Formula 1. For example, the hole transport layer may include the organic compound represented by Formula 1. For example, the organic compound represented by Formula 1 may be a charge-generating material, for example, a p-dopant.

According to one or more embodiments, the emission layer in the interlayer of the light-emitting device may include a dopant and a host, and the host and/or the dopant may include the organic compound represented by Formula 1. For example, the organic compound may act as a host and/or a dopant. The emission layer may emit red light, green light, blue light, and/or white light. For example, the emission layer may emit blue light. The blue light may have a maximum emission wavelength in a range of, for example, about 400 nanometer (nm) to about 490 nm. The blue light may emit light having a wavelength of, for example, about 430 nm to about 480 nm. The red light may have a maximum emission wavelength in a range of about 600 nm to about 750 nm. The green light may have a maximum emission wavelength in the range of about 490 nm to about 600 nm.

In one or more embodiments, the light-emitting device may further include a capping layer outside (e.g., and on) the first electrode or outside (e.g., and on) the second electrode.

In one or more embodiments, the light-emitting device may further include at least one of (e.g., selected from among) a first capping layer outside (e.g., and on) the first electrode and a second capping layer outside (e.g., and on) the second electrode, wherein the organic compound represented by Formula 1 may be included in at least one of (e.g., selected from among) the first capping layer and the second capping layer. More details on the first capping layer and/or the second capping layer may be referred to the descriptions provided herein.

According to one or more embodiments, the light-emitting device may further include a first capping layer outside (e.g., and on) the first electrode. For example, the first capping layer may include the organic compound represented by Formula 1.

According to another embodiment, the light-emitting device may further include a second capping layer outside (e.g., and on) the second electrode. For example, the second capping layer may include the organic compound represented by Formula 1.

According to another embodiment, the light-emitting device may further include a first capping layer outside (e.g., and on) the first electrode and a second capping layer outside (e.g., and on) the second electrode. For example, at least one of (e.g., selected from among) the first capping layer and the second capping layer may include the organic compound represented by Formula 1.

The expression “(interlayer and/or capping layer) includes an organic compound” as used herein may be understood as “(interlayer and/or capping layer) may include one (e.g., kind of) organic compound represented by Formula 1 or two or more different (e.g., kinds of) organic compound, each represented by Formula 1.”

For example, the interlayer and/or the capping layer may include only compound 1 as the organic compound. In this regard, compound 1 may be present in the hole transport region or emission layer of the light-emitting device. In one or more embodiments, the interlayer may include, as the organic compound, compound 1 and compound 2. In this regard, compound 1 and compound 2 may be present in the same layer (e.g., compound 1 and compound 2 may both (e.g., simultaneously) be present in the hole transport region) or in different layers (e.g., compound 1 may be present in the emission layer and compound 2 may be present in the hole transport region).

In one or more embodiments,
the emission layer in the light-emitting device may include
a second compound including a group represented by Formula 20, a third compound including at least one π electron-deficient nitrogen-containing C₁-C₆₀ cyclic group, a fourth compound including a transition metal, or any combination thereof, wherein
the second compound, the third compound, and the fourth compound may be different from each other:

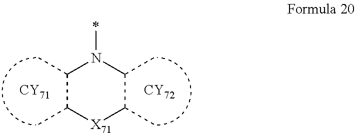

wherein ring CY₇₁ and ring CY₇₂ in Formula 20 may each independently be a π electron-rich C₃-C₆₀ cyclic group or a pyridine group,
X₇₁ in Formula 20 may be a single bond (e.g., a single covalent bond), or a linking group including O, S, N, B, C, Si, or any combination thereof,
* in Formula 20 is a bonding site to a neighboring atom in Formula 20, and
the compounds CBP and mCBP may be excluded from (options for) the second compound:

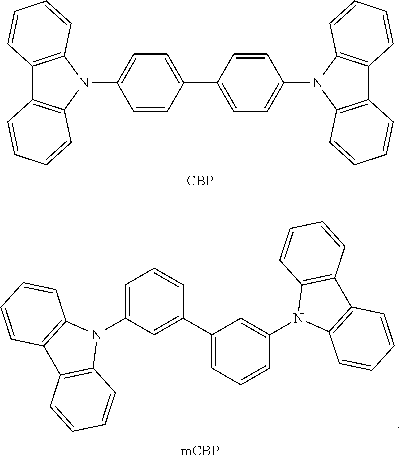 [SMILES: c1ccc2c(c1)c1ccccc1n2-c1ccc(-c2ccc(-n3c4ccccc4c4ccccc43)cc2)cc1 ; "CBP" ; c1cc(-c2cccc(-n3c4ccccc4c4ccccc43)c2)cc(-n2c3ccccc3c3ccccc32)c1]

According to one or more embodiments, the first compound represented by Formula 1 may be included in the hole injection layer, and the second compound, the third compound, the fourth compound, or any combination thereof may be included in the emission layer.

According to one or more embodiments, the emission layer may include at least one of (e.g., selected from among) the second compound and the third compound.

In another embodiment, the emission layer may include the second compound and the fourth compound.

In another embodiment, the emission layer may include the third compound and the fourth compound.

In another embodiment, the emission layer may include each (e.g., all) of the second compound to the fourth compound.

In one or more embodiments, the second compound may include a compound represented by Formula 20-1, a compound represented by Formula 20-2, a compound represented by Formula 20-3, a compound represented by Formula 20-4, a compound represented by Formula 20-5, or any combination thereof:

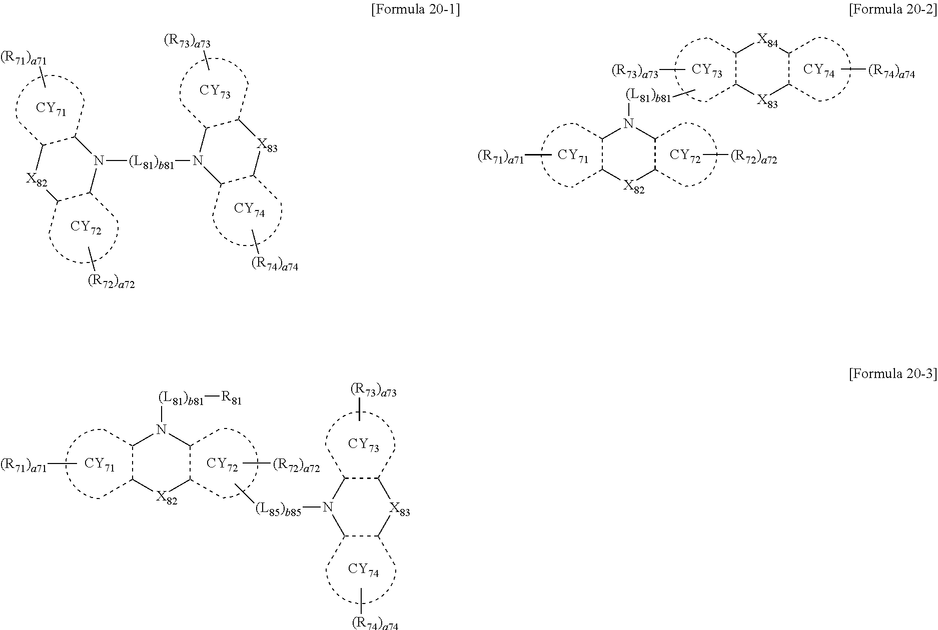

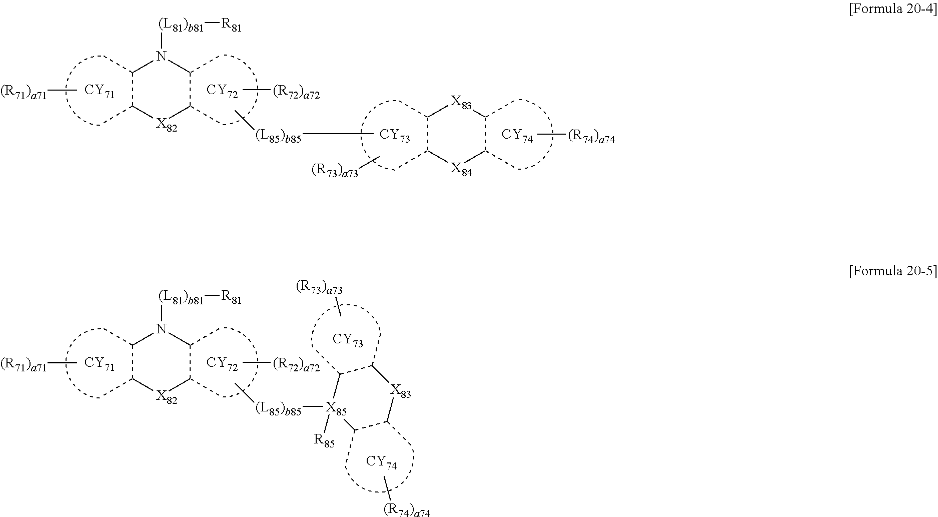

wherein, in Formulae 20-1 to 20-5,
ring CY₇₁ to ring CY₇₄ may each independently be a π electron-rich C₃-C₆₀ cyclic group or a pyridine group,
X₈₂ may be a single bond (e.g., a single covalent bond), O, S, N-[(L₈₂)_{b82}-R₈₂], C(R₈₂ₐ)(R_{82b}), or Si(R₈₂ₐ)(R_{82b}),
X₈₃ may be a single bond (e.g., a single covalent bond), O, S, N-[(L₈₃)_{b83}-R₈₃], C(R₈₃ₐ)(R_{83b}), or Si(R₈₃ₐ)(R_{83b}),
X₈₄ may be O, S, N-[(L₈₄)_{b84}-R₈₄], C(R₈₄ₐ)(R_{84b}), or Si(R₈₄ₐ)(R_{84b}),
X₈₅ may be C or Si,
L₈₁ to L₈₅ may each independently be a single bond (e.g., a single covalent bond), *—C(Q₄)(Q₅)-*′, *—Si(Q₄)(Q₅)-*′, a π electron-rich C₃-C₆₀ cyclic group unsubstituted or substituted with at least one R₁₀ₐ, or a pyridine group unsubstituted or substituted with at least one R₁₀ₐ, wherein Q₄ and Q₅ may each be understood by referring to the description of Q₁ provided herein,
b81 to b85 may each independently be an integer from 1 to 5,
R₇₁ to R₇₄, R₈₁ to R₈₅, R₈₂ₐ, R_{82b}, R₈₃ₐ, R_{83b}, R₈₄ₐ, and R_{84b} may each be as described herein,
a71 to a74 may each independently be an integer from 0 to 20, and
R₁₀ₐ may be as described herein.

The third compound may include a pyridine group, a pyrimidine group, a pyrazine group, a pyridazine group, a triazine group, or any combination thereof.

In one or more embodiments, the third compound may include a compound represented by Formula 30:

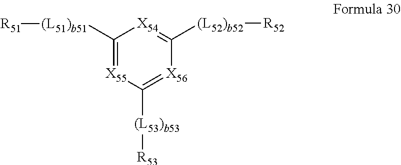

wherein, in Formula 30,
L₅₁ to L₅₃ may each independently be a single bond (e.g., a single covalent bond), a C₃-C₆₀ carbocyclic group unsubstituted or substituted with at least one R₁₀ₐ, or a C₁-C₆₀ heterocyclic group unsubstituted or substituted with at least one R₁₀ₐ,
b51 to b53 may each independently be an integer from 1 to 5,
X₅₄ may be N or C(R₅₄), X₅₅ may be N or C(R₅₅), X₅₆ may be N or C(R₅₆), and at least one of X₅₄ to X₅₆ may be N,
R₅₁ to R₅₆ are respectively as those described in the present specification, and

R₁₀ₐ and Q₁ to Q₃ are each as described herein.

In one or more embodiments, the fourth compound may include a group represented by Formula 401:

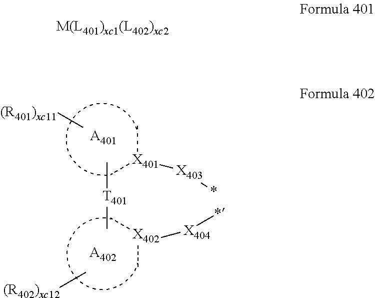

wherein, in Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, when xc1 is 2 or more, two or more of L₄₀₁ may be substantially identical to or different from each other,
L₄₀₂ may be an organic ligand, and xc2 may be 0, 1, 2, 3, or 4, wherein, when xc2 is 2 or more, two or more of L₄₀₂ may be substantially identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond (e.g., a single covalent bond), *—O—*′, *—S—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)=*′, or *═C═*′,
X₄₀₃ and X₄₀₄ may each independently be a chemical bond (e.g., a covalent bond), O, S, N(Q₄₁₃), B(Q₄₁₃), P(Q₄₁₃), C(Q₄₁₃)(Q₄₁₄), or Si(Q₄₁₃)(Q₄₁₄),
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
xc11 and xc12 may each independently be an integer from 0 to 10,
Q₄₁₁ to Q₄₁₄ and Q₄₀₁ to Q₄₀₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof,
* and *′ in Formula 402 each indicate a binding site to M in Formula 401, and
R₁₀ₐ may be as described herein.

### Description of Formulae 20, 20-1 to 20-5, and 30

In one or more embodiments, a group represented by

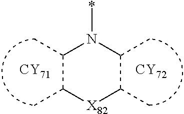

in Formulae 20-1 and 20-2 may be a group represented by any one of Formulae CY71-1(1) to CY71-1(8), and/or
a group represented by

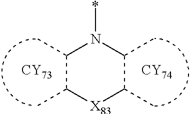

in Formulae 20-1 and 20-3 may be a group represented by any one of Formulae CY71-2(1) to CY71-2(8), and/or
a group represented by

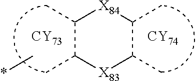

in Formulae 20-2 and 20-4 may be a group represented by any one of Formulae CY71-3(1) to CY71-3(32), and/or
a group represented by

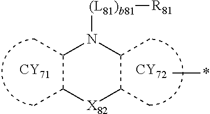

in Formulae 20-3 to 20-5 may be a group represented by any one of Formulae CY71-4(1) to CY71-4(32), and/or
a group represented by

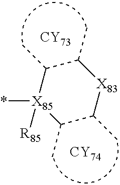

in Formula 20-5 may be a group represented by any one of Formulae CY71-5(1) to CY71-5(8):

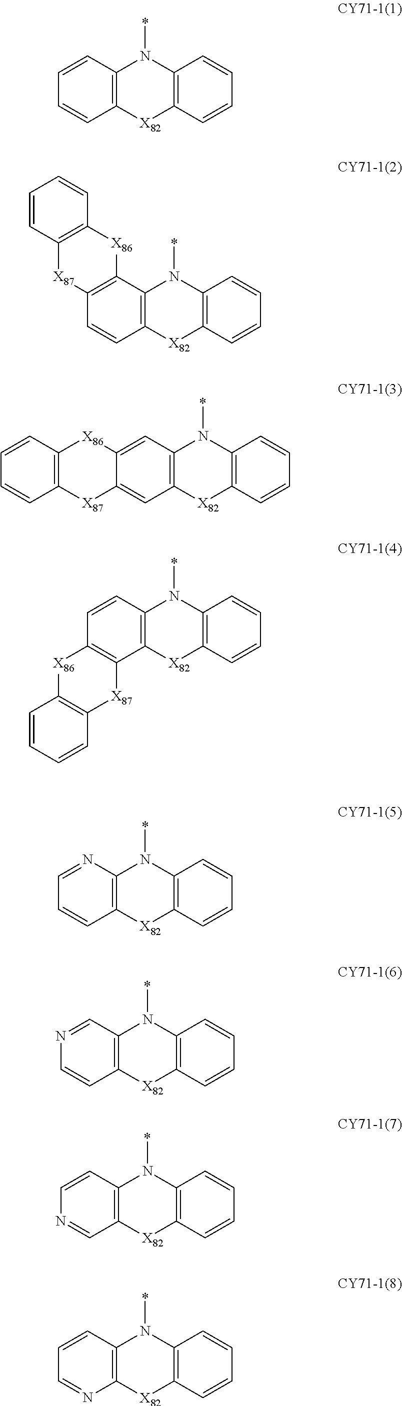

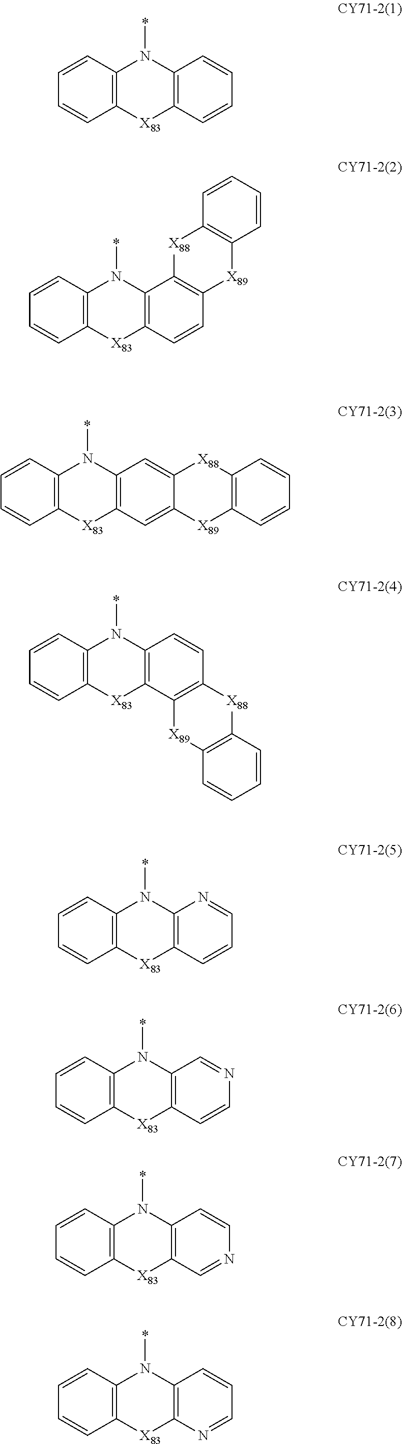

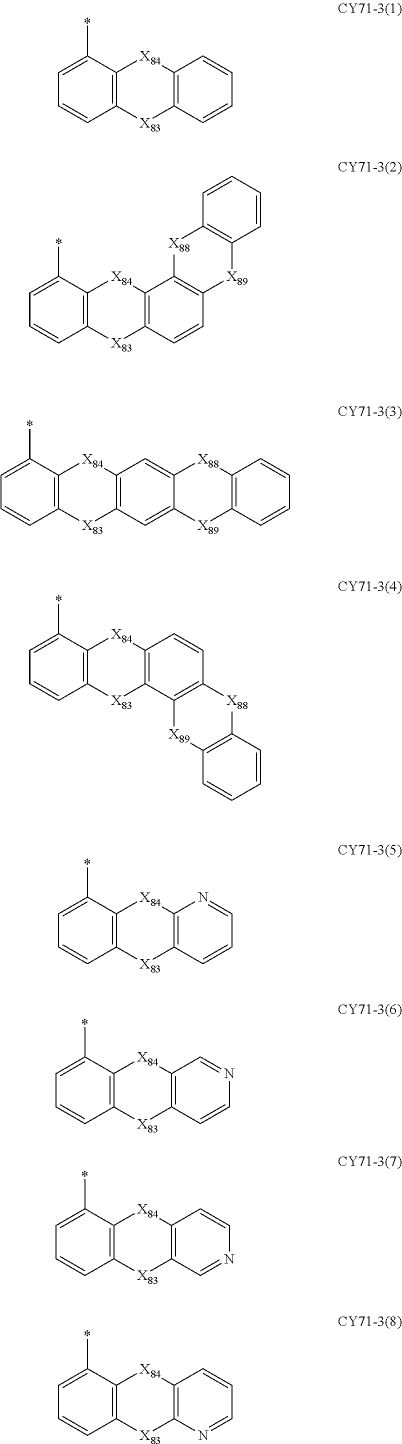

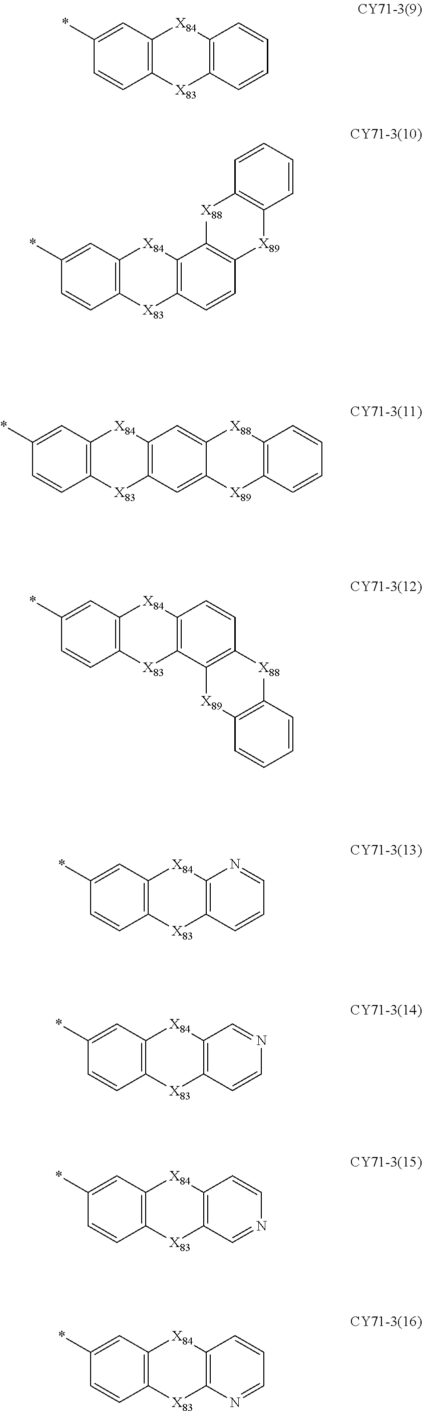

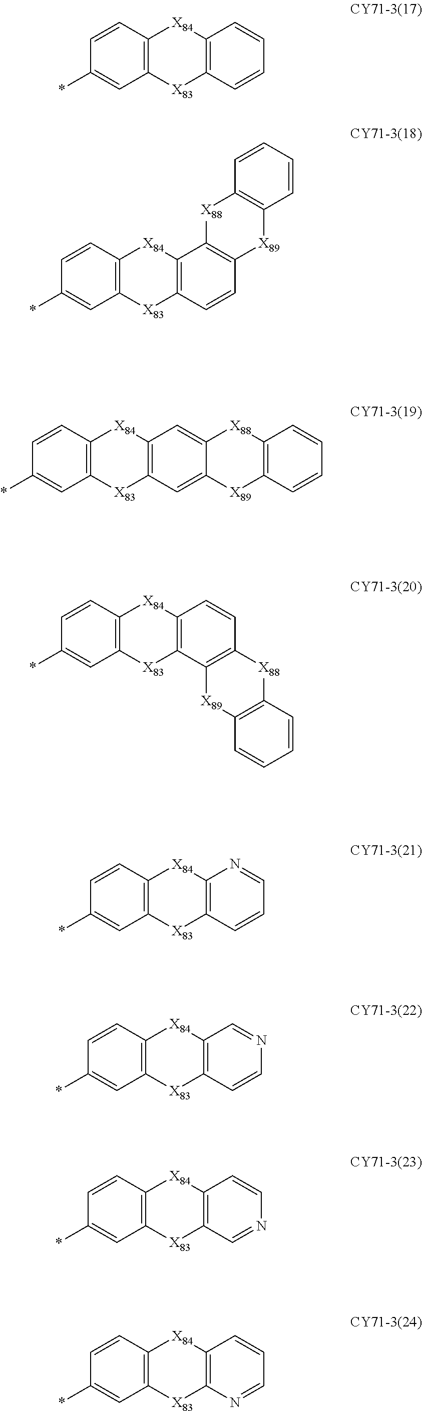

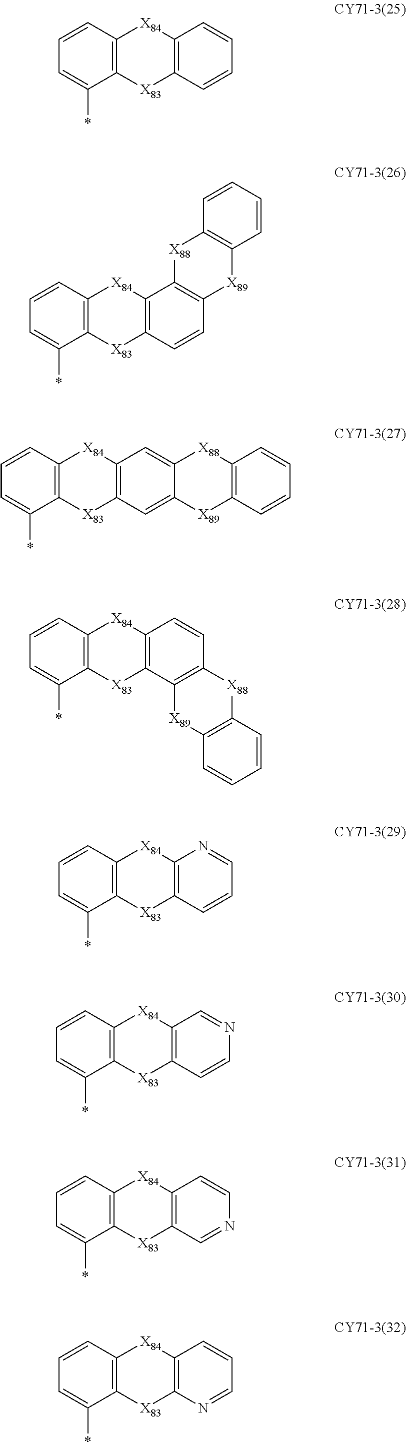

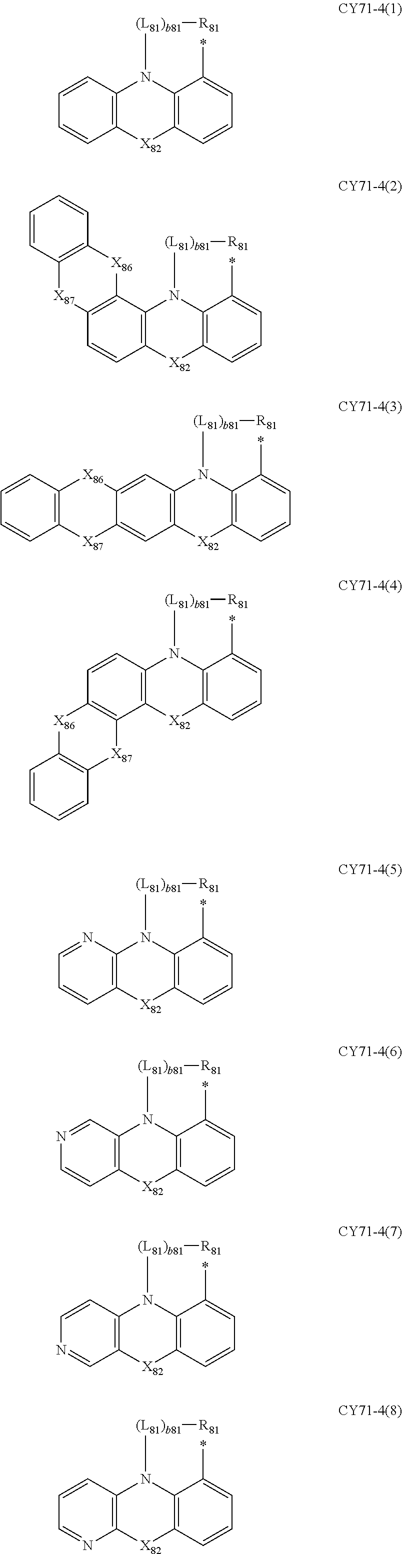

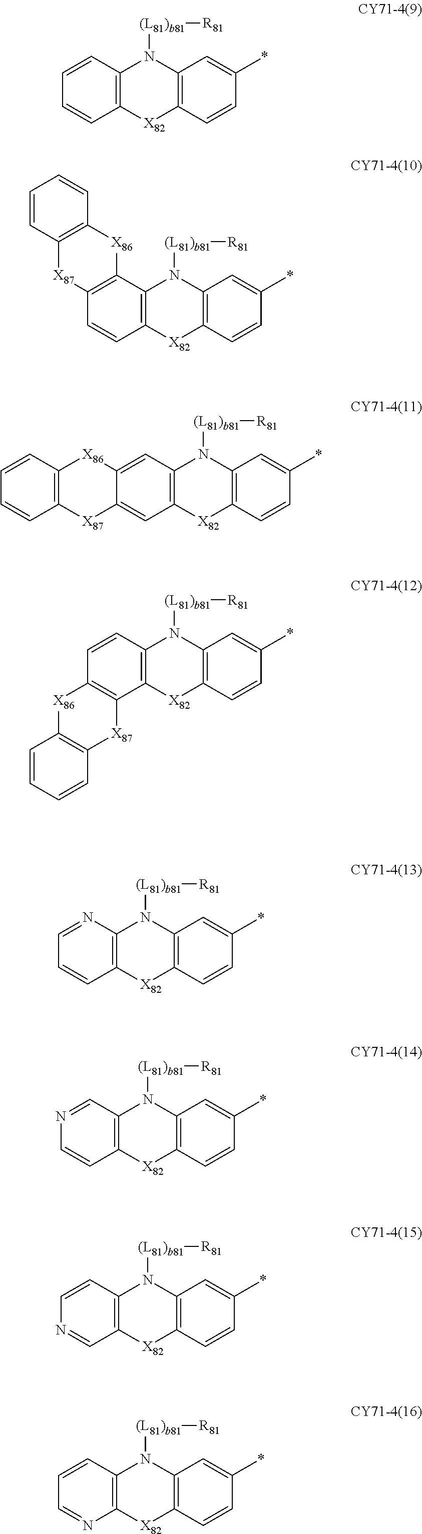

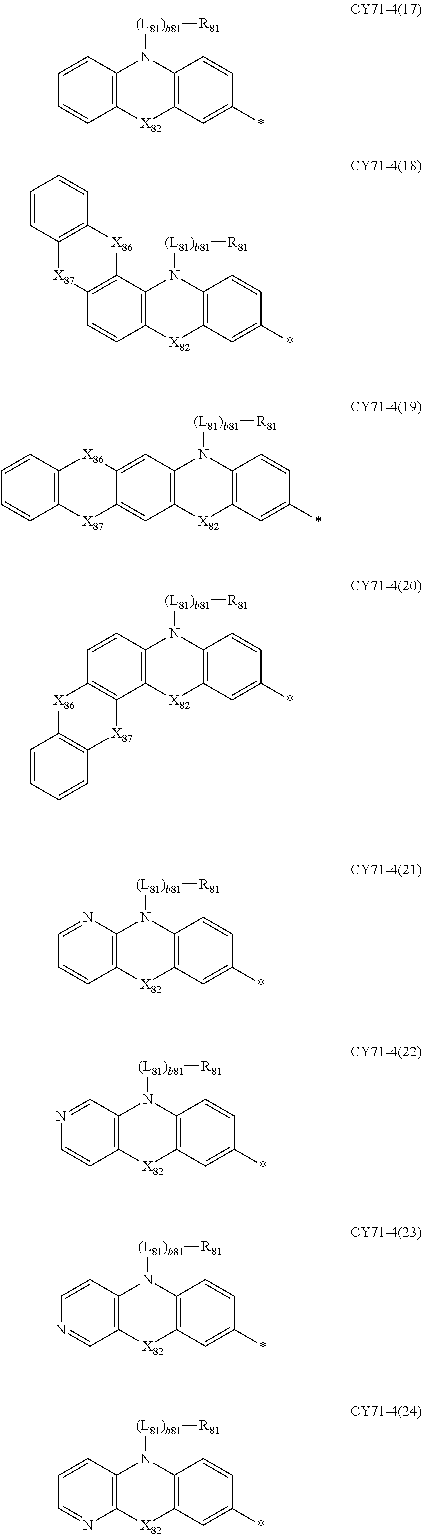

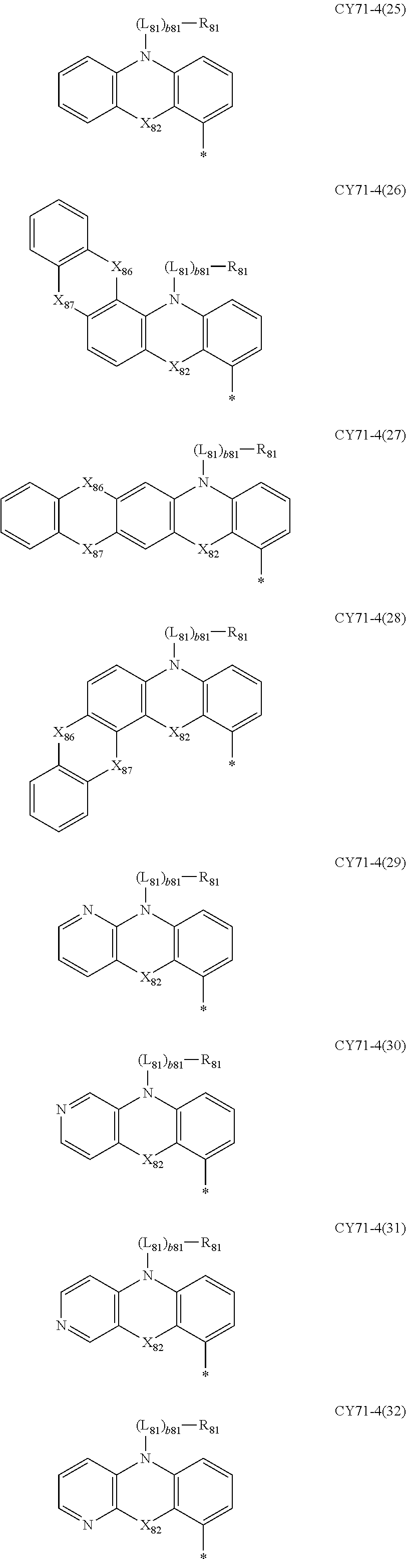

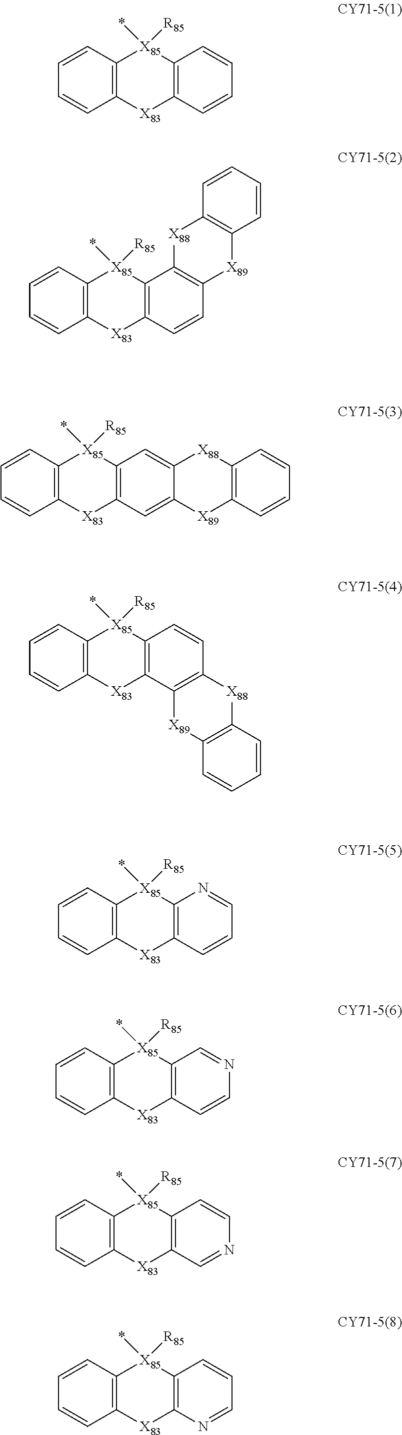

wherein, in Formulae CY71-1(1) to CY71-1(8), CY71-2(1) to CY71-2(8), CY71-3(1) to CY71-3(32), CY71-4(1) to CY71-4(32), and CY71-5(1) to CY71-5(8),
X₈₁ to X₈₅, L₈₁, b81, R₈₁, and R₈₅ may each be as described herein,
X₈₆ may be a single bond (e.g., a single covalent bond), O, S, N(R₈₆), B(R₈₆), C(R₈₆ₐ)(R_{86b}), or Si(R₈₆ₐ)(R_{86b}),
X₈₇ may be a single bond (e.g., a single covalent bond), O, S, N(R₈₇), B(R₈₇), C(R₈₇ₐ)(R_{87b}), or Si(R₈₇ₐ)(R_{87b}),
each of X₈₆ and X₈₇ in Formulae CY71-1(1) to CY71-1(8) and CY71-4(1) to CY71-4(32) may not be a single bond (e.g., a single covalent bond) at the same time (e.g., simultaneously),
X₈₈ may be a single bond (e.g., a single covalent bond), O, S, N(R₈₈), B(R₈₈), C(R₈₈ₐ)(R_{88b}), or Si(R₈₈ₐ)(R_{88b}),
X₈₉ may be a single bond (e.g., a single covalent bond), O, S, N(R₈₉), B(R₈₉), C(R₈₉ₐ)(R_{89b}), or Si(R₈₉ₐ)(R_{89b}),
each of X₈₈ and X₈₉ in Formulae CY71-2(1) to CY71-2(8), CY71-3(1) to CY71-3(32), and CY71-5(1) to CY71-5(8) may not be a single bond (e.g., a single covalent bond) at the same time (e.g., simultaneously), and
R₈₆ to R₈₉, R₈₆ₐ, R_{86b}, R₈₇ₐ, R_{87b}, R₈₈ₐ, R_{88b}, R₈₉ₐ, and R_{89b} may each be as described in connection with R₈₁.
b51 to b53 in Formula 30 indicate numbers of L₅₁ to L₅₃, respectively, and may each be an integer from 1 to 5. When b51 is 2 or more, two or more of L₅₁ may be substantially identical to or different from each other, when b52 is 2 or more, two or more of L₅₂ may be substantially identical to or different from each other, and when b53 is 2 or more, two or more of L₅₃ may be substantially identical to or different from each other. In one or more embodiments, b51 to b53 may each independently be 1 or 2.
L₅₁ to L₅₃ in Formula 30 may each independently be:
a single bond (e.g., a single covalent bond); or
a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a dibenzooxacilline group, a dibenzothiacilline group, a dibenzodihydroazacilline group, a dibenzodihydrodicilline group, a dibenzodihydrocilline group, a dibenzodioxane group, a dibenzooxathiene group, a dibenzooxazine group, a dibenzopyran group, a dibenzodithiine group, a dibenzothiazine group, a dibenzothiopyran group, a dibenzocyclohexadiene group, a dibenzodihydropyridine group, or a dibenzodihydropyrazine group, each unsubstituted or substituted with deuterium, —F, —Cl, —Br, —I, a hydroxyl group, a cyano group, a nitro group, a C₁-C₂₀ alkyl group, a C₁-C₂₀ alkoxy group, a phenyl group, a naphthyl group, a pyridinyl group, a pyrimidinyl group, a triazinyl group, a fluorenyl group, a dimethylfluorenyl group, a diphenylfluorenyl group, a carbazolyl group, a phenylcarbazolyl group, a dibenzofuranyl group, a dibenzothiophenyl group, a dibenzosilolyl group, a dimethyldibenzosilolyl group, a diphenyldibenzosilolyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof, and
Q₃₁ to Q₃₃ may each independently be hydrogen, deuterium, a C₁-C₂₀ alkyl group, a C₁-C₂₀ alkoxy group, a phenyl group, a biphenyl group, a terphenyl group, a pyridinyl group, a pyrimidinyl group, a pyridazinyl group, a pyrazinyl group, or a triazinyl group.

In one or more embodiments, in Formula 30, a bond between L₅₁ and R₅₁, a bond between L₅₂ and R₅₂, a bond between L₅₃ and R₅₃, a bond between two or more L₅₁(s), a bond between two or more L₅₂(s), a bond between two or more L₅₃(s), a bond between L₅₁ and carbon between X₅₄ and X₅₅ in Formula 30, a bond between L₅₂ and carbon between X₅₄ and X₅₆ in Formula 30, and a bond between L₅₃ and carbon between X₅₅ and X₅₆ in Formula 30 may each be a “carbon-carbon single bond (e.g., a single covalent bond)”.

In Formula 30, X₅₄ may be N or C(R₅₄), X₅₅ may be N or C(R₅₅), X₅₆ may be N or C(R₅₆), and at least one of X₅₄ to X₅₆ may be N. R₅₄ to R₅₆ may each be as described herein. In one or more embodiments, two or three among X₅₄ to X₅₆ may each be N.

R₄₁ to R₄₅, R₅₁ to R₅₆, R₇₁ to R₇₄, R₈₁ to R₈₅, R₈₂ₐ, R_{82b}, R₈₃ₐ, R_{83b}, R₈₄ₐ, and R_{84b} in the present specification may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂). Q₁ to Q₃ may each be as described herein.

For example, i) R₄₁ to R₄₅, R₅₁ to R₅₆, R₇₁ to R₇₄, R₈₁ to R₈₅, R₈₂ₐ, R_{82b}, R₈₃ₐ, R_{83b}, R₈₄ₐ, and R_{84b} in Formulae 20-1 to 20-5 and 30, and ii) R₁₀ₐ may each independently be:
hydrogen, deuterium, —F, —Cl, —Br, —I, a hydroxyl group, a cyano group, a nitro group, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, or a group represented by Formula 91, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or
—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
Q₁ to Q₃ and Q₃₁ to Q₃₃ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂, or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with deuterium, a C₁-C₁₀ alkyl group, a phenyl group, a biphenyl group, a pyridinyl group, a pyrimidinyl group, a pyridazinyl group, a pyrazinyl group, a triazinyl group, or any combination thereof.

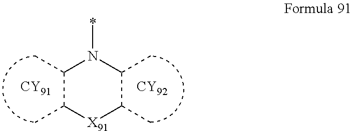

wherein, in Formula 91,
ring CY₉₁ and ring CY₉₂ may each independently be a C₅-C₃₀ carbocyclic group unsubstituted or substituted with at least one R₁₀ₐ or a C₁-C₃₀ heterocyclic group unsubstituted or substituted with at least one R₁₀ₐ,
X₉₁ may be a single bond (e.g., a single covalent bond), O, S, N(R₉₁), B(R₉₁), C(R₉₁ₐ)(R_{91b}), or Si(R₉₁ₐ)(R_{91b}),
R₉₁, R₉₁ₐ, and R_{91b} may be as described in connection with R₈₂, R₈₂ₐ, and R_{82b}, respectively,
R₁₀ₐ may be as described herein, and
* indicates a binding site to a neighboring atom.

For example, in Formula 91,
ring CY₉₁ and ring CY₉₂ may each independently be a benzene group, a pyridine group, a pyrimidine group, a pyrazine group, a pyridazine group, or a triazine group, each unsubstituted or substituted with at least one R₁₀ₐ, and
R₉₁, R₉₁ₐ, and R_{91b} may each independently be:
hydrogen or a C₁-C₁₀ alkyl group; or
a phenyl group, a pyridinyl group, a pyrimidinyl group, a pyridazinyl group, a pyrazinyl group, or a triazinyl group, each unsubstituted or substituted with deuterium, a C₁-C₁₀ alkyl group, a phenyl group, a biphenyl group, a pyridinyl group, a pyrimidinyl group, a pyridazinyl group, a pyrazinyl group, a triazinyl group, or any combination thereof.

In one or more embodiments, i) in Formulae 20-1 to 20-5, 30, and 40, R₄₁ to R₄₅, R₅₁ to R₅₆, R₇₁ to R₇₄, R₈₁ to R₈₅, R₈₂ₐ, R_{82b}, R₈₃ₐ, R_{83b}, R₈₄ₐ and R_{84b}, and ii) R₁₀ₐ may each independently be hydrogen, deuterium, —F, a cyano group, a nitro group, —CH₃, —CD₃, —CD₂H, —CDH₂, —CF₃, —CF₂H, —CFH₂, a group represented by one of Formulae 9-1 to 9-67, a group represented by one of Formulae 10-1 to 10-154 and 10-201 to 10-368, —C(Q₁)(Q₂)(Q₃), —Si(Q₁)(Q₂)(Q₃), or —P(═O)(Q₁)(Q₂)(Q₁ to Q₃ are as described herein):

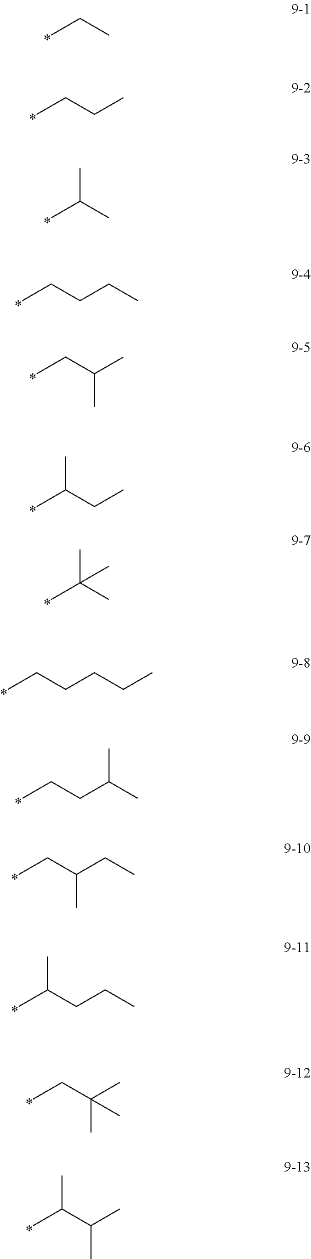

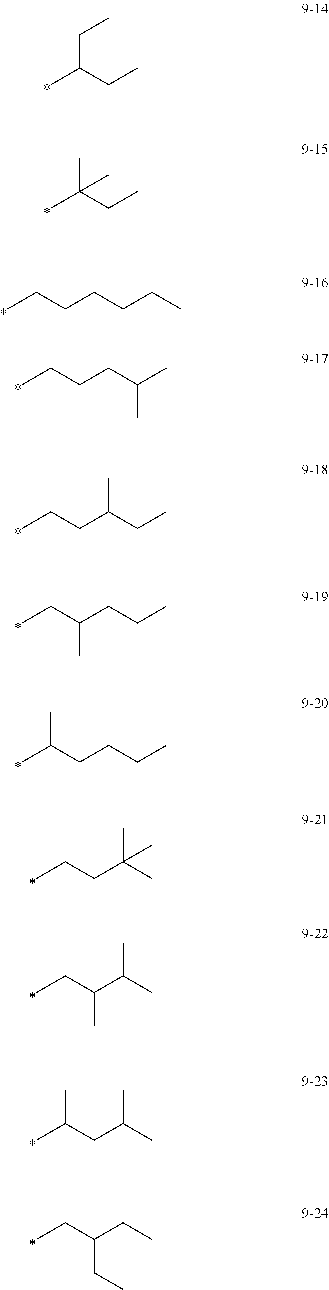

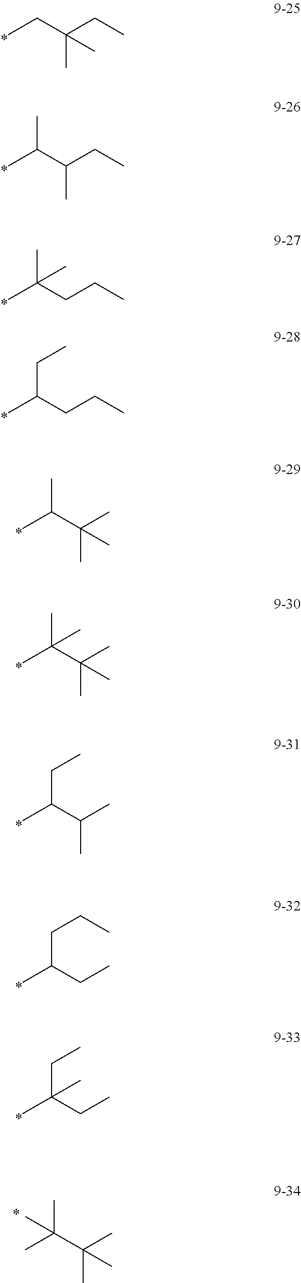

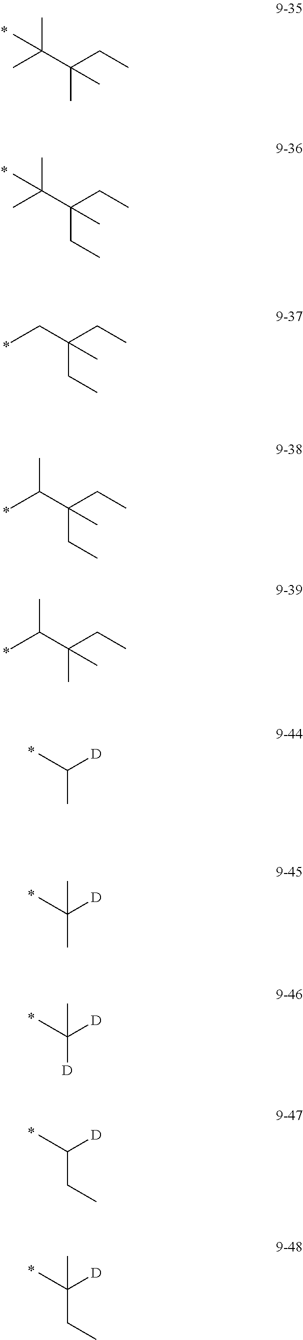

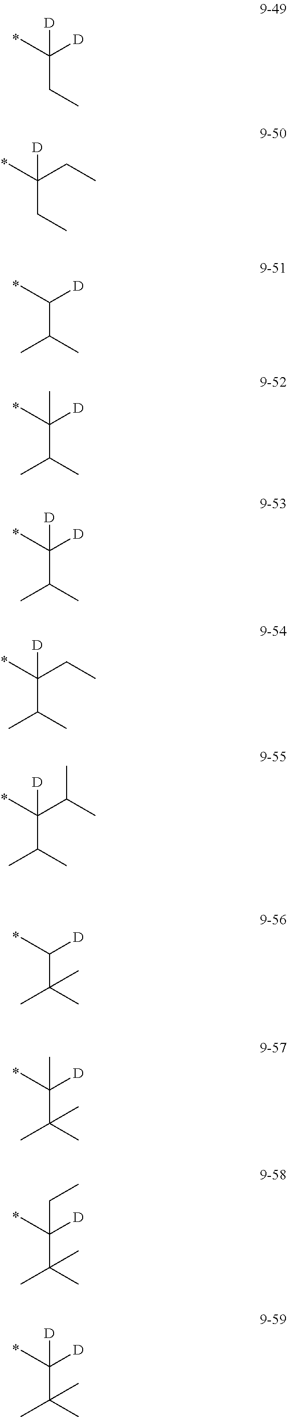

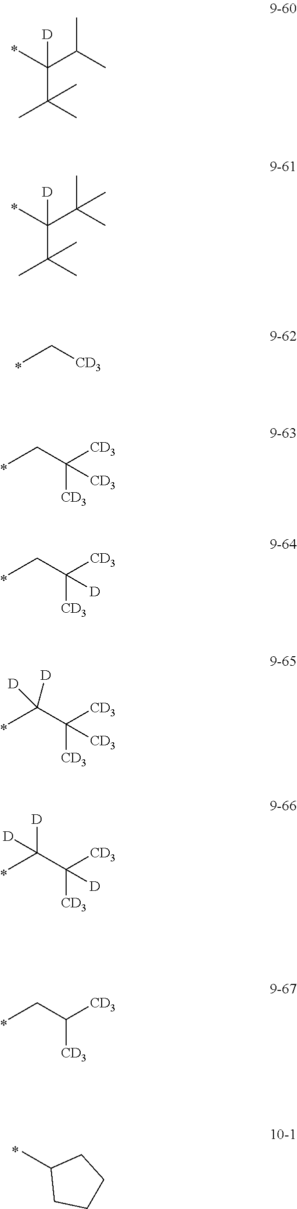

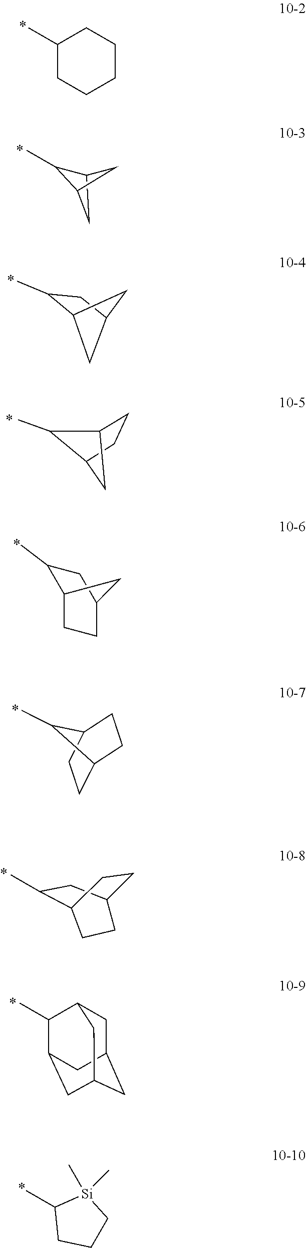

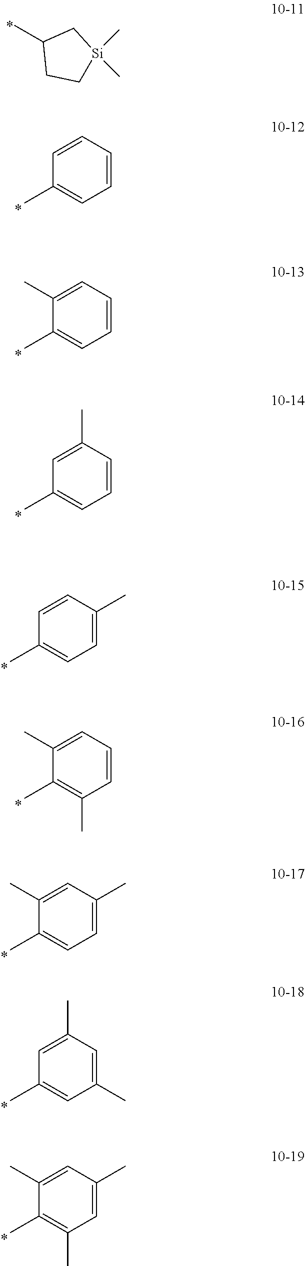

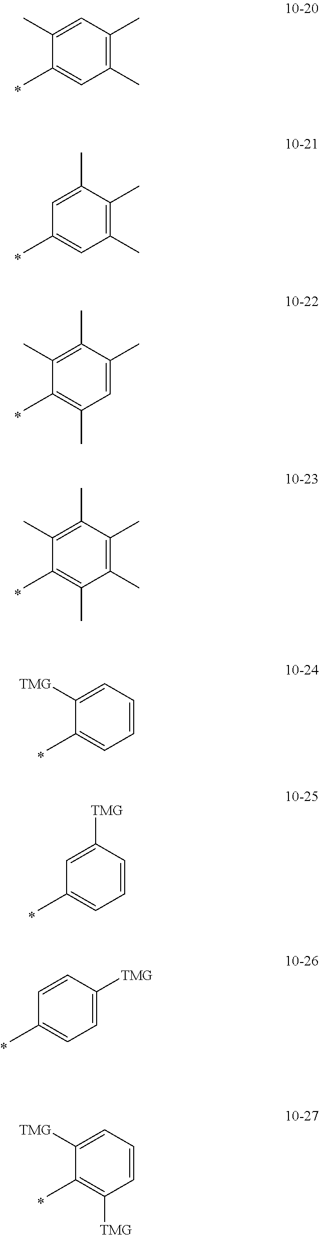

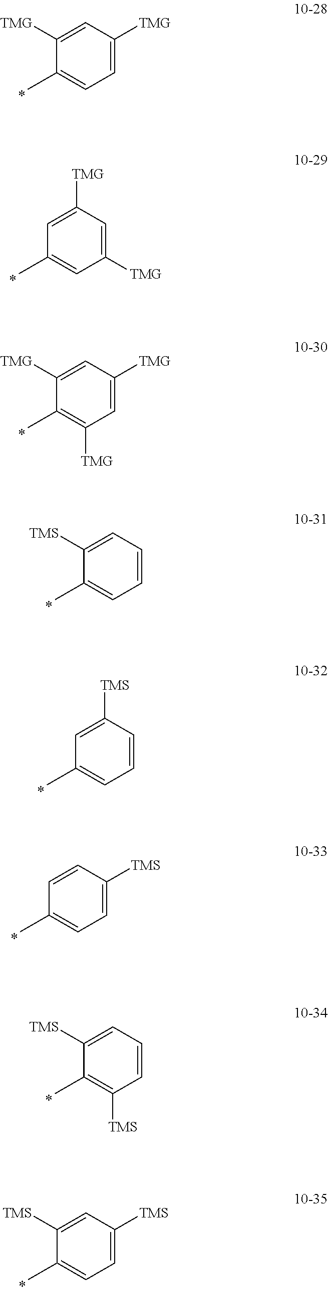

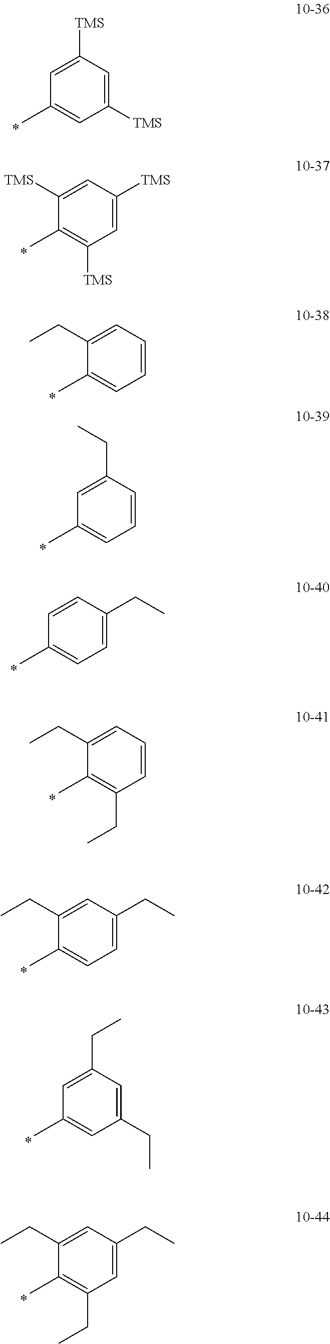

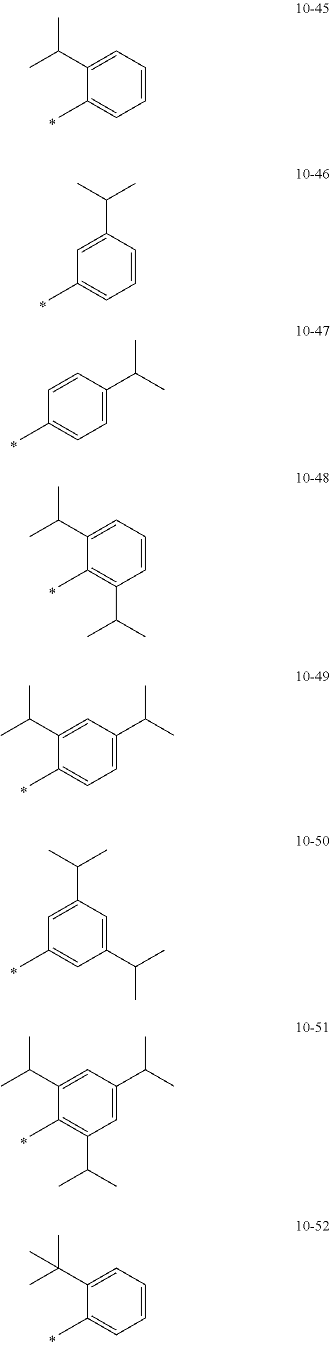

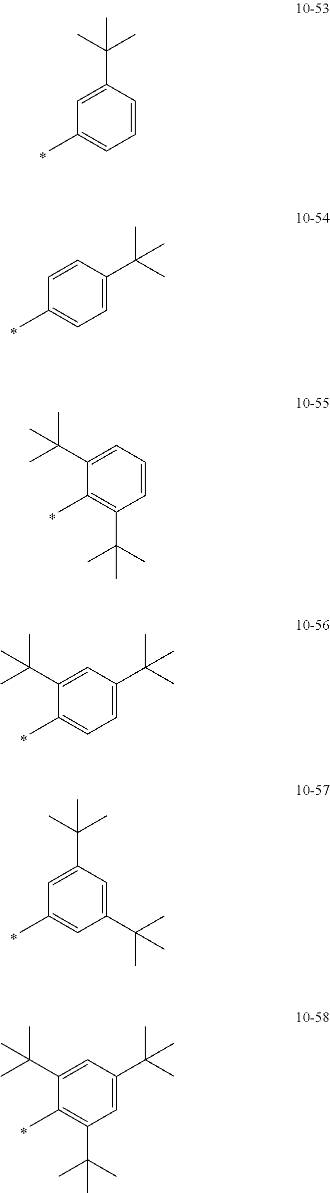

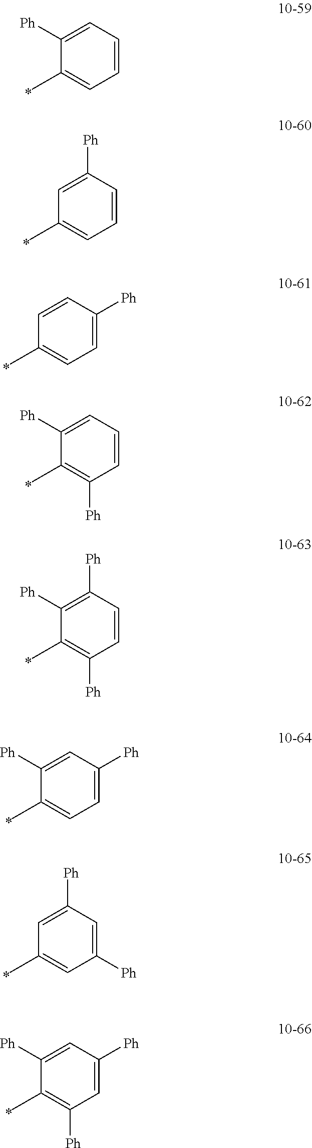

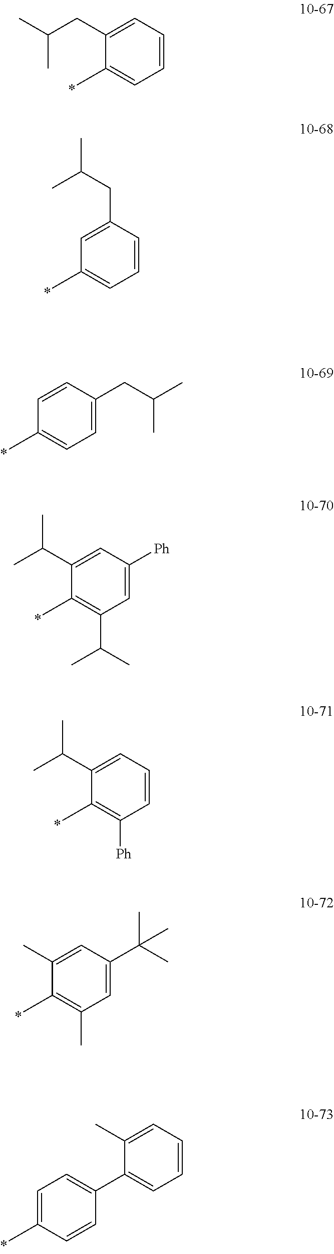

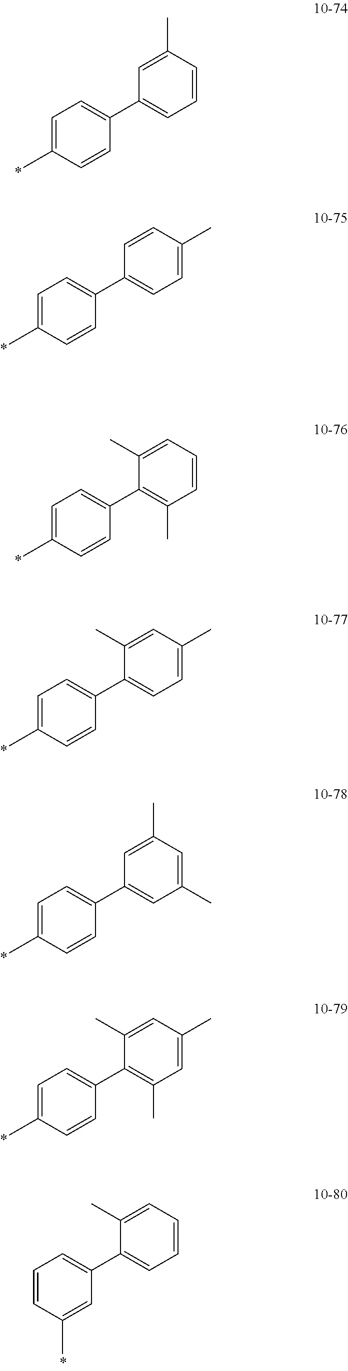

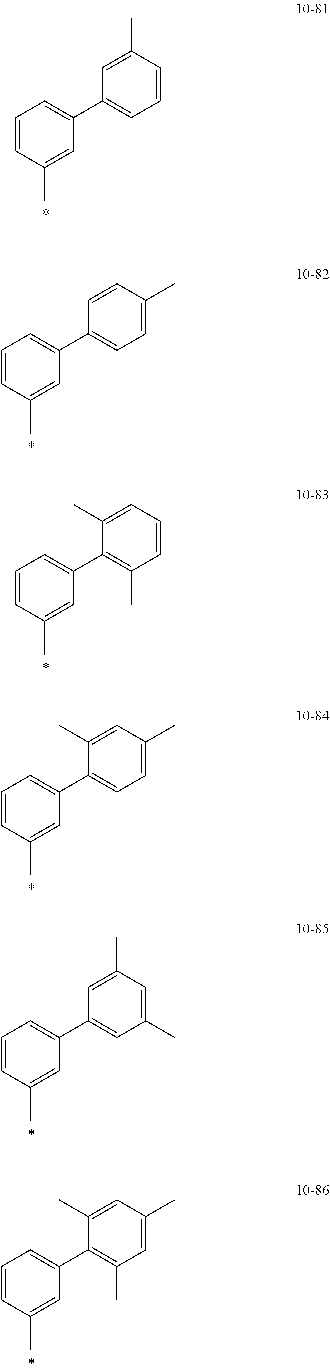

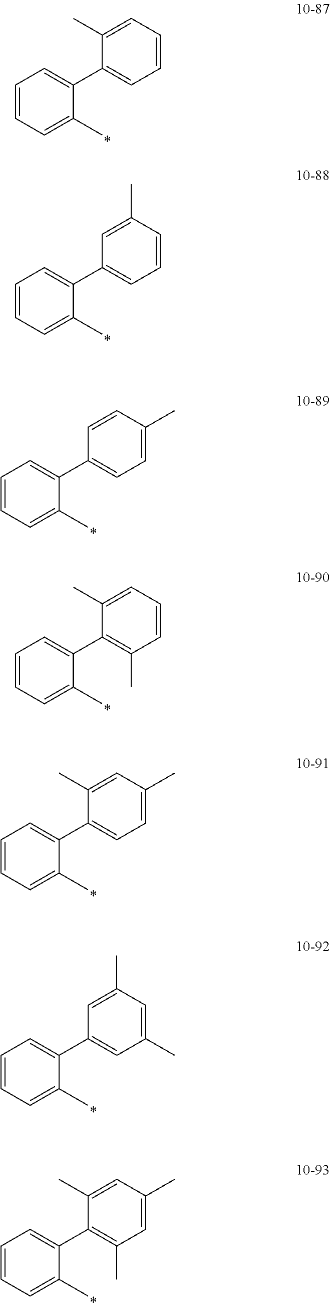

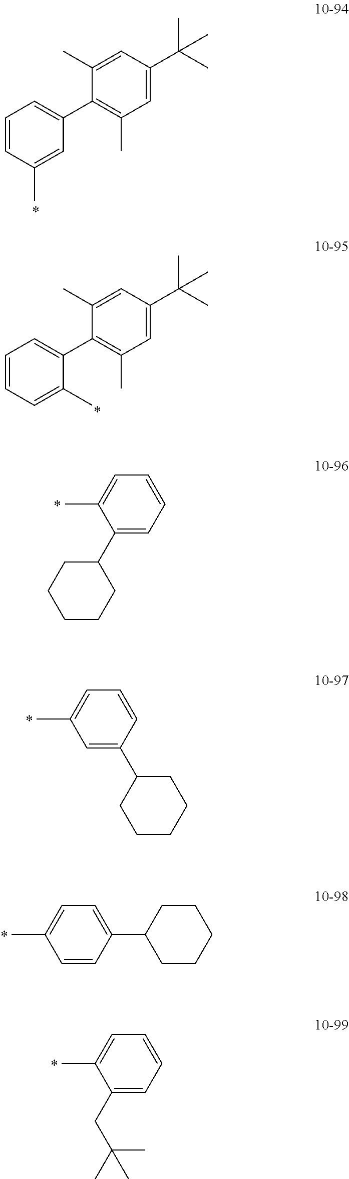

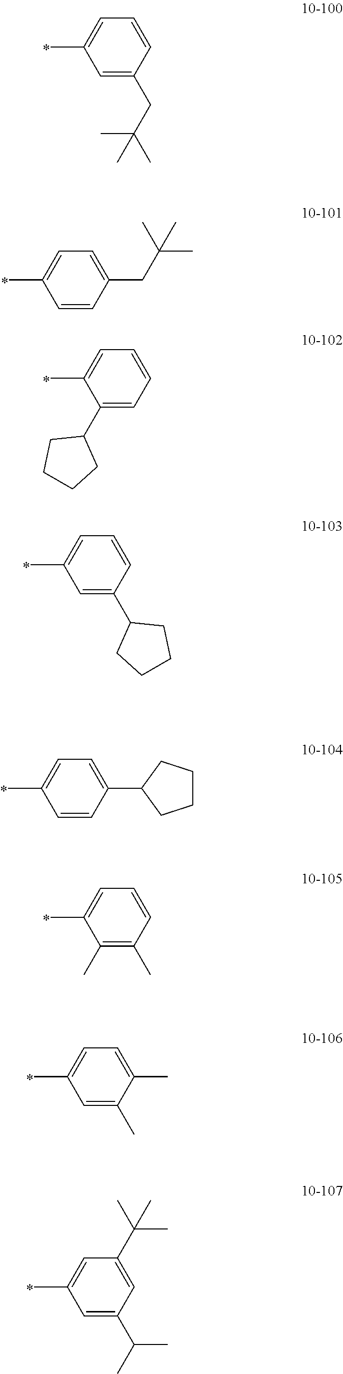

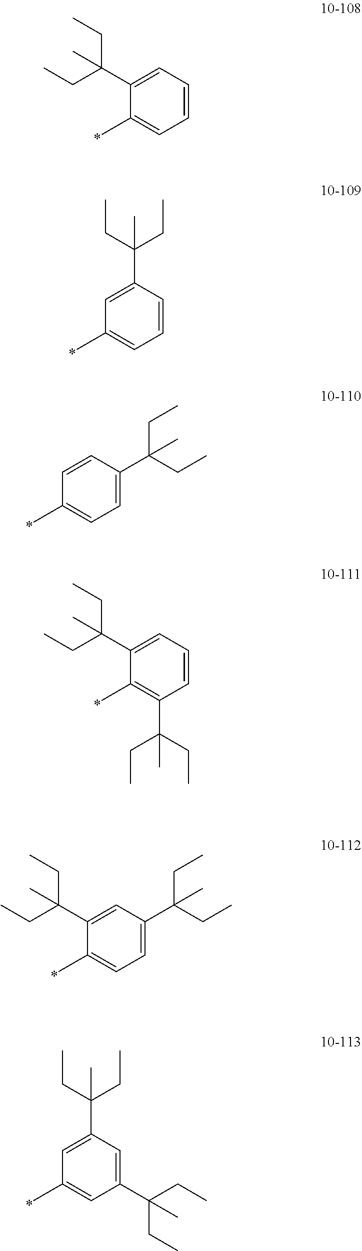

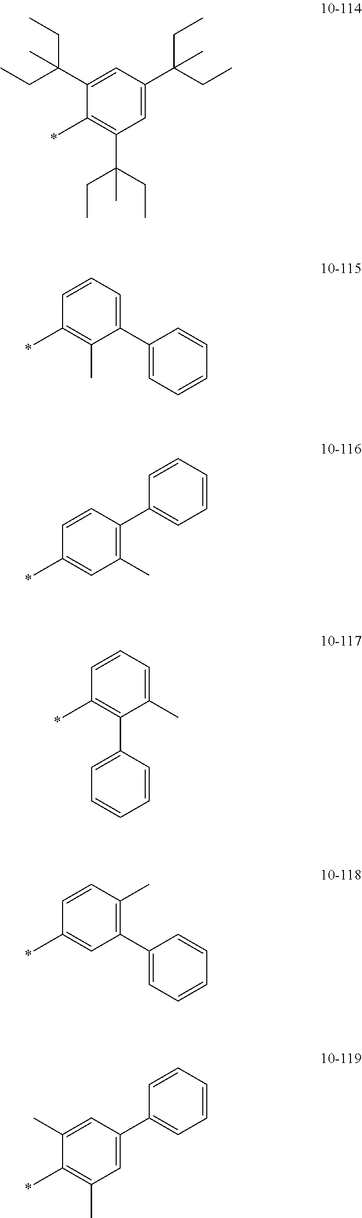

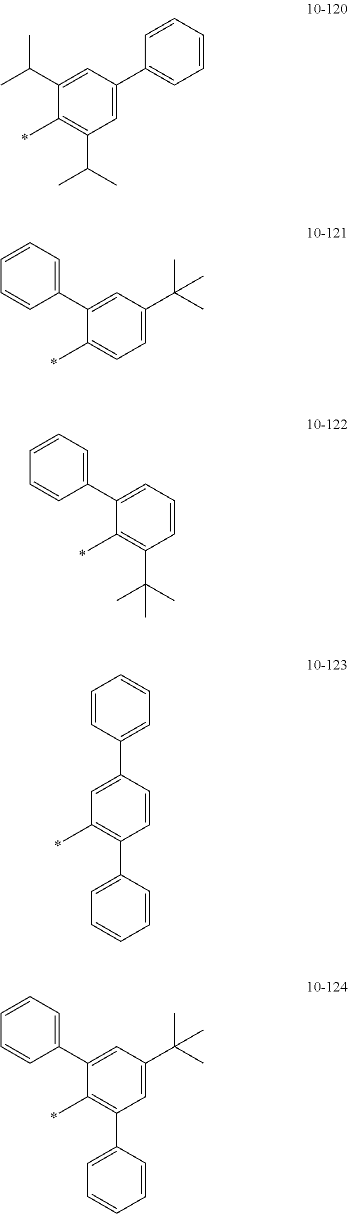

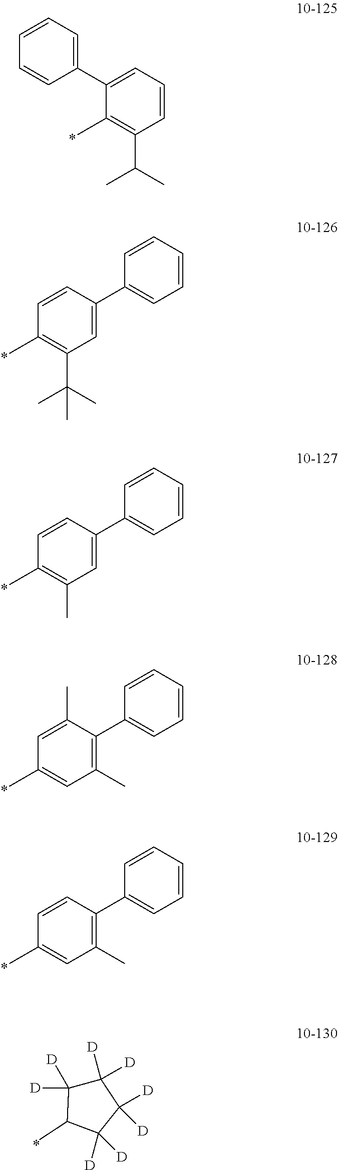

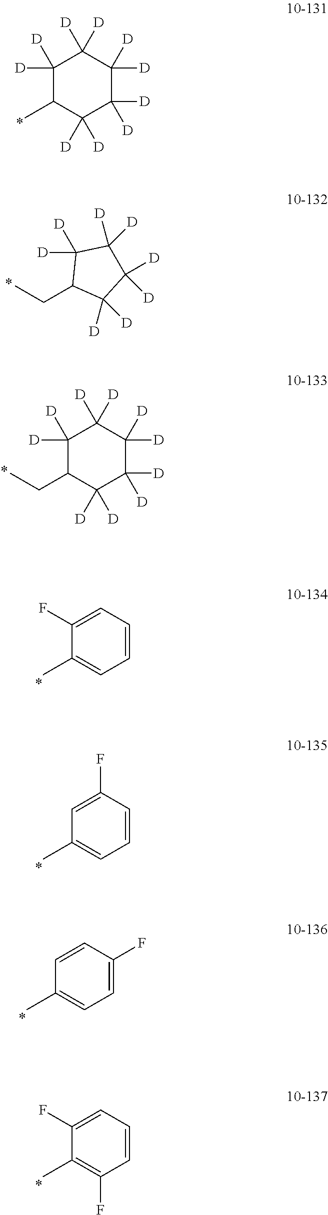

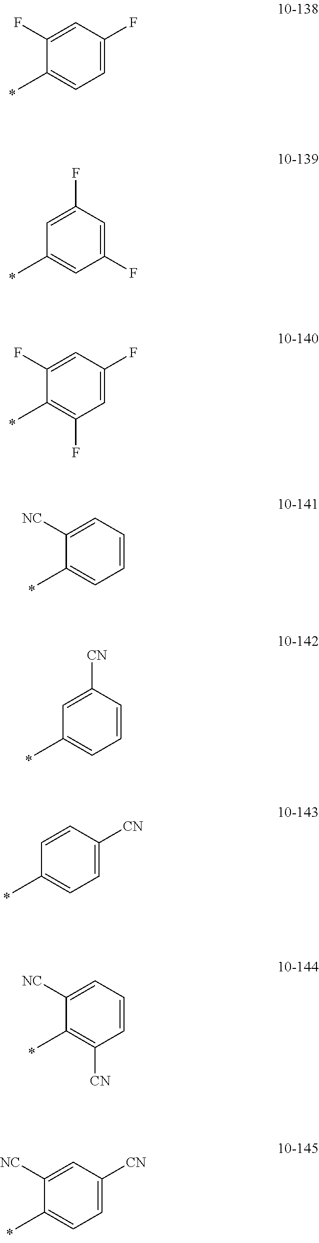

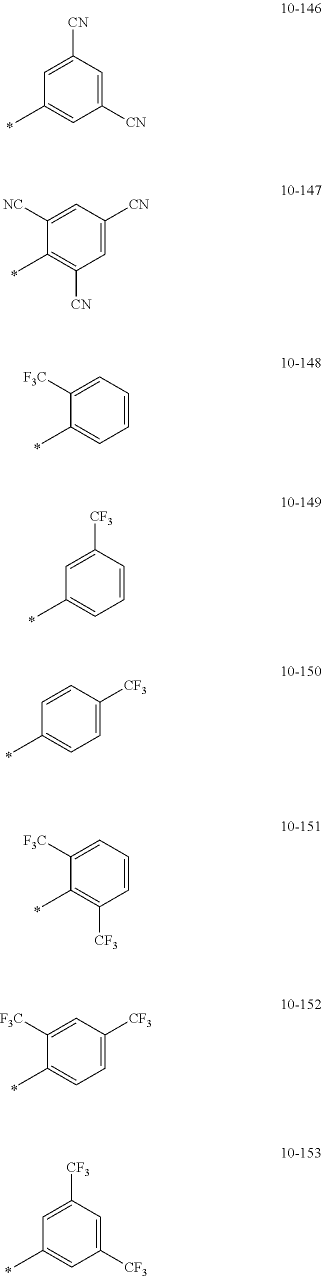

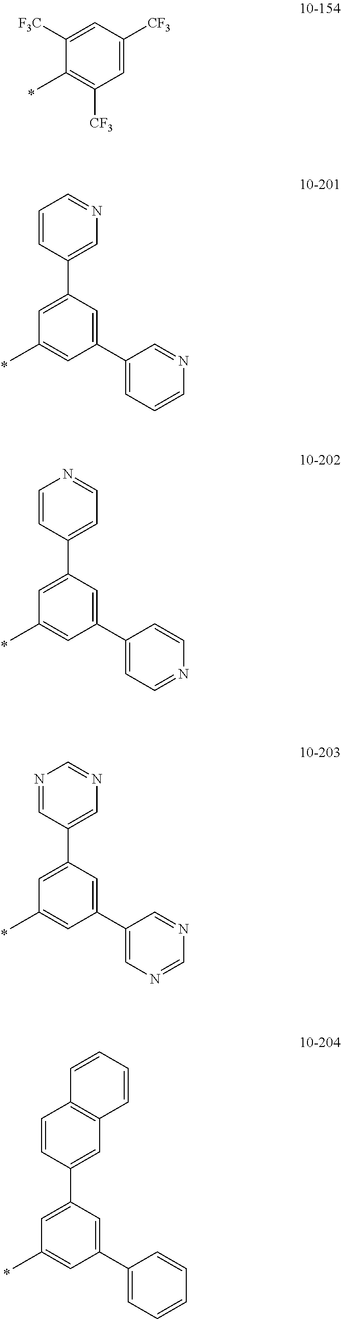

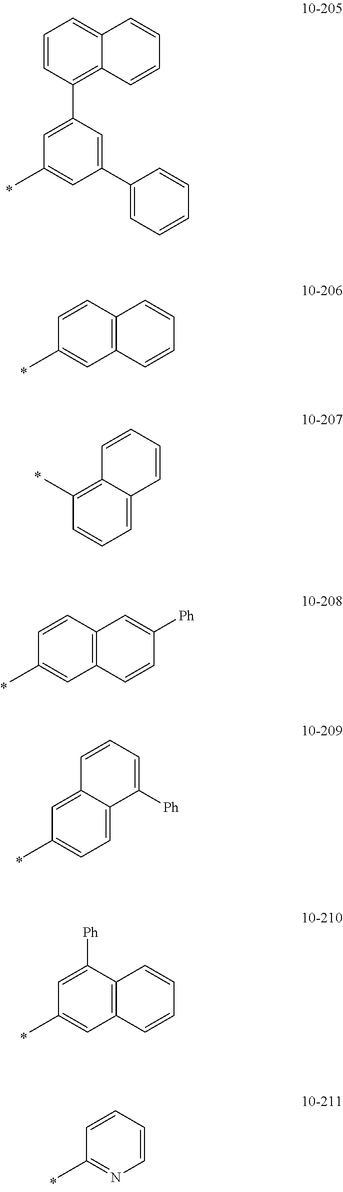

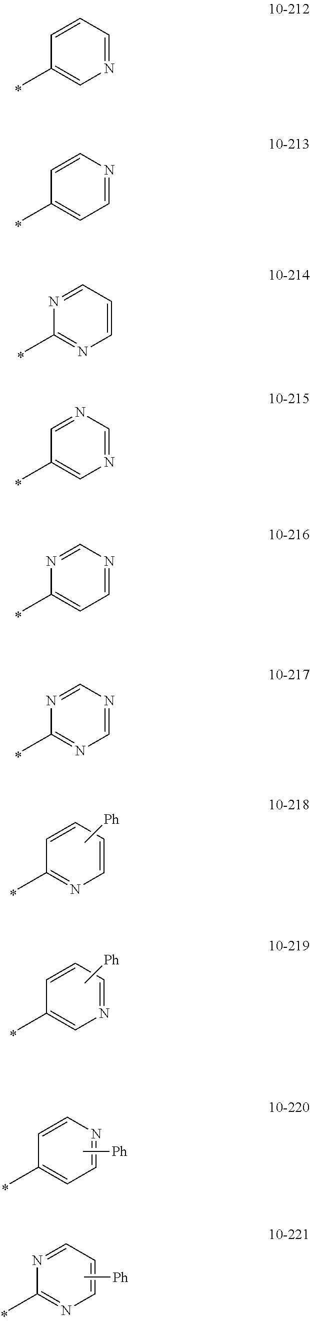

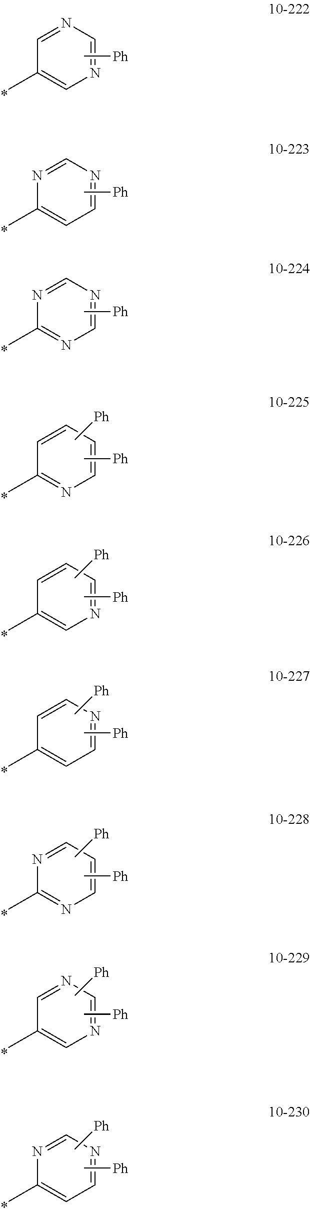

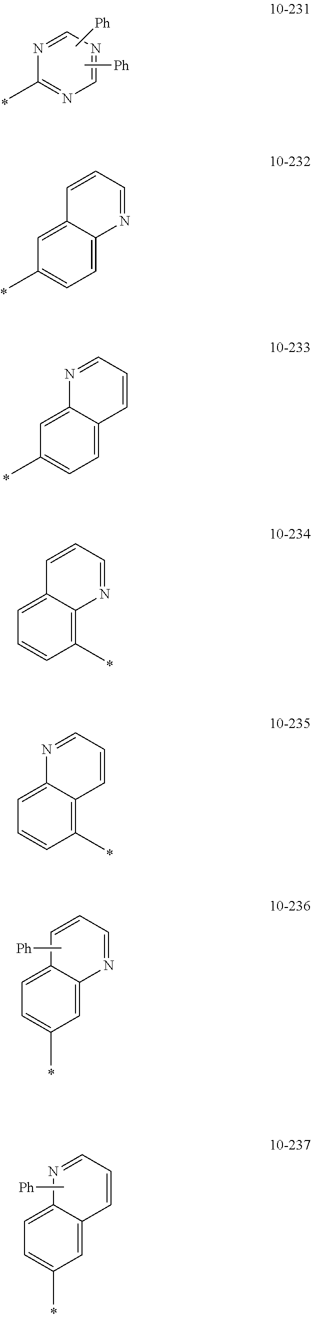

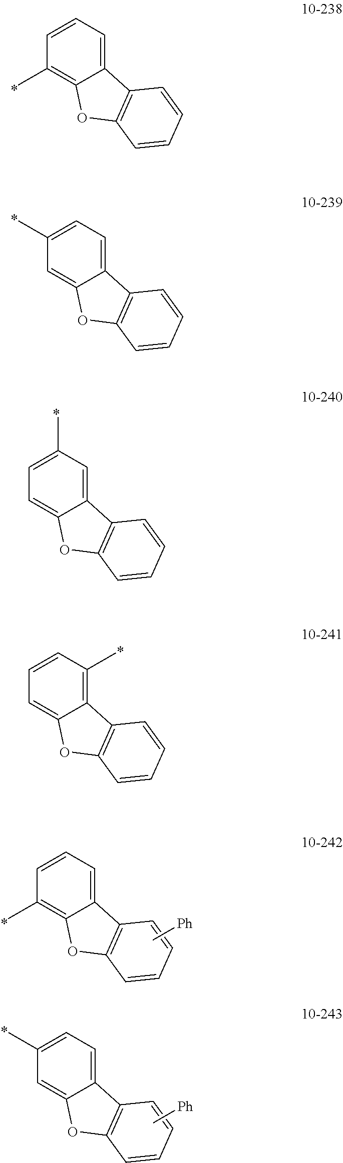

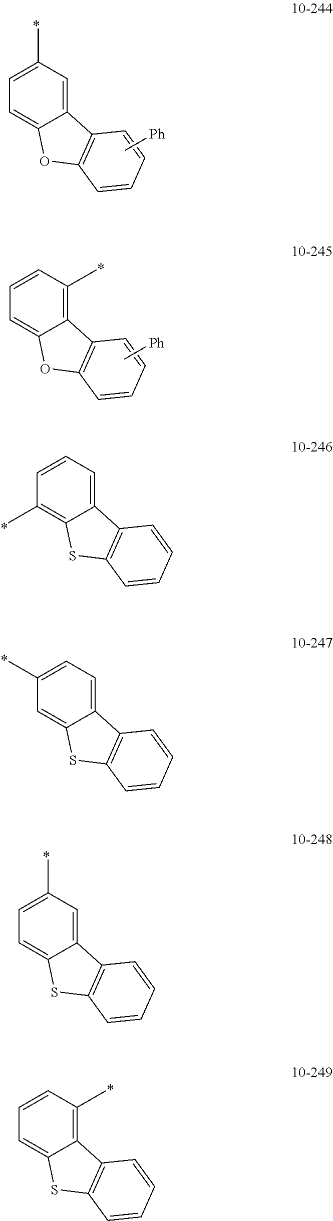

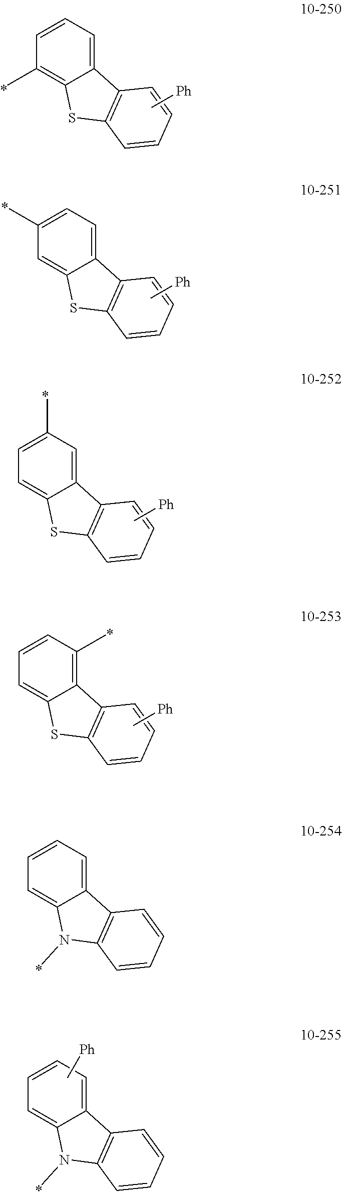

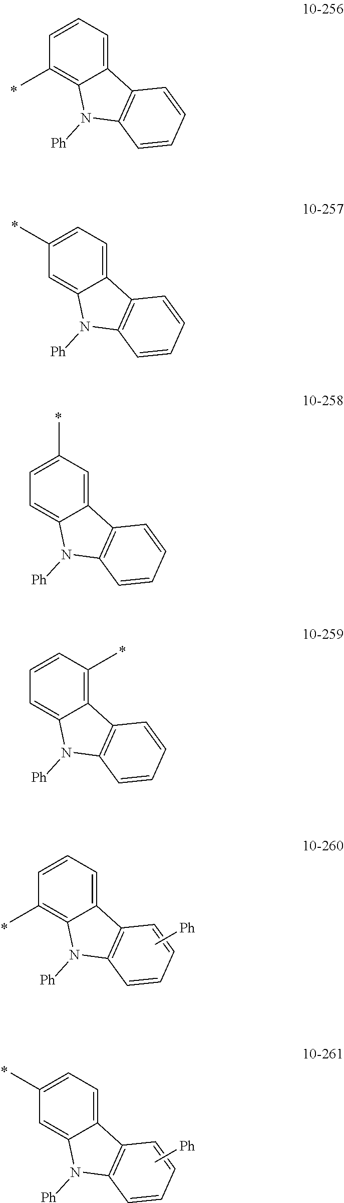

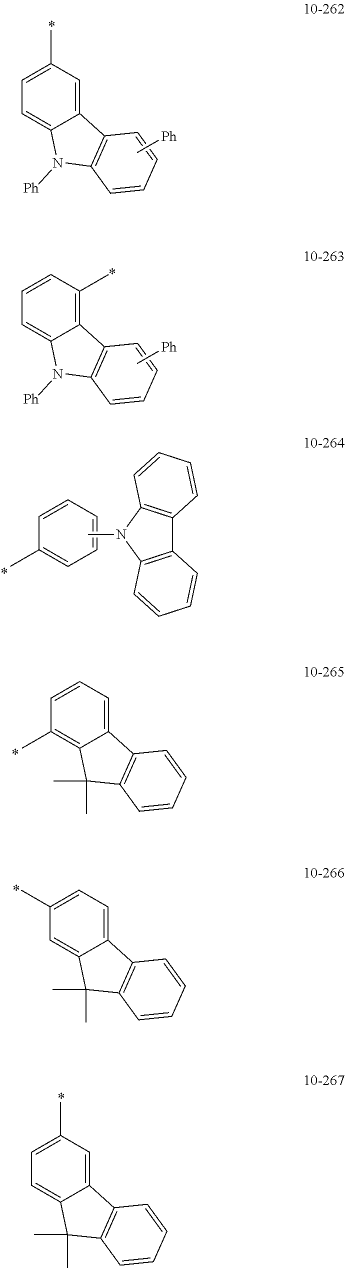

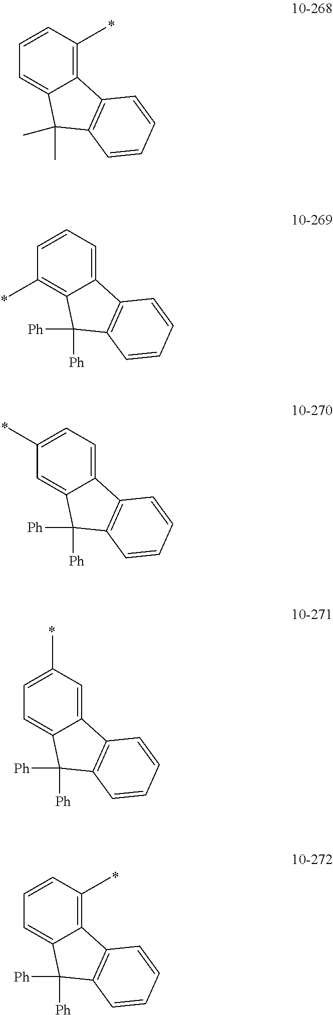

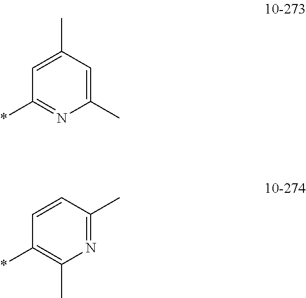

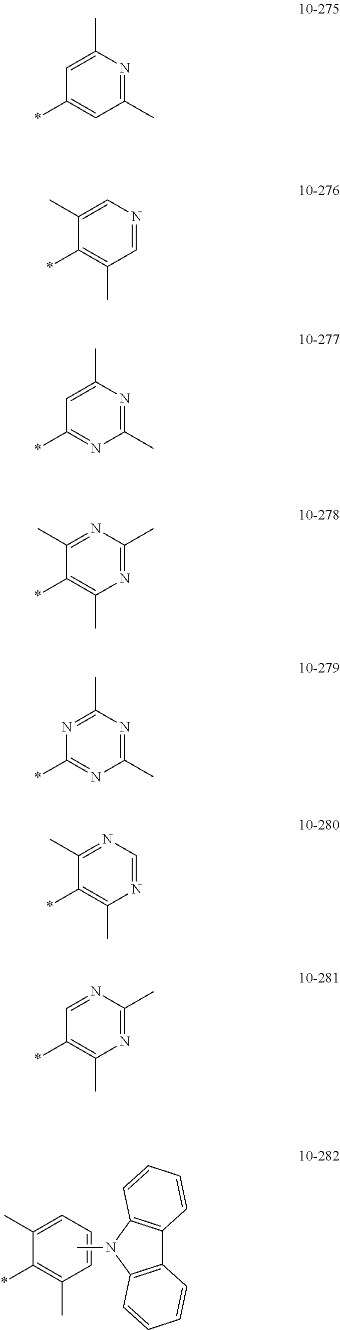

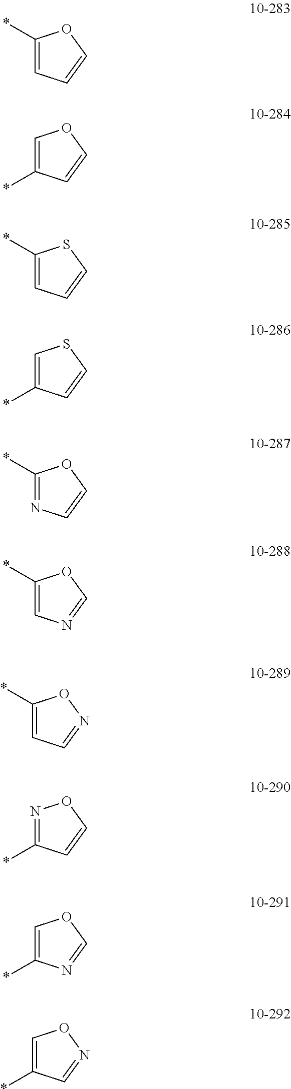

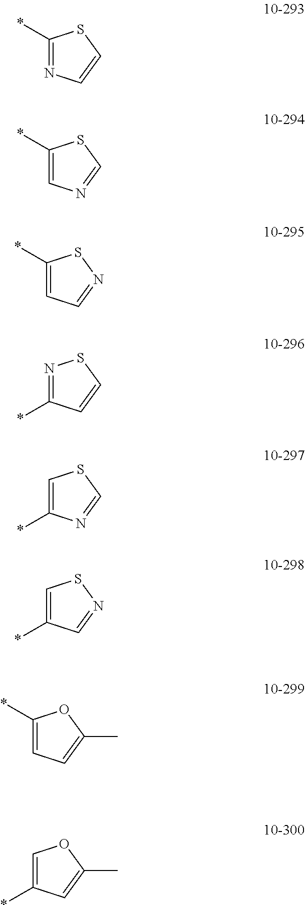

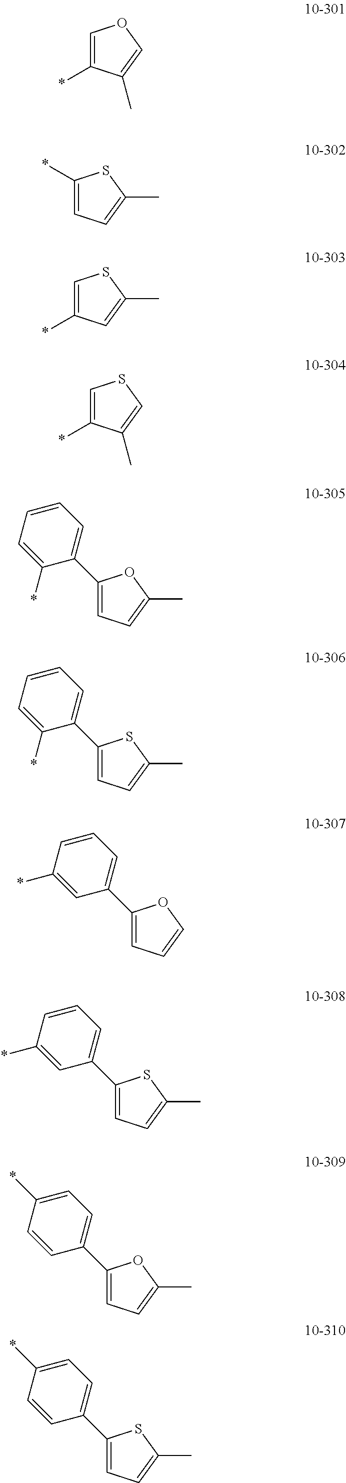

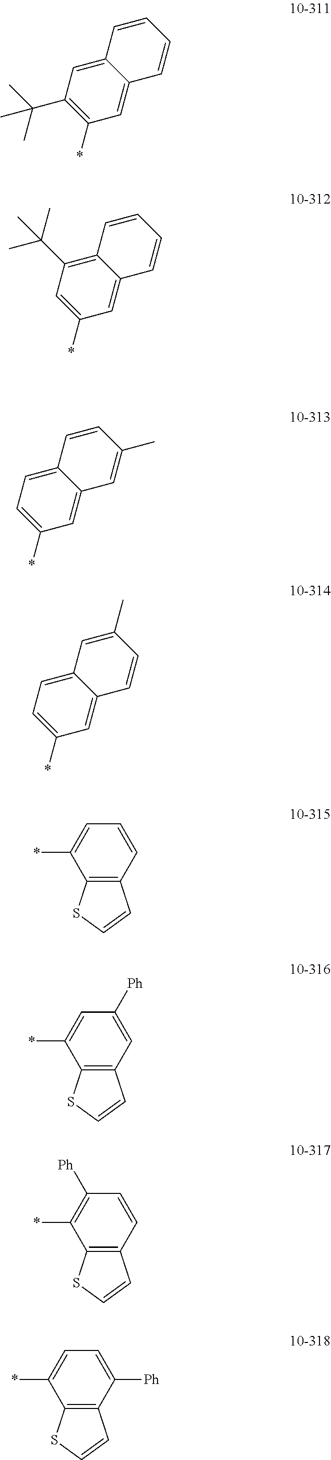

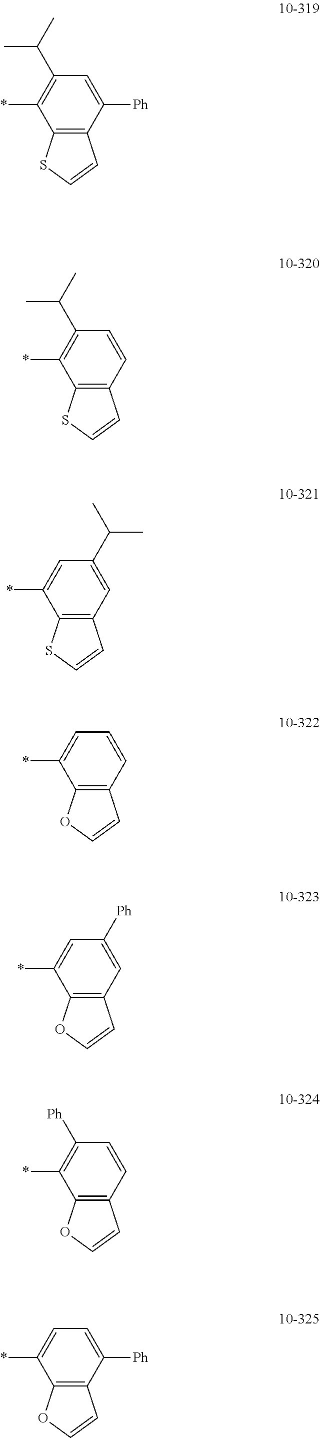

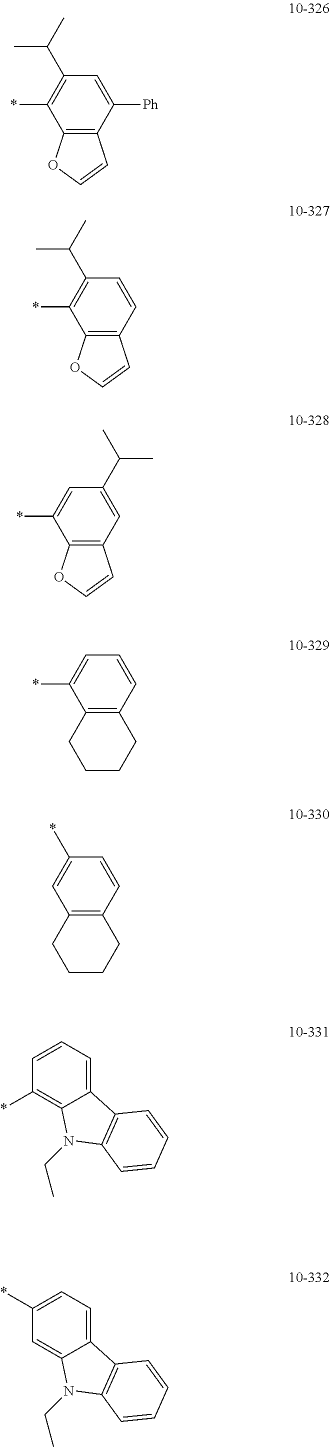

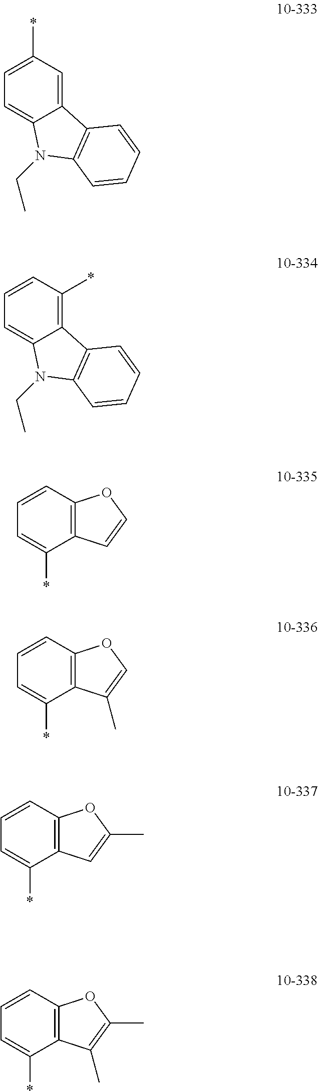 [SMILES: *c1ccc2c(c1)c1ccccc1n2CC ; *c1cccc2c1c1ccccc1n2CC ; *c1cccc2occc12 ; *c1cccc2occ(C)c12 ; *c1cccc2oc(C)cc12 ; *c1cccc2oc(C)c(C)c12]

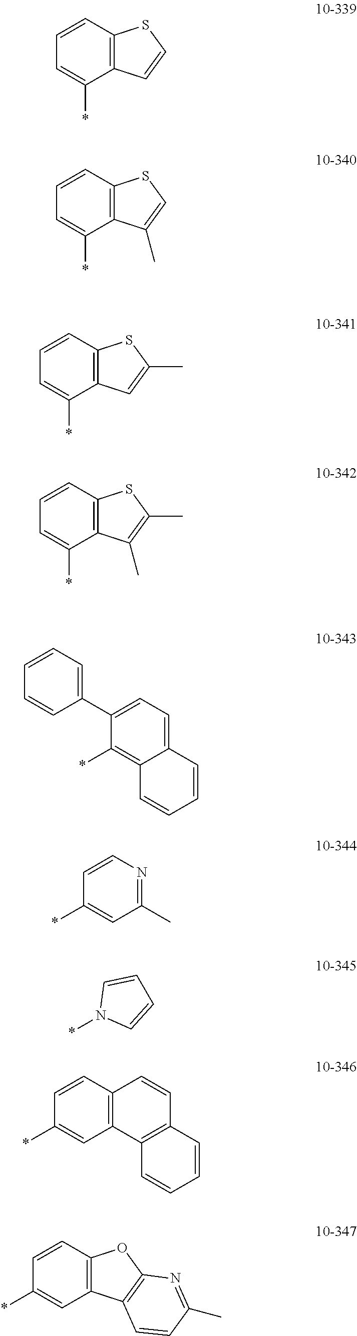

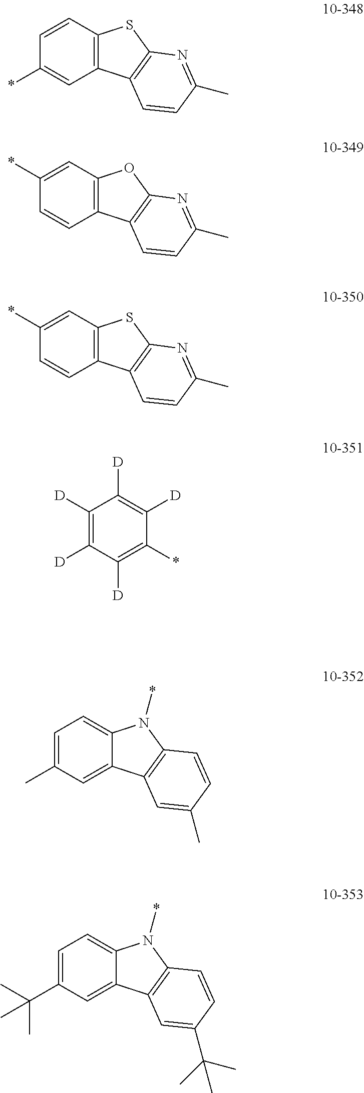

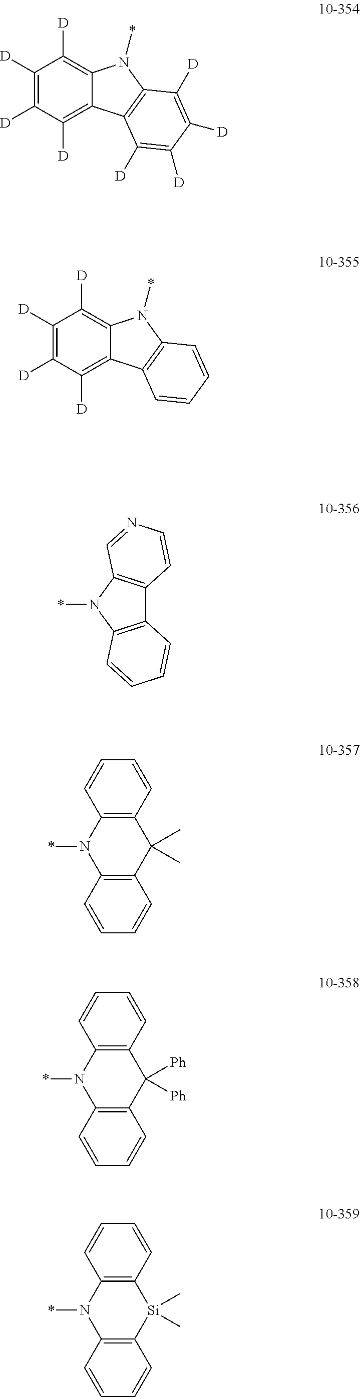 [SMILES: *n1c2c([2H])c([2H])c([2H])c([2H])c2c2c([2H])c([2H])c([2H])c([2H])c21 ; *n1c2ccccc2c2c([2H])c([2H])c([2H])c([2H])c21 ; *n1c2ccccc2c2ccncc21 ; *N1c2ccccc2C(C)(C)c2ccccc21 ; *N1c2ccccc2C(c2ccccc2)(c2ccccc2)c2ccccc21 ; *N1c2ccccc2[Si](C)(C)c2ccccc21]

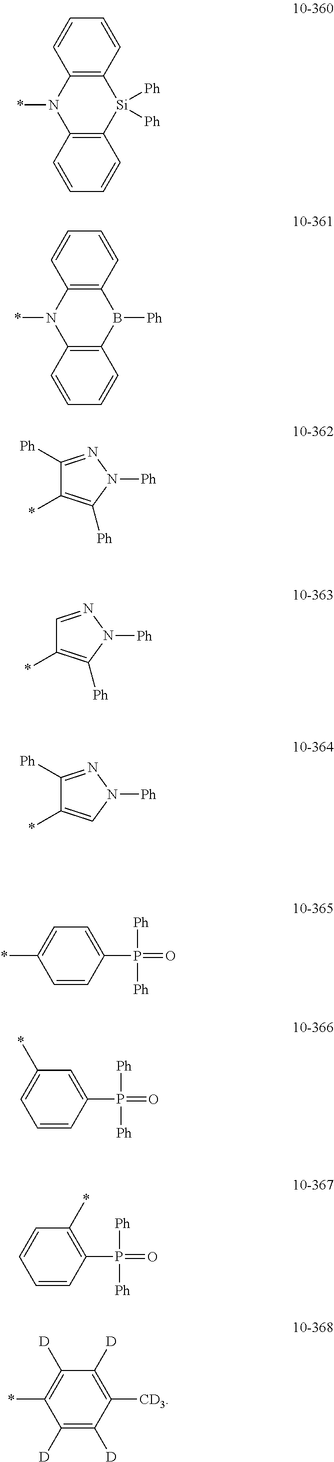

In Formulae 9-1 to 9-61, 9-201 to 9-237, 10-1 to 10-154, and 10-201 to 10-368, * indicates a binding site to a neighboring atom, Ph is a phenyl group, TMS is a trimethylsilyl group, and TMG is a trimethylgermyl group.

a71 to a74 and a501 to a504 in Formulae 20-1 to 20-5 respectively indicate numbers of R₇₁ to R₇₄, and may each independently be an integer from 0 to 20. When a71 is 2 or more, two or more of R₇₁ may be substantially identical to or different from each other, when a72 is 2 or more, two or more of R₇₂ may be substantially identical to or different from each other, when a73 is 2 or more, two or more of R₇₃ may be substantially identical to or different from each other, and when a74 is 2 or more, two or more of R₇₄ may be substantially identical to or different from each other. a71 to a74 may each independently be an integer from 0 to 8.

In Formula 30, a group represented by *-(L₅₁)_{b51}-R₅₁ and a group represented by *-(L₅₂)_{b52}-R₅₂ may each not be a phenyl group.

In one or more embodiments, in Formula 30, a group represented by *-(L₅₁)_{b51}-R₅₁ and a group represented by *-(L₅₂)_{b52}-R₅₂ may be identical to each other.

In one or more embodiments, in Formula 30, a group represented by *-(L₅₁)_{b51}-R₅₁ and a group represented by *-(L₅₂)_{b52}-R₅₂ may be different from each other.

In one or more embodiments, in Formula 30, b51 and b52 may each independently be 1, 2, or 3, and L₅₁ and L₅₂ may each independently be a benzene group, a pyridine group, a pyrimidine group, a pyridazine group, a pyrazine group, or a triazine group, each unsubstituted or substituted with at least one R₁₀ₐ.

In one or more embodiments, R₅₁ and R₅₂ in Formula 30 may each independently be a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —C(Q₁)(Q₂)(Q₃), or —Si(Q₁)(Q₂)(Q₃), and
Q₁ to Q₃ may each independently be a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments,
a group represented by *-(L₅₁)_{b51}-R₅₁ in Formula 30 may be a group represented by one of Formulae CY51-1 to CY51-26, and/or
a group represented by *-(L₅₂)_{b52}-R₅₂ in Formula 30 may be a group represented by one of Formulae CY52-1 to CY52-26, and/or
a group represented by *-(L₅₃)_{b53}-R₅₃ in Formula 30 may be a group represented by one of Formulae CY53-1 to CY53-27, —C(Q₁)(Q₂)(Q₃), or —Si(Q₁)(Q₂)(Q₃):

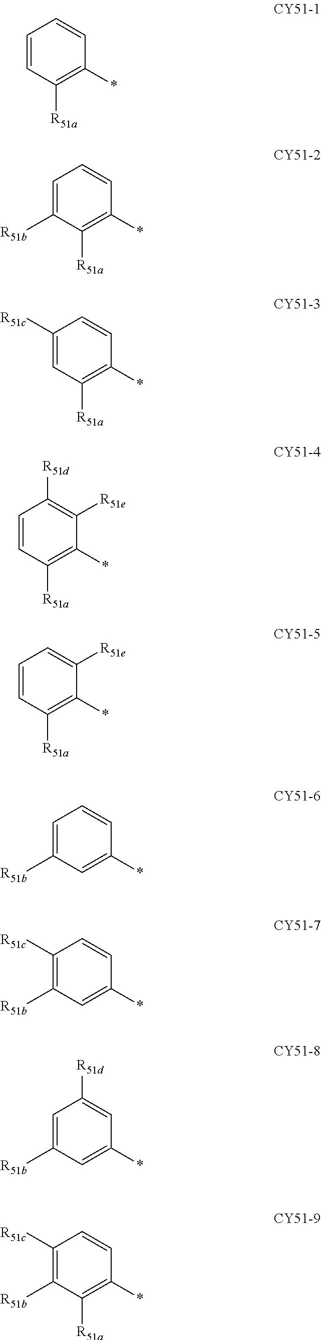

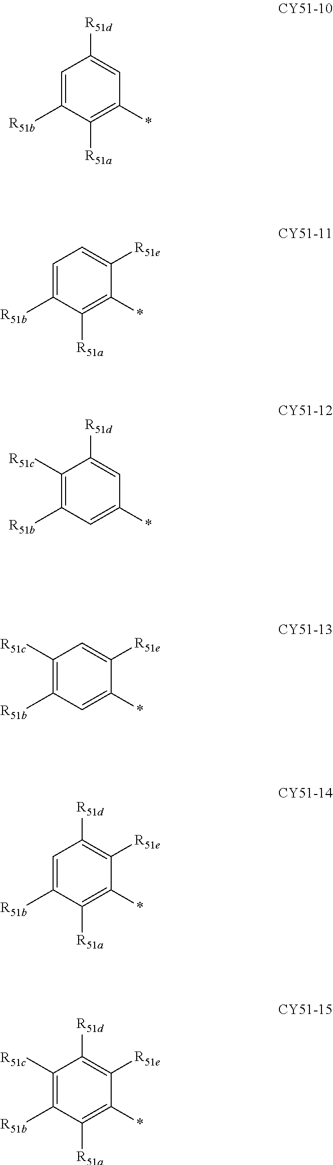

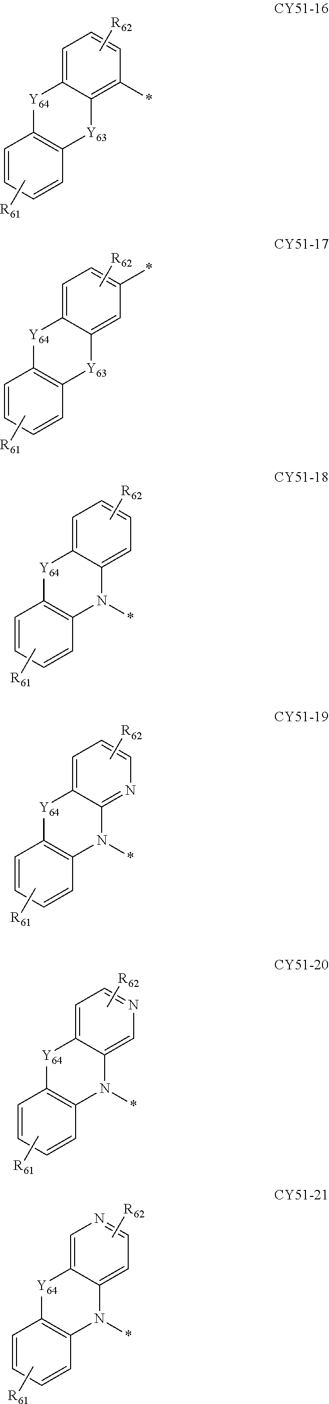

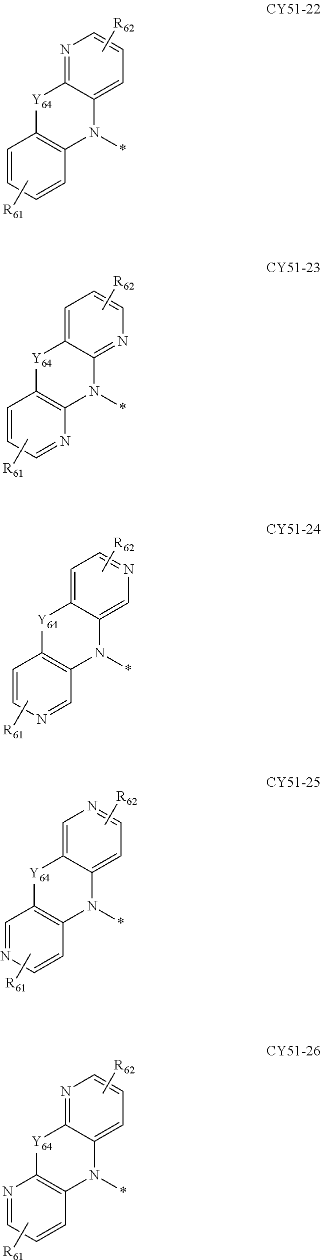

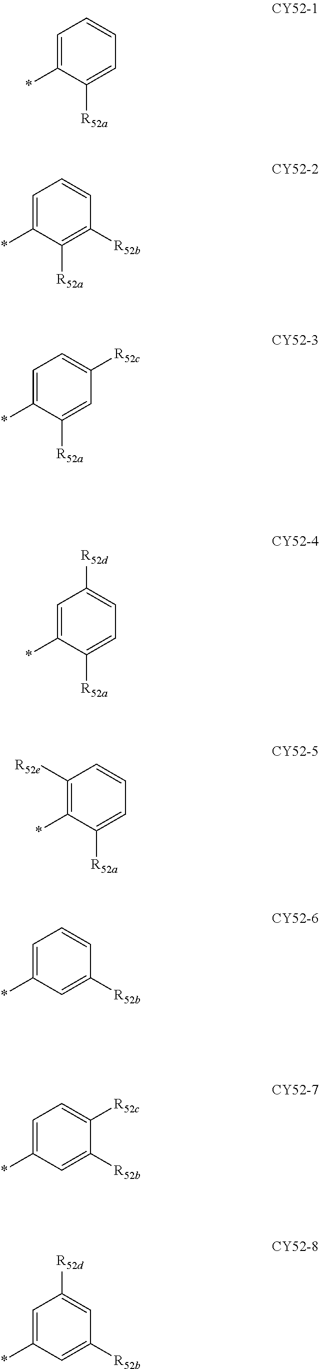

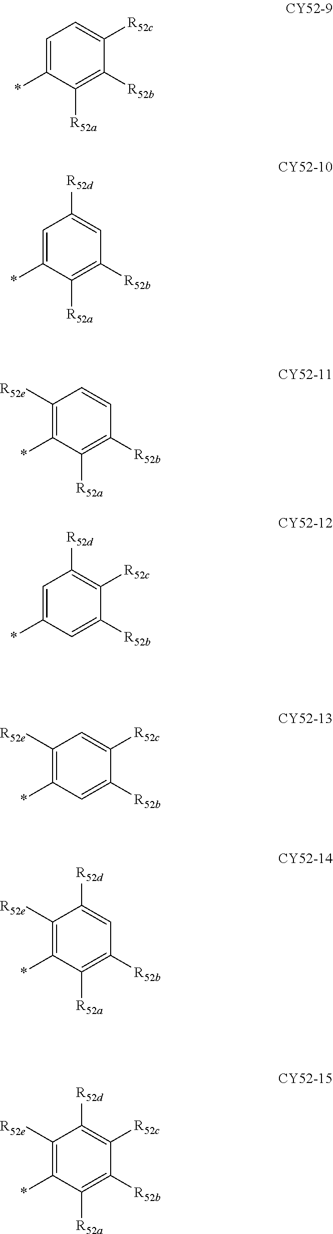

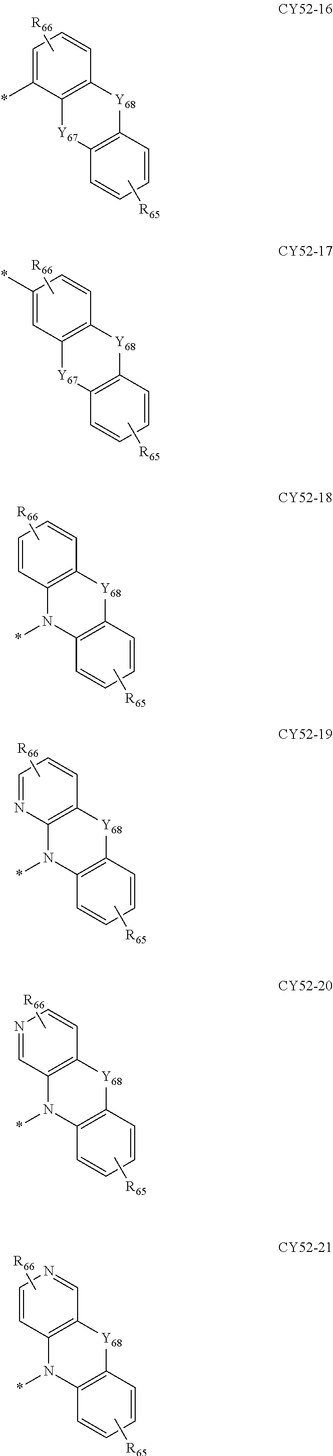

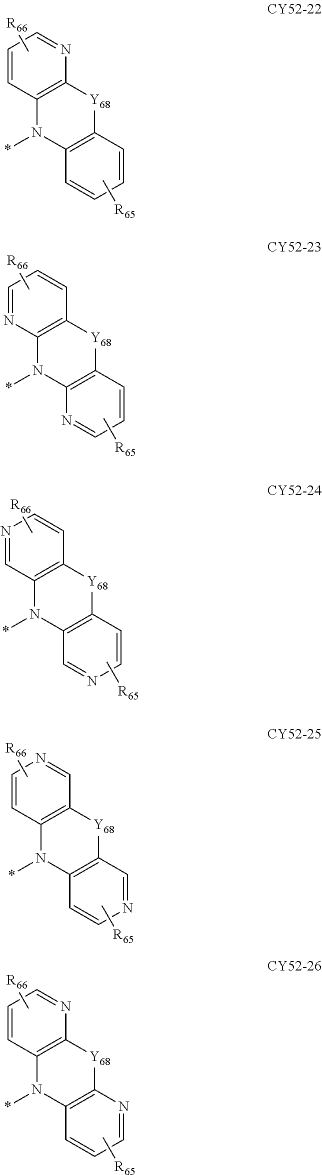

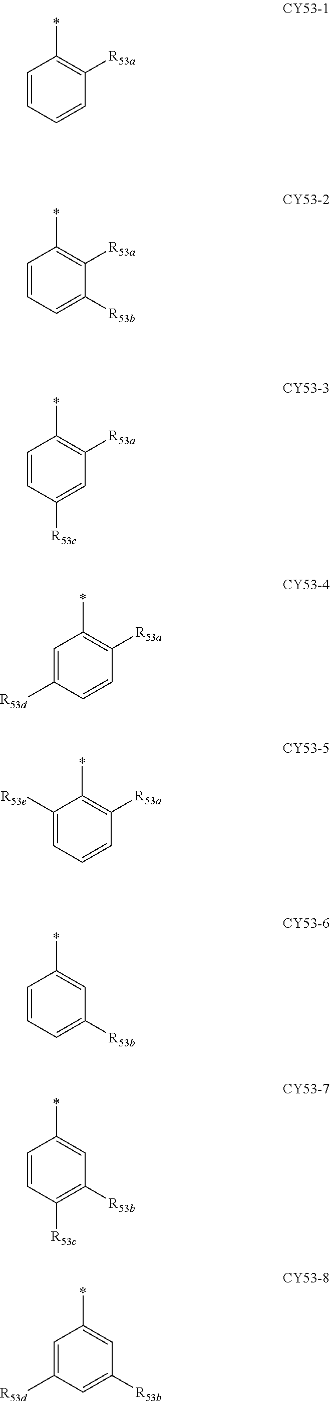

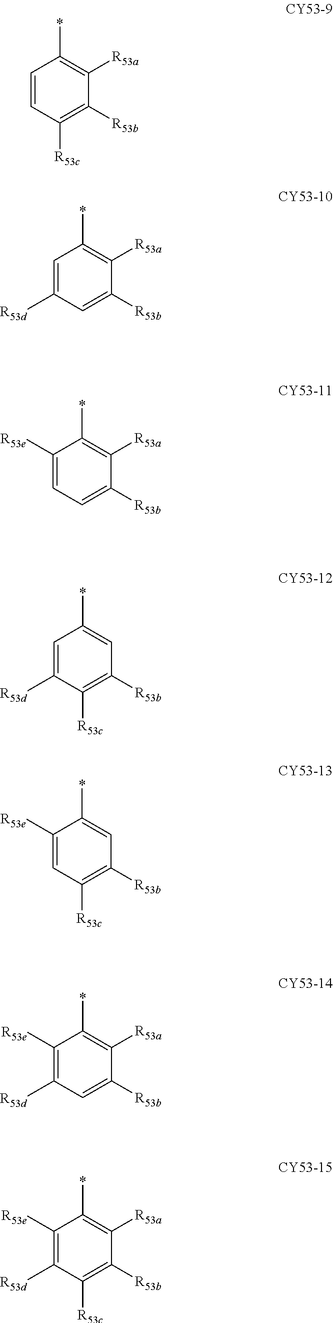

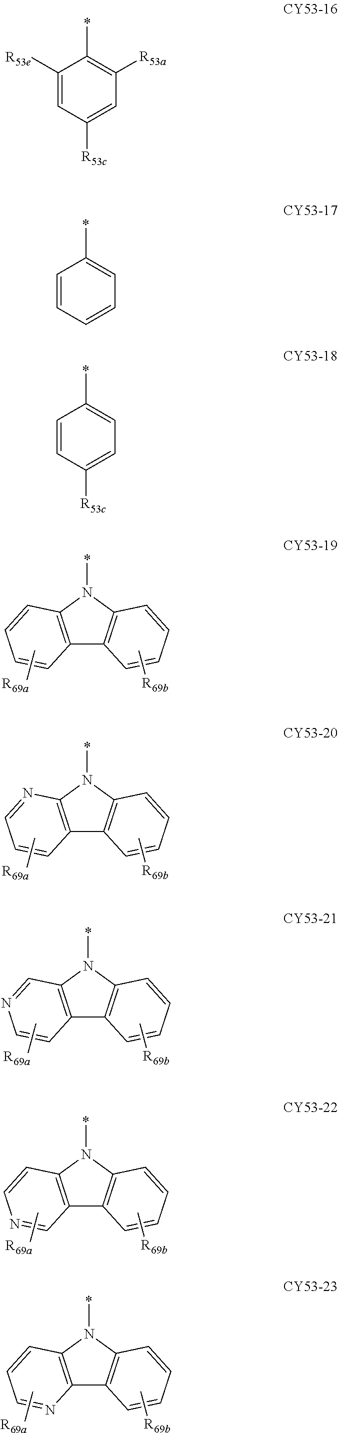

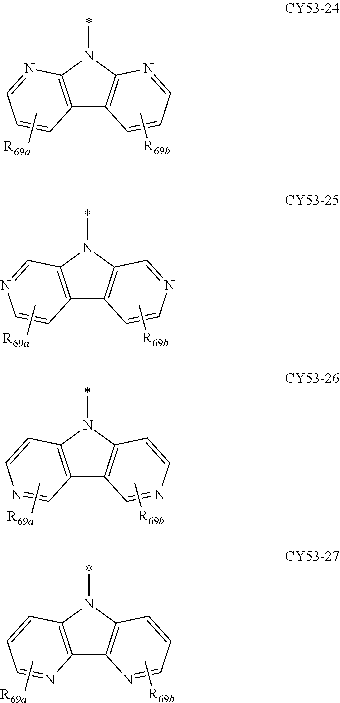

wherein, in Formulae CY51-1 to CY51-26, CY52-1 to CY52-26, and CY53-1 to CY53-27,
Y₆₃ may be a single bond (e.g., a single covalent bond), O, S, N(R₆₃), B(R₆₃), C(R₆₃ₐ)(R_{63b}), or Si(R₆₃ₐ)(R_{63b}),
Y₆₄ may be a single bond (e.g., a single covalent bond), O, S, N(R₆₄), B(R₆₄), C(R₆₄ₐ)(R_{64b}), or Si(R₆₄ₐ)(R_{64b}),
Y₆₇ may be a single bond (e.g., a single covalent bond), O, S, N(R₆₇), B(R₆₇), C(R₆₇ₐ)(R_{67b}), or Si(R₆₇ₐ)(R_{67b}),
Y₆₈ may be a single bond (e.g., a single covalent bond), O, S, N(R₆₈), B(R₆₈), C(R₆₈ₐ)(R_{68b}), or Si(R₆₈ₐ)(R_{68b}),
each of Y₆₃ and Y₆₄ in Formulae CY51-16 and CY51-17 may not be a single bond (e.g., a single covalent bond) at the same time (e.g., simultaneously),
each of Y₆₇ and Y₆₈ in Formulae CY52-16 and CY52-17 may not be a single bond (e.g., a single covalent bond) at the same time (e.g., simultaneously),
R₅₁ₐ to R₅₁ₑ, R₆₁ to R₆₄, R₆₃ₐ, R_{63b}, R₆₄ₐ, and R_{64b} may each be as described in connection with R₅₁, wherein R₅₁ₐ to R₅₁ₑ may each not be hydrogen,
R₅₂ₐ to R₅₂ₑ, R₆₅ to R₆₈, R₆₇ₐ, R_{67b}, R₆₈ₐ, and R_{68b} may each be as described in connection with R₅₂, wherein R₅₂ₐ to R₅₂ₑ may each not be hydrogen,
R₅₃ₐ to R₅₃ₑ, R₆₉ₐ, and R_{69b} may each be as described in connection with R₅₃, wherein R₅₃ₐ to R₅₃ₑ may each not be hydrogen, and
* indicates a binding site to a neighboring atom.

In one or more embodiments,
in Formulae CY51-1 to CY51-26 and CY52-1 to 52-26, R₅₁ₐ to R₅₁ₑ and R₅₂ₐ to R₅₂ₑ may each independently be:
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, or a group represented by Formula 91, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or any combination thereof; or
—C(Q₁)(Q₂)(Q₃) or —Si(Q₁)(Q₂)(Q₃),
Q₁ to Q₃ may each independently be a phenyl group, a naphthyl group, a pyridinyl group, a pyrimidinyl group, a pyridazinyl group, a pyrazinyl group, or a triazinyl group, each unsubstituted or substituted with deuterium, a C₁-C₁₀ alkyl group, a phenyl group, a biphenyl group, a pyridinyl group, a pyrimidinyl group, a pyridazinyl group, a pyrazinyl group, a triazinyl group, or any combination thereof,
in Formulae CY51-16 and CY51-17, i) Y₆₃ may be O or S, and Y₆₄ may be Si(R₆₄ₐ)(R_{64b}), or ii) Y₆₃ may be Si(R₆₃ₐ)(R_{63b}), and Y₆₄ may be O or S, and
in Formulae CY52-16 and CY52-17, i) Y₆₇ may be O or S, and Y₆₈ may be Si(R₆₈ₐ)(R_{68b}), or ii) Y₆₇ may be Si(R₆₇ₐ)(R_{67b}), and Y₆₈ may be O or S.

In Formulae 3-1 to 3-5, L₈₁ to L₈₅ may each independently be:
a single bond (e.g., a single covalent bond);

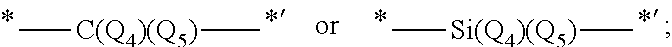

a
a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, or a benzothiadiazole group, each unsubstituted or substituted with deuterium, —F, —Cl, —Br, —I, a hydroxyl group, a cyano group, a nitro group, a C₁-C₂₀ alkyl group, a C₁-C₂₀ alkoxy group, a phenyl group, a naphthyl group, a pyridinyl group, a pyrimidinyl group, a triazinyl group, a fluorenyl group, a dimethylfluorenyl group, a diphenylfluorenyl group, a carbazolyl group, a phenylcarbazolyl group, a dibenzofuranyl group, a dibenzothiophenyl group, a dibenzosilolyl group, a dimethyldibenzosilolyl group, a diphenyldibenzosilolyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof, and
Q₄, Q₅, and Q₃₁ to Q₃₃ may each independently be hydrogen, deuterium, a C₁-C₂₀ alkyl group, a C₁-C₂₀ alkoxy group, a phenyl group, a biphenyl group, a terphenyl group, a pyridinyl group, a pyrimidinyl group, a pyridazinyl group, a pyrazinyl group, or a triazinyl group.

In one or more embodiments, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 402 is 2 or more, two ring A₄₀₁(s) in two or more of L₄₀₁(s) may optionally be linked to each other via T₄₀₂, which is a linking group, or two ring A₄₀₂(s) may optionally be linked to each other via T₄₀₃, which is a linking group (see compounds PD1 to PD4 and PD7). T₄₀₂ and T₄₀₃ are each as described in connection with T₄₀₁.

L₄₀₂ in Formula 401 may be an organic ligand. In one or more embodiments, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, and/or the like), or any combination thereof.

### Detailed Examples of Second Compound to Fourth Compound

According to one or more embodiments, the second compound may include at least one of compound HTH1 to compound HTH57:

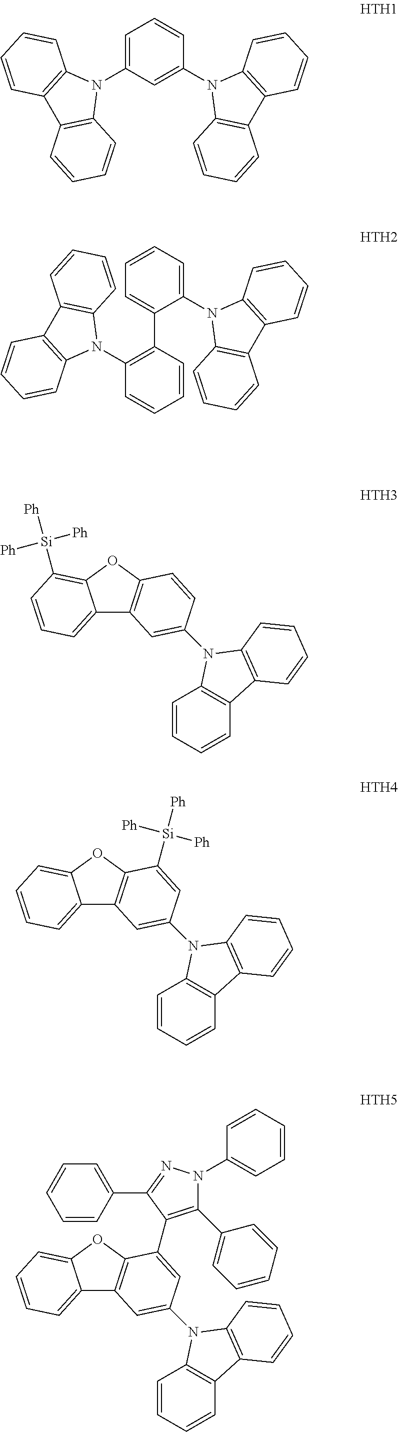 [SMILES: c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1]

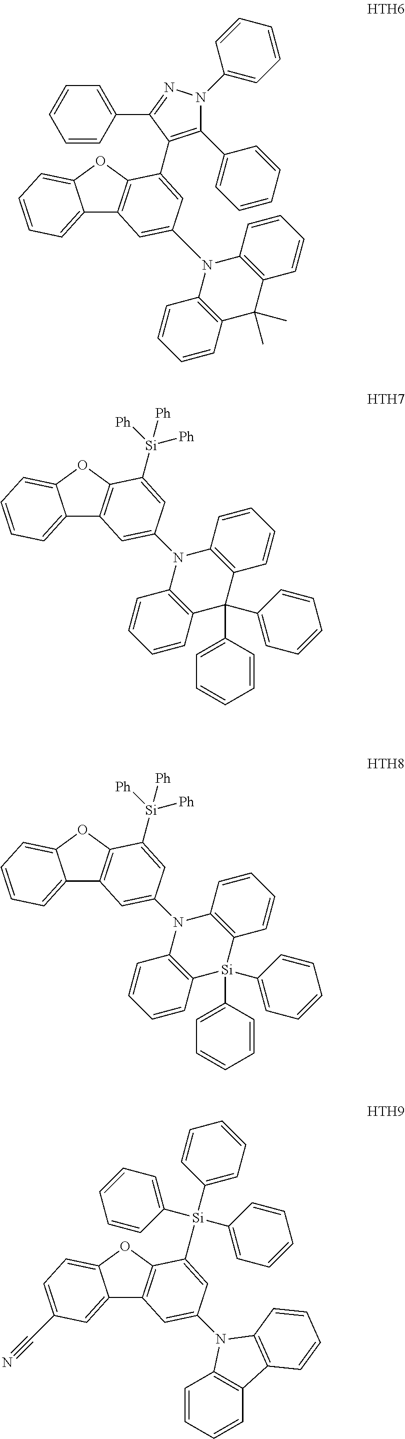 [SMILES: CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1]

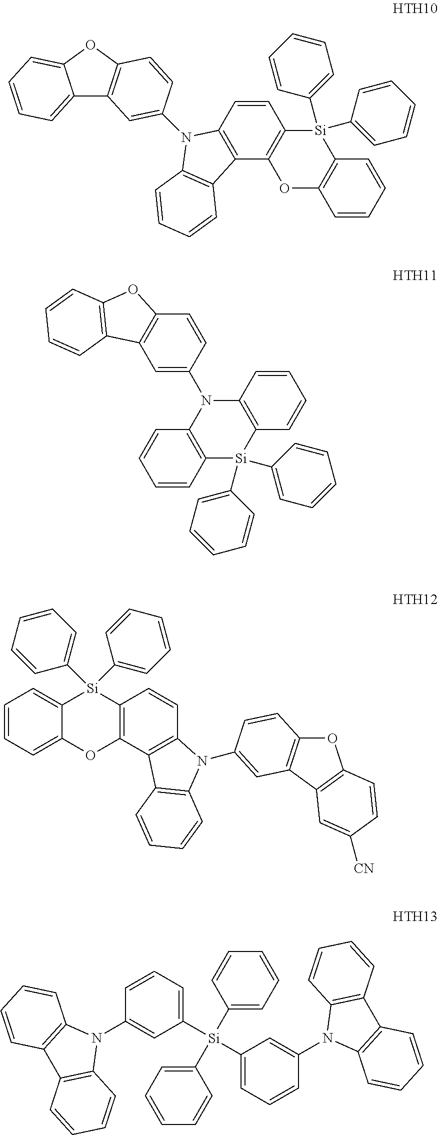 [SMILES: c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1]

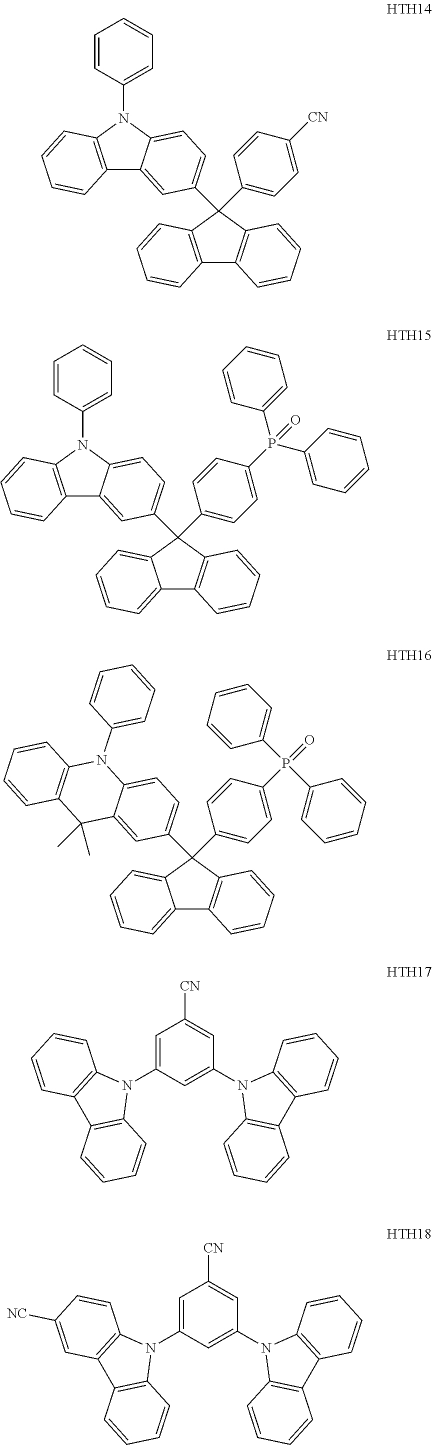 [SMILES: N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1]

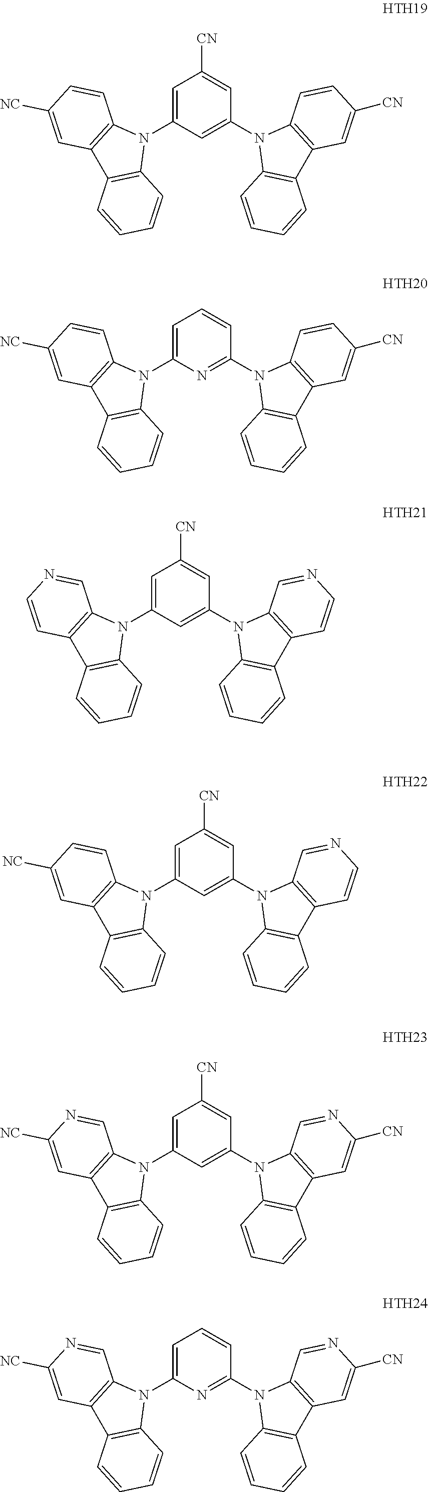 [SMILES: N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1]

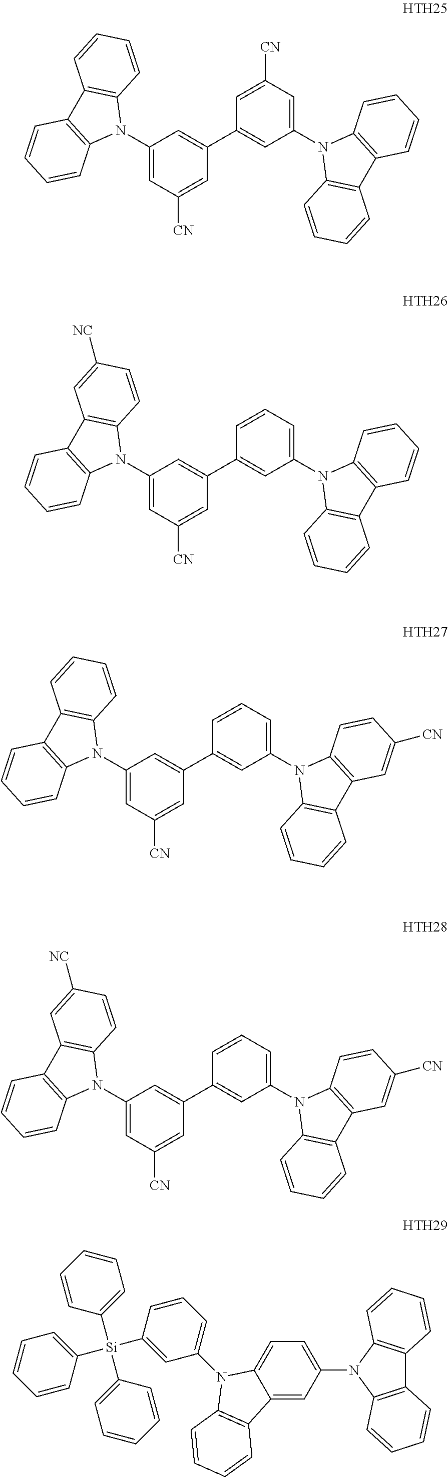 [SMILES: N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1 ; c1ccc([Si](c2ccccc2)(c2ccccc2)c2cccc(-n3c4ccccc4c4cc(-n5c6ccccc6c6ccccc65)ccc43)c2)cc1]

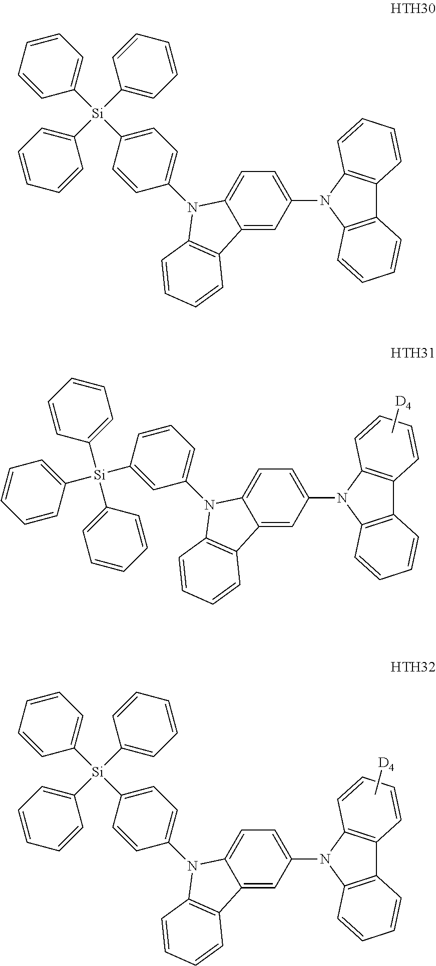

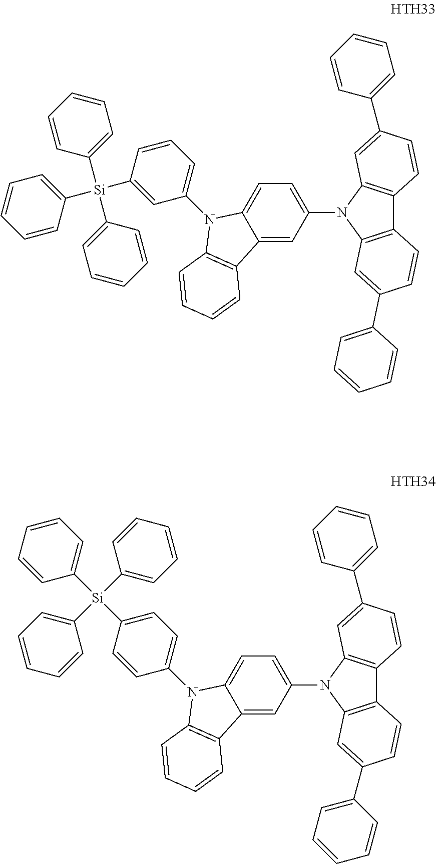 [SMILES: c1ccc(-c2ccc3c4ccc(-c5ccccc5)cc4n(-c4ccc5c(c4)c4ccccc4n5-c4cccc([Si](c5ccccc5)(c5ccccc5)c5ccccc5)c4)c3c2)cc1 ; c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1]

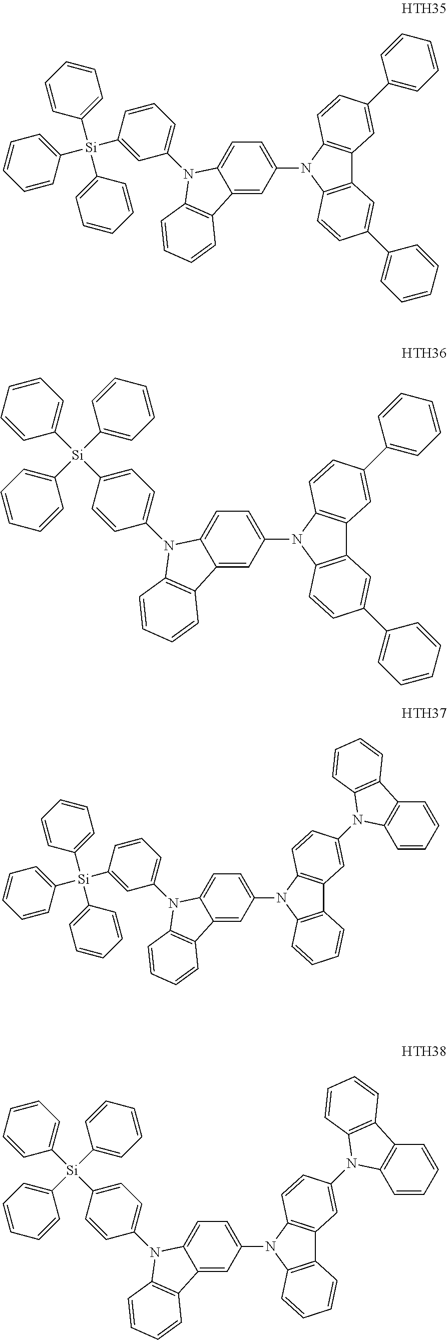 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1]

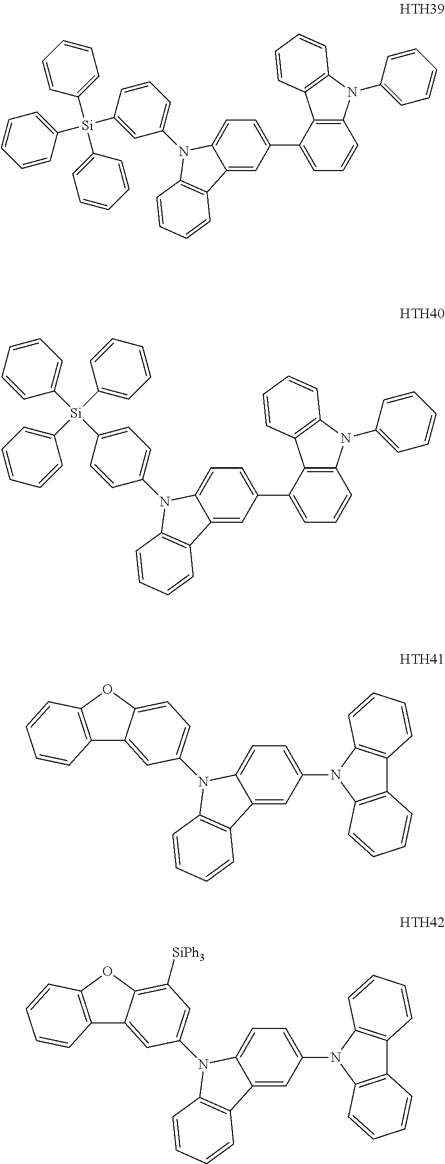

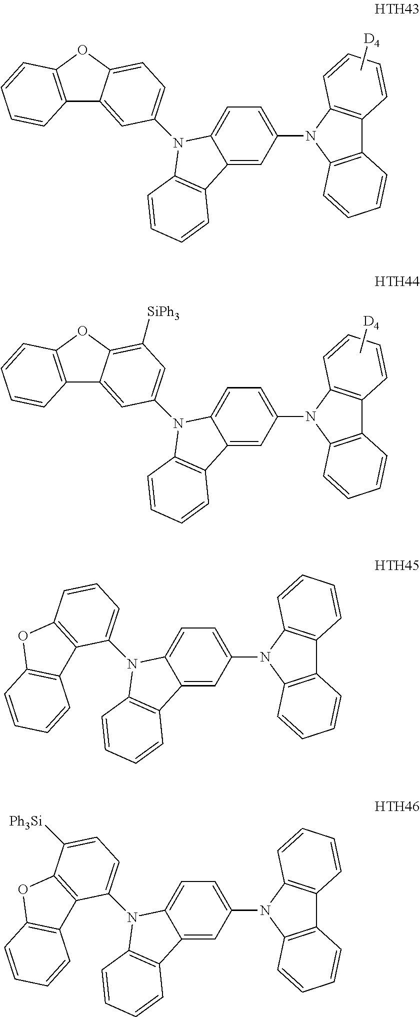

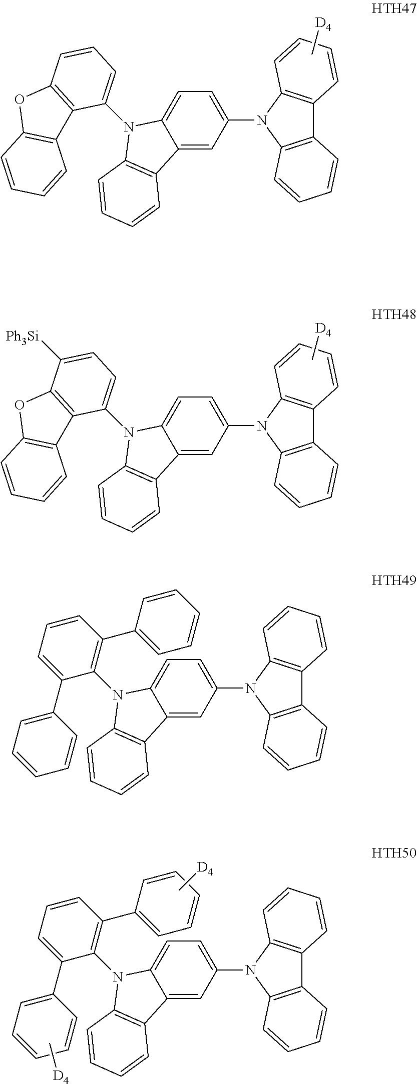

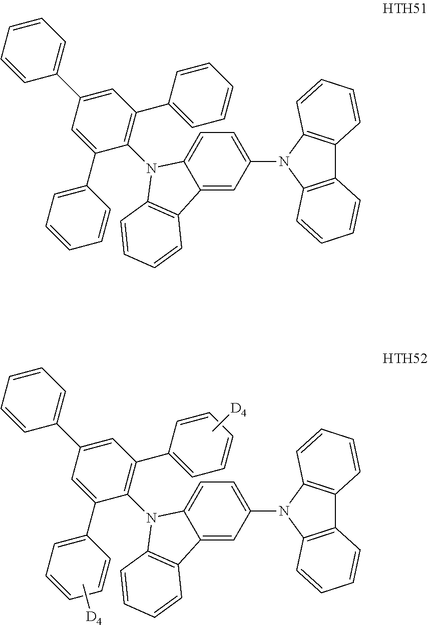

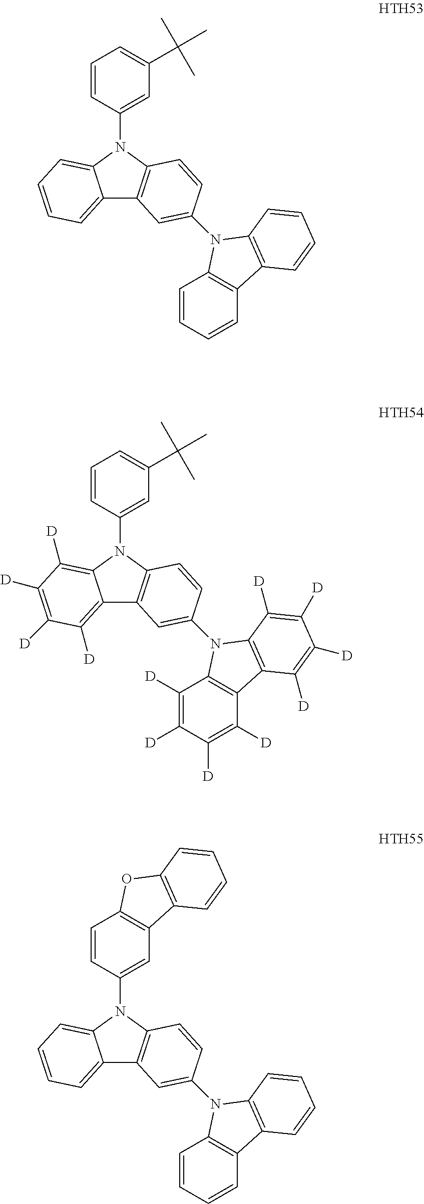 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

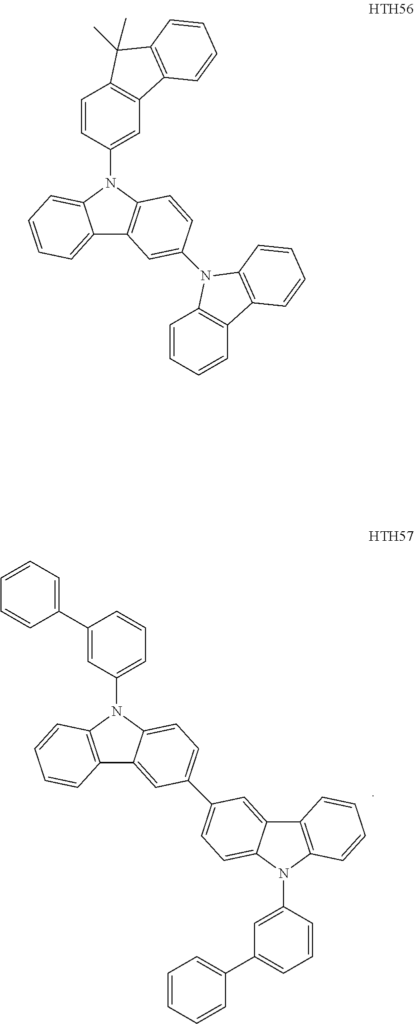 [SMILES: CC1(C)c2ccccc2-c2cc(-n3c4ccccc4c4cc(-n5c6ccccc6c6ccccc65)ccc43)ccc21 ; c1ccc(-c2cccc(-n3c4ccccc4c4cc(-c5ccc6c(c5)c5ccccc5n6-c5cccc(-c6ccccc6)c5)ccc43)c2)cc1]

In another embodiment, the third compound may include at least one of compound ETH1 to compound ETH87:

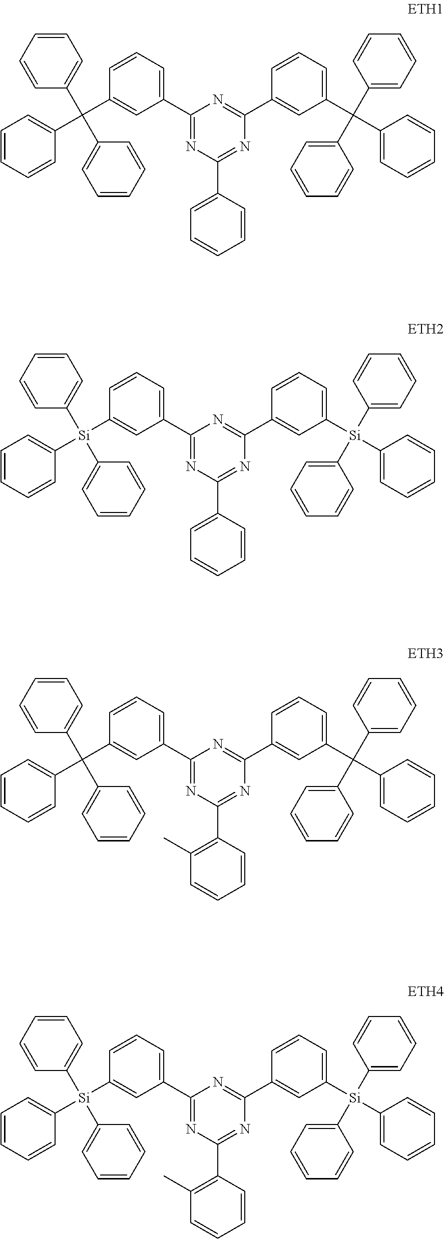 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

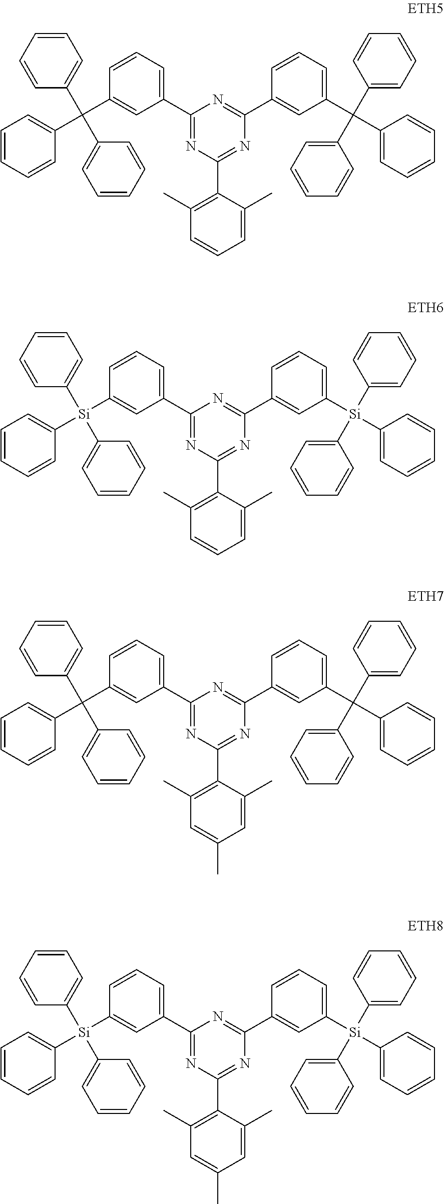 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

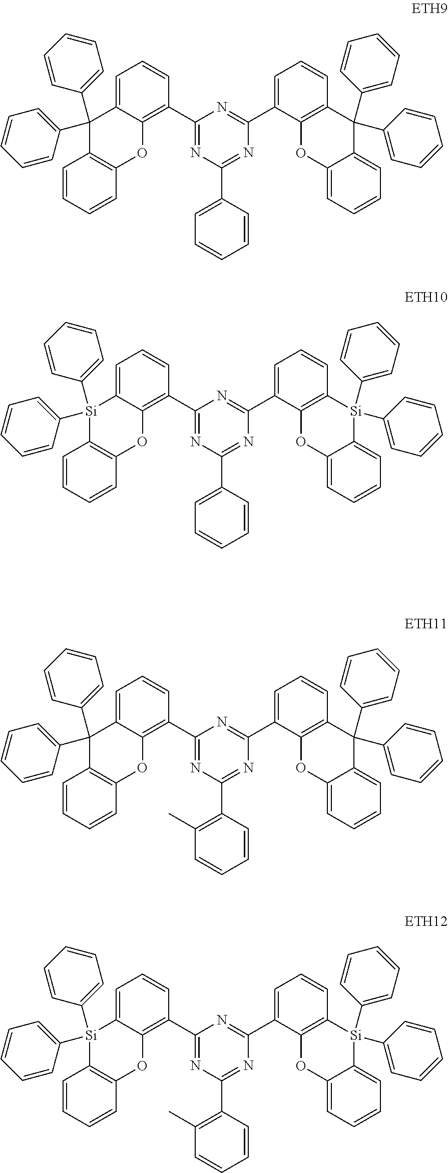 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

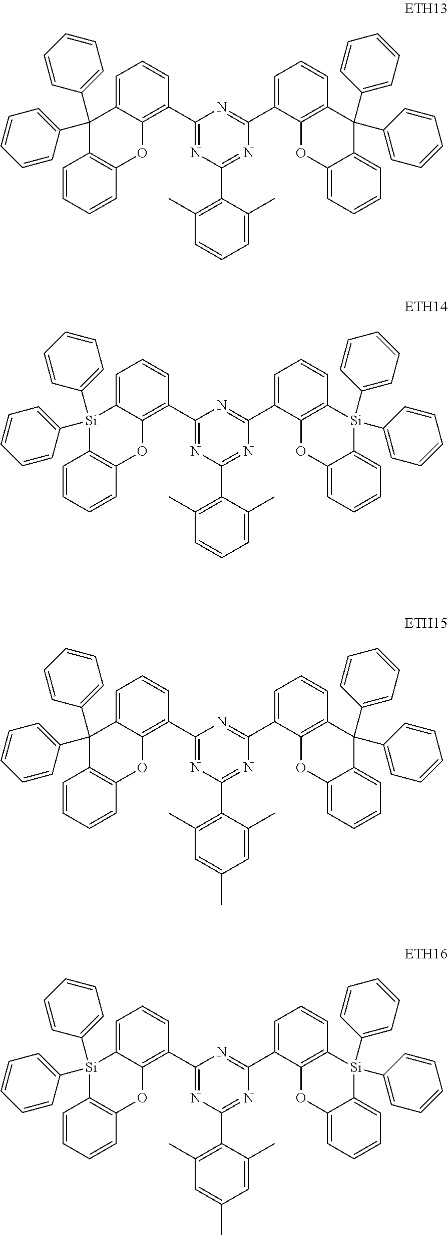 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

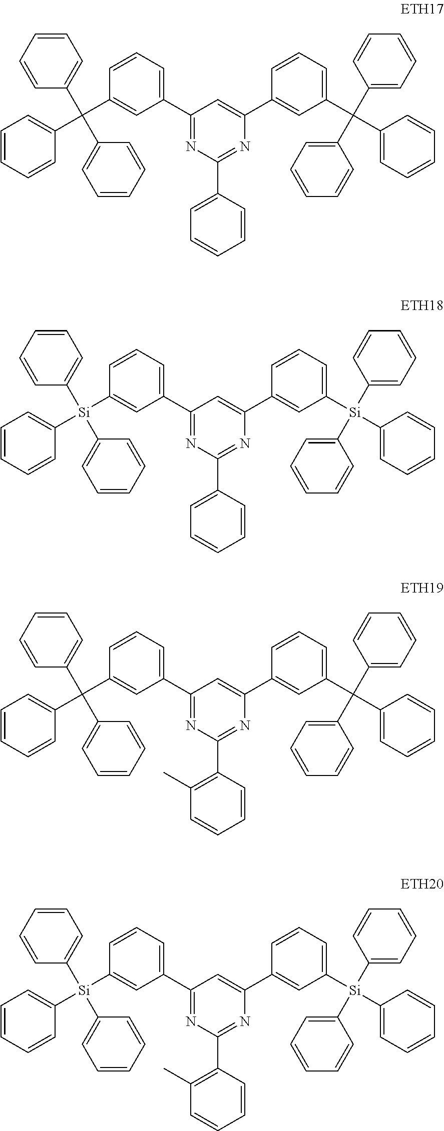 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

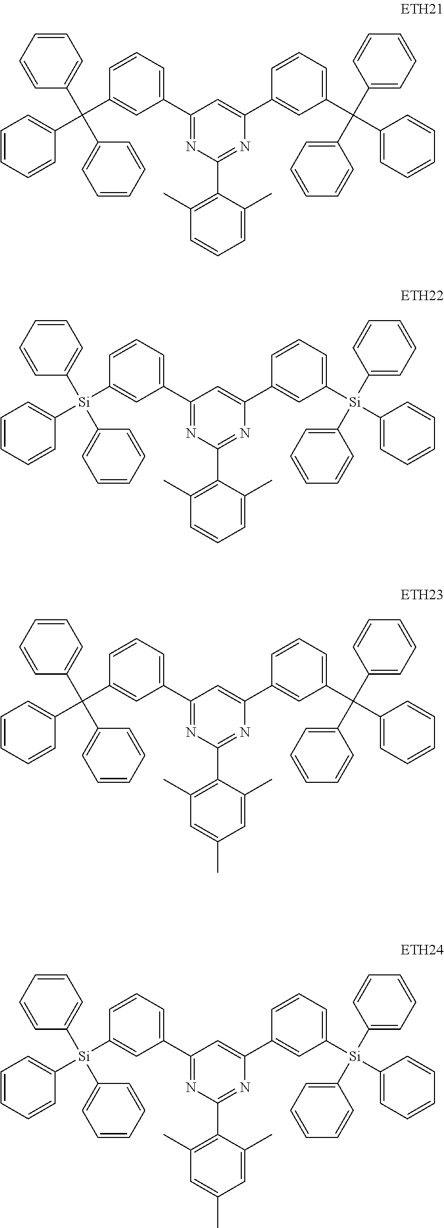 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

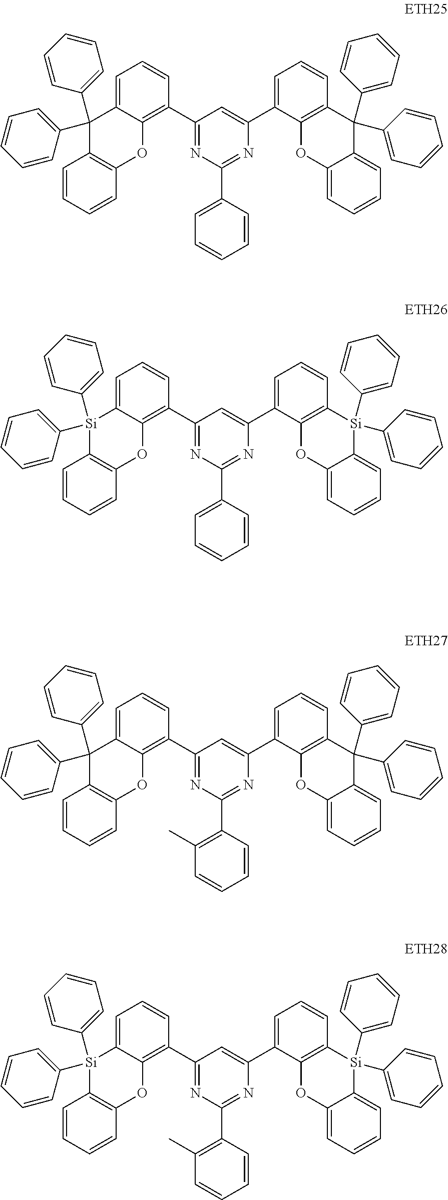 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

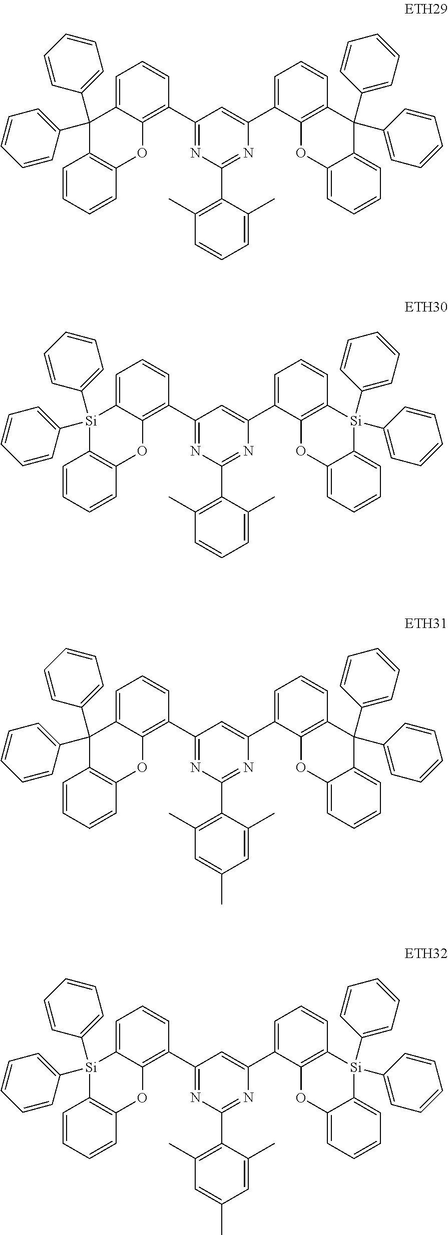 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

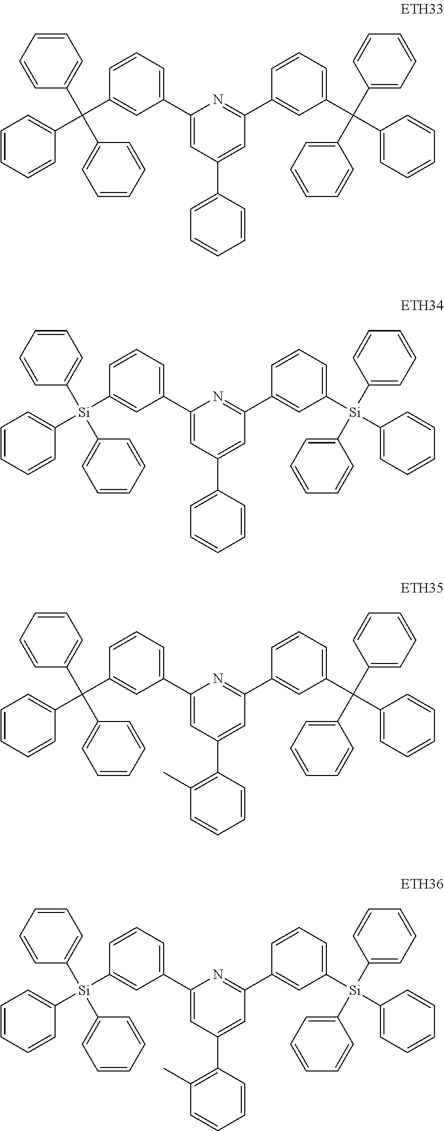 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

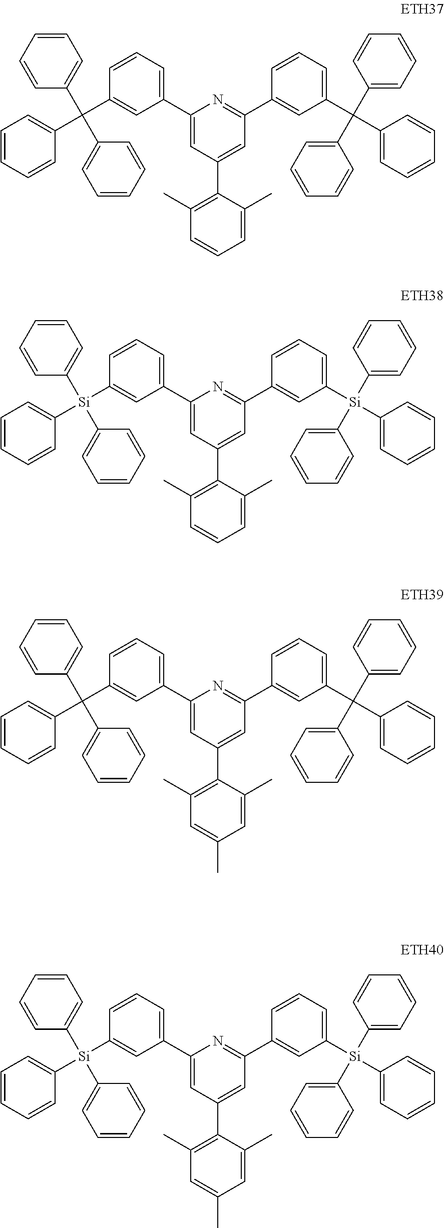 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

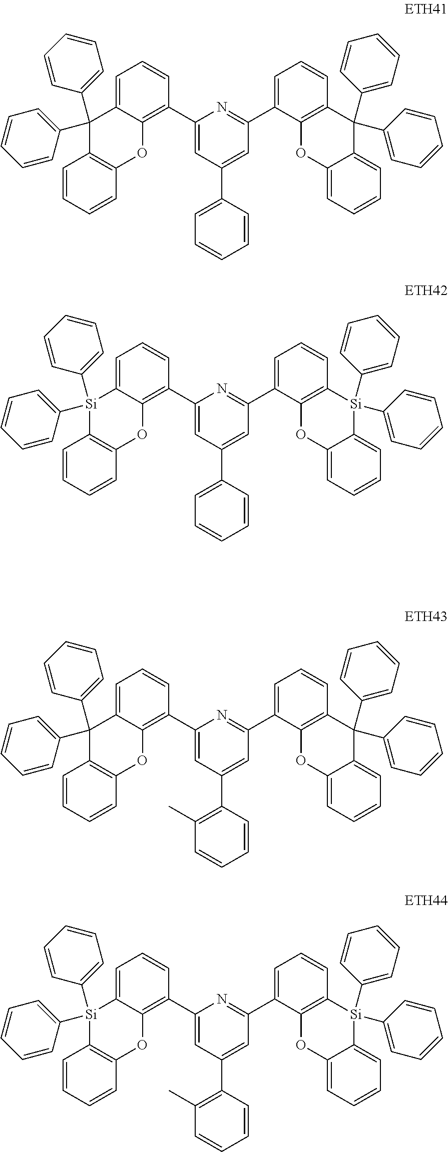 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

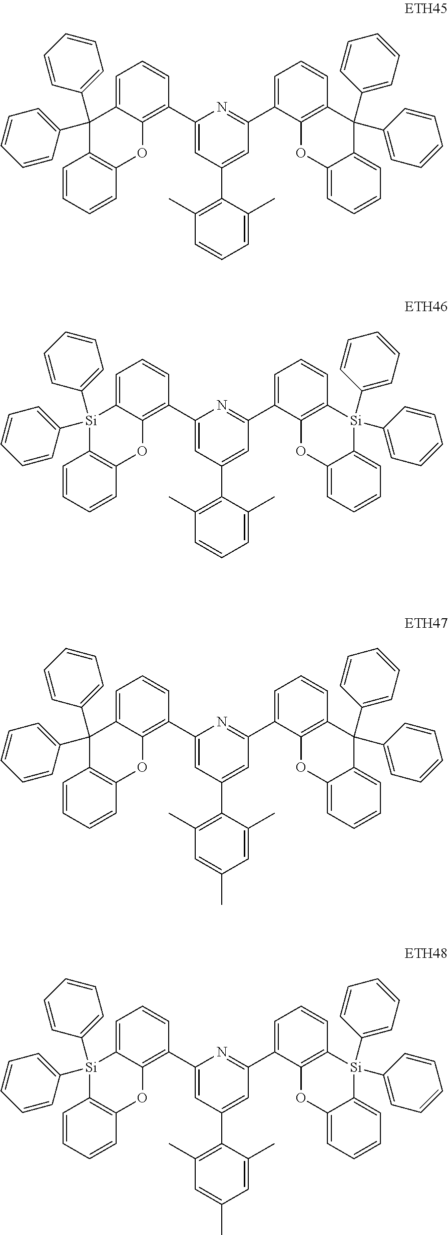 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

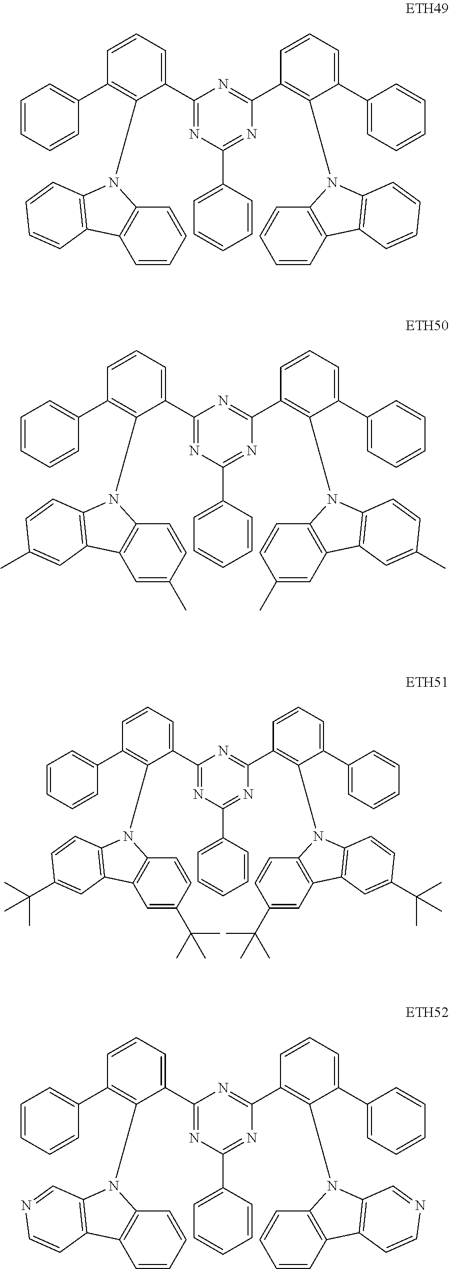

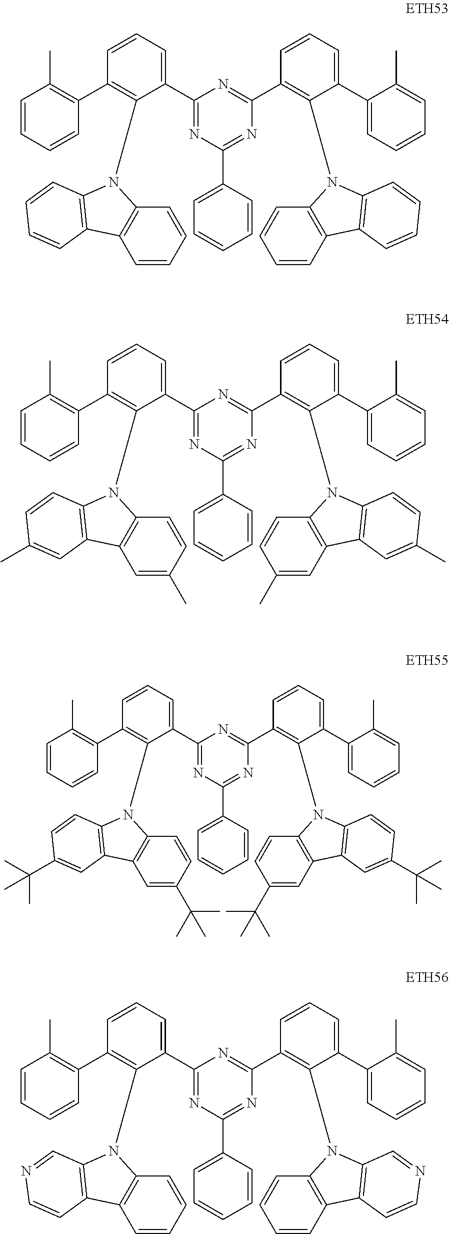

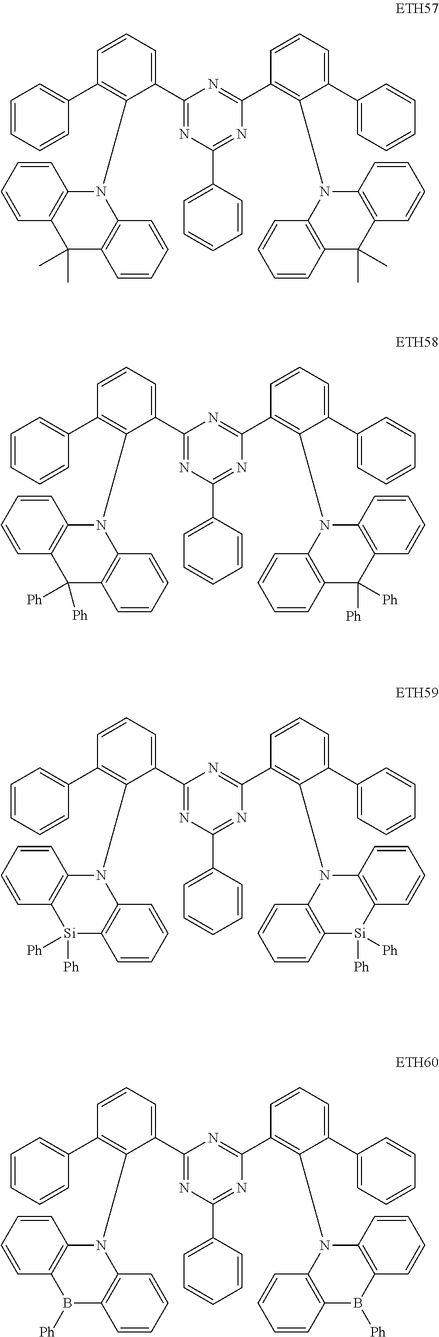

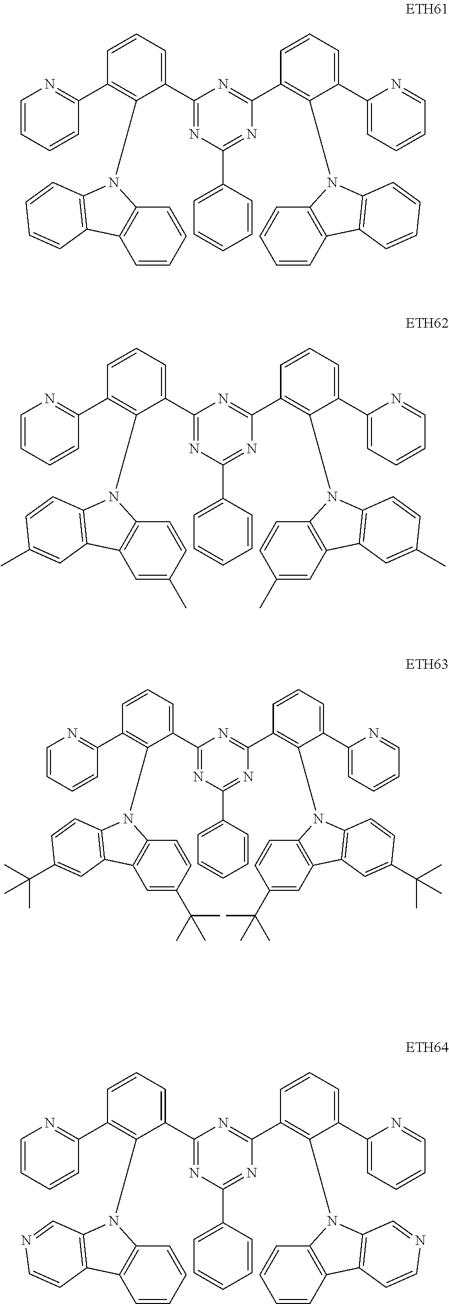

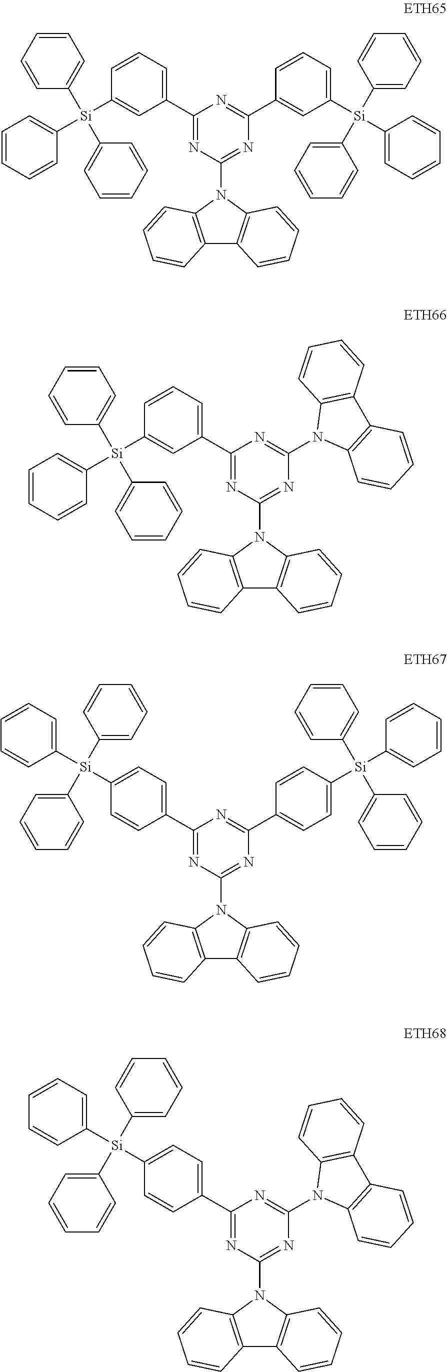 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

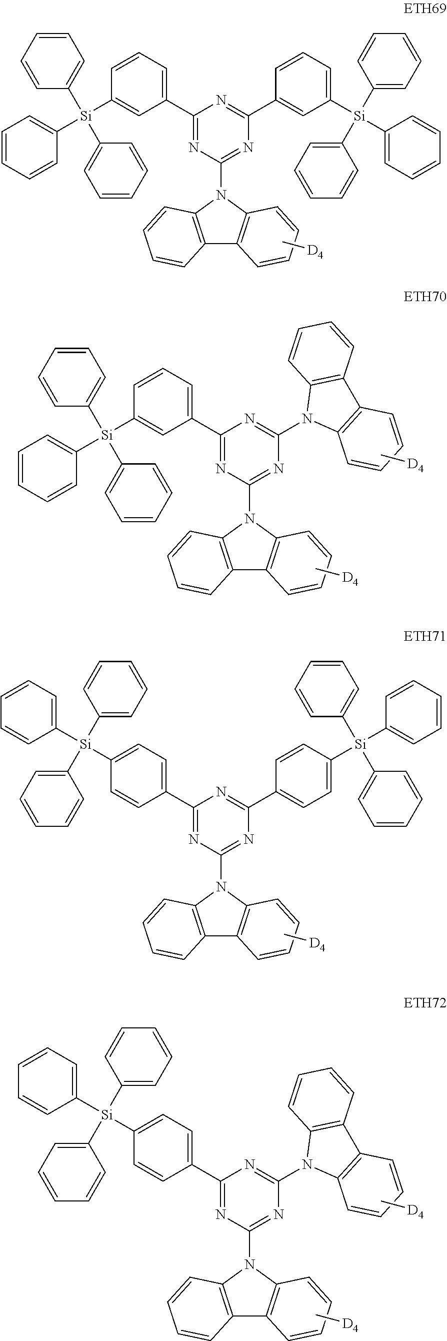

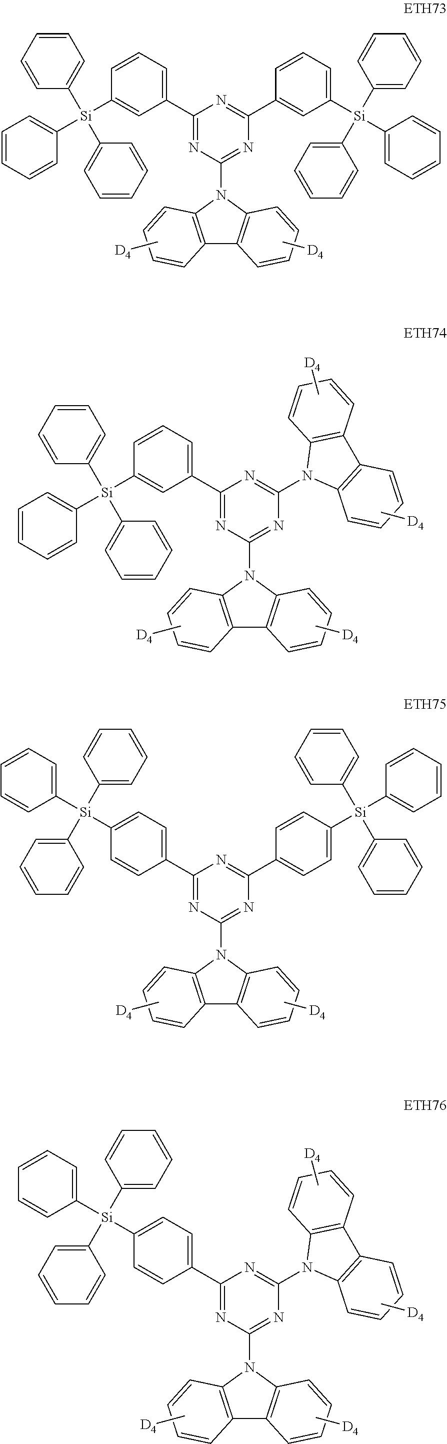

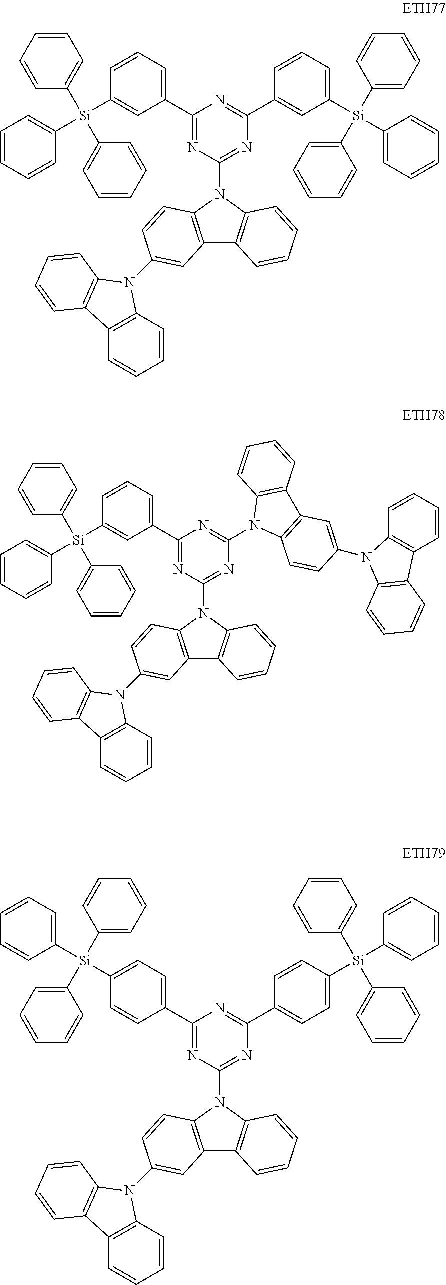 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

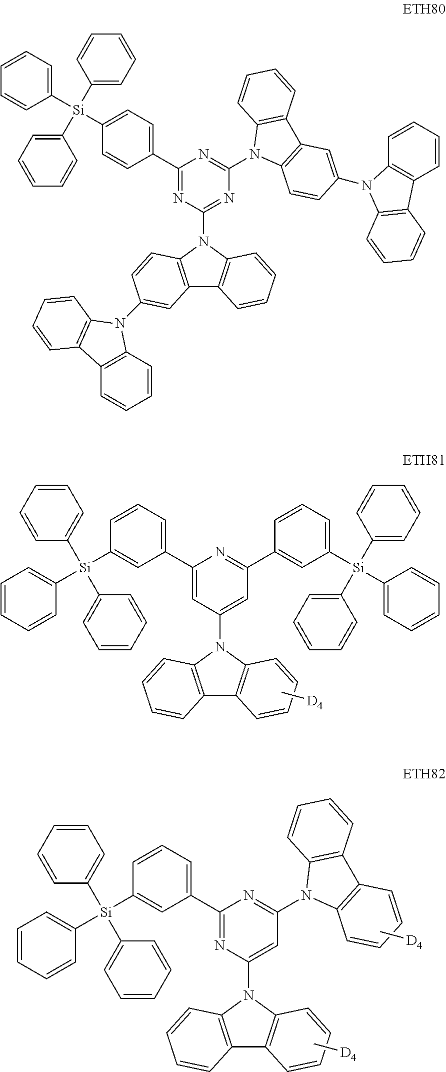

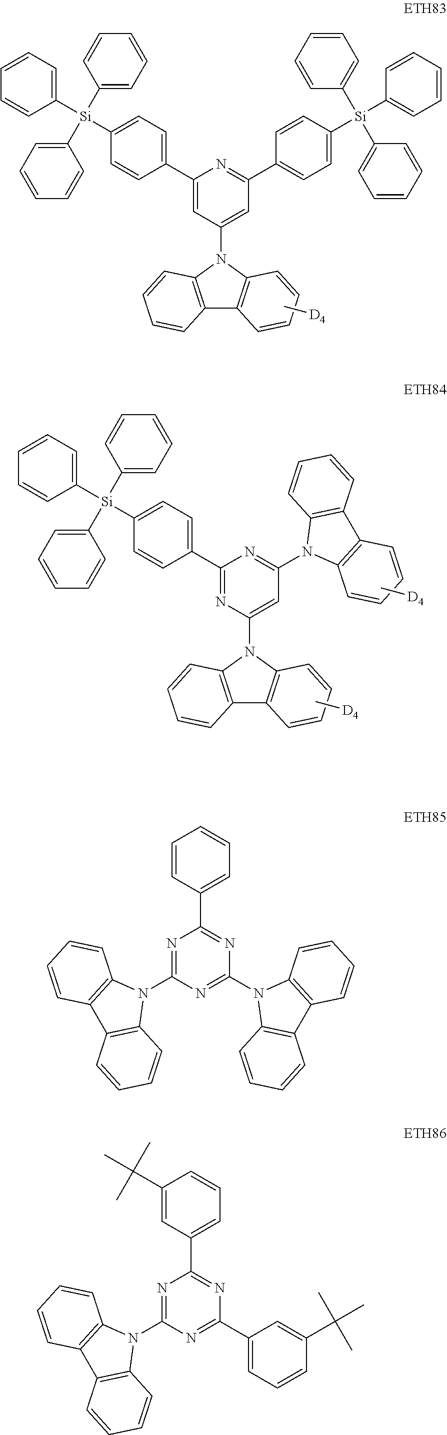

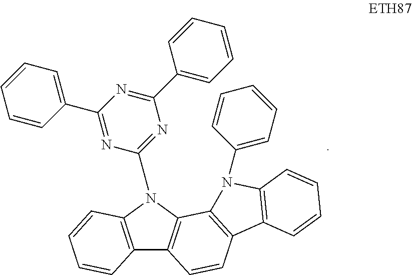 [SMILES: c1ccc(-c2nc(-c3ccccc3)nc(-n3c4ccccc4c4ccc5c6ccccc6n(-c6ccccc6)c5c43)n2)cc1]

In another embodiment, the fourth compound may include at least one of compound PD1 to compound PD42:

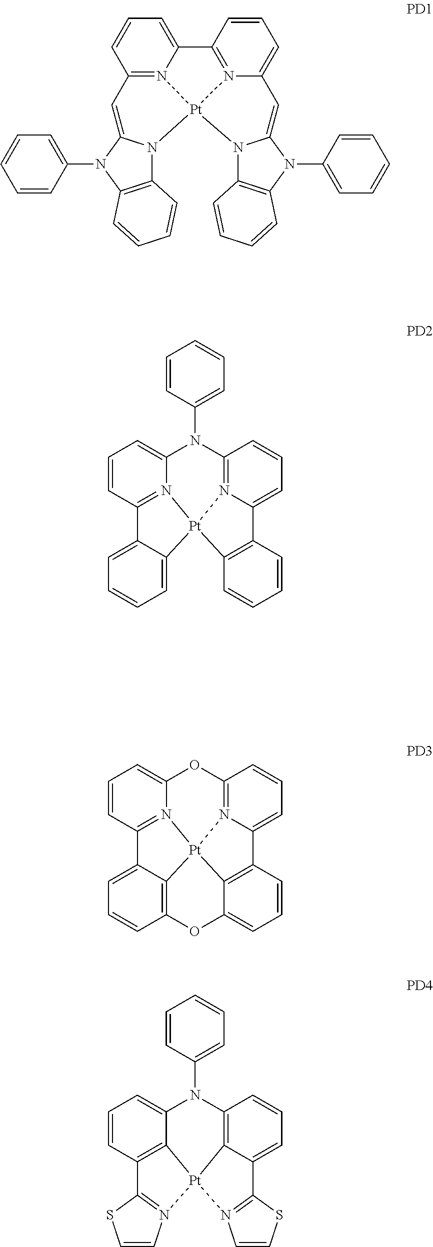

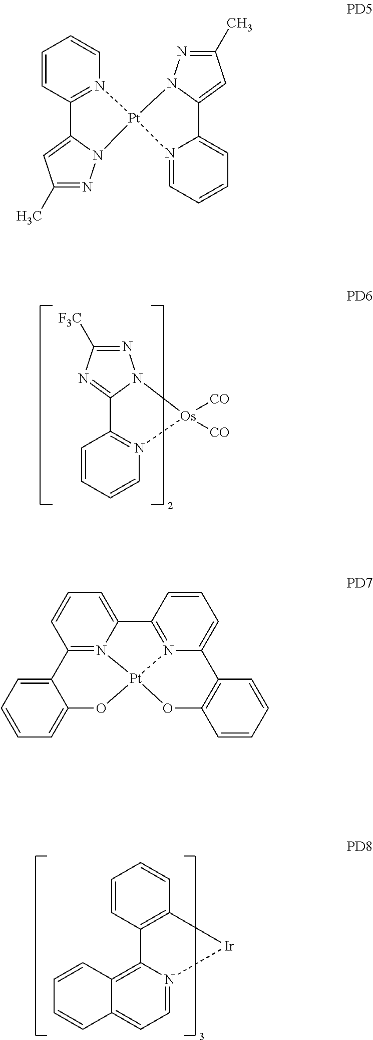

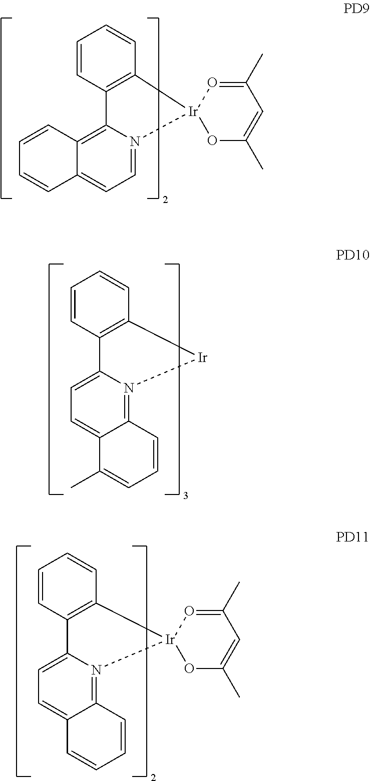

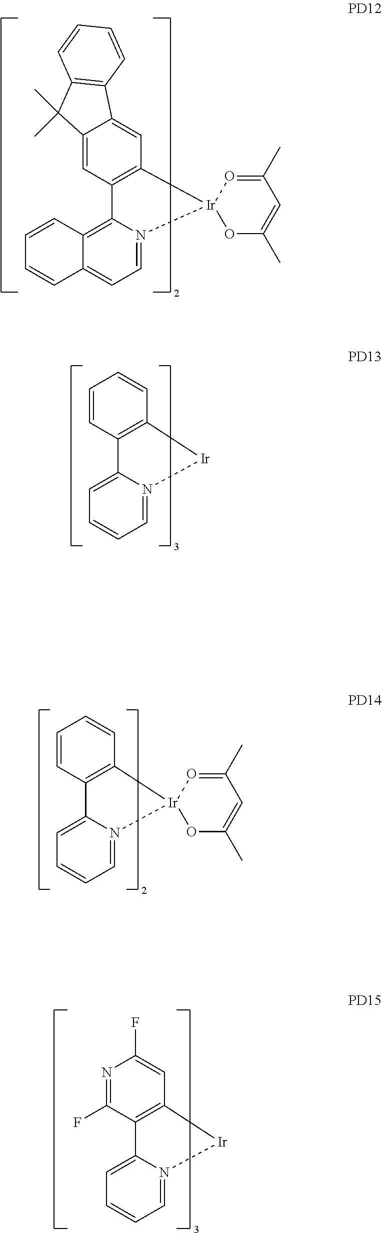

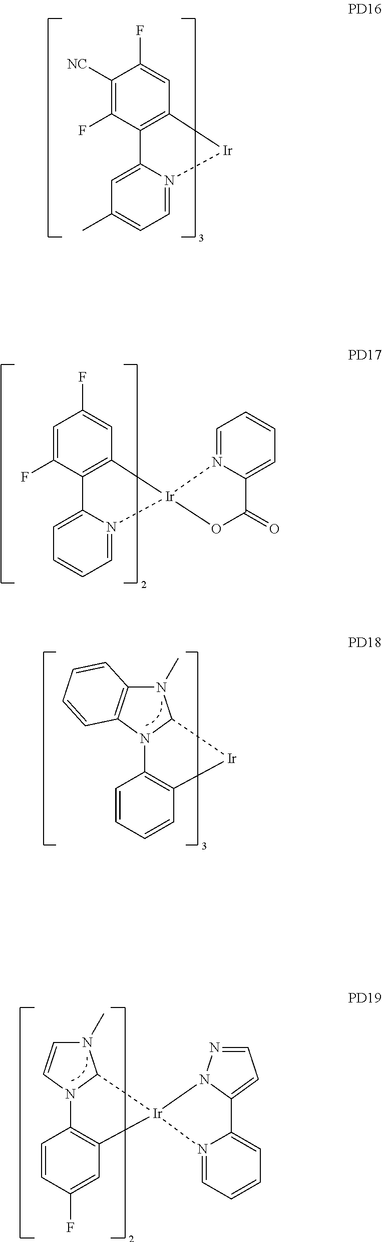

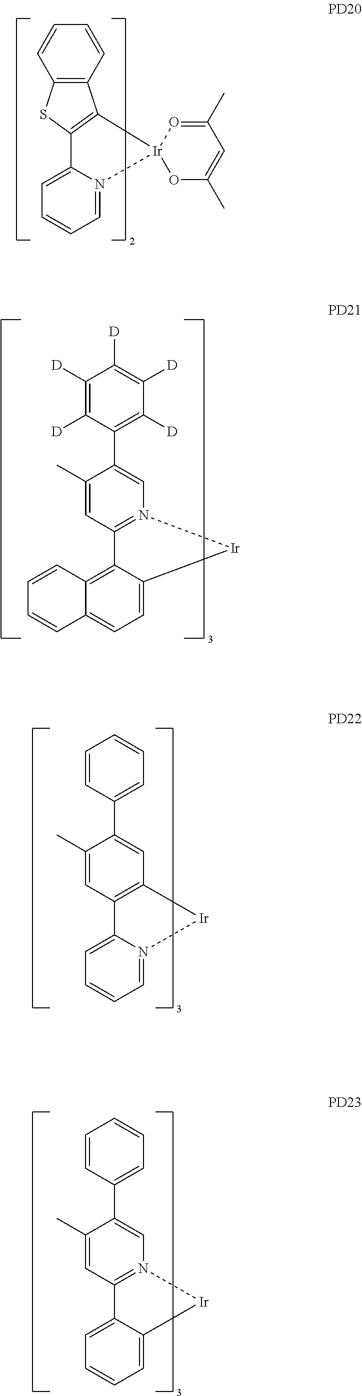

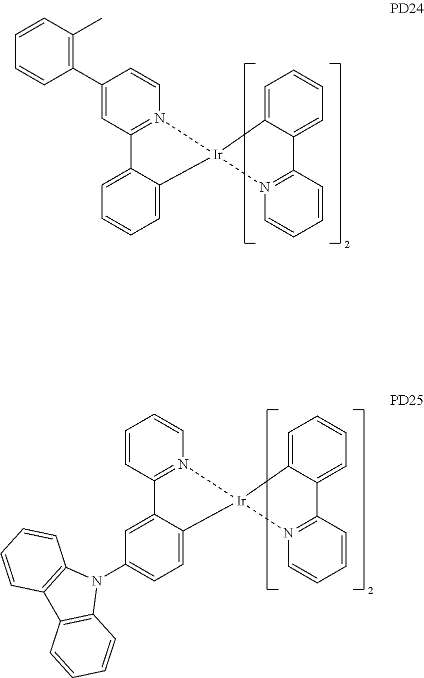

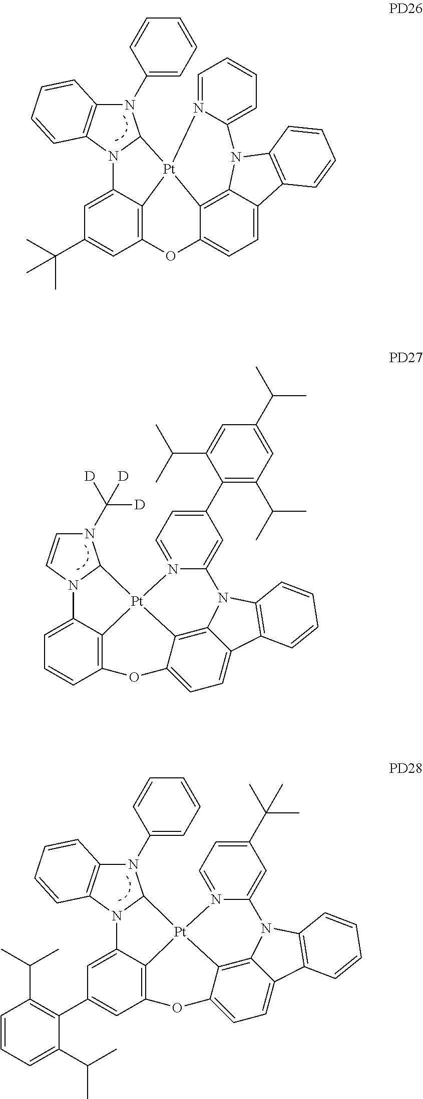

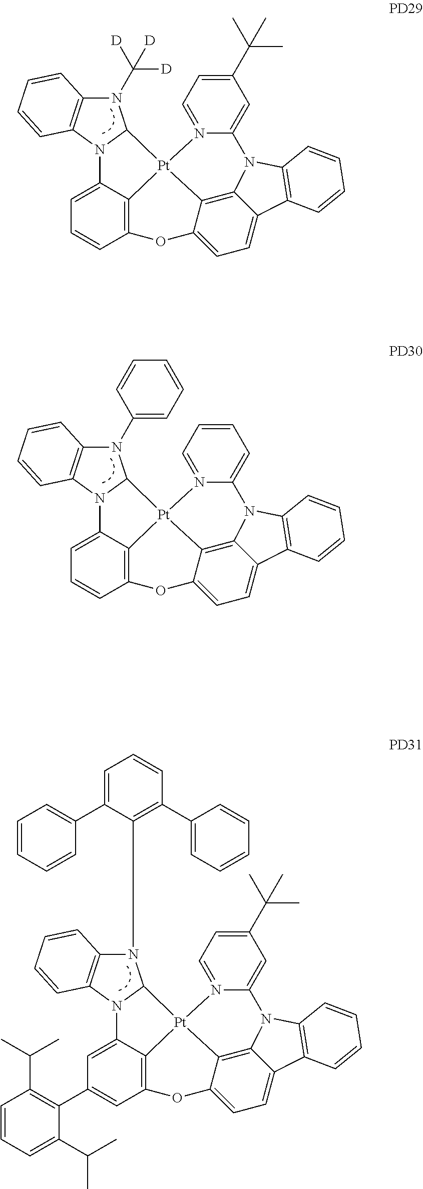

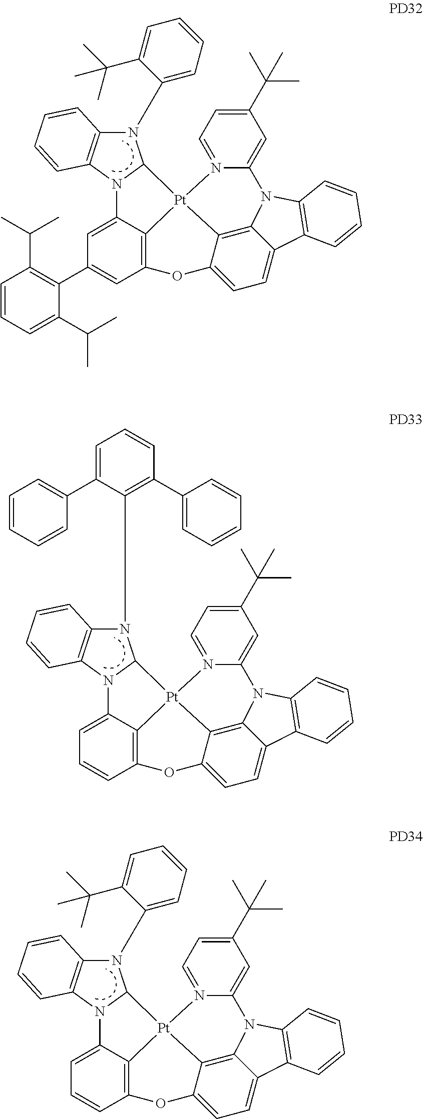

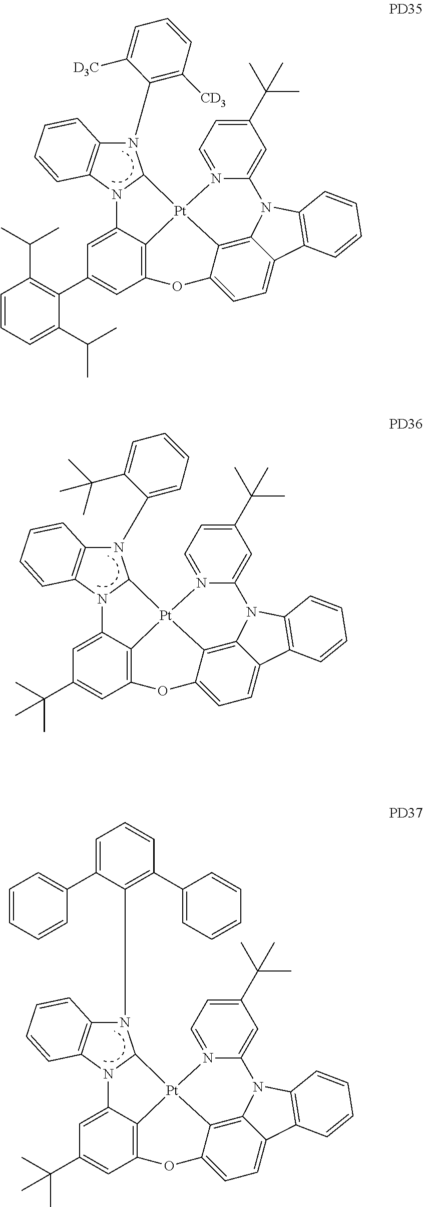

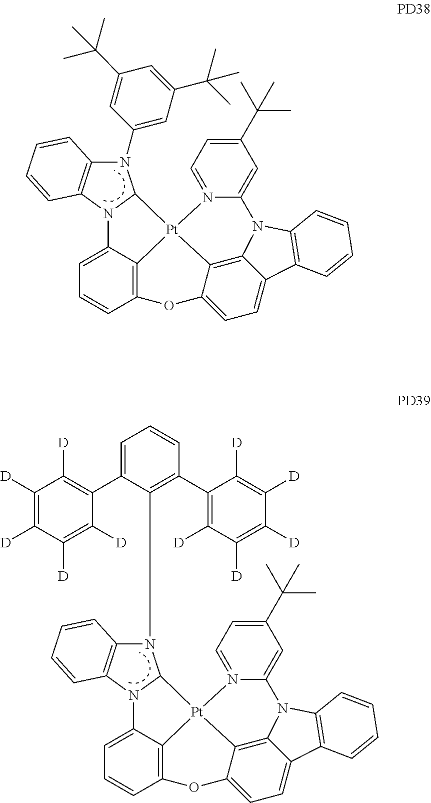

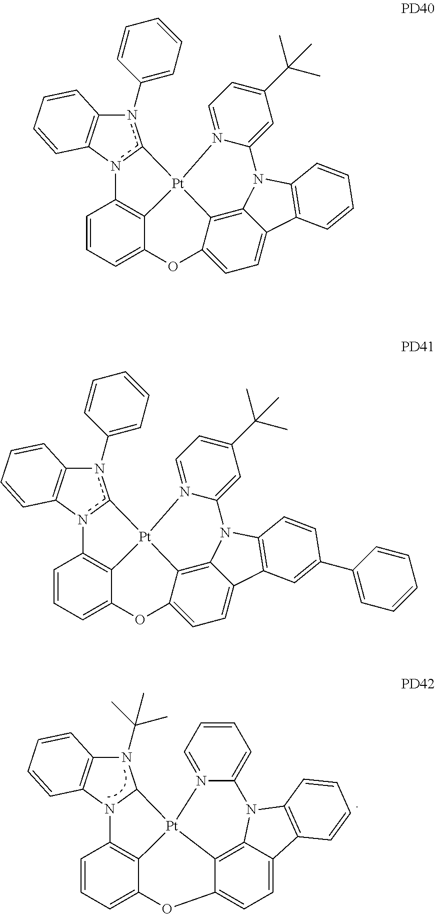

In the preceding compounds, Ph represents a phenyl group, D₅ represents substitution with five deuterium, and D₄ represents substitution with four deuterium. For example, a group represented by

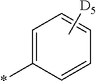

may be identical to a group represented by

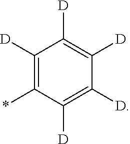

In one or more embodiments, the light-emitting device may satisfy at least one of Condition 1 to Condition 4:

### Condition 1

Lowest unoccupied molecular orbital (LUMO) energy level (electron volt (eV)) of the second compound >LUMO energy level (eV) of the fourth compound

### Condition 2

LUMO energy level (eV) of the fourth compound >LUMO energy level (eV) of the third compound

### Condition 3

Highest occupied molecular orbital (HOMO) energy level (eV) of the fourth compound >HOMO energy level (eV) of the second compound

### Condition 4

HOMO energy level (eV) of the second compound >HOMO energy level (eV) of the third compound.

Each of the HOMO energy level and LUMO energy level of each of the first compound, the second compound, and the third compound may be a negative value, and may be measured according to a known method.

In one or more embodiments, an absolute value of a difference between the LUMO energy level of the fourth compound and the LUMO energy level of the third compound may be about 0.1 eV or more and about 1.0 eV or less, an absolute value of a difference between the LUMO energy level of the fourth compound and the LUMO energy level of the second compound may be about 0.1 eV or more and about 1.0 eV or less, an absolute value of a difference between the HOMO energy level of the fourth compound and the HOMO energy level of the third compound may be about 1.25 eV or less (for example, about 1.25 eV or less and about 0.2 eV or more), and an absolute value of a difference between the HOMO energy level of the fourth compound and the HOMO energy level of the second compound may be about 1.25 eV or less (for example, about 1.25 eV or less and about 0.2 eV or more).

When the relationships between the LUMO energy level and HOMO energy level satisfy the aforementioned conditions, a balance between holes and electrons injected into the emission layer may be achieved.

The light-emitting device may have a structure of a first embodiment or a second embodiment:

### Description of First Embodiment

According to a first embodiment, the first compound may be included in a hole transport region of an interlayer of the light-emitting device, the fourth compound may be included in an emission layer of the interlayer of the light-emitting device, the emission layer may further include a host, the first compound and the host are different from each other, and the emission layer may emit phosphorescent light or fluorescent light emitted from the fourth compound. For example, according to the first embodiment, the fourth compound may be a dopant or an emitter.

The phosphorescent light or the fluorescent light emitted from the fourth compound may be red light, green light, or blue light.

The emission layer may further include an auxiliary dopant. The auxiliary dopant may act as a sensitizer to effectively transfer energy to the fourth compound, which is a dopant or an emitter, thereby enhancing the luminescence efficiency from the fourth compound.

The auxiliary dopant may be different from each of the fourth compound and the host.

For example, the auxiliary dopant may be a phosphorescent or fluorescent dopant.

### Description of Second Embodiment

According to a second embodiment, the first compound may be included in a hole transport region of an interlayer of the light-emitting device, the fourth compound may be included in an emission layer of an interlayer of the light-emitting device, the emission layer may further include a host and a dopant, and the fourth compound, the host, and the dopant may be different from each other, and the emission layer may emit phosphorescent light or fluorescent light (e.g., delayed fluorescence) emitted from the dopant.

For example, the fourth compound in the second embodiment may act as an auxiliary dopant that transfers energy to the dopant (or emitter), rather than a dopant.

As another example, the fourth compound of the second embodiment may act as an emitter and also act as an auxiliary dopant that transfers energy to the dopant (or emitter).

For example, the phosphorescence or fluorescence emitted from the dopant (or emitter) in the second embodiment may be red, green or blue phosphorescence or red, green or blue fluorescence (e.g., green or blue delayed fluorescence).

In the second embodiment, the dopant (or emitter) may be a phosphorescent dopant material (e.g., an organometallic compound represented by Formula 401 in the present specification) or any fluorescent dopant material (e.g., a compound represented by Formula 501 in the present specification, a compound represented by Formula 502, a compound represented by Formula 503, a delayed fluorescent compound, or any combination thereof).

The blue light in the first embodiment and the second embodiment may be blue light having a maximum emission wavelength in a range of about 390 nm to about 500 nm, about 410 nm to about 490 nm, about 430 nm to about 480 nm, about 440 nm to about 475 nm, or about 455 nm to about 470 nm. The green light may be green light having a maximum emission wavelength in the range of about 490 nm to about 560 nm. The red light may be red light having a maximum emission wavelength in the range of about 600 nm to about 750 nm.

The auxiliary dopant in the first embodiment may include, for example, the fourth compound represented by Formula 401 in this specification.

In one or more embodiments, the host in the first embodiment and the second embodiment may be any host material (e.g., a compound represented by Formula 301, a compound represented by 301-1, a compound represented by Formula 301-2, or any combination thereof).

In one or more embodiments, the host in the first embodiment and the second embodiment may be the second compound, the third compound, or any combination thereof.

Another aspect of the disclosure provides an electronic apparatus including the light-emitting device. The electronic apparatus may further include a thin-film transistor. For example, the electronic apparatus may further include a thin-film transistor including a source electrode and a drain electrode, wherein the first electrode of the light-emitting device may be electrically connected (coupled) to the source electrode or the drain electrode. In one or more embodiments, the electronic apparatus may further include a color filter, a color conversion layer, a touch screen layer, a polarizing layer, or any combination thereof. More details on the electronic apparatus may be referred to the descriptions provided herein.

### Description of Formula 1

According to one or more embodiments, the organic compound according to the disclosure may be represented by Formula 1:

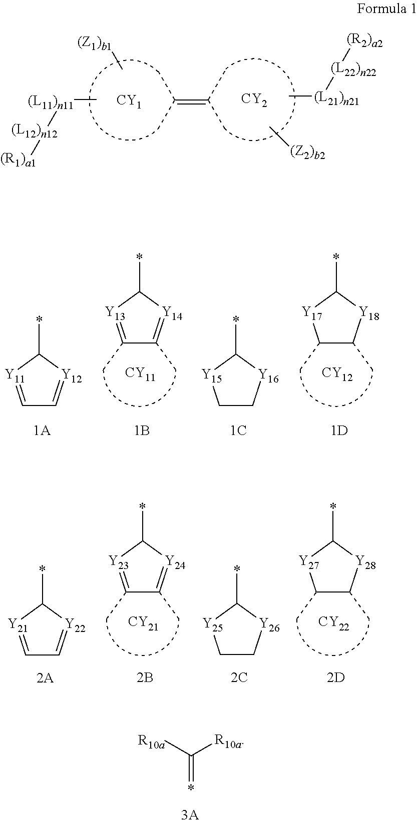

In Formulae 1, 1A to 1D, 2A to 2D, and 3A,
ring CY₁ may be represented by any one of Formulae 1A to 1D,
ring CY₂ may be represented by any one of Formulae 2A to 2D, and
ring CY₁₁, ring CY₁₂, ring CY₂₁, and ring CY₂₂ may each independently be a C₅-C₆₀ carbocyclic group or a C₁-C₆₀ heterocyclic group.

According to one or more embodiments, the organic compound represented by Formula 1 may satisfy any one of condition 1 to condition 16:

### Condition 1

Ring CY₁ may be represented by Formula 1A, and ring CY₂ may be represented by Formula 2A.

### Condition 2

Ring CY₁ may be represented by Formula 1A, and ring CY2 may be represented by Formula 2B.

### Condition 3

Ring CY₁ may be represented by Formula 1A, and ring CY₂ may be represented by Formula 2C.

### Condition 4

Ring CY₁ may be represented by Formula 1A, and ring CY₂ may be represented by Formula 2D.

### Condition 5

Ring CY₁ may be represented by Formula 1B, and ring CY₂ may be represented by Formula 2A.

### Condition 6

Ring CY₁ may be represented by Formula 1B, and ring CY₂ may be represented by Formula 2B.

### Condition 7

Ring CY₁ may be represented by Formula 1B, and ring CY₂ may be represented by Formula 2C.

### Condition 8

Ring CY₁ may be represented by Formula 1B, and ring CY₂ may be represented by Formula 2D.

### Condition 9

Ring CY₁ may be represented by Formula 1C, and ring CY₂ may be represented by Formula 2A.

### Condition 10

Ring CY₁ may be represented by Formula 1C, and ring CY₂ may be represented by Formula 2B.

### Condition 11

Ring CY₁ may be represented by Formula 1C, and ring CY₂ may be represented by Formula 2C.

### Condition 12

Ring CY₁ may be represented by Formula 1C, and ring CY₂ may be represented by Formula 2D.

### Condition 13

Ring CY₁ may be represented by Formula 1A, and ring CY₂ may be represented by Formula 2A.

### Condition 14

Ring CY₁ may be represented by Formula 1A, and ring CY₂ may be represented by Formula 2B.

### Condition 15

Ring CY₁ may be represented by Formula 1A, and ring CY₂ may be represented by Formula 2C.

### Condition 16

Ring CY₁ may be represented by Formula 1A, and ring CY₂ may be represented by Formula 2D.

In one or more embodiments, ring CY₁₁, ring CY₁₂, ring CY₂₁, and ring CY₂₂ may each independently be
a benzene group, a naphthalene group, an anthracene group, a phenanthrene group, a triphenylene group, a pyrene group, a chrysene group, a cyclopentene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a dibenzooxasiline group, a dibenzothiasiline group, a dibenzodihydroazasiline group, a dibenzodihydrodihydrodisiline group, a dibenzodihydrosiline group, a dibenzodioxine group, a dibenzooxathiine group, a dibenzooxazine group, a dibenzopyran group, a dibenzodithiine group, a dibenzothiazine group, a dibenzothiopyran group, a dibenzocyclohexadiene group, a dibenzodihydropyridine group, or a dibenzodihydropyrazine group.

In one or more embodiments, ring CY₁₁, ring CY₂₁, ring CY₁₂, and ring CY₂₂ may each independently be a benzene group, a cyclopentene group, a cyclopentadiene group, a furan group, or a thiophene group.

In one or more embodiments, Y₁₁ may be C(R₁₁), Si(R₁₁), or N.

In one or more embodiments, Y₁₂ may be C(R₁₂), Si(R₁₂), or N.

In one or more embodiments, Y₁₃ may be C(R₁₃), Si(R₁₃), or N.

In one or more embodiments, Y₁₄ may be C(R₁₄), Si(R₁₄), or N.

In one or more embodiments, Y₁₅ may be C(R₁₅)(R₁₅′), Si(R₁₅)(R₁₅′), N(R₁₅), O, S, or Se.

In one or more embodiments, Y₁₆ may be C(R₁₆)(R₁₆′), Si(R₁₆)(R₁₆′), N(R₁₆), O, S, or Se.

In one or more embodiments, Y₁₇ may be C(R₁₇)(R₁₇′), Si(R₁₇)(R₁₇′), N(R₁₇), O, S, or Se.

In one or more embodiments, Y₁₈ may be C(R₁₈)(R₁₈′), Si(R₁₈)(R₁₈′), N(R₁₈), O, S, or Se.

In one or more embodiments, Y₂₁ may be C(R₂₁), Si(R₂₁), or N.

In one or more embodiments, Y₂₂ may be C(R₂₂), Si(R₂₂), or N.

In one or more embodiments, Y₂₃ may be C(R₂₃), Si(R₂₃), or N.

In one or more embodiments, Y₂₄ may be C(R₂₄), Si(R₂₄), or N.

In one or more embodiments, Y₂₅ may be C(R₂₅)(R₂₅′), Si(R₂₅)(R₂₅′), N(R₂₅), O, S, or Se.

In one or more embodiments, Y₂₆ may be C(R₂₆)(R₂₆′), Si(R₂₆)(R₂₆′), N(R₂₆), O, S, or Se.

In one or more embodiments, Y₂₇ may be C(R₂₇)(R₂₇′), Si(R₂₇)(R₂₇′), N(R₂₇), O, S, or Se.

In one or more embodiments, Y₂₈ may be C(R₂₈)(R₂₈′), Si(R₂₈)(R₂₈′), N(R₂₈), O, S, or Se.

According to one or more embodiments, L₁₁, L₁₂, L₂₁, and L₂₂ may each independently be a single bond (e.g., a single covalent bond), *—C(R₃)(R₄)—*′, *—Si(R₃)(R₄)—*′, *—Ge(R₃)(R₄)—*′, *—S—*′, *—Se—*′, *—O—*′, *—C(═O)—*′, *—S(═O)—*′, *—S(═O)₂—*′, *═C(R₃)—*′, *—C(R₃)═C(R₄)—*′, *—C(═S)—*′, *—C≡C—*′, a C₅-C₆₀ carbocyclic group unsubstituted or substituted with at least one R₁₀ₐ, or a C₁-C₆₀ heterocyclic group unsubstituted or substituted with at least one R₁₀ₐ.

In one or more embodiments, L₁₁, L₁₂, L₂₁, and L₂₂ may each independently be
a single bond (e.g., a single covalent bond), *—C(R₃)(R₄)—*′, *—Si(R₃)(R₄)—*′, *—Ge(R₃)(R₄)—*′, *—S—*′, *—Se*′, *—O—*′, *—C(═O)—*′, *—S(═O)—*′, *—S(═O)₂—*′, *═C(R₃)—*′, *—C(R₃)═C(R₄)—*′, *—C(═S)—*′, *—C≡C—*′, a benzene group, a naphthalene group, a phenanthrene group, a fluorene group, a cyclopentene group, a cyclopentadiene group, a carbazole group, a dibenzofuran group, a dibenzothiophene group, an indole group, a pyridine group, a pyrimidine group, a triazine group, an azacarbazole group, an azadibenzofuran group, or an azadibenzothiophene group, each unsubstituted or substituted with at least one R₁₀ₐ.

In one or more embodiments, L₁₁, L₁₂, L₂₁, and L₂₂ may each independently be
a single bond (e.g., a single covalent bond), *—C(R₃)(R₄)—*′, *—S—*′, *—C(═O)—*′, *═C(R₃)*′, *—C(═S)—*′, or Formulae 2-1 to 2-15:

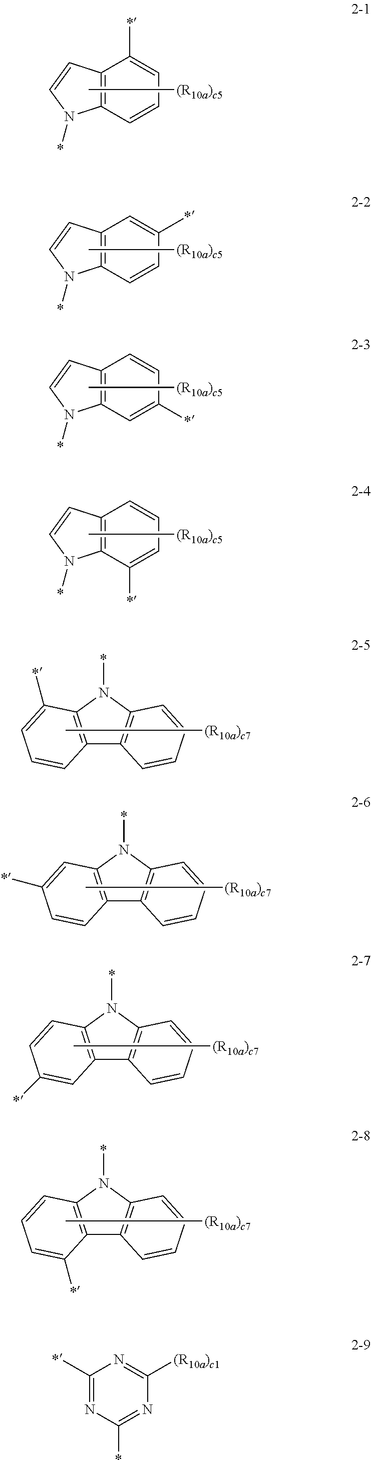

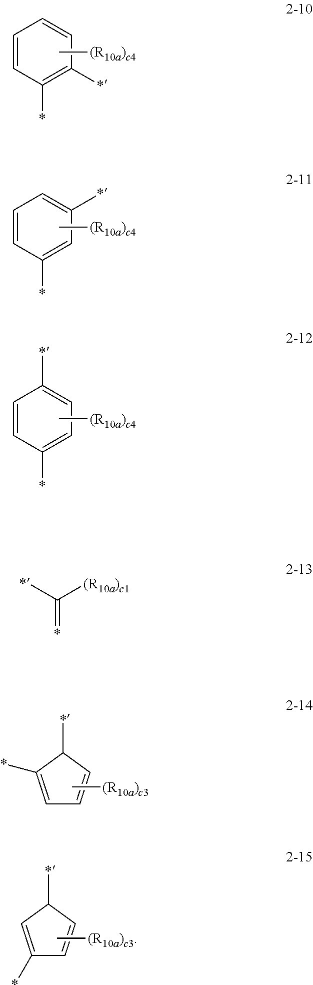

In Formulae 2-1 to 2-15,
R₁₀ₐ is as described herein,
c1 may be an integer from 0 to 1,
c3 may be an integer from 0 to 3,
c4 may be an integer from 0 to 4,
c5 may be an integer from 0 to 5,
c7 may be an integer from 0 to 7, and
* and *′ each indicate a binding site to a neighboring atom.

According to one or more embodiments, n11, n12, n21, and n22 may each independently be an integer from 1 to 10. For example, n11, n12, n21, and n22 may each independently be an integer from 1 to 5.

According to one or more embodiments, R₁ to R₄, R₁₁ to R₁₈, R₁5′ to R₁₈′, R₂₁ to R₂₈, R25′ to R₂₈′, Z₁, and Z₂ may each independently be a group represented by Formula 3A, hydrogen, deuterium, —F, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group, a C₆-C₆₀ arylthio group unsubstituted or substituted with at least one R₁₀ₐ, a C₆-C₆₀ arylseleno group unsubstituted or substituted with at least one R₁₀ₐ, a C₇-C₆₀ arylalkyl group unsubstituted or substituted with at least one R₁₀ₐ, a C₂-C₆₀ heteroaryl alkyl group unsubstituted or substituted with at least one R₁₀ₐ, —Si(Q₁)(Q₂)(Q₃), —Ge(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In one or more embodiments, R₁ to R₄, R₁₁ to R₁₈, R₁₅′ to R₁₈′, R₂₁ to R₂₈, R₂₅′ to R₂₈′, Z₁, and Z₂ may each independently be selected from:
a group represented by Formula 3A, hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a phenyl group, a biphenyl group, and a terphenyl group;
a C₁-C₆₀ alkyl group, a C₁-C₆₀ cycloalkyl group, a C₁-C₆₀ heterocycloalkyl group, a phenyl group, a biphenyl group, a terphenyl group, a naphthyl group, an anthracenyl group, a phenanthrenyl group, a phenalenyl group, a triphenylenyl group, a pyrenyl group, a chrysenyl group, a pyrrolyl group, a furanyl group, a thiophenyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a dibenzofuranyl group, a benzothiophenyl group, a dibenzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a carbazolyl group, a benzocarbazolyl group, a fluorenyl group, a benzoisothiazolyl group, a benzoxazolyl group, a benzoisoxazolyl group, a phenoxazinyl group, an acridinyl group, and a xanthenyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a phenyl group, a biphenyl group, a terphenyl group, or any combination thereof; and

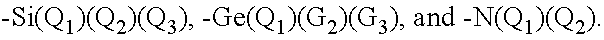

According to one or more embodiments, two or more adjacent substituents among R₁ to R₄, R₁₁ to R₁₈, R₁₅′ to R₁₈′, R₂₁ to R₂₈, R₂₅′ to R₂₈′, Z₁, and Z₂ may be optionally bonded to each other to form (provide) a C₅-C₃₀ carbocyclic group unsubstituted or substituted with at least one R₁₀ₐ, or a C₂-C₃₀ heterocyclic group unsubstituted or substituted with at least one R₁₀ₐ.

In one or more embodiments, a1 may be an integer from 0 to 30.

In one or more embodiments, a2 may be an integer from 0 to 30.

In one or more embodiments, b1 may be an integer from 0 to 10.

In one or more embodiments, b2 may be an integer from 0 to 10.

In one or more embodiments, * may indicate a binding site to a double bond (e.g., a double covalent bond) of Formula 1 and may indicate a binding site to a neighboring atom.

According to one or more embodiments, the organic compound represented by Formula 1 may include at least one cyano group.

In one or more embodiments, R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

According to one or more embodiments, the organic compound represented by Formula 1 may be represented by any one of Formulae 1-1 to 1-5:

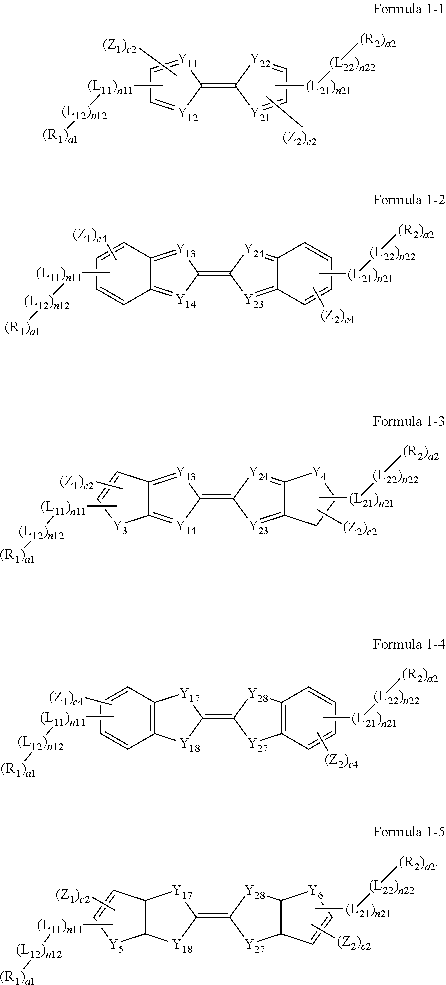

In Formulae 1-1 to 1-5,
Y₁₁, Y₁₂, Y₂₁, Y₂₂, Y₁₃, Y₁₄, Y₂₃, Y₂₄, Y₁₇, Y₁₈, Y₂₇, Y₂₈, L₁₁, L₁₂, L₂₁, L₂₁, n11, n12, n21, n22, R₁, R₂, a1, a2, Z₁, and Z₂ are as described herein,
Y₃ may be C(R₃₁)(R₃₂), N(R₃₁), O, or S,
Y₄ may be C(R₄₁)(R₄₂), N(R₄₁), O, or S,
Y₅ may be C(R₅₁)(R₅₂), N(R₅₁), O, or S,
Y₆ may be C(R₆₁)(R₆₂), N(R₆₁), O, or S,
c2 may be an integer from 0 to 2,
c4 may be an integer from 0 to 4, and
R₃₁, R₃₂, R₄₁, R₄₂, R₅₁, R₅₂, R₆₁, and R₆₂ are as described in connection with R₁.

According to one or more embodiments, the organic compound represented by Formula 1 may be represented by any one of (e.g., selected from among) Formula 1-1-A to Formula 1-1-D, Formula 1-2-A to Formula 1-2-D, Formula 1-3-A to 1-3-C, Formula 1-4-A, and Formula 1-5-A:

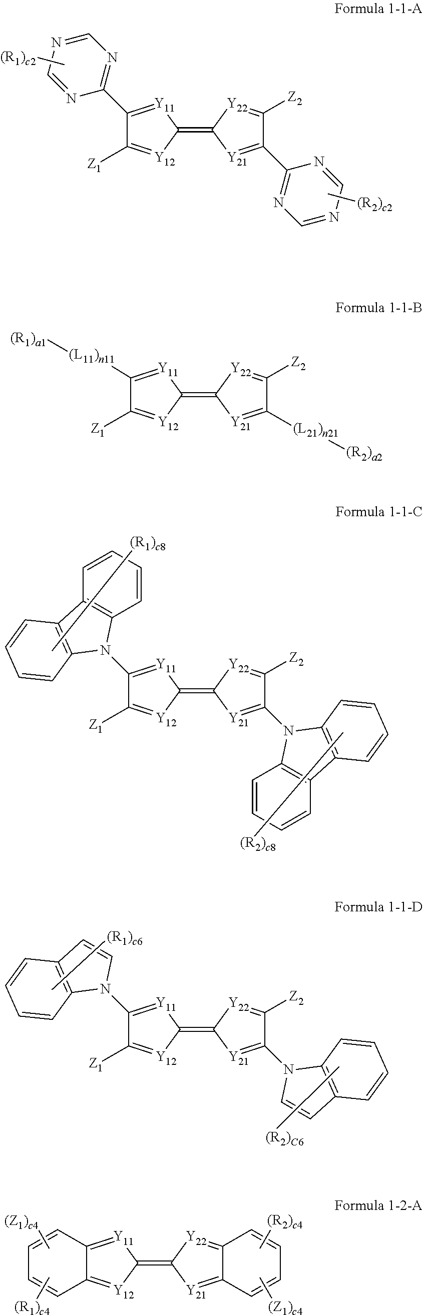

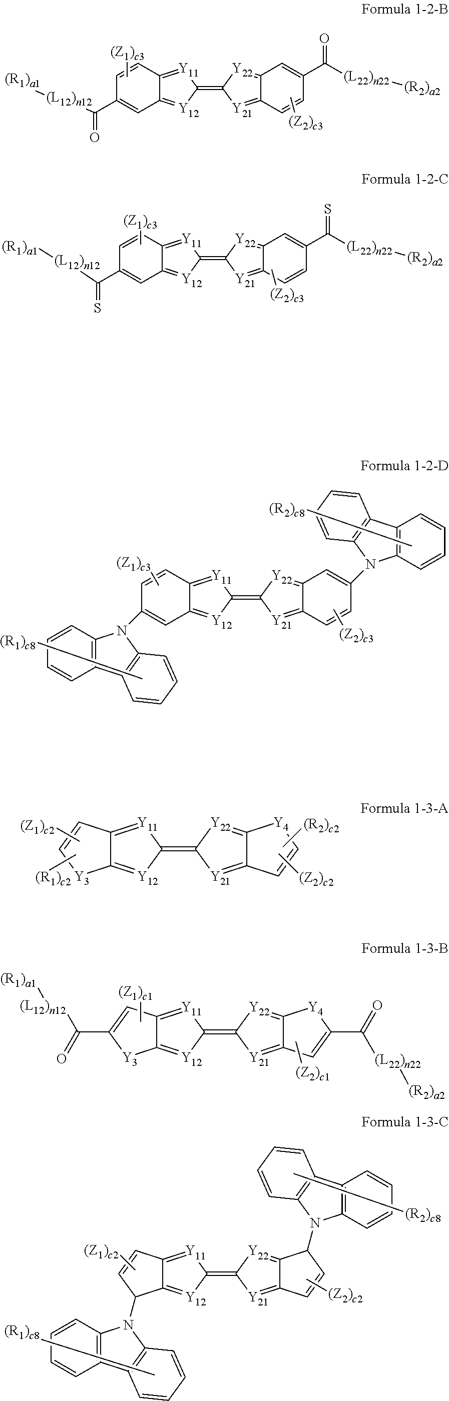

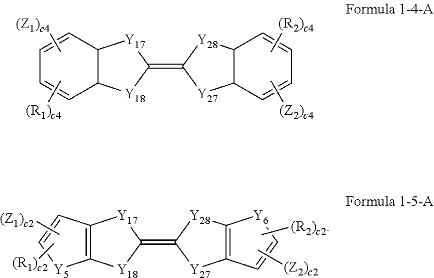

In Formula 1-1-A to Formula 1-1-D, Formula 1-2-A to Formula 1-2-D, Formula 1-3-A to 1-3-C, and Formula 1-4-A, and Formula 1-5-A,
Y₁₁, Y₁₂, Y₂₁, Y₂₂, Y₁₃, Y₁₄, Y₂₃, Y₂₄, Y₁₇, Y₁₈, Y₂₇, Y₂₈, L₁₁, L₁₂, L₂₁, L₂₁, n11, n12, n21, n22, R₁, R₂, a1, a2, Z₁, and Z₂ are as described herein,
Y₃ may be C(R₃₁)(R₃₂), N(R₃₁), O, or S,
Y₄ may be C(R₄₁)(R₄₂), N(R₄₁), O, or S,
Y₅ may be C(R₅₁)(R₅₂), N(R₅₁), O, or S,
Y₆ may be C(R₆₁)(R₆₂), N(R₆₁), O, or S,
c1 may be an integer from 0 to 1,
c2 may be an integer from 0 to 2,
c3 may be an integer from 0 to 3,
c4 may be an integer from 0 to 4,
c6 may be an integer from 0 to 6,
c8 may be an integer from 0 to 8, and
R₃₁, R₃₂, R₄₁, R₄₂, R₅₁, R₅₂, R₆₁, and R₆₂ are as described in connection with R₁.

According to one or more embodiments, the organic compound represented by Formula 1 may include
i) at least one indene group,
ii) at least one benzimidazole group,
iii) at least one carbazole group,
iv) at least one triazine group,
v) at least one cyclopentadiene group,
vi) at least one benzene group,
vii) at least one indole group, or
viii) any combination of i) to vii).

According to one or more embodiments, the organic compound represented by Formula 1 may have point symmetry.

According to one or more embodiments, the organic compound represented by Formula 1 may have a glass transition temperature of 105° C. or higher.

According to one or more embodiments, the organic compound represented by Formula 1 may have a LUMO energy level of −5.0 eV or less. For example, the organic compound represented by Formula 1 may have a LUMO energy level of about −6.0 eV to about −5.0 eV.

The organic compound represented by Formula 1 according to the disclosure may include a double bond (e.g., a double covalent bond) and at least one cyano group.

The double bond (e.g., double covalent bond) may form a conjugated structure and a structure similar thereto, and the cyano group is an excellent electron withdrawing group (EWG) that reduces the electron density within the conjugated structure, so that when the organic compound is employed in a light-emitting device, excellent hole injection ability may be achieved.

Additionally, because the organic compound has a high glass transition temperature, when employed in a light-emitting device, the obtained light-emitting device may have high thermal stability.

Therefore, light-emitting devices including the organic compound may achieve excellent low driving voltage and long life characteristics.

### Specific Example of First Compound

According to one or more embodiments, the organic compound represented by Formula 1 (i.e., the first compound) may be represented by or include at least one of compounds 1 to 78:

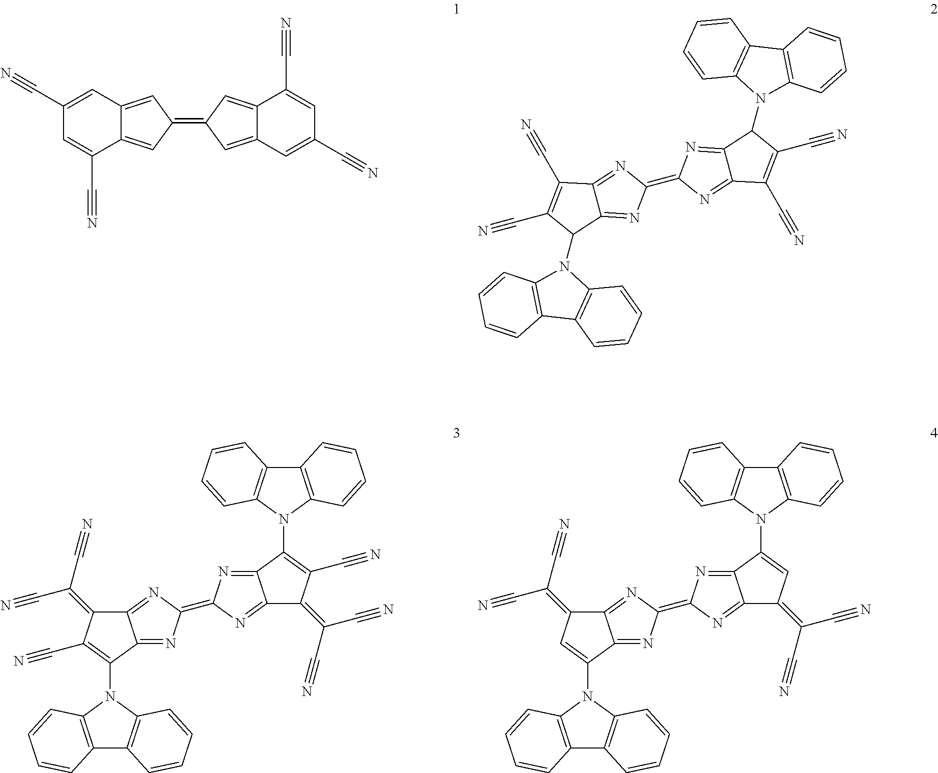 [SMILES: N#Cc1cc(C#N)c2c(c1)=C/C(=C1/C=c3cc(C#N)cc(C#N)c3=C1)C=2 ; N#CC1=C(C#N)C(n2c3ccccc3c3ccccc32)C2=N/C(=C3\N=C4C(=N3)C(n3c5ccccc5c5ccccc53)C(C#N)=C4C#N)N=C21 ; N#CC(C#N)=C1C2=N/C(=C3/N=C4C(=N3)C(n3c5ccccc5c5ccccc53)=C(C#N)C4=C(C#N)C#N)N=C2C(n2c3ccccc3c3ccccc32)=C1C#N ; N#CC(C#N)=C1C=C(n2c3ccccc3c3ccccc32)C2=N/C(=C3/N=C4C(n5c6ccccc6c6ccccc65)=CC(=C(C#N)C#N)C4=N3)N=C21]

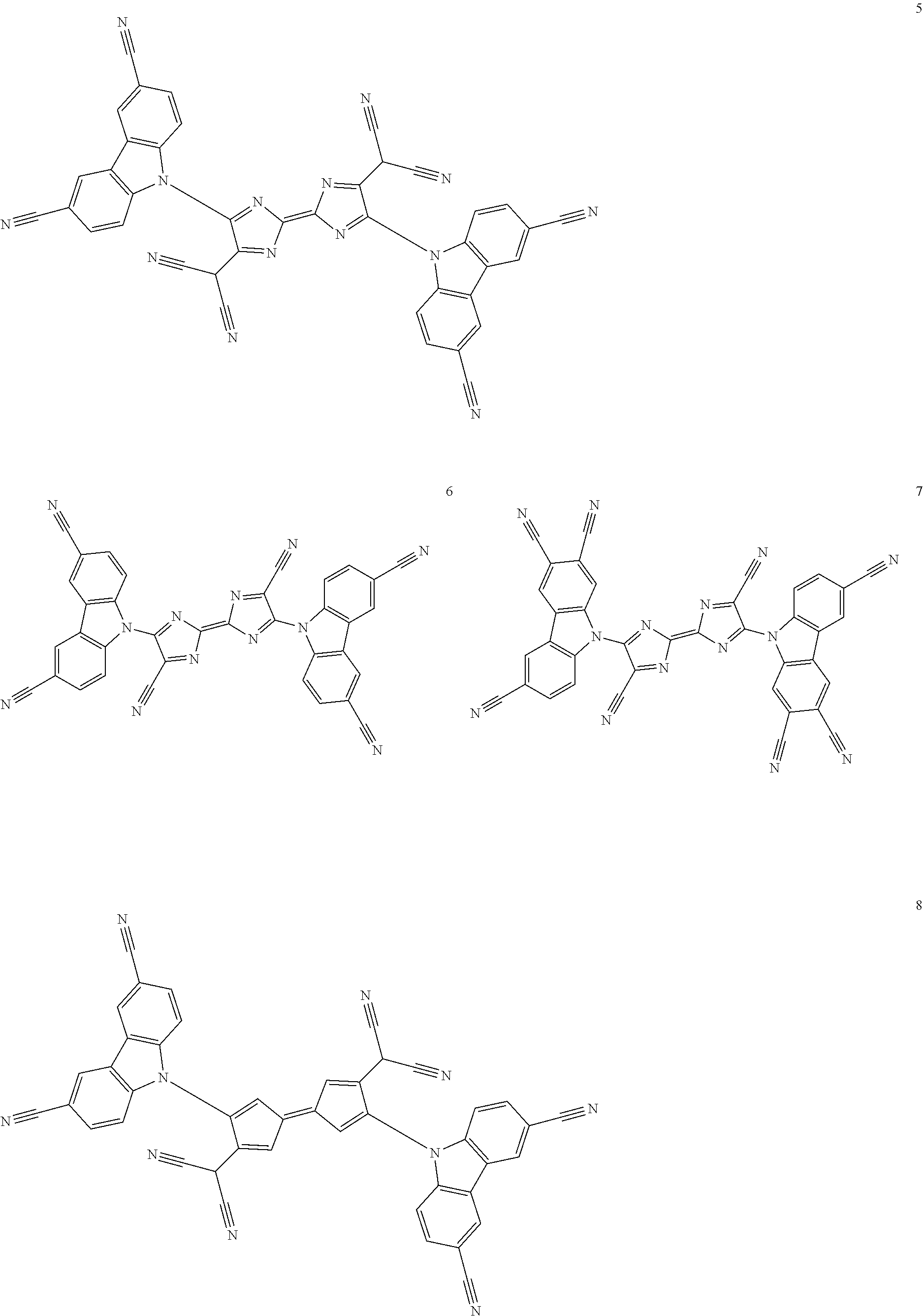 [SMILES: N#Cc1ccc2c(c1)c1cc(C#N)ccc1n2C1=N/C(=C2\N=C(C(C#N)C#N)C(n3c4ccc(C#N)cc4c4cc(C#N)ccc43)=N2)N=C1C(C#N)C#N ; N#CC1=N/C(=C2/N=C(C#N)C(n3c4ccc(C#N)cc4c4cc(C#N)ccc43)=N2)N=C1n1c2ccc(C#N)cc2c2cc(C#N)ccc21 ; N#CC1=N/C(=C2/N=C(C#N)C(n3c4ccc(C#N)cc4c4cc(C#N)c(C#N)cc43)=N2)N=C1n1c2ccc(C#N)cc2c2cc(C#N)c(C#N)cc21 ; N#Cc1ccc2c(c1)c1cc(C#N)ccc1n2C1=C/C(=C2\C=C(C(C#N)C#N)C(n3c4ccc(C#N)cc4c4cc(C#N)ccc43)=C2)C=C1C(C#N)C#N]

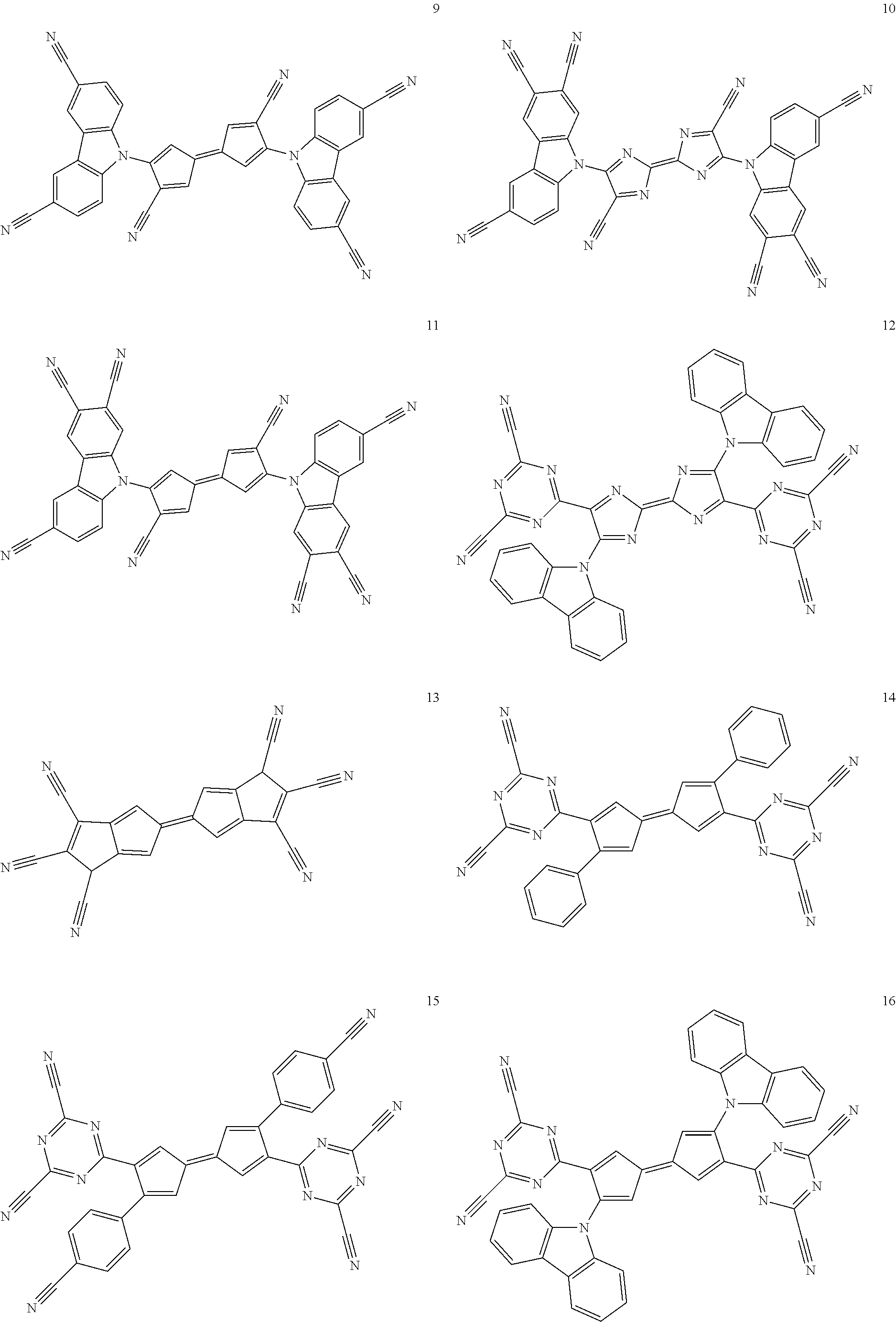

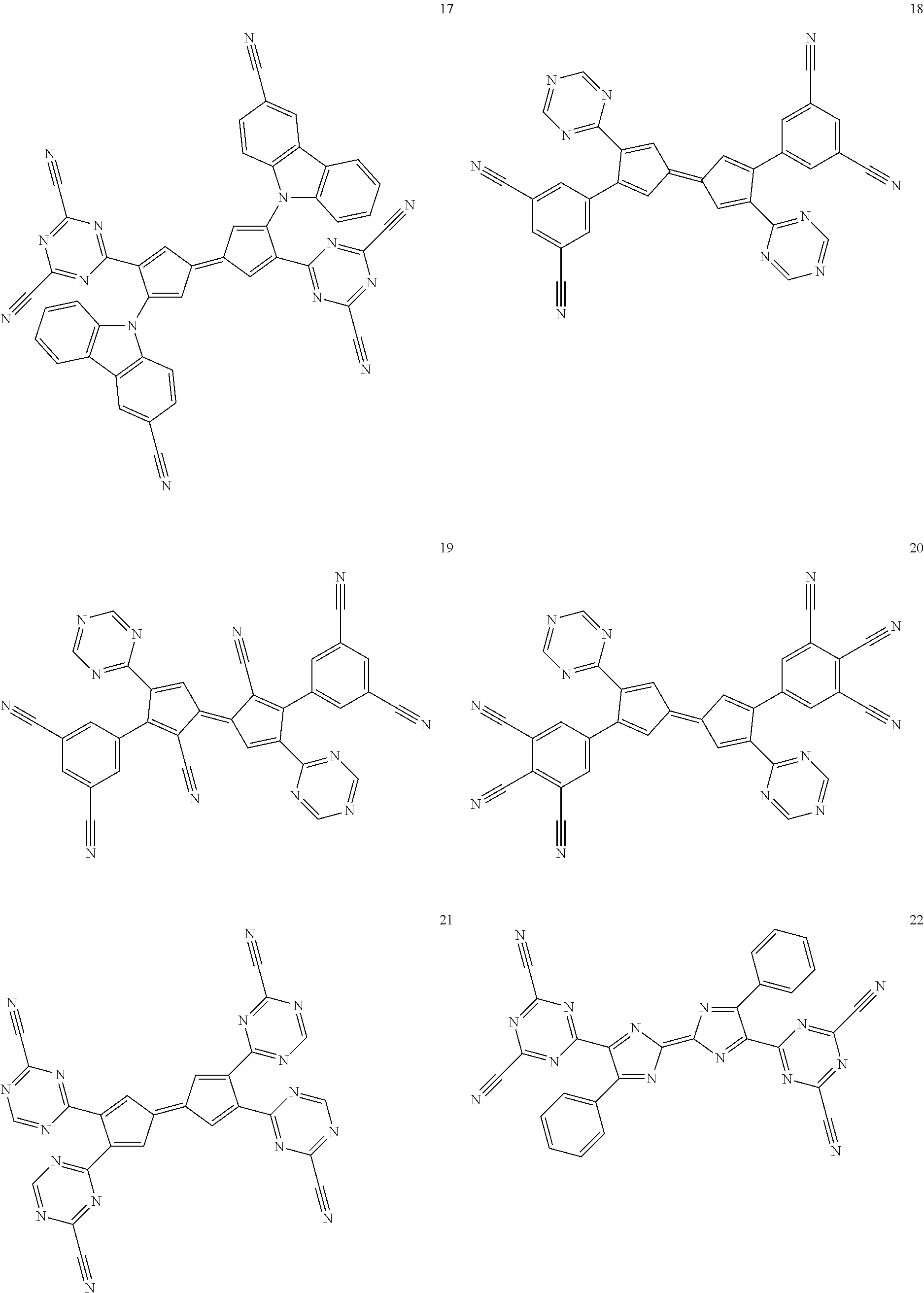

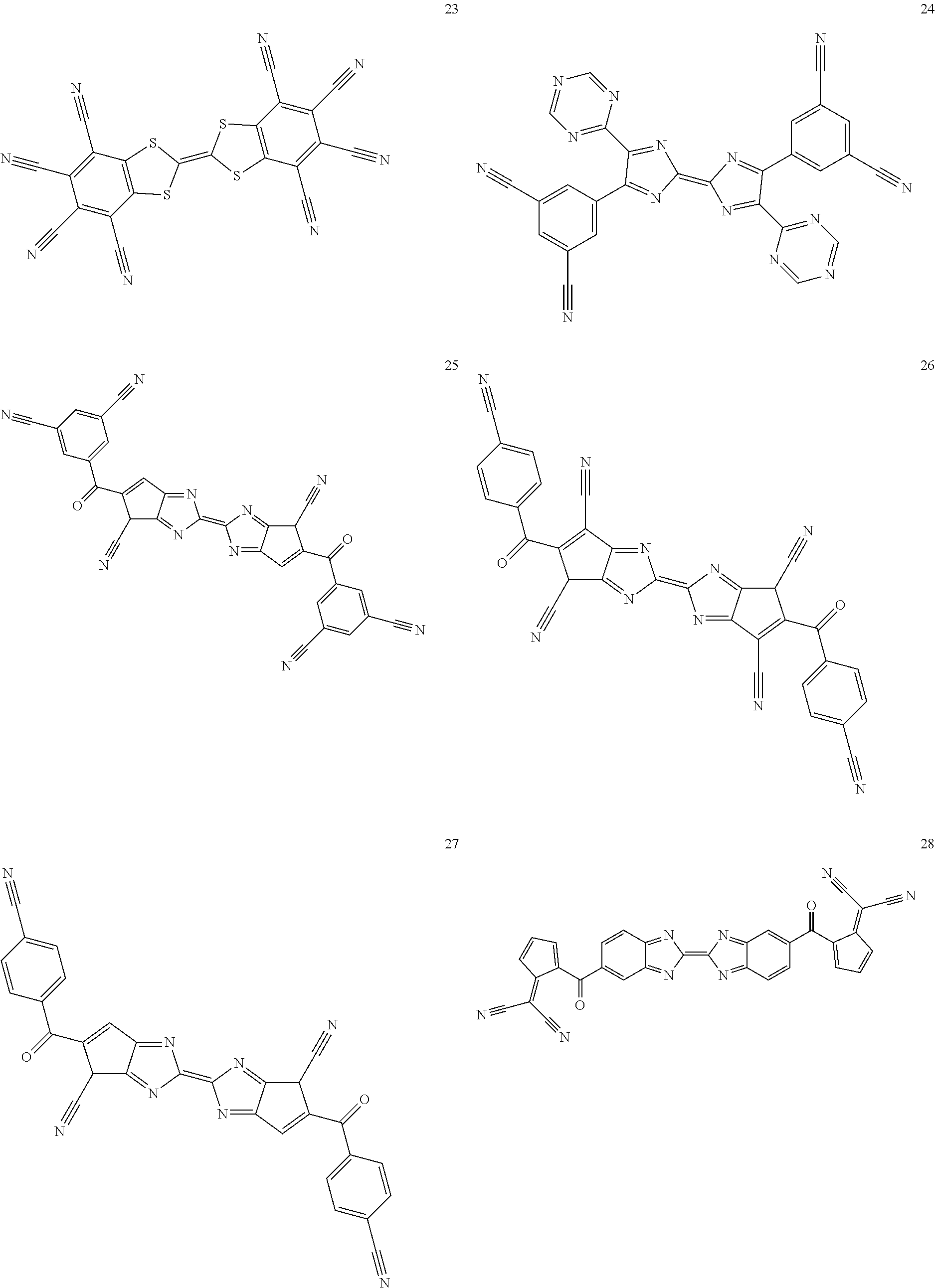

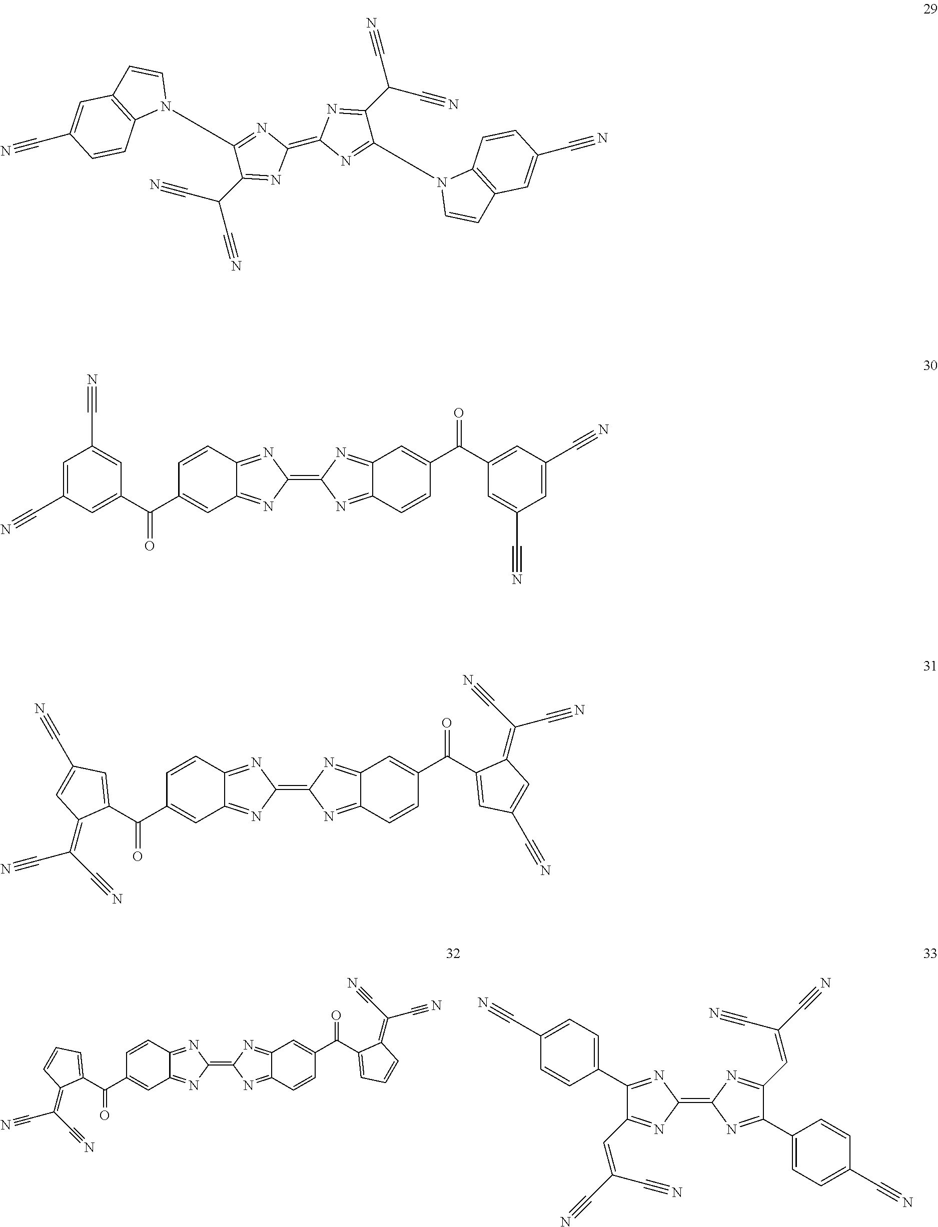 [SMILES: N#Cc1ccc2c(ccn2C2=N/C(=C3\N=C(C(C#N)C#N)C(n4ccc5cc(C#N)ccc54)=N3)N=C2C(C#N)C#N)c1 ; N#Cc1cc(C#N)cc(C(=O)c2ccc3c(c2)=N/C(=C2\N=c4ccc(C(=O)c5cc(C#N)cc(C#N)c5)cc4=N2)N=3)c1 ; N#CC1=CC(=C(C#N)C#N)C(C(=O)c2ccc3c(c2)=N/C(=C2\N=c4ccc(C(=O)C5=CC(C#N)=CC5=C(C#N)C#N)cc4=N2)N=3)=C1 ; N#CC(C#N)=C1C=CC=C1C(=O)c1ccc2c(c1)=N/C(=C1\N=c3ccc(C(=O)C4=CC=CC4=C(C#N)C#N)cc3=N1)N=2 ; N#CC(C#N)=CC1=N/C(=C2/N=C(C=C(C#N)C#N)C(c3ccc(C#N)cc3)=N2)N=C1c1ccc(C#N)cc1]

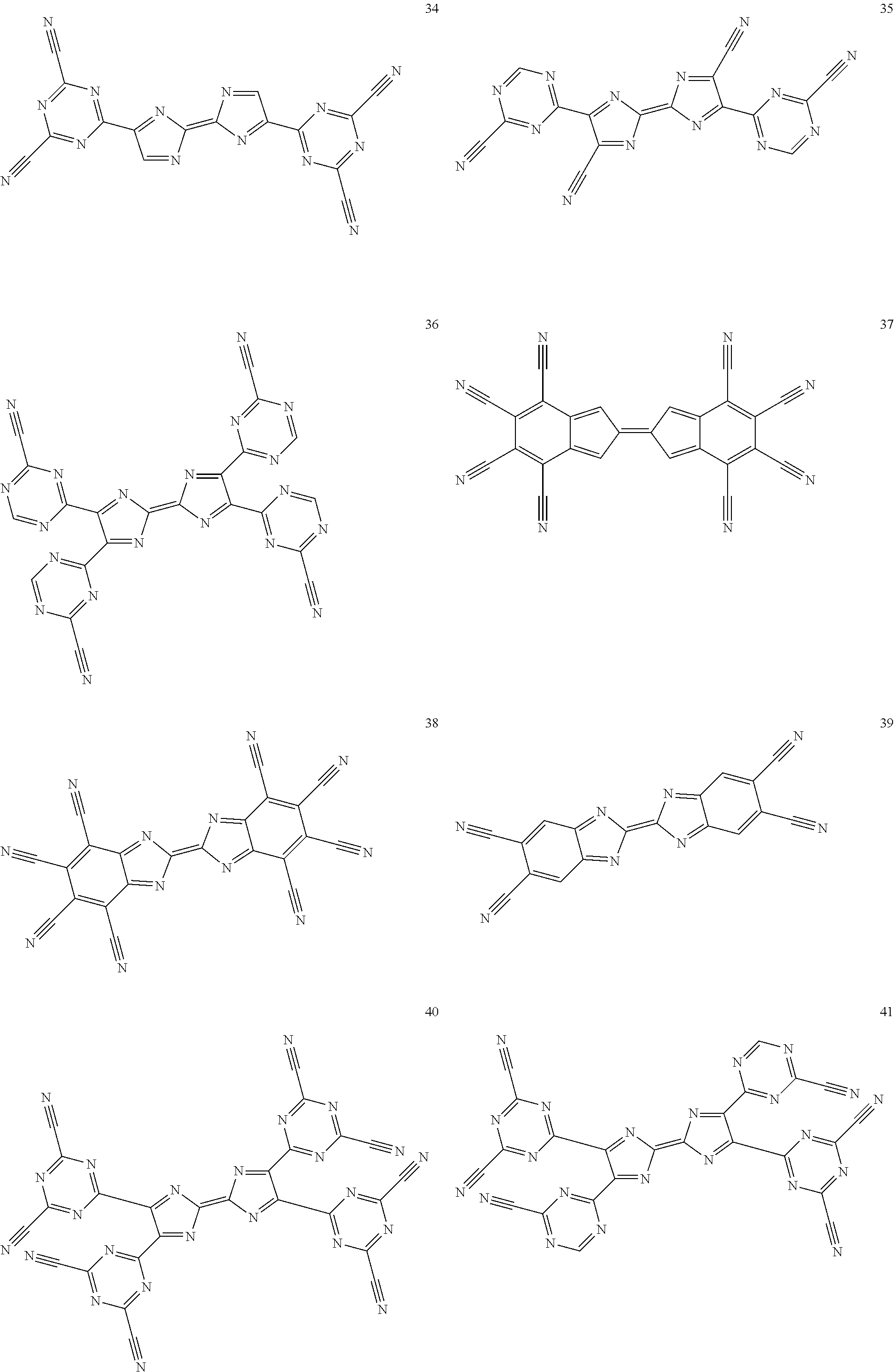

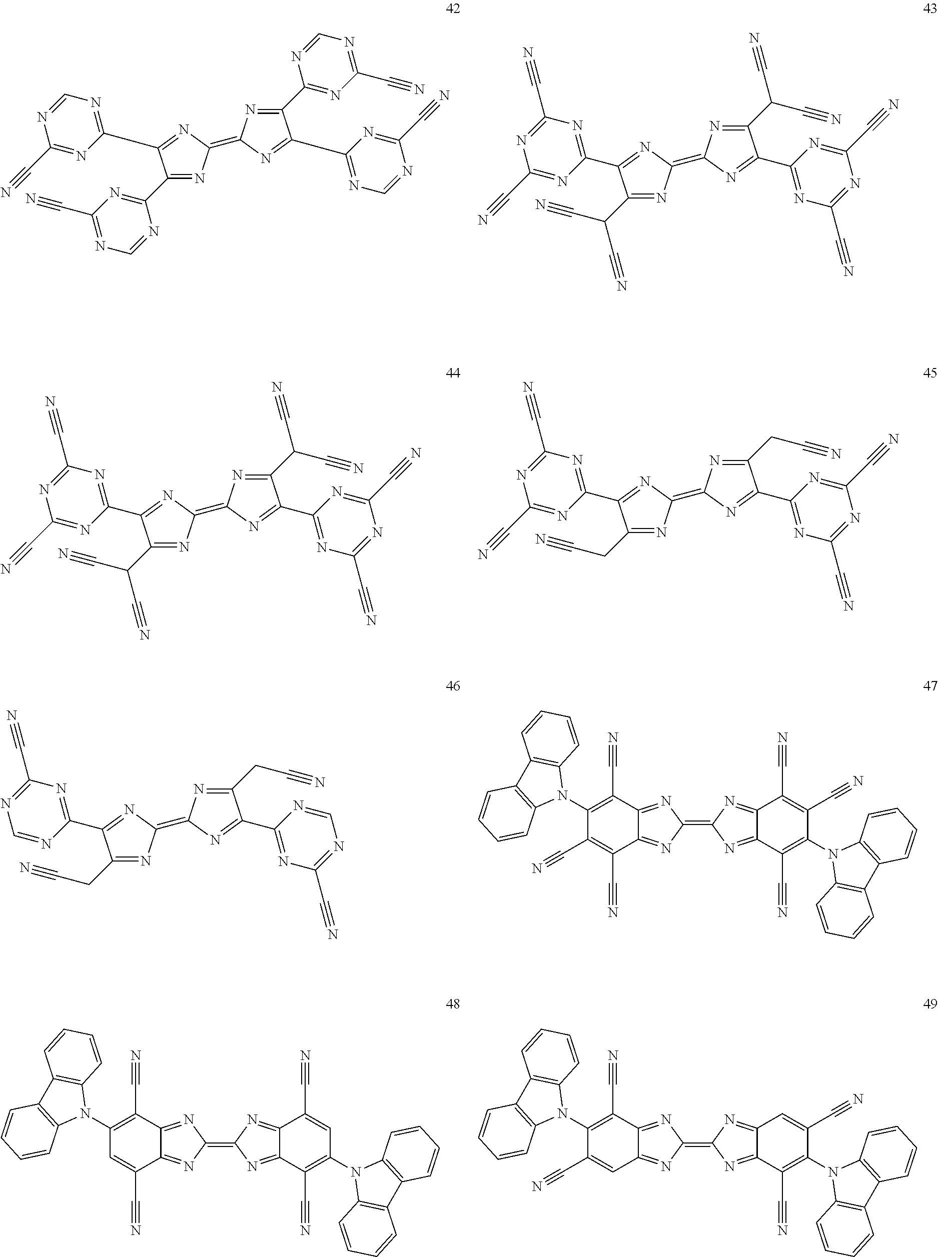

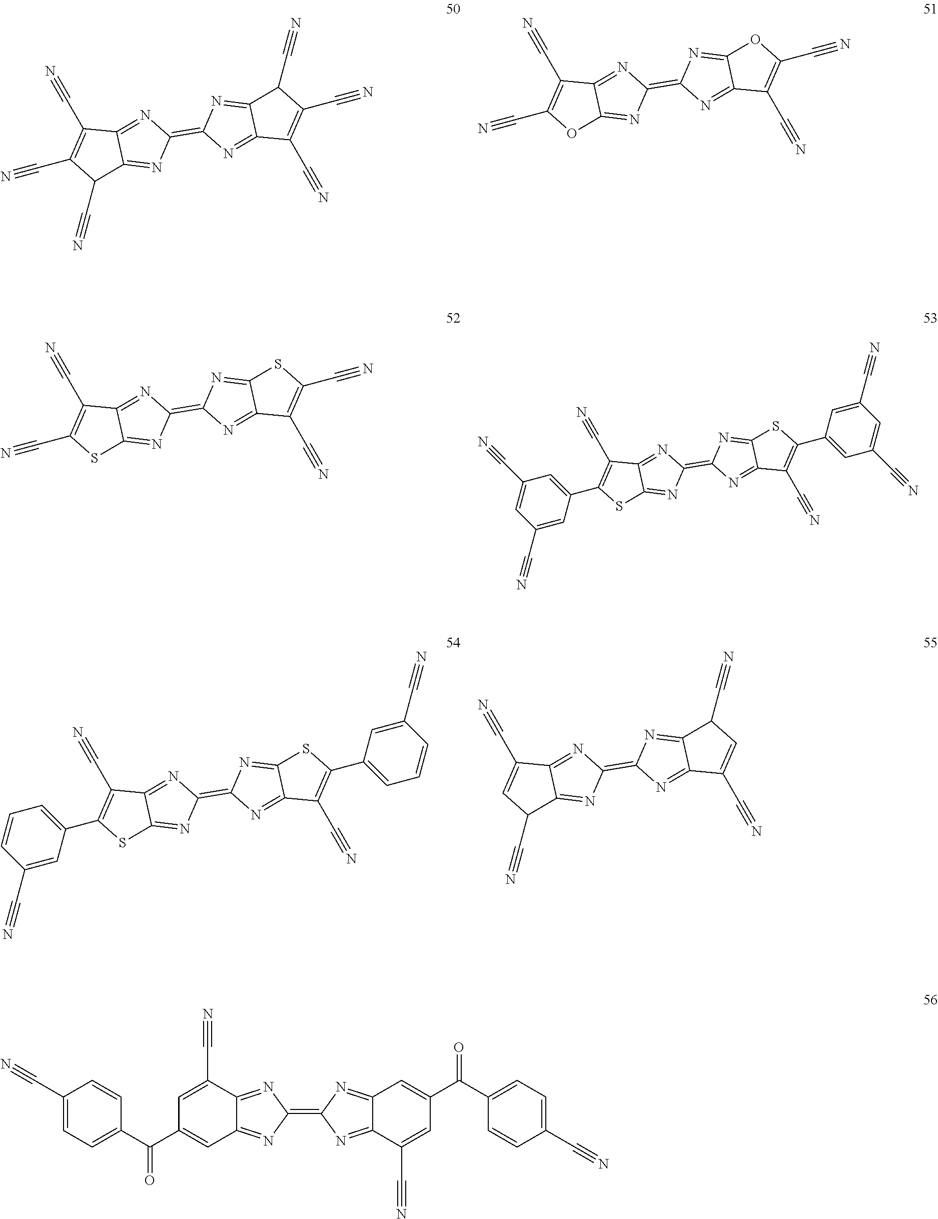

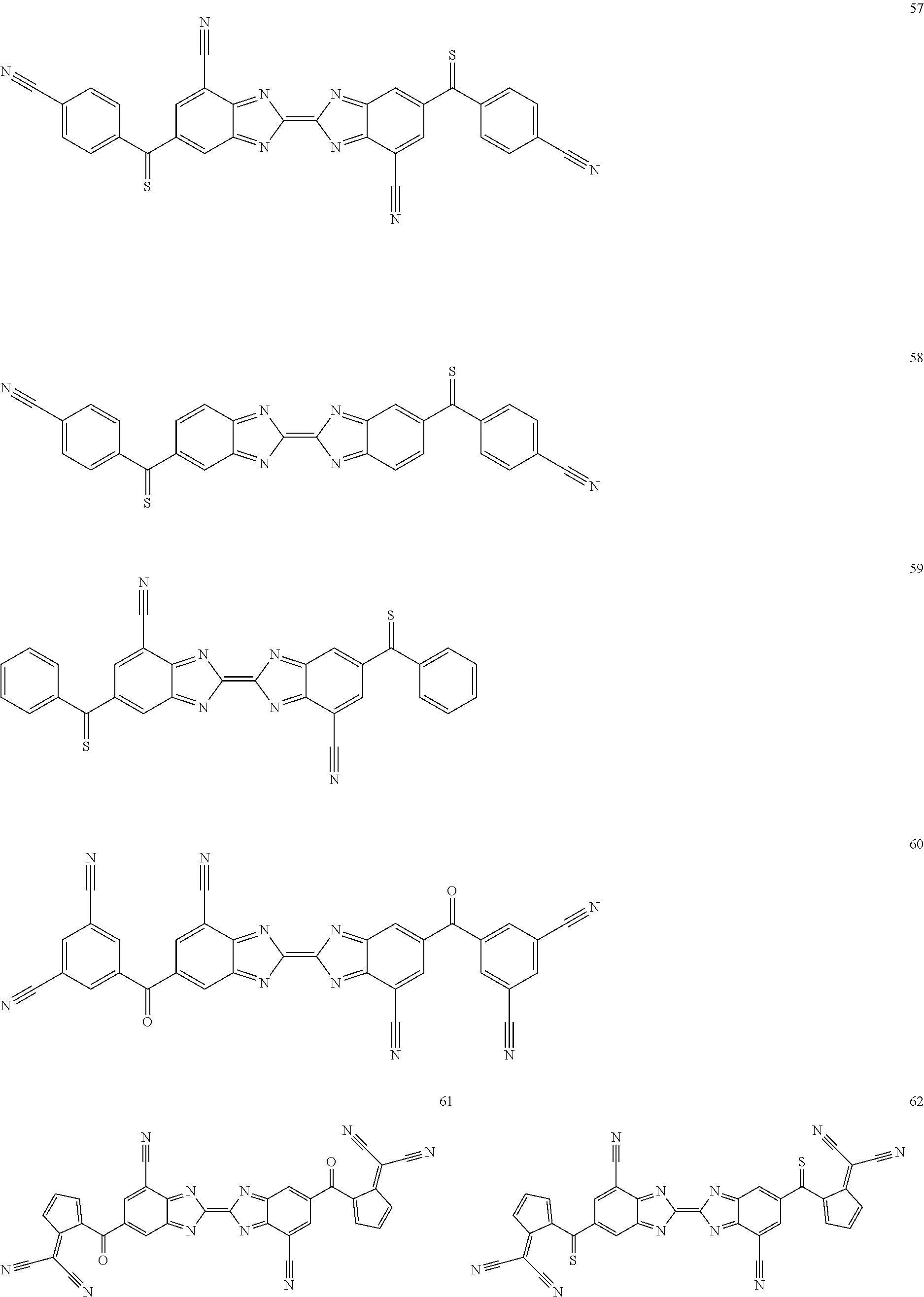

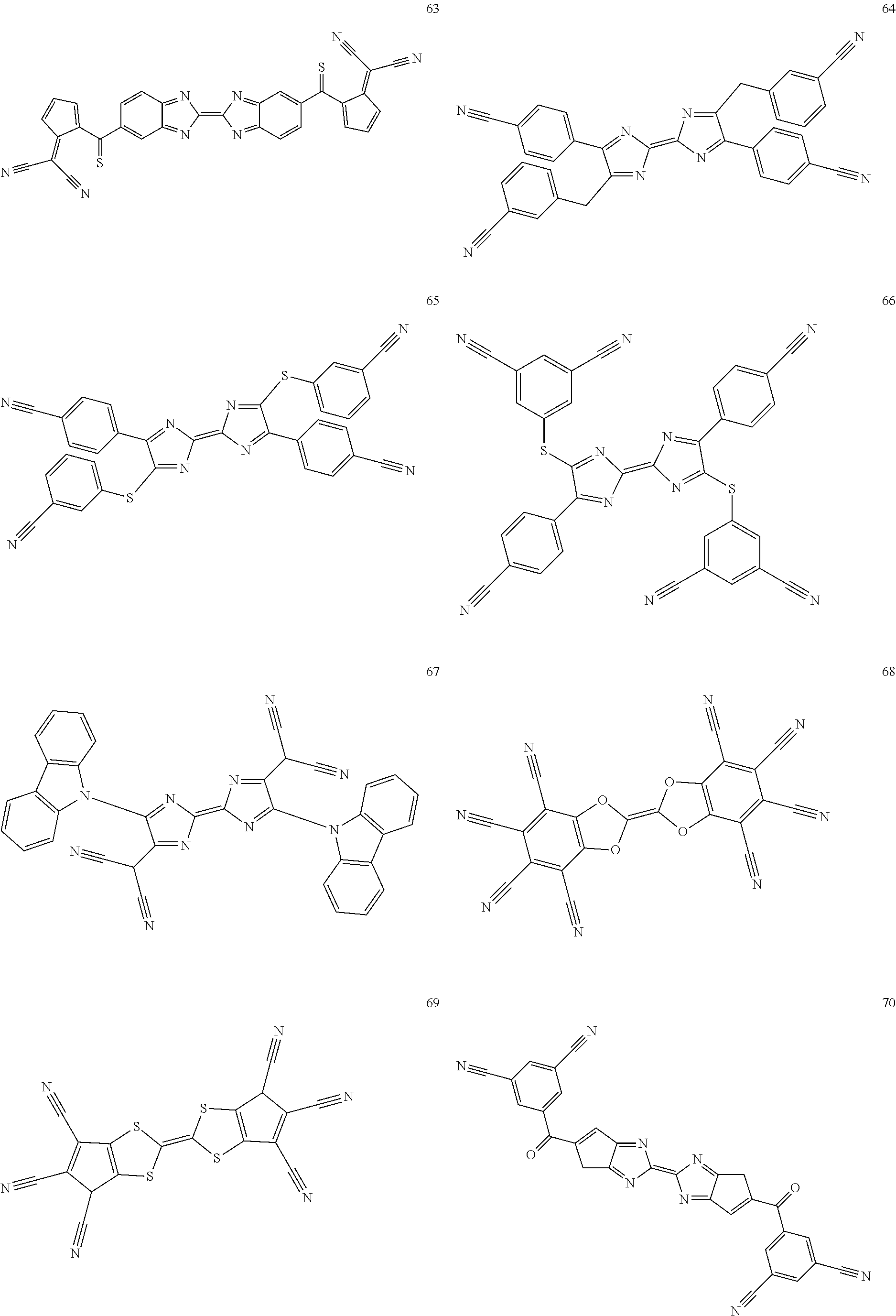

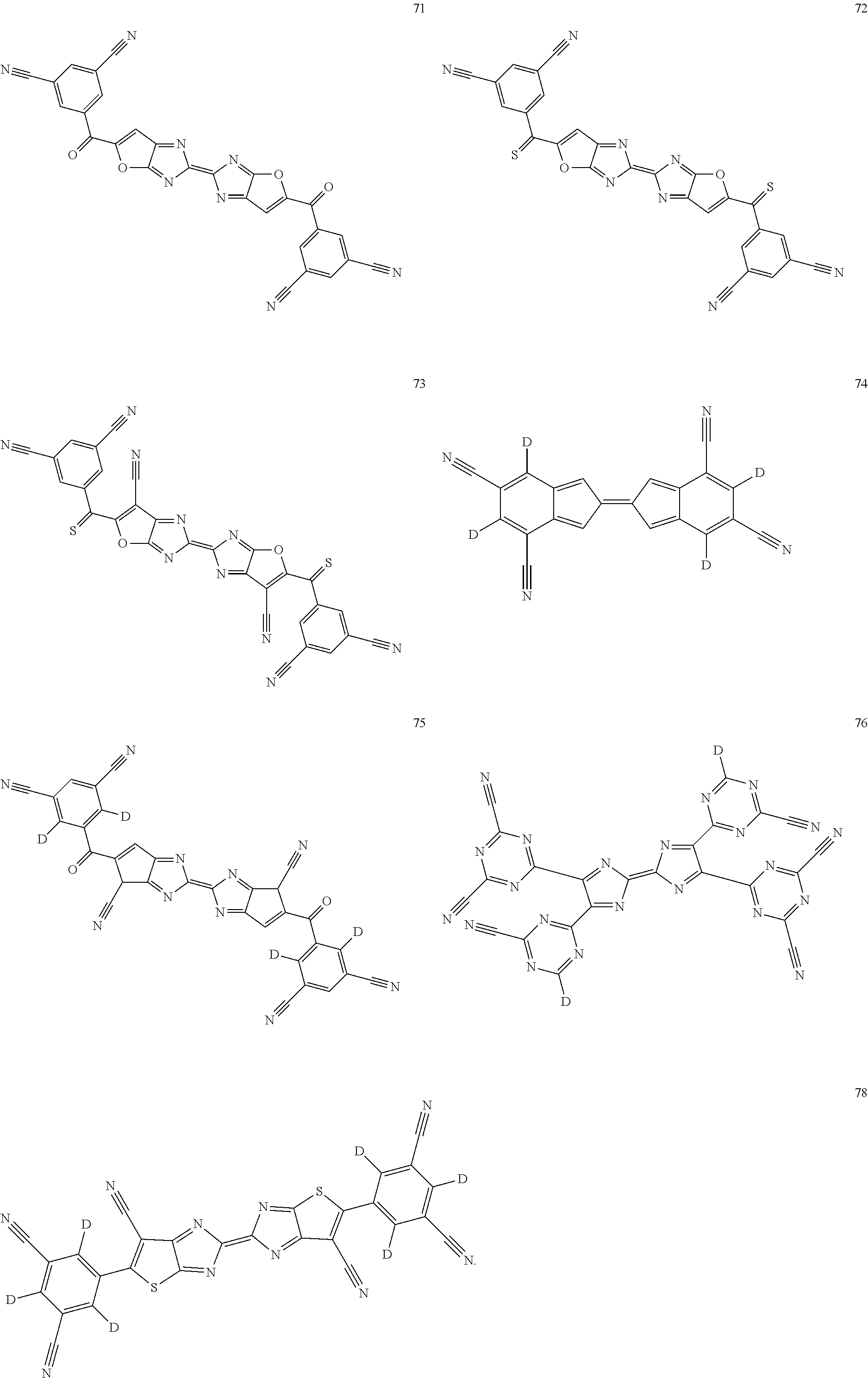

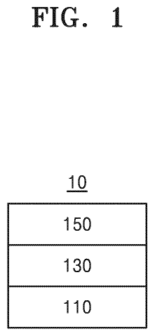

### Description of FIG. 1

FIG. 1 is a schematic cross-sectional view of a light-emitting device 10 according to one or more embodiments. The light-emitting device 10 includes a first electrode 110, an interlayer 130, and a second electrode 150.

Hereinafter, a structure of the light-emitting device 10 according to one or more embodiments and a method of manufacturing the light-emitting device 10 are described with reference to FIG. 1.

### First Electrode 110

In FIG. 1, a substrate may be additionally under the first electrode 110 or on the second electrode 150. As the substrate, a glass substrate or a plastic substrate may be used. In one or more embodiments, the substrate may be a flexible substrate and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a transflective electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a transflective electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layer structure including (e.g., consisting of) a single layer or a multilayer structure including a plurality of layers. In one or more embodiments, the first electrode 110 may have a three-layer structure of ITO/Ag/ITO.

### Interlayer 130

The interlayer 130 is on the first electrode 110. The interlayer 130 includes the emission layer.

The interlayer 130 may further include a hole transport region between the first electrode 110 and the emission layer, and an electron transport region between the emission layer and the second electrode 150.

The interlayer 130 may include, in addition to one or more suitable organic substances, inorganic substances such as metal-containing compounds and quantum dots.

In one or more embodiments, the interlayer 130 may include, i) two or more emitting units sequentially stacked between the first electrode 110 and the second electrode 150, and ii) a charge generation layer between adjacent emitting units among the two or more emitting units. When the interlayer 130 includes emitting units and a charge generation layer as described herein, the light-emitting device 10 may be a tandem light-emitting device.

### Hole Transport Region in Interlayer 130

The hole transport region may include the organic compound represented by Formula 1.

The hole transport region may have: i) a single-layer structure including (e.g., consisting of) a single layer including (e.g., consisting of) a single material, ii) a single-layer structure including (e.g., consisting of) a single layer including (e.g., consisting of) a plurality of materials that are different from each other, or iii) a multilayer structure including a plurality of layers including a plurality of materials that are different from each other.

The hole transport region may include a hole injection layer, a hole transport layer, an emission auxiliary layer, an electron-blocking layer, or any combination thereof.

For example, the hole transport region may have a multi-layer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, wherein layers in each structure are sequentially stacked from the first electrode 110.

The hole transport region may include a compound represented by Formula 201, a compound represented by Formula 202, or any combination thereof:

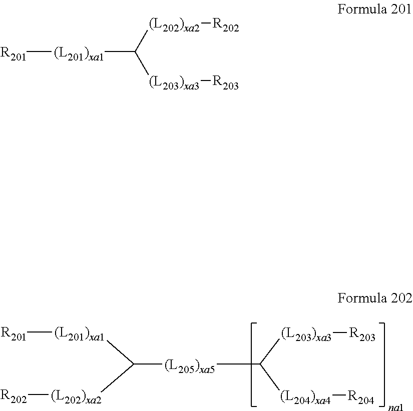

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond (e.g., a single covalent bond), a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (e.g., a carbazole group and/or the like) unsubstituted or substituted with at least one R₁₀ₐ (e.g., see compound HT16 and/or the like),
R₂₀₃ and R₂₀₄ may optionally be linked to each other via a single bond (e.g., a single covalent bond), a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group that is unsubstituted or substituted with at least one R₁₀ₐ, and
na1 may be an integer from 1 to 4.

In one or more embodiments, each of Formulae 201 and 202 may include at least one of groups represented by Formulae CY201 to CY217:

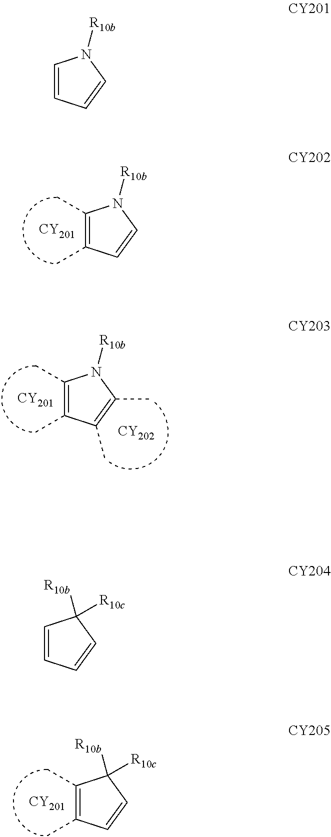

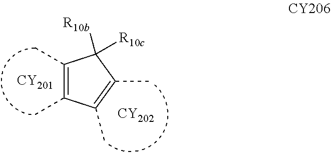

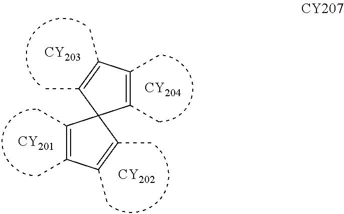

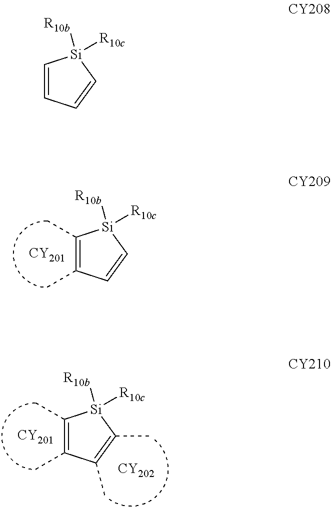

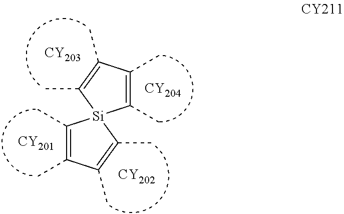

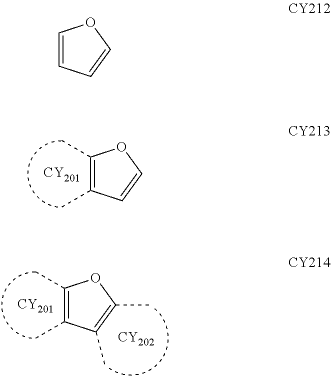

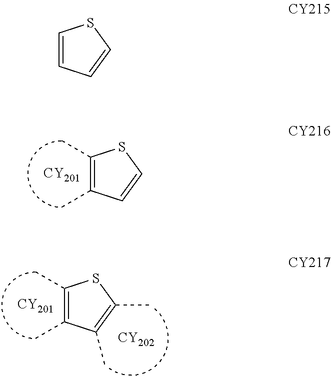

wherein, in Formulae CY201 to CY217, R_{10b}, and R_{10c} are each as described in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one of groups represented by Formulae CY201 to CY203.

In one or more embodiments, Formula 201 may include at least one of groups represented by Formulae CY201 to CY203 and at least one of groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, each of Formulae 201 and 202 may not include groups represented by Formulae CY201 to CY203.

In one or more embodiments, each of Formulae 201 and 202 may not include groups represented by Formulae CY201 to CY203 and may include at least one of groups represented by Formulae CY204 to CY217.

In one or more embodiments, each of Formulae 201 and 202 may not include groups represented by Formulae CY201 to CY217.

In one or more embodiments, the hole transport region may include at least one of compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzene sulfonic acid (PANI/DBSA), poly(3,4-ethylene dioxythiophene)/poly(4-styrene sulfonate) (PEDOT/PSS), polyaniline/camphor sulfonic acid (PANI/CSA), polyaniline/poly(4-styrene sulfonate) (PANI/PSS), or any combination thereof:

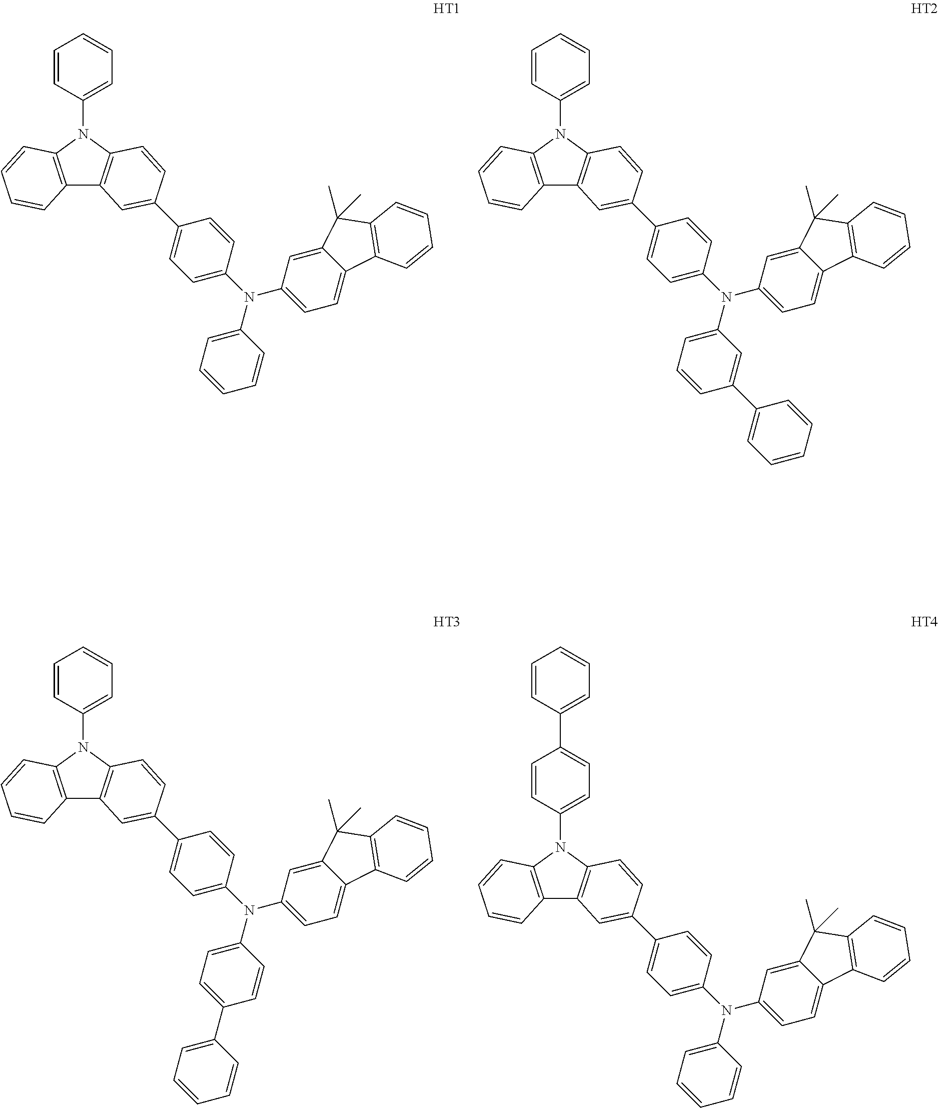 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

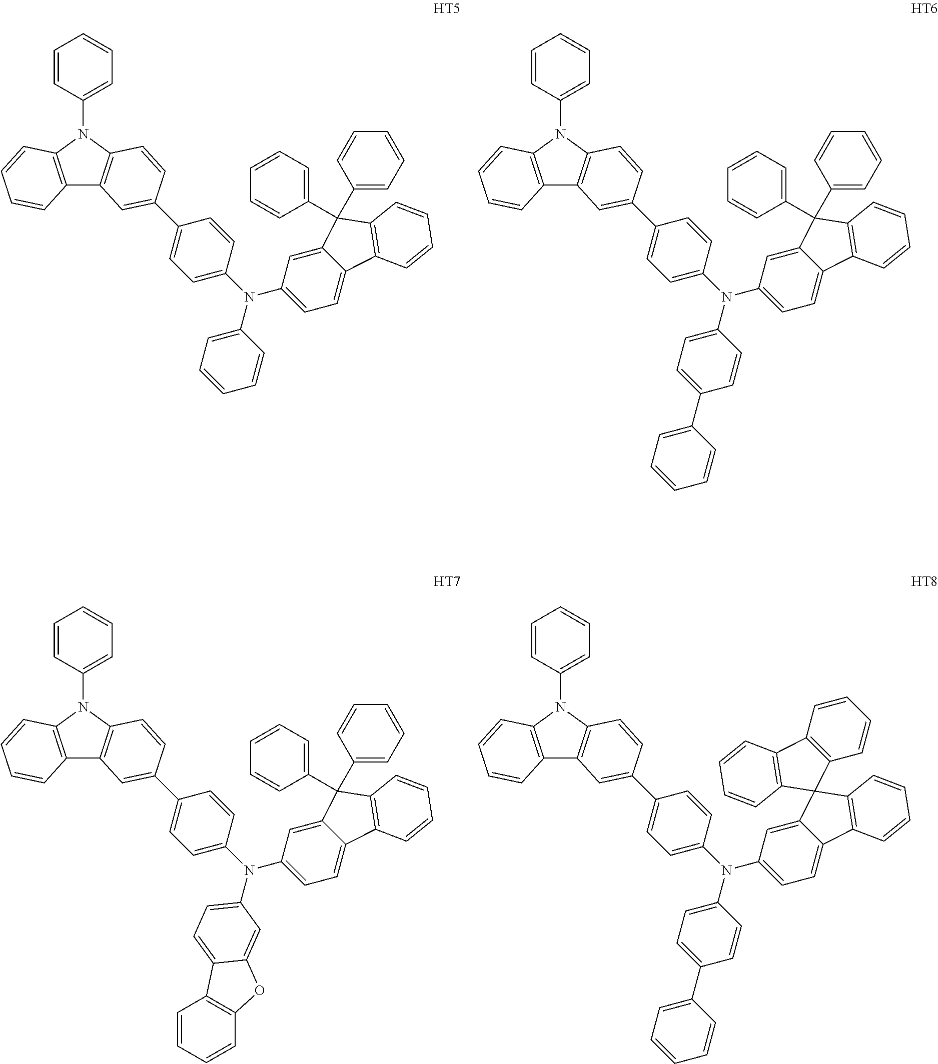 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

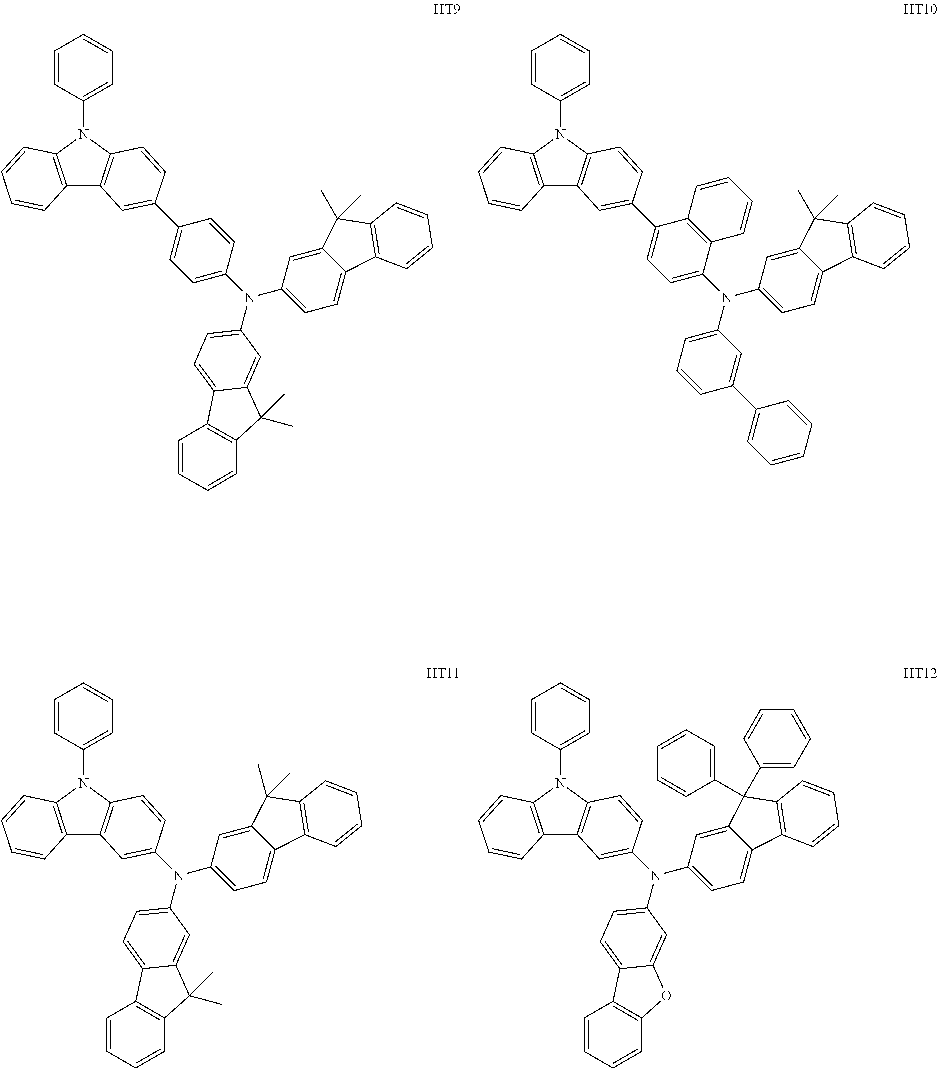 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

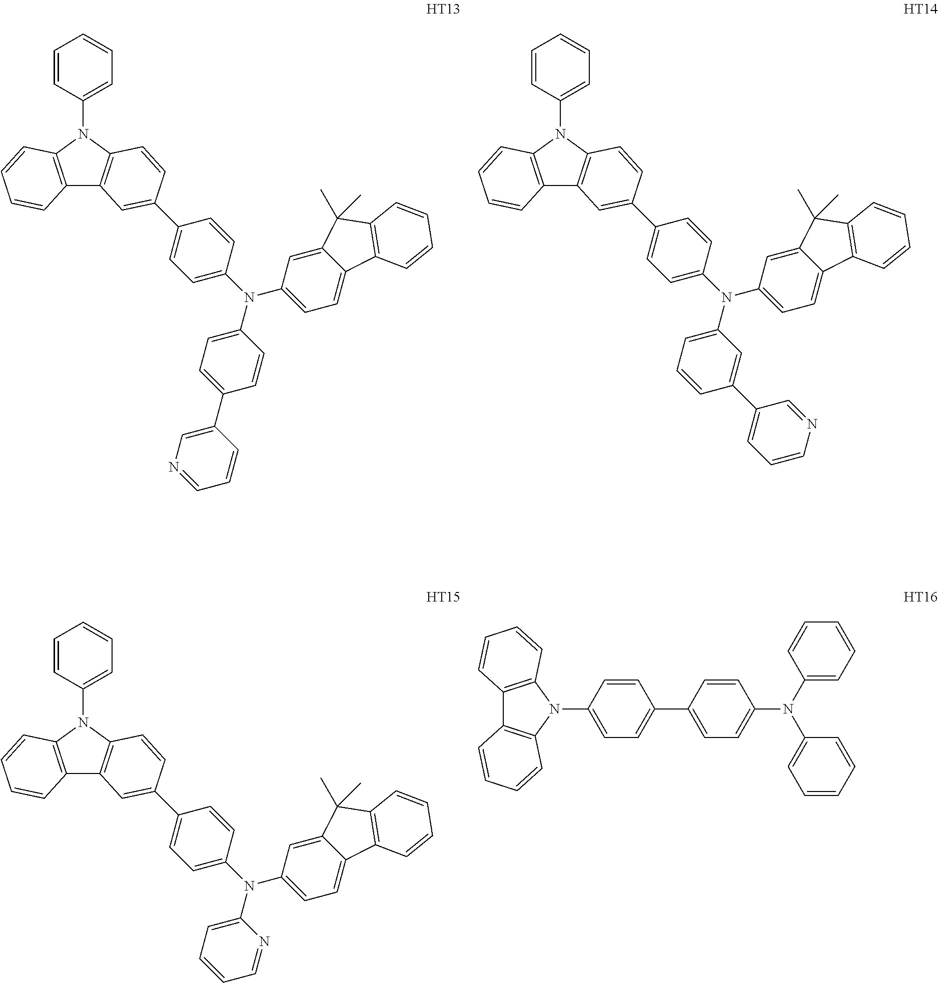 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

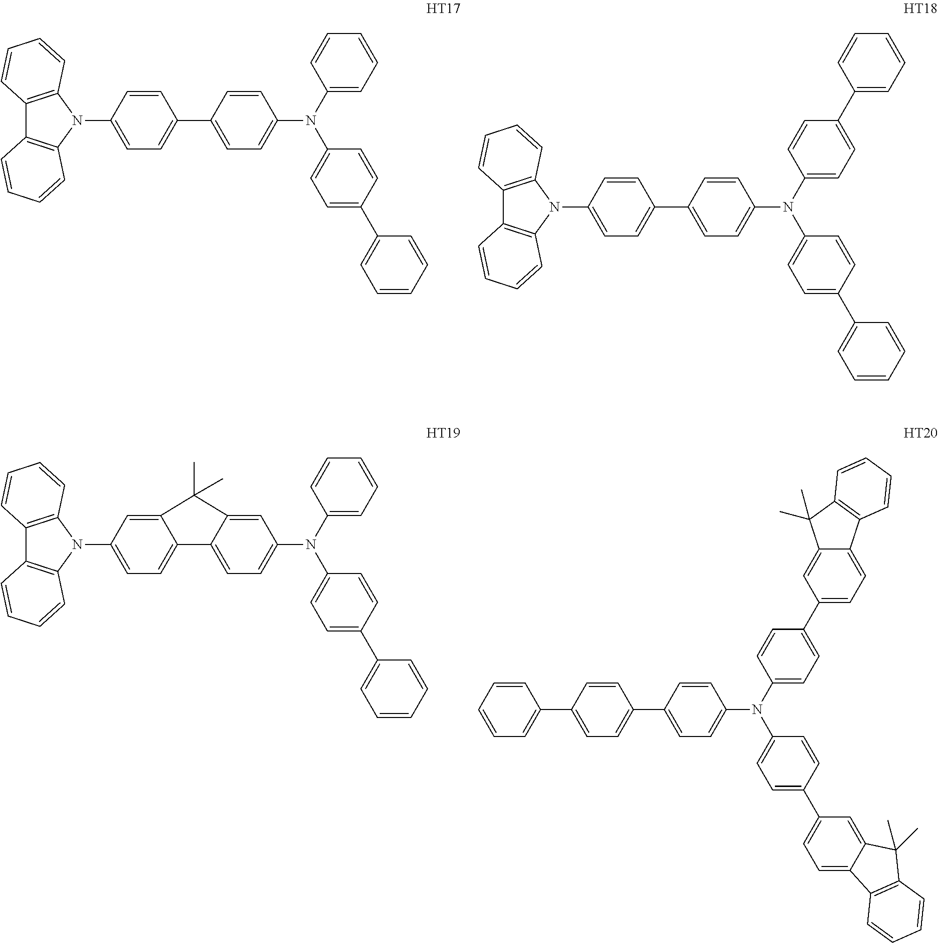 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

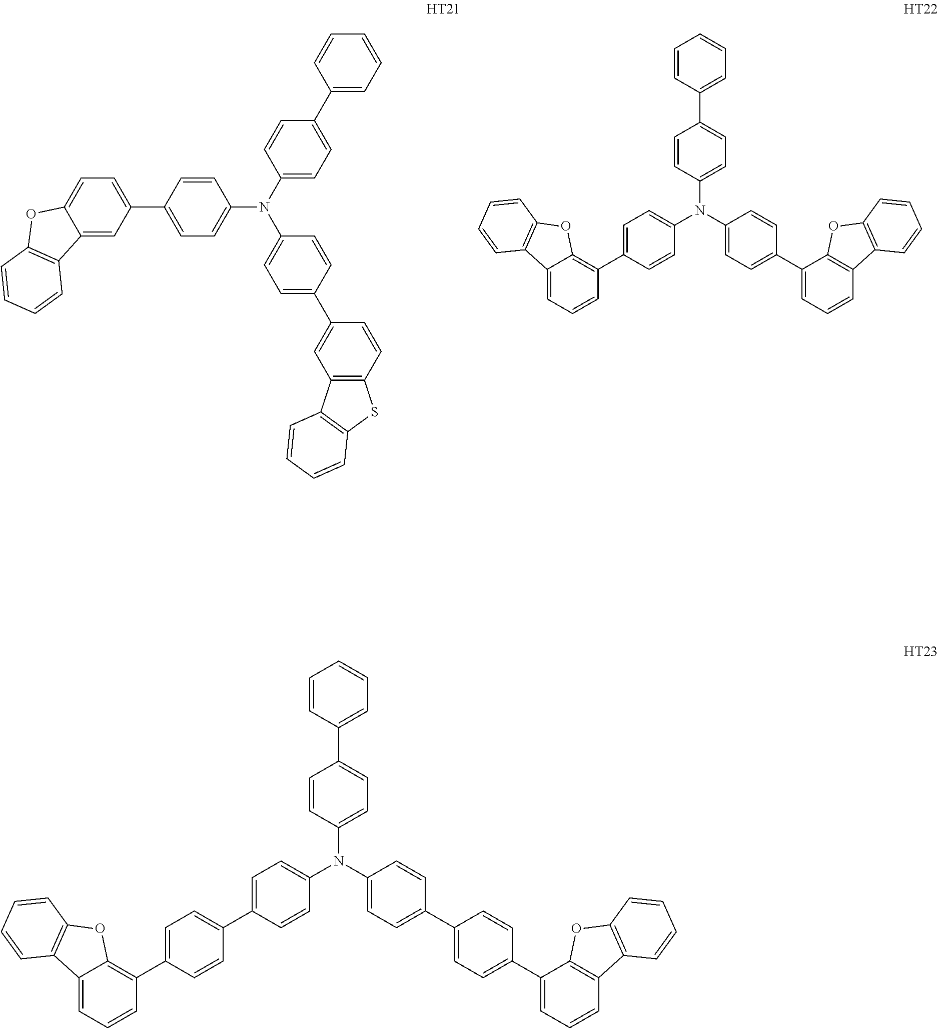 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

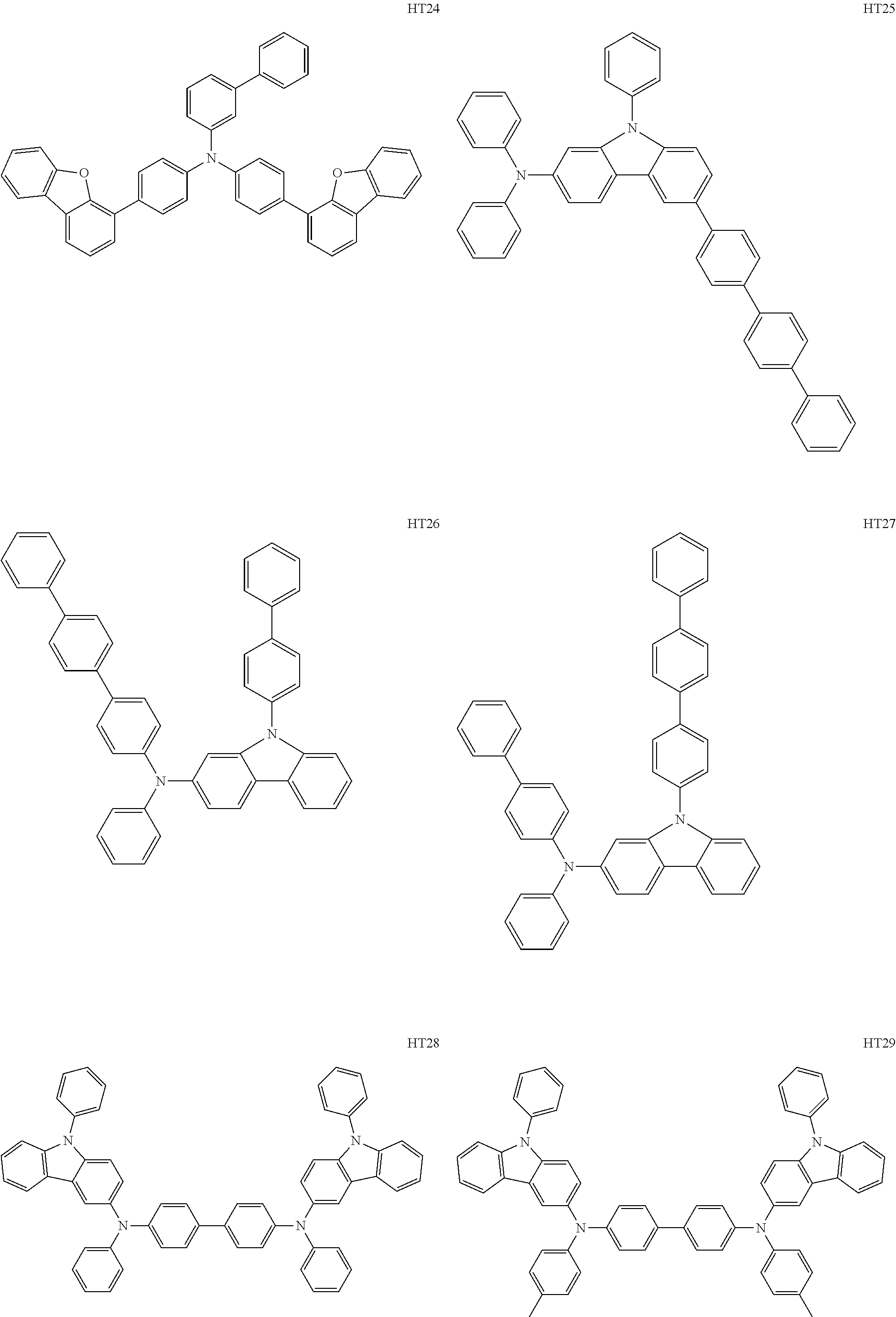

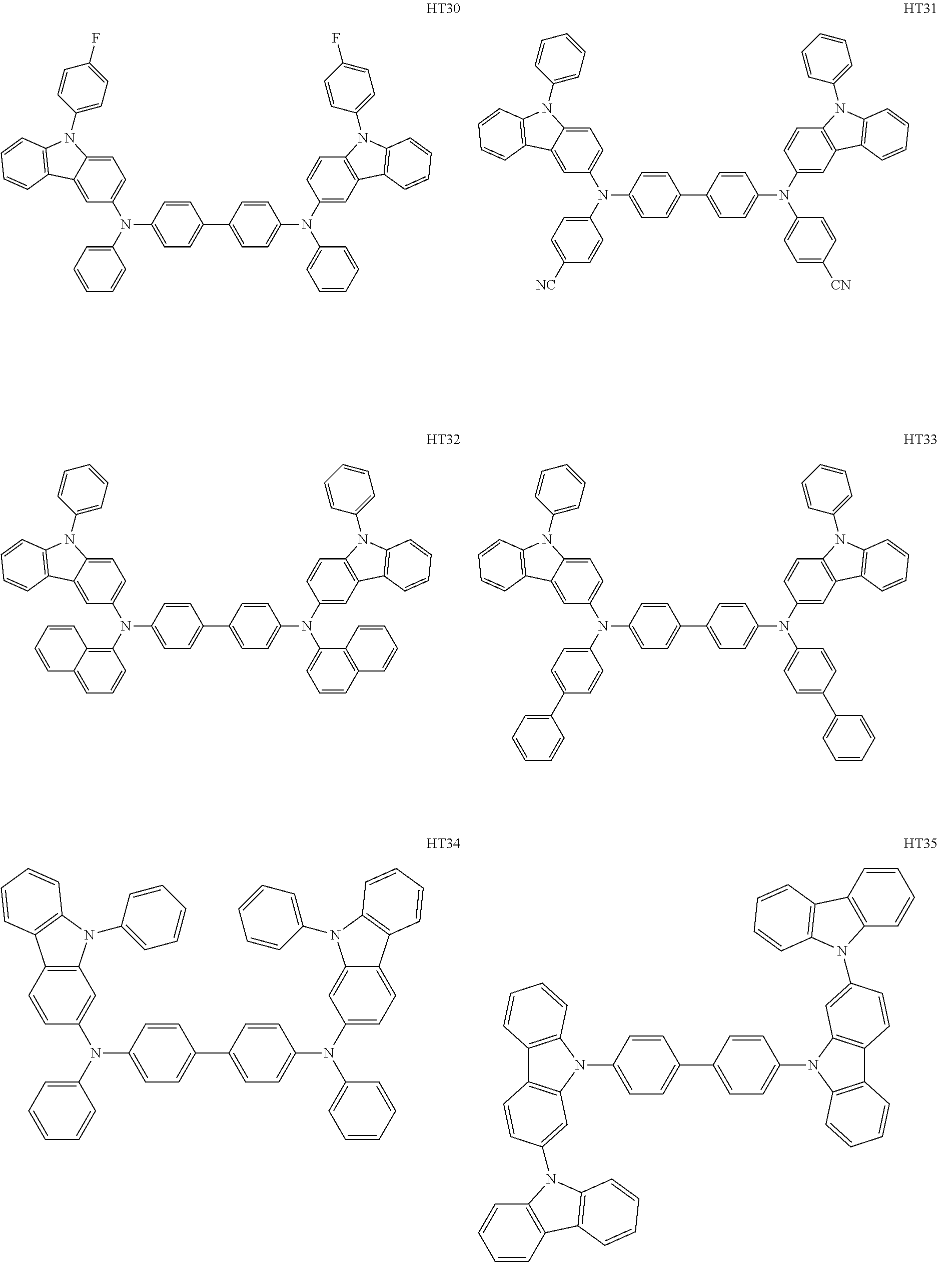

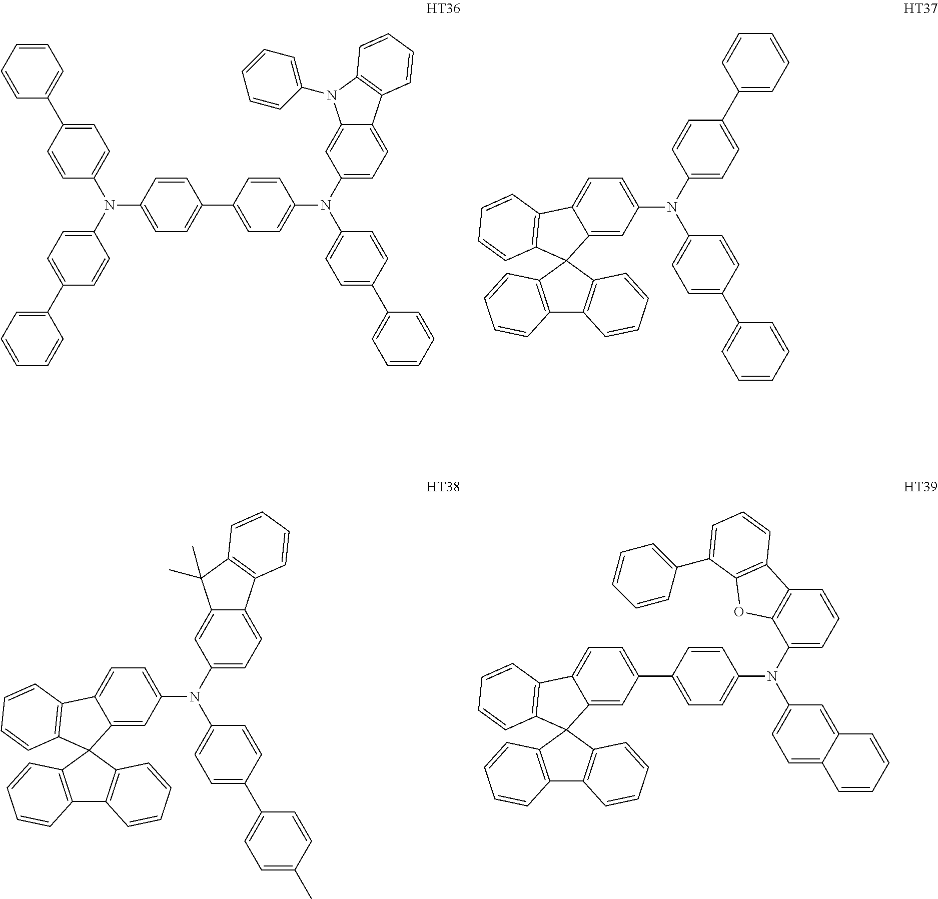 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

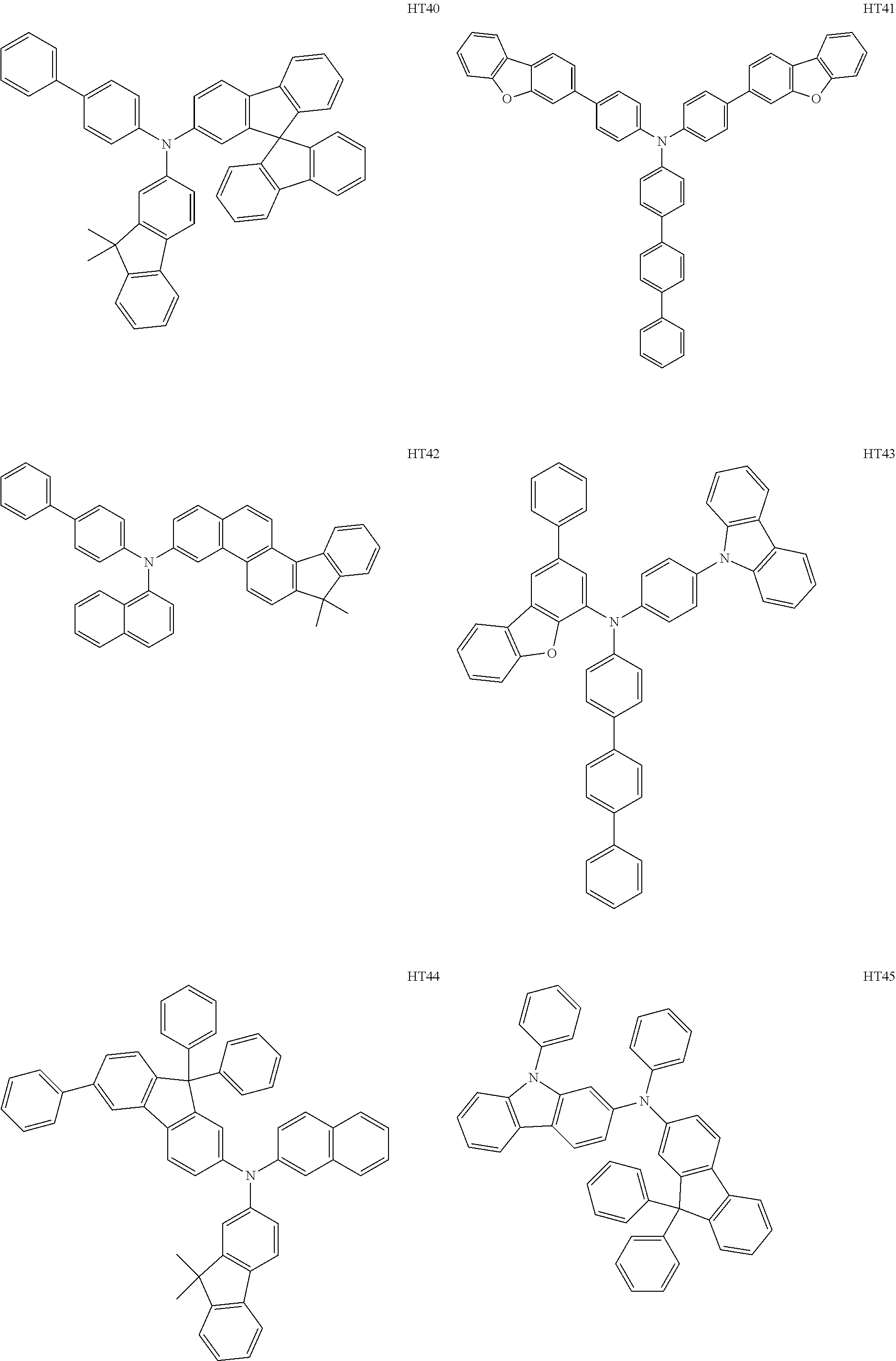

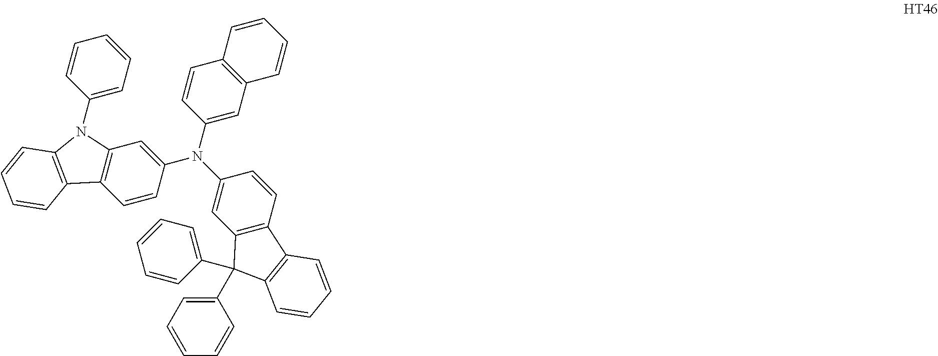 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

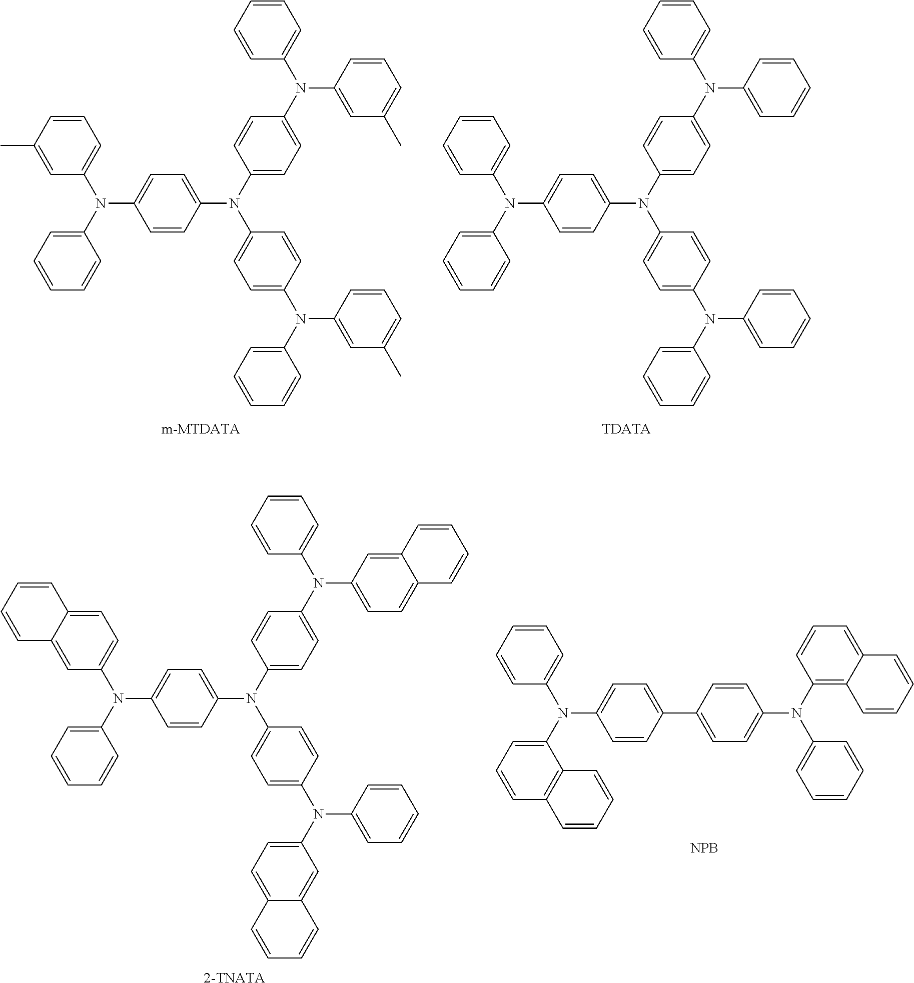

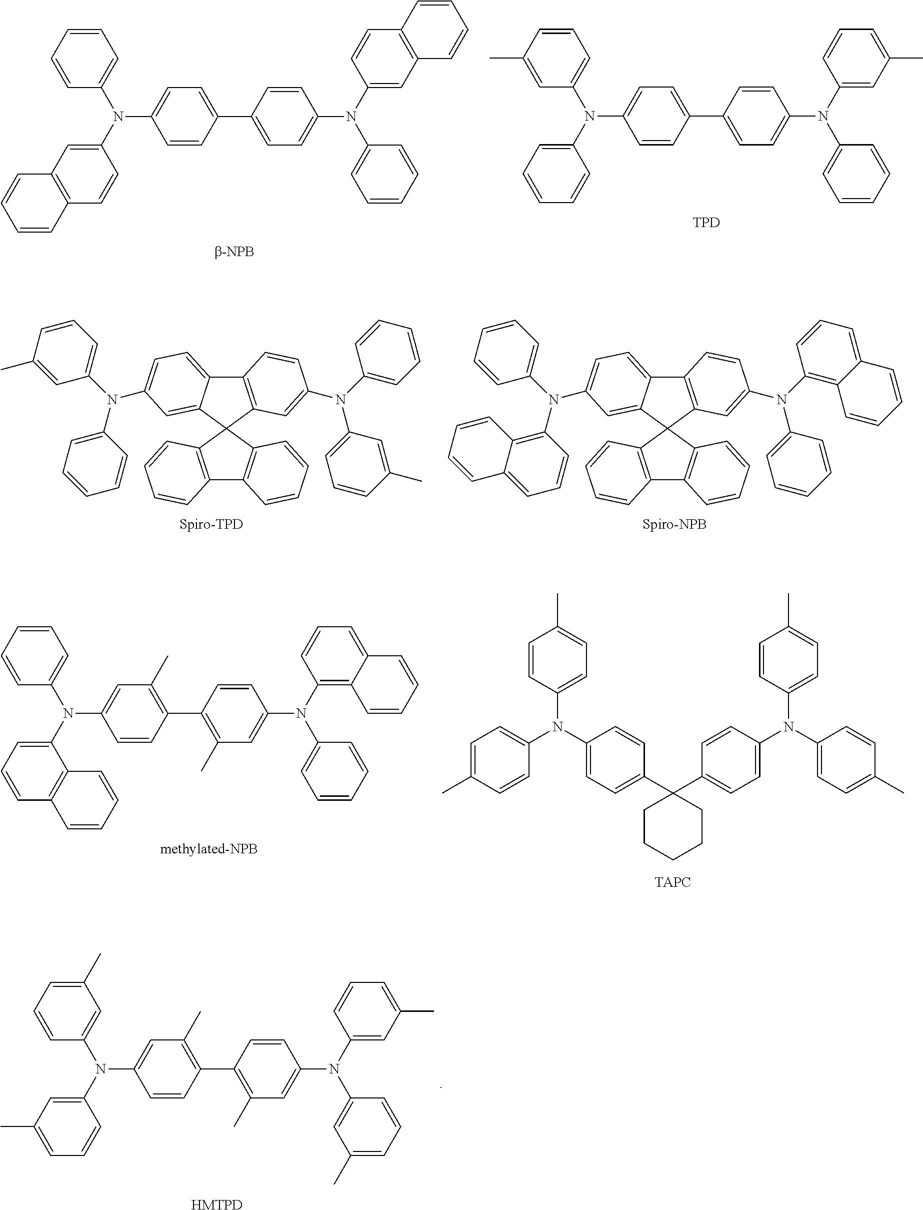

The thickness of the hole transport region may be about 50 angstrom (A) to about 10,000 Å, for example, about 100 Å to about 4,000 Å. When the hole transport region includes a hole injection layer, a hole transport layer, or any combination thereof, the thickness of the hole injection layer may be about 100 Å to about 9,000 Å, for example, about 100 Å to about 1,000 Å, and the thickness of the hole transport layer may be about 50 Å to about 2,000 Å, for example, about 100 Å to about 1,500 Å. When the thicknesses of the hole transport region, the hole injection layer, and the hole transport layer are within the ranges described herein, satisfactory hole transporting characteristics may be obtained without a substantial increase in driving voltage.

The emission auxiliary layer may increase light emission efficiency by compensating for an optical resonance distance according to the wavelength of light emitted by the emission layer, and the electron-blocking layer may block or reduce the leakage of electrons from the emission layer to the hole transport region. Materials that may be included in the hole transport region may be included in the emission auxiliary layer and the electron-blocking layer.

p-Dopant

The hole transport region may further include, in addition to these materials, a charge-generating material for the improvement of conductive properties. The charge-generating material may be uniformly (e.g., substantially uniformly) or non-uniformly (e.g., substantially non-uniformly) dispersed in the hole transport region (for example, in the form of a single layer including (e.g., consisting of) a charge-generating material).

The charge-generating material may be, for example, a p-dopant.

The organic compound represented by Formula 1 may be a charge-generating material.

In one or more embodiments, the organic compound represented by Formula 1 may be, for example, a p-dopant.

For example, the LUMO energy of the p-dopant may be less than or equal to −3.5 eV. For example, the LUMO energy level of the p-dopant may be −5.0 eV or less.

According to one or more embodiments, the p-dopant may include, in addition to the organic compound represented by Formula 1, a quinone derivative, a cyano group-containing compound, an element EL1 and an element EL2-containing compound, or any combination thereof.

Examples of the quinone derivative may include TCNQ and F4-TCNQ.

Examples of the cyano group-containing compound may include HAT-CN and a compound represented by Formula 221.

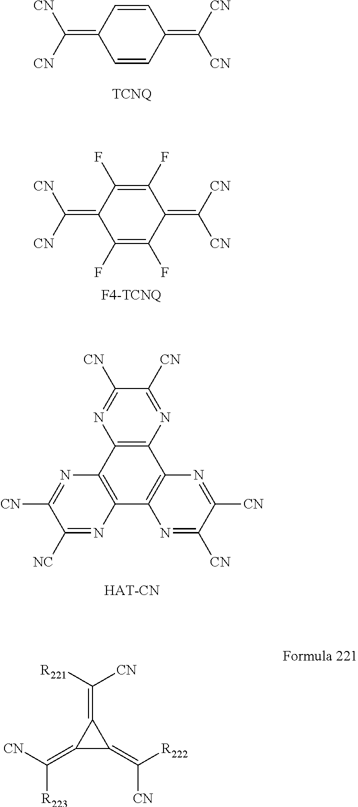

### In Formula 221,

R₂₂₁ to R₂₂₃ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —CI; —Br; —I; a C₁-C₂₀ alkyl group substituted with a cyano group, —F, —Cl, —Br, —I, or any combination thereof; or any combination thereof.

In the compound including the element EL1 and the element EL2, the element EL1 may be a metal, a metalloid, or a combination thereof, and the element EL2 may be a non-metal, a metalloid, or a combination thereof.

Examples of the metal may include an alkali metal (for example, lithium (Li), sodium (Na), potassium (K), rubidium (Rb), cesium (Cs), and/or the like); an alkaline earth metal (for example, beryllium (Be), magnesium (Mg), calcium (Ca), strontium (Sr), barium (Ba), and/or the like); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); a post-transition metal (for example, zinc (Zn), indium (In), tin (Sn), and/or the like); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like).

Examples of the metalloid may include silicon (Si), antimony (Sb), and tellurium (Te).

Examples of the non-metal may include oxygen (O) and halogen (for example, F, Cl, Br, I, and/or the like).

Examples of the compound including the element EL1 and the element EL2 may include a metal oxide, a metal halide (for example, a metal fluoride, a metal chloride, a metal bromide, a metal iodide, and/or the like), a metalloid halide (for example, a metalloid fluoride, a metalloid chloride, a metalloid bromide, a metalloid iodide, and/or the like), a metal telluride, or any combination thereof.

Examples of the metal oxide may include a tungsten oxide (for example, WO, W₂O₃, WO₂, WO₃, W₂O₅, and/or the like), a vanadium oxide (for example, VO, V₂O₃, VO₂, V₂O₅, and/or the like), a molybdenum oxide (MoO, Mo₂O₃, MoO₂, MoO₃, Mo₂O₅, and/or the like), and a rhenium oxide (for example, ReO₃, and/or the like).

Examples of the metal halide may include an alkali metal halide, an alkaline earth metal halide, a transition metal halide, a post-transition metal halide, and a lanthanide metal halide.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, and BaI₂.

Examples of the transition metal halide may include a titanium halide (for example, TiF₄, TiCl₄, TiBr₄, TiI₄, and/or the like), a zirconium halide (for example, ZrF₄, ZrCl₄, ZrBr₄, ZrI₄, and/or the like), a hafnium halide (for example, HfF₄, HfCl₄, HfBr₄, HfI₄, and/or the like), a vanadium halide (for example, VF₃, VCl₃, VBr₃, VI₃, and/or the like), a niobium halide (for example, NbF₃, NbCl₃, NbBr₃, NbI₃, and/or the like), a tantalum halide (for example, TaF₃, TaCl₃, TaBr₃, TaI₃, and/or the like), a chromium halide (for example, CrF₃, CrCl₃, CrBr₃, CrI₃, and/or the like), a molybdenum halide (for example, MoF₃, MoCl₃, MoBr₃, MoI₃, and/or the like), a tungsten halide (for example, WF₃, WCl₃, WBr₃, WI₃, and/or the like), a manganese halide (for example, MnF₂, MnCl₂, MnBr₂, MnI₂, and/or the like), a technetium halide (for example, TcF₂, TcCl₂, TcBr₂, TcI₂, and/or the like), a rhenium halide (for example, ReF₂, ReCl₂, ReBr₂, ReI₂, and/or the like), an iron halide (for example, FeF₂, FeCl₂, FeBr₂, FeI₂, and/or the like), a ruthenium halide (for example, RuF₂, RuCl₂, RuBr₂, RuI₂, and/or the like), an osmium halide (for example, OsF₂, OsCl₂, OsBr₂, OsI₂, and/or the like), a cobalt halide (for example, CoF₂, CoCl₂, CoBr₂, CoI₂, and/or the like), a rhodium halide (for example, RhF₂, RhCl₂, RhBr₂, RhI₂, and/or the like), an iridium halide (for example, IrF₂, IrCl₂, IrBr₂, IrI₂, and/or the like), a nickel halide (for example, NiF₂, NiCl₂, NiBr₂, NiI₂, and/or the like), a palladium halide (for example, PdF₂, PdCl₂, PdBr₂, PdI₂, and/or the like), a platinum halide (for example, PtF₂, PtCl₂, PtBr₂, PtI₂, and/or the like), a copper halide (for example, CuF, CuCl, CuBr, CuI, and/or the like), a silver halide (for example, AgF, AgCl, AgBr, AgI, and/or the like), and a gold halide (for example, AuF, AuCl, AuBr, AuI, and/or the like).

Examples of the post-transition metal halide may include a zinc halide (for example, ZnF₂, ZnCl₂, ZnBr₂, ZnI₂, and/or the like), an indium halide (for example, InI₃, and/or the like), and a tin halide (for example, SnI₂, and/or the like).

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

Examples of the metalloid halide may include an antimony halide (for example, SbCl₅, and/or the like).

Examples of the metal telluride may include an alkali metal telluride (for example, Li₂Te, Na₂Te, K₂Te, Rb₂Te, Cs₂Te, and/or the like), an alkaline earth metal telluride (for example, BeTe, MgTe, CaTe, SrTe, BaTe, and/or the like), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and/or the like), a post-transition metal telluride (for example, ZnTe, and/or the like), and a lanthanide metal telluride (for example, LaTe, CeTe, PrTe, NdTe, PmTe, EuTe, GdTe, TbTe, DyTe, HoTe, ErTe, TmTe, YbTe, LuTe, and/or the like).

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and a blue emission layer, in which the two or more layers contact each other or are separated from each other, to emit white light. In one or more embodiments, the emission layer may include two or more materials of a red light-emitting material, a green light-emitting material, and a blue light-emitting material, in which the two or more materials are mixed with each other in a single layer, to emit white light.

In one or more embodiments, the emission layer may include a host and a dopant (or an emitter). In one or more embodiments, the emission layer may further include an auxiliary dopant that promotes energy transfer to a dopant (or an emitter), in addition to the host and the dopant (or an emitter). When the emission layer includes the dopant (or an emitter) and the auxiliary dopant, the dopant (or an emitter) and the auxiliary dopant are different from each other.

An amount (weight) of the dopant (or an emitter) in the emission layer may be in a range of about 0.01 parts by weight to about 15 parts by weight based on 100 parts by weight of the host.

In one or more embodiments, the emission layer may include a quantum dot.

In one or more embodiments, the emission layer may include a delayed fluorescence material. The delayed fluorescence material may act as a host or a dopant in the emission layer.

The thickness of the emission layer may be about 100 Å to about 1,000 Å, for example, about 200 Å to about 600 Å. When the thickness of the emission layer is within the range described herein, excellent luminescence characteristics may be obtained without a substantial increase in driving voltage.

### Host

In one or more embodiments, the host may include a compound represented by Formula 301:

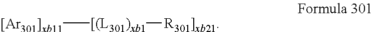

wherein, in Formula 301,
Ar₃₀₁ and L₃₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ are each as described in connection with Q₁.

In one or more embodiments, when xb11 in Formula 301 is 2 or more, two or more of Ar₃₀₁ may be linked to each other via a single bond (e.g., a single covalent bond).

In one or more embodiments, the host may include a compound represented by Formula 301-1, a compound represented by Formula 301-2, or any combination thereof:

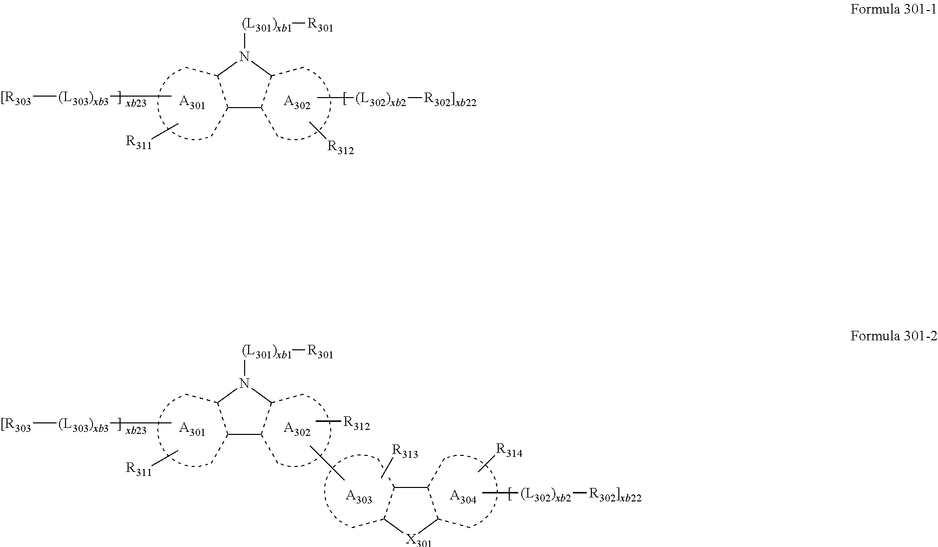

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ are each as described in the present specification,
L₃₀₂ to L₃₀₄ are each independently as described in connection with L₃₀₁,
xb2 to xb4 are each independently as described in connection with xb1, and
R₃₀₂ to R₃₀₅ and R₃₁₁ to R₃₁₄ are each as described in connection with R₃₀₁.

In one or more embodiments, the host may include an alkali earth metal complex, a post-transition metal complex, or any combination thereof. In one or more embodiments, the host may include a Be complex (for example, compound H55), an Mg complex, a Zn complex, or any combination thereof.

In one or more embodiments, the host may include: at least one of compounds H1 to H128; 9,10-di(2-naphthyl)anthracene (ADN); 2-methyl-9,10-bis(naphthalen-2-yl)anthracene (MADN); 9,10-di(2-naphthyl)-2-t-butyl-anthracene (TBADN); 4,4′-bis(N-carbazolyl)-1,1′-biphenyl (CBP); 1,3-di-9-carbazolylbenzene (mCP); 1,3,5-tri(carbazol-9-yl)benzene (TCP); or any combination thereof:

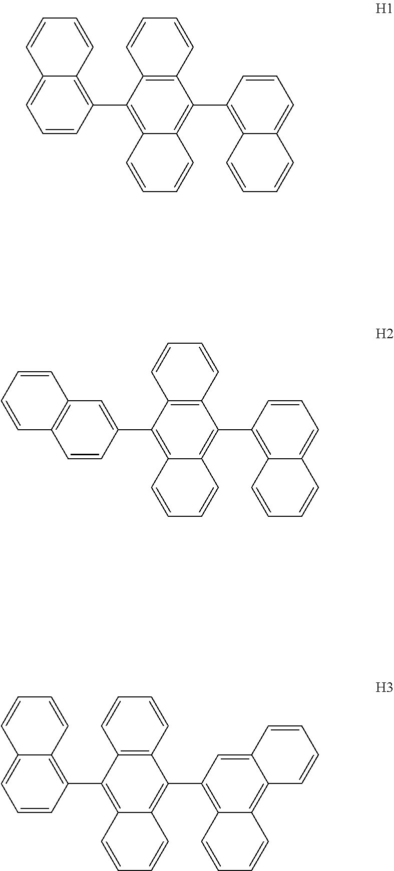

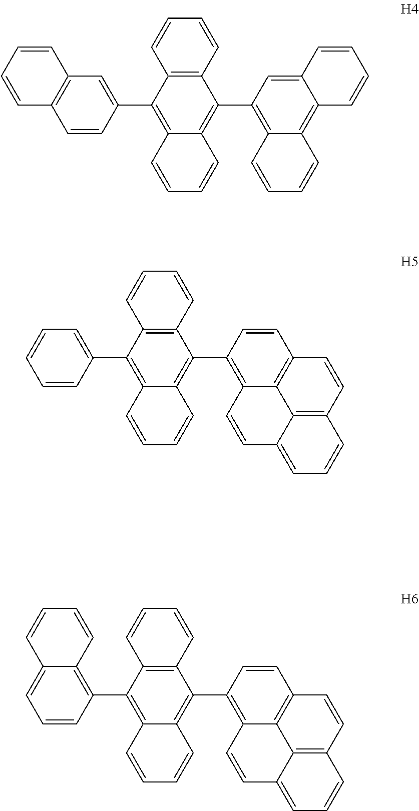

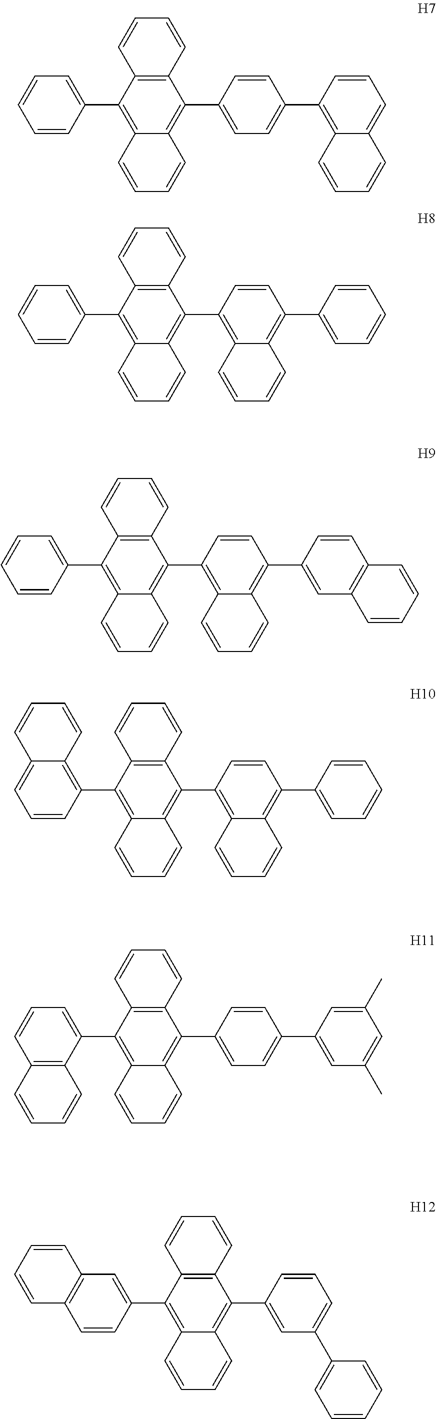

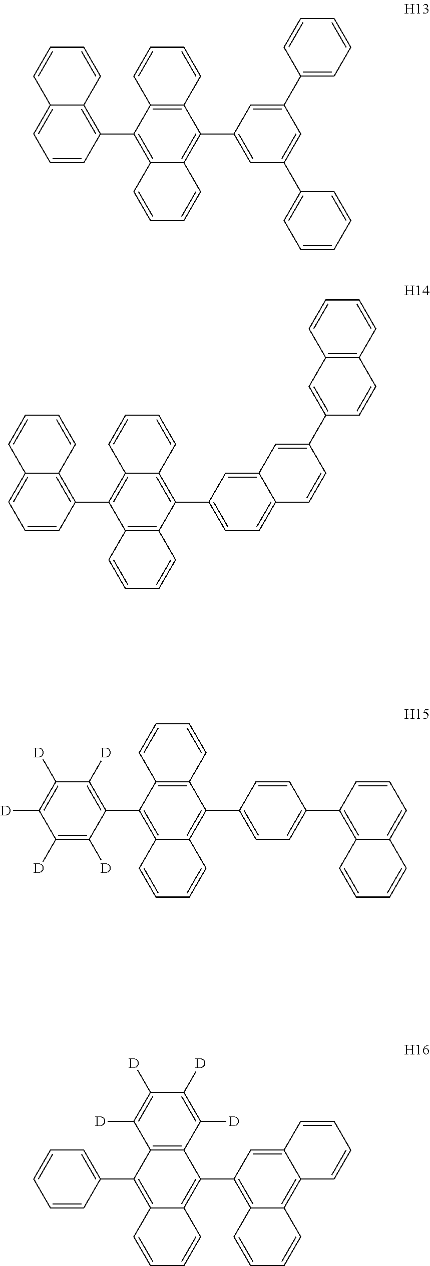

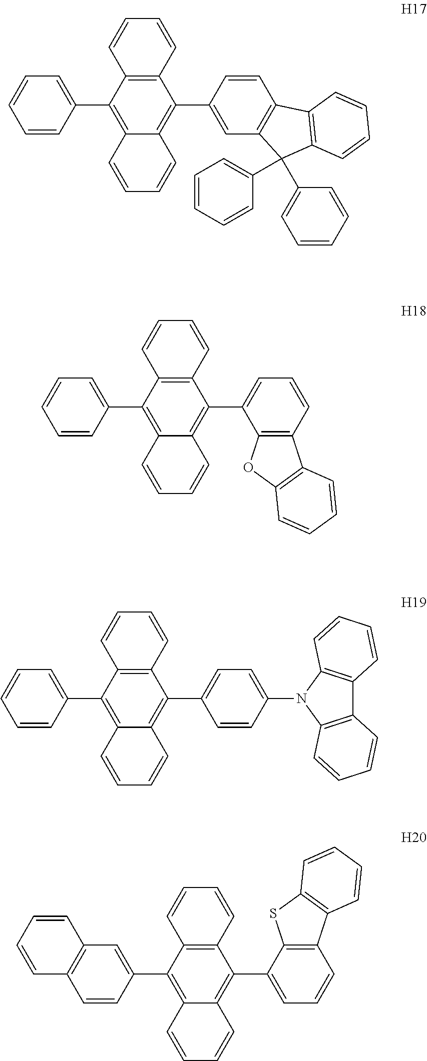

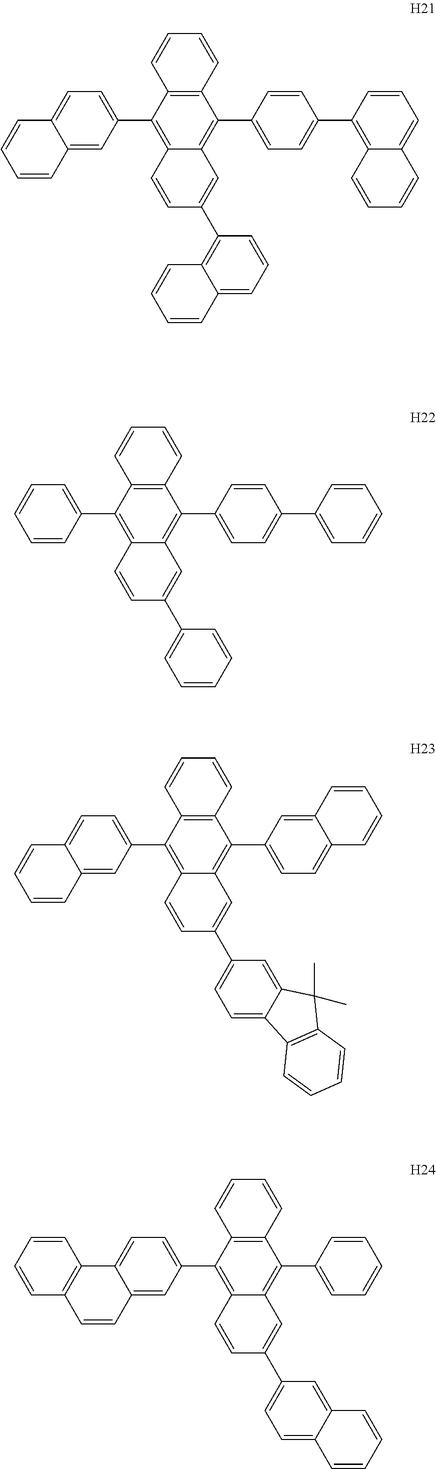

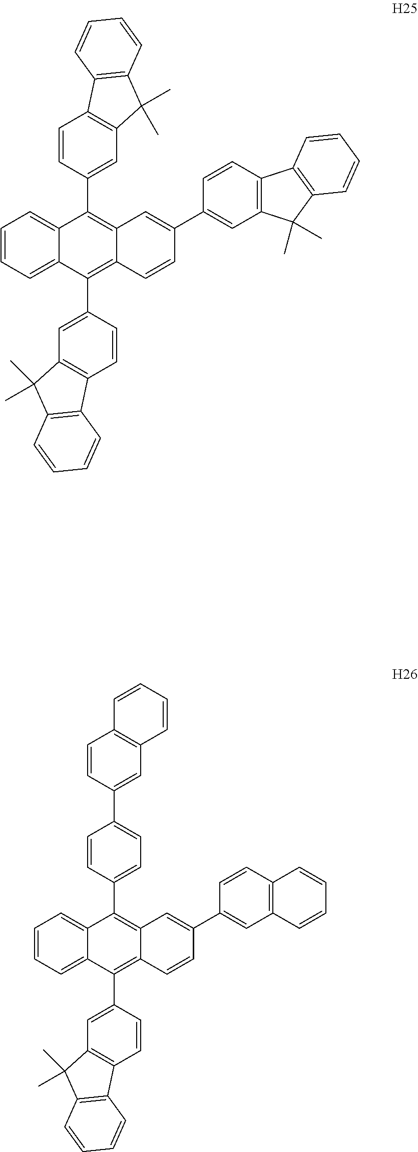

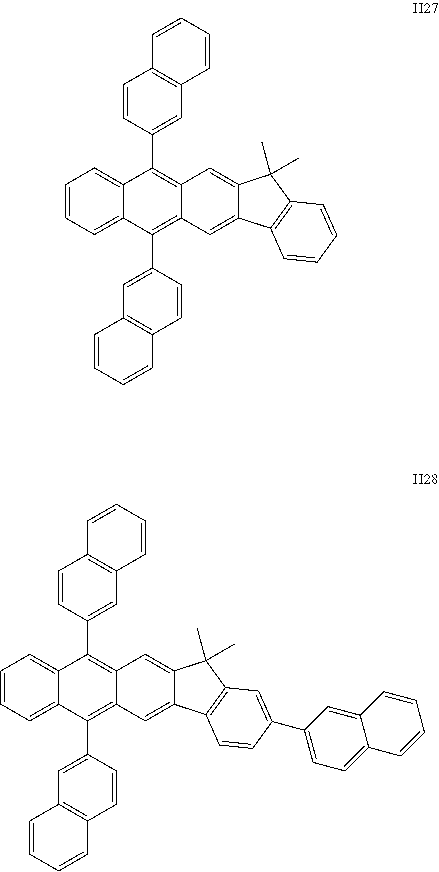

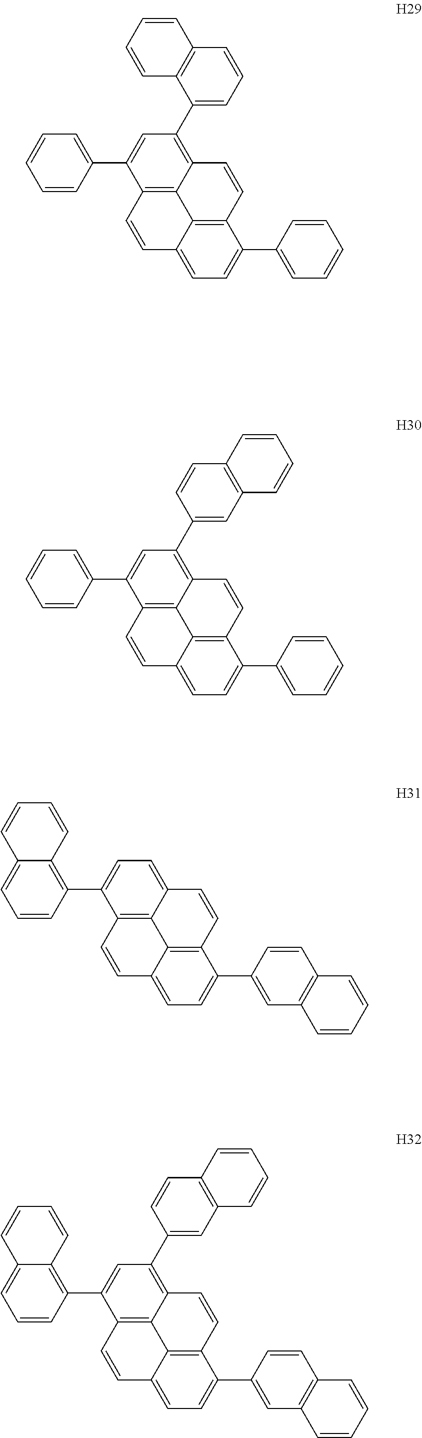

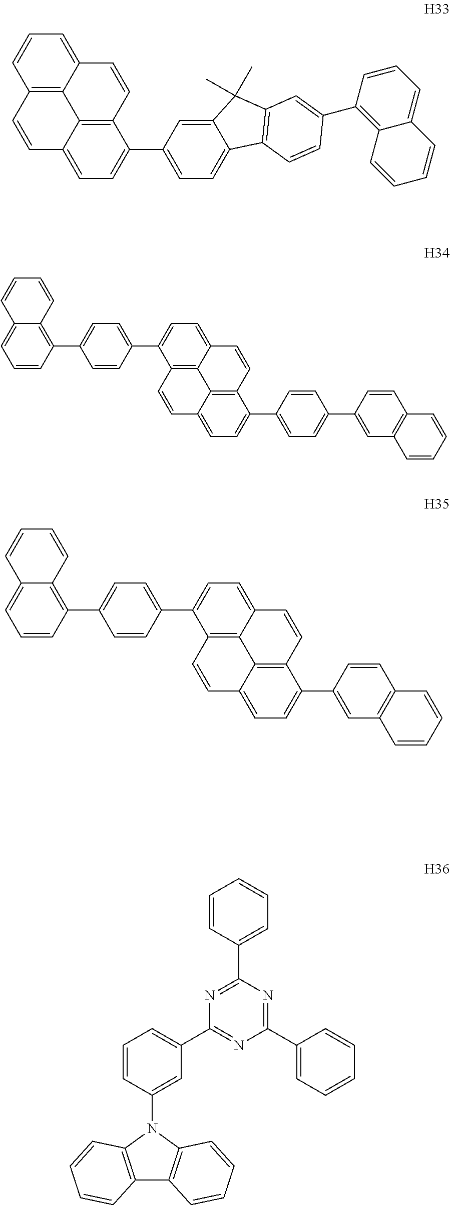

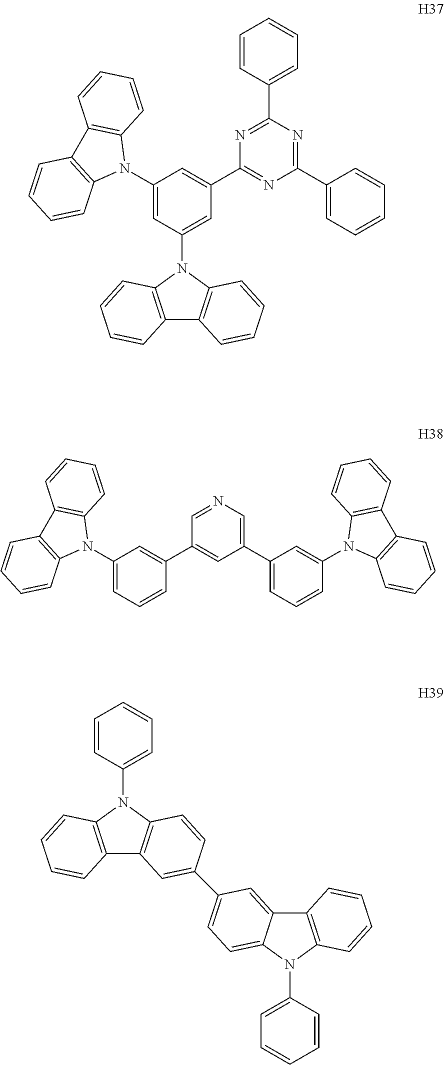 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1]

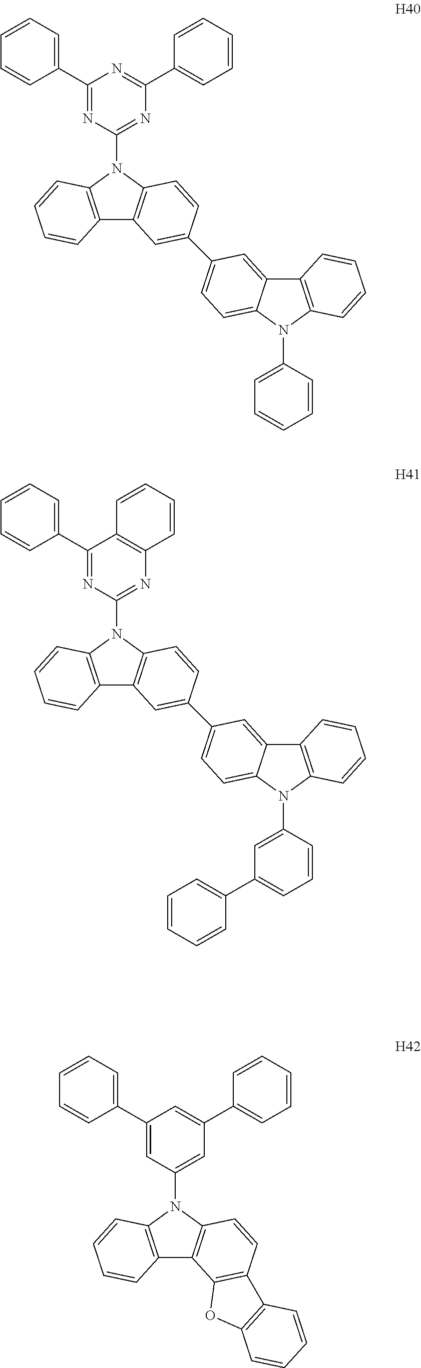 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

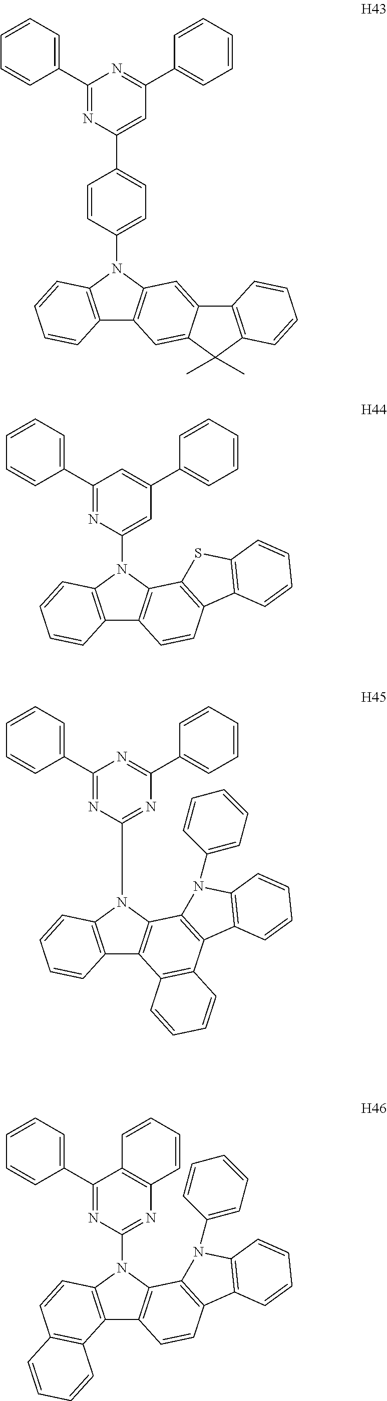 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

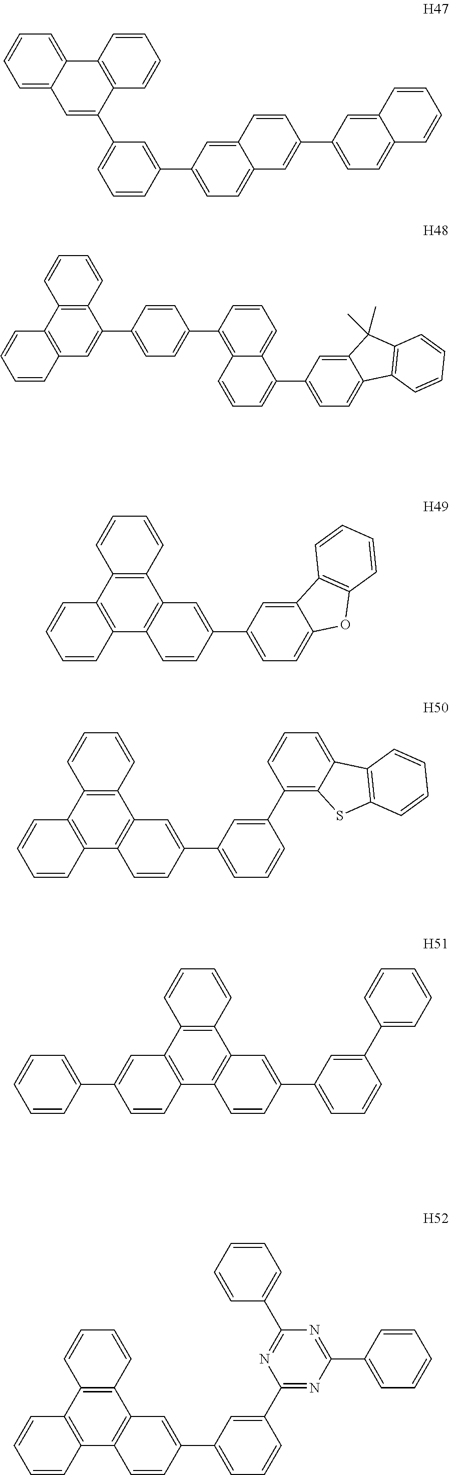

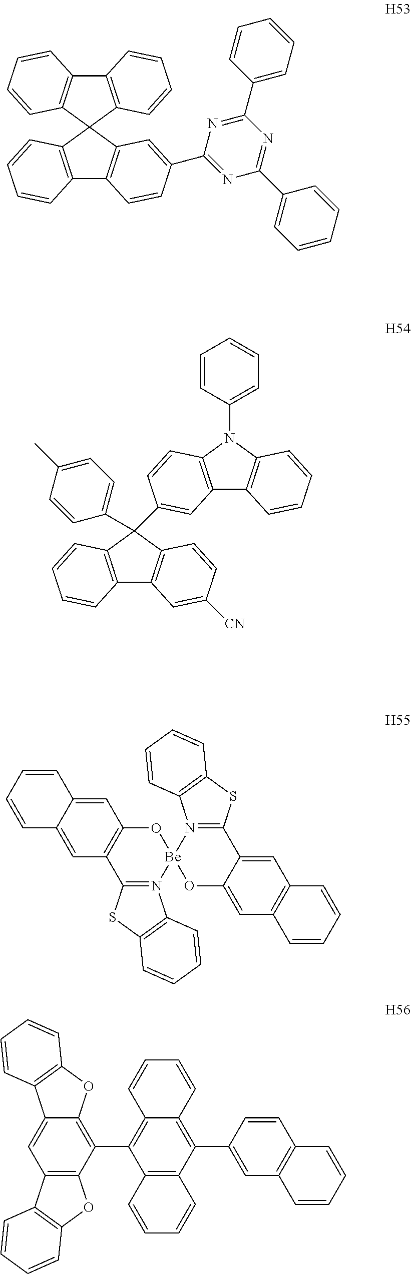

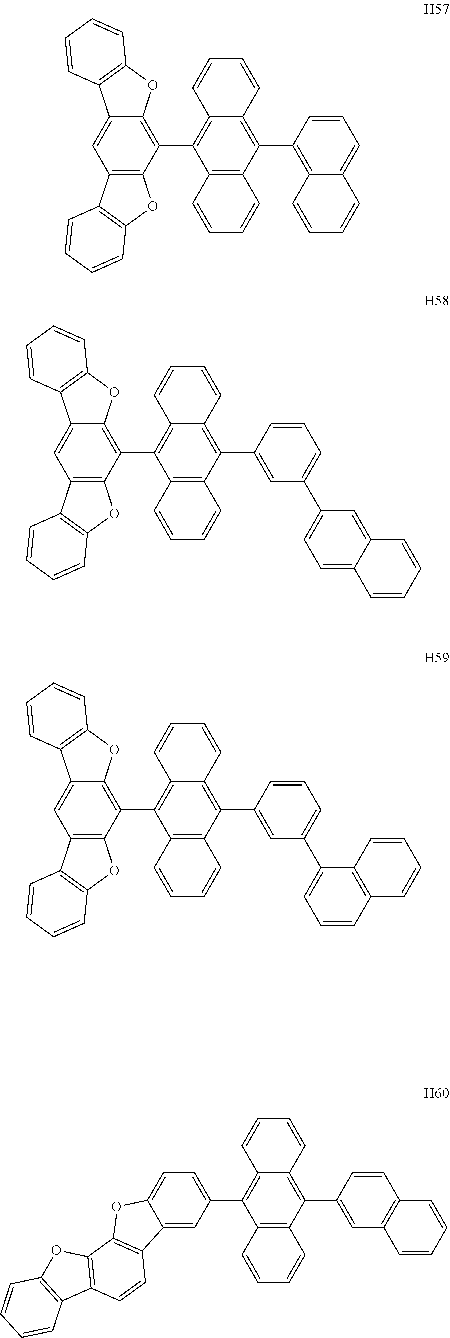 [SMILES: c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1]

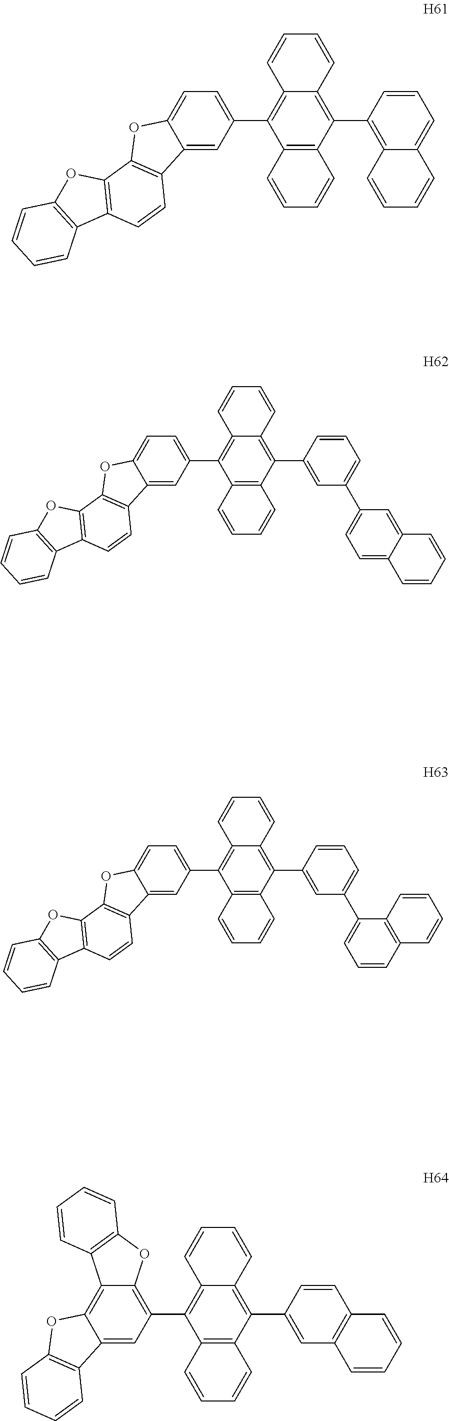 [SMILES: c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1]

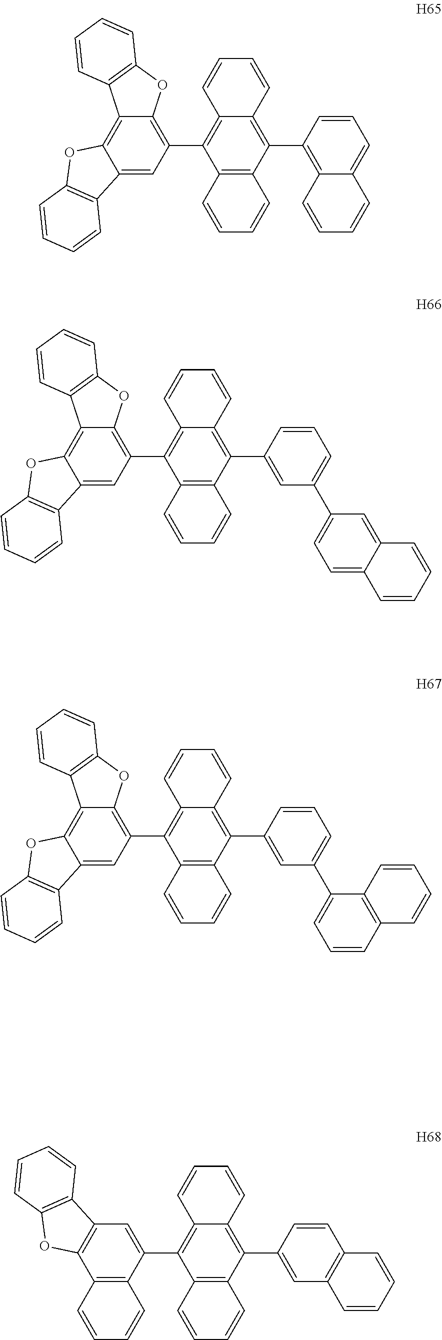 [SMILES: c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1]

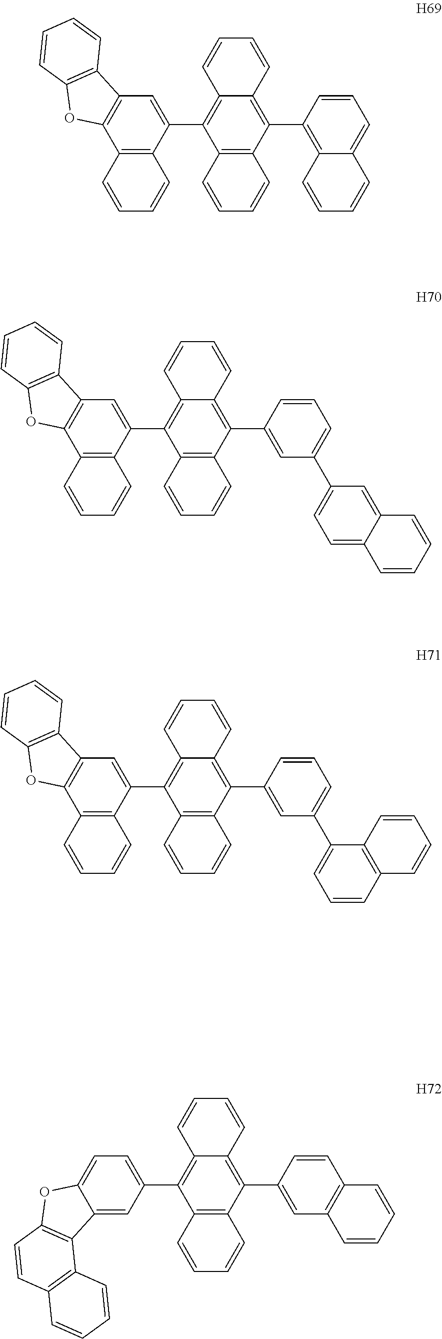 [SMILES: c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1]

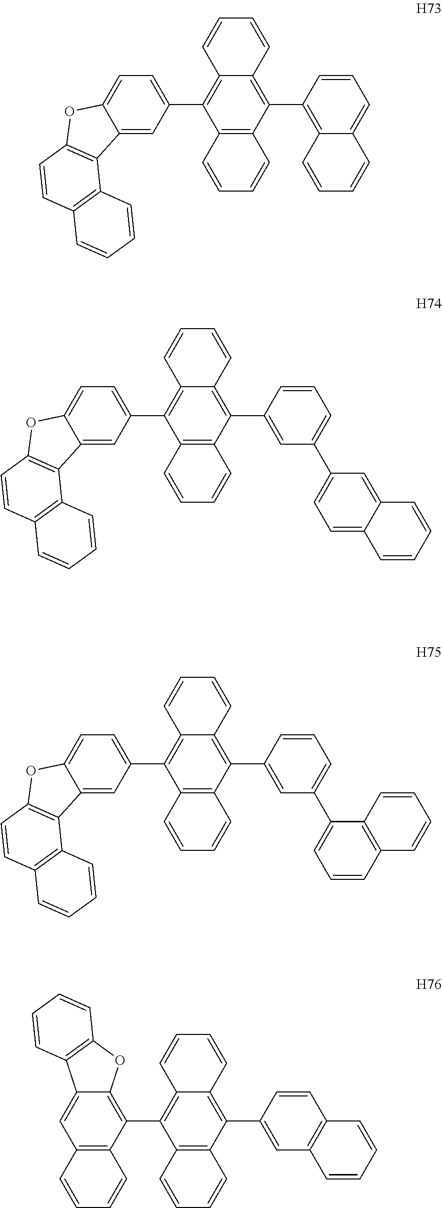 [SMILES: c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1]

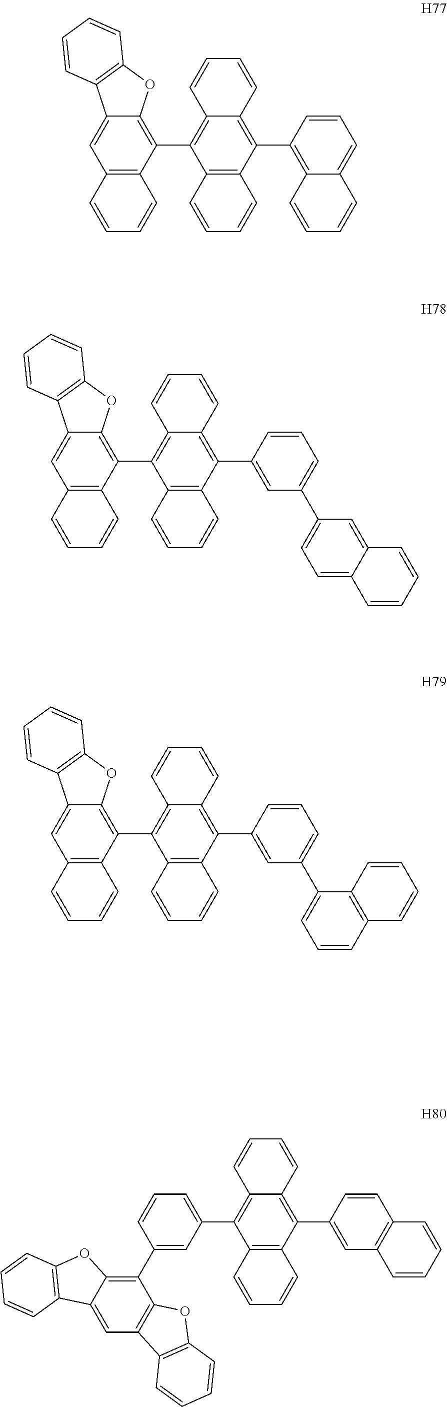 [SMILES: c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1]

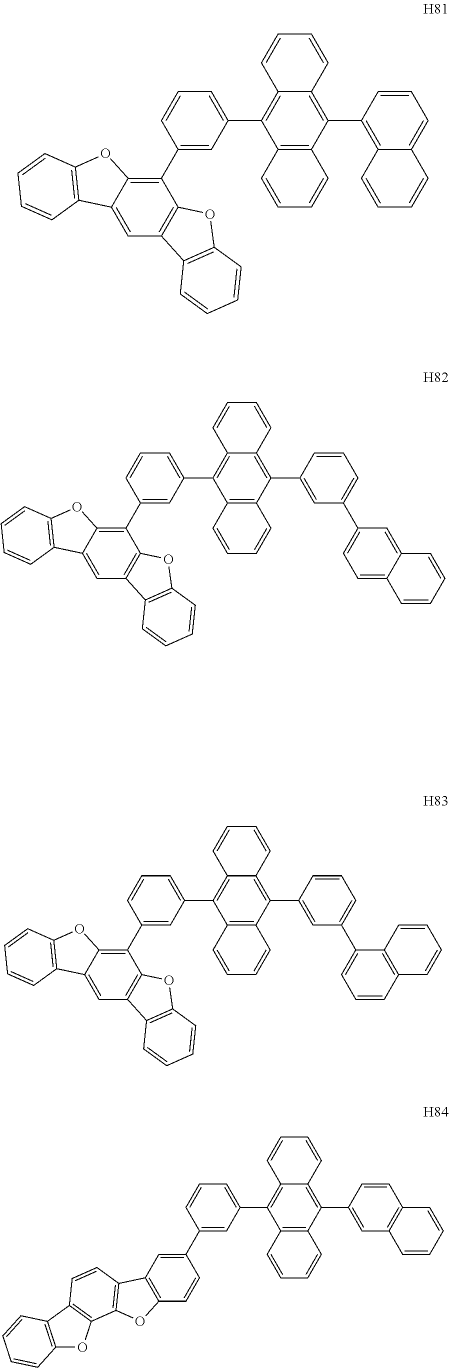 [SMILES: c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1]

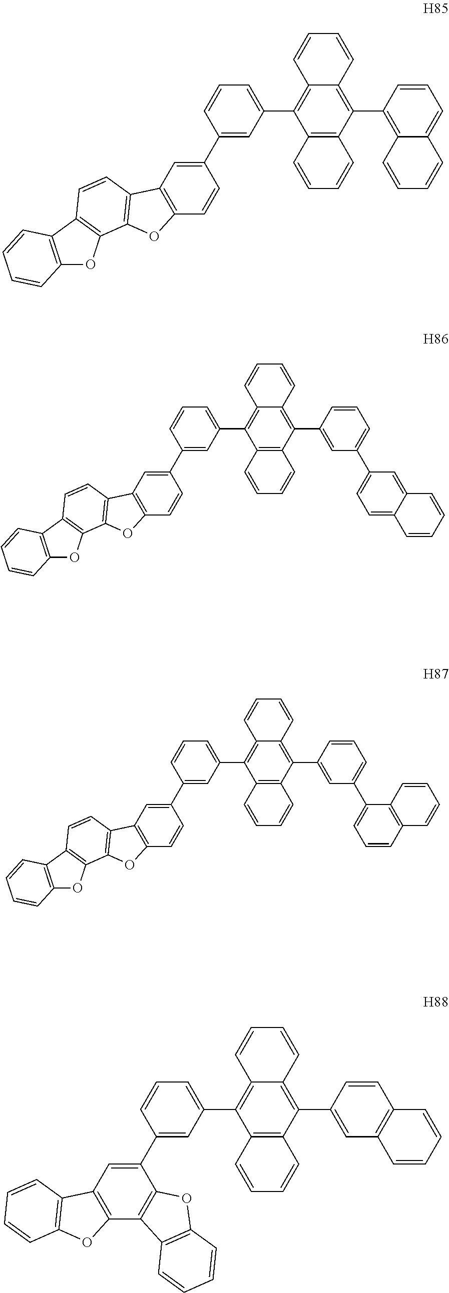 [SMILES: c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1]

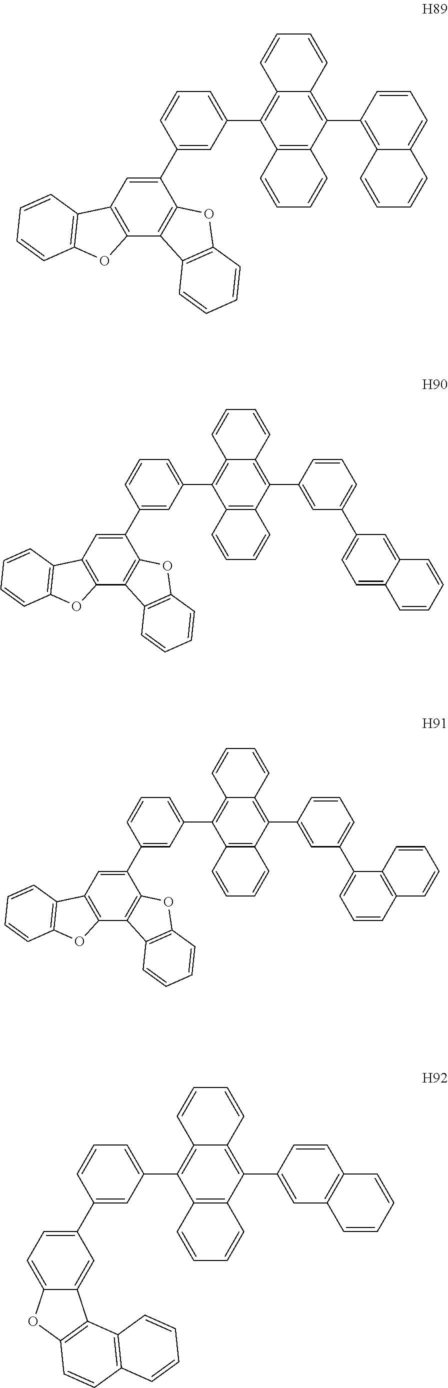 [SMILES: c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1]

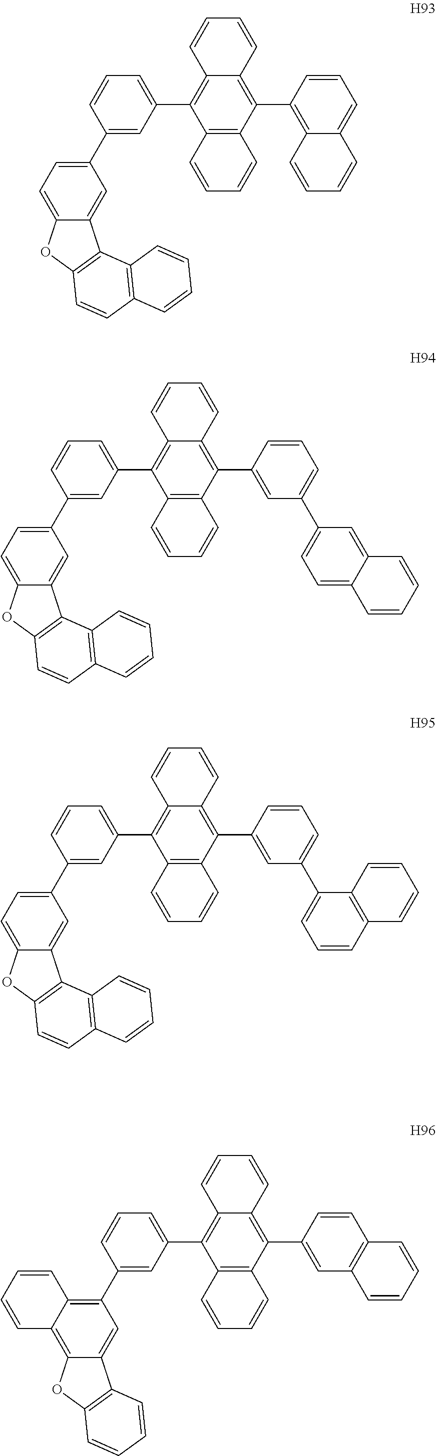 [SMILES: c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1]

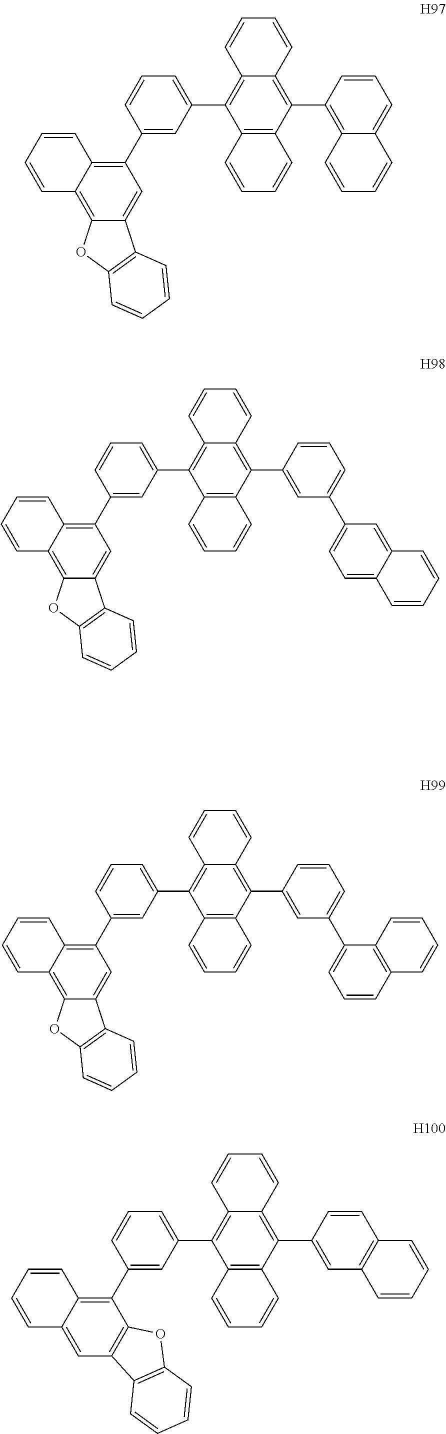 [SMILES: c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1]

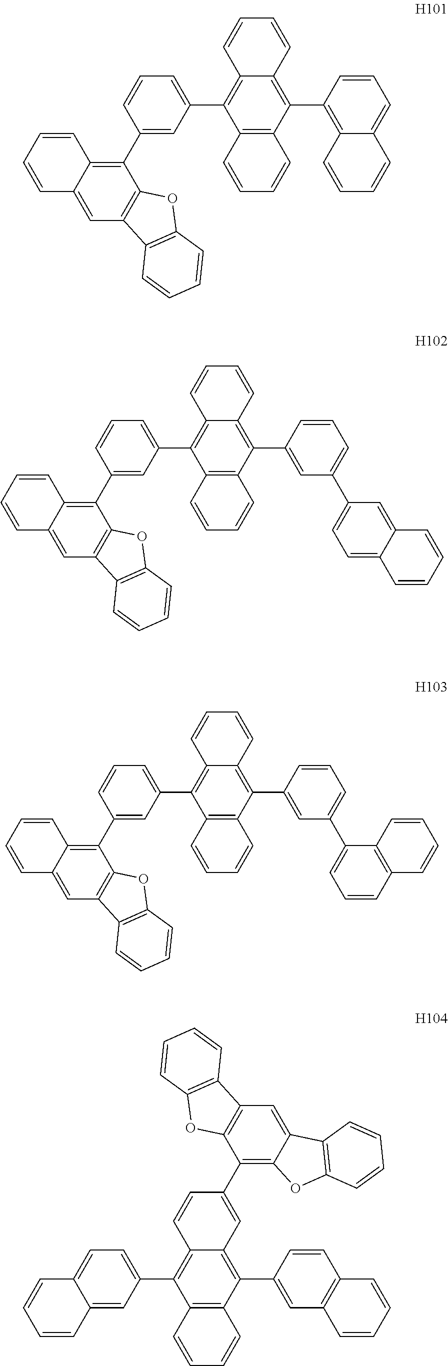 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

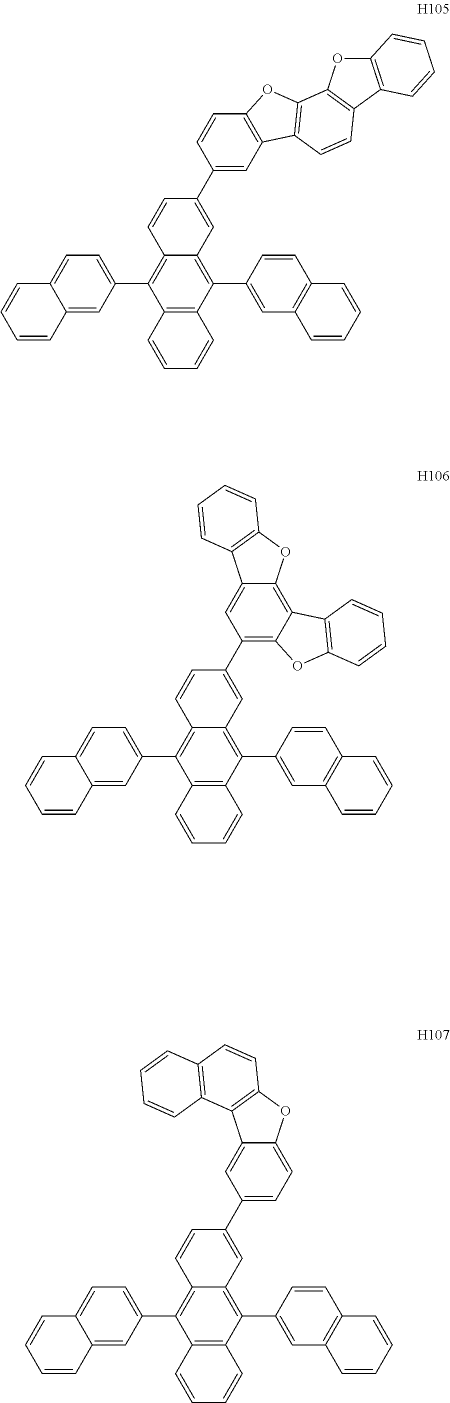 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1]

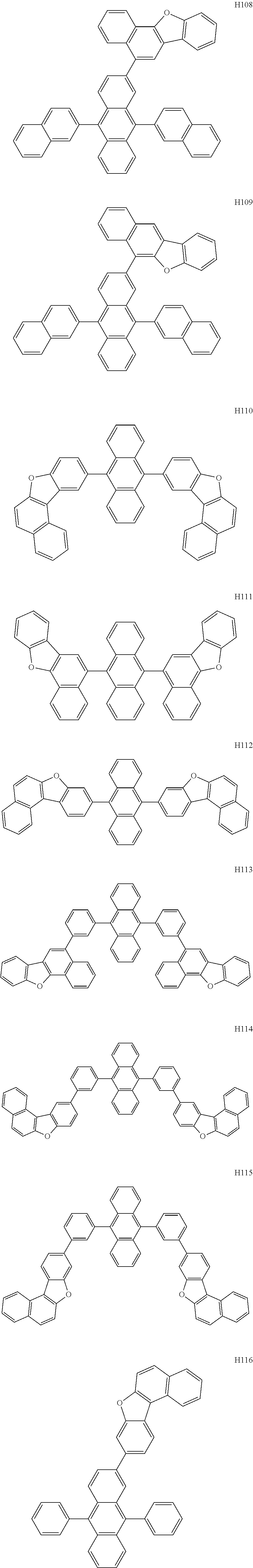

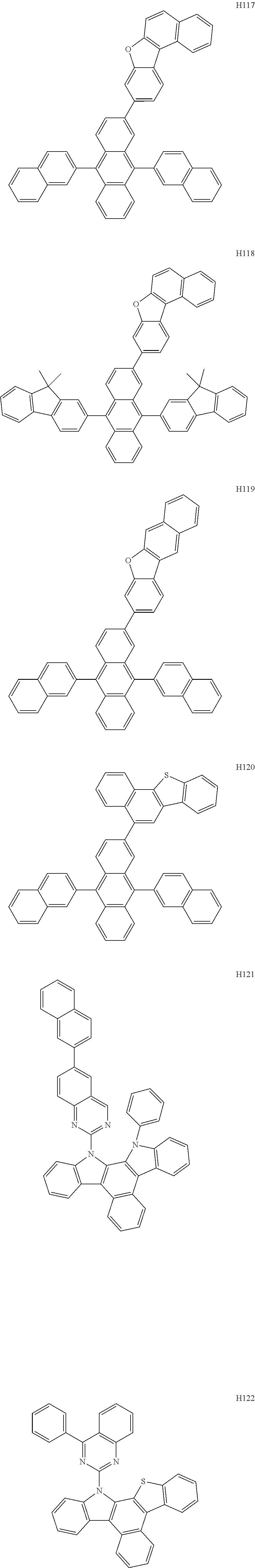

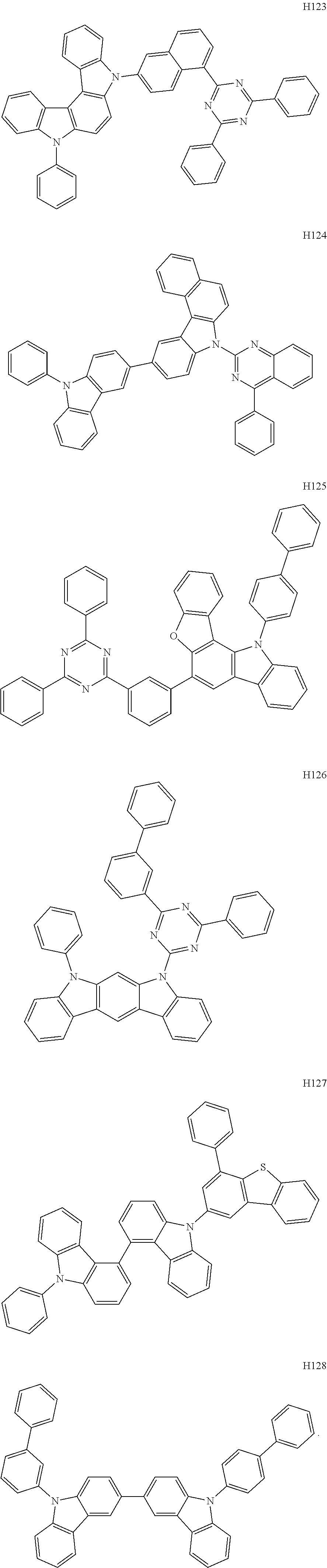

In one or more embodiments, the host may include a silicon-containing compound, a phosphine oxide-containing compound, or any combination thereof.

The host may have one or more suitable modifications. For example, the host may include only one (e.g., kind of) compound, or may include two or more kinds of different compounds.

### Phosphorescent Dopant

The emission layer may include a phosphorescent dopant.

The phosphorescent dopant may include at least one transition metal as a central metal. Therefore, the phosphorescent dopant may correspond to the fifth compound.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

In one or more embodiments, the phosphorescent dopant may include an organometallic compound represented by Formula 401:

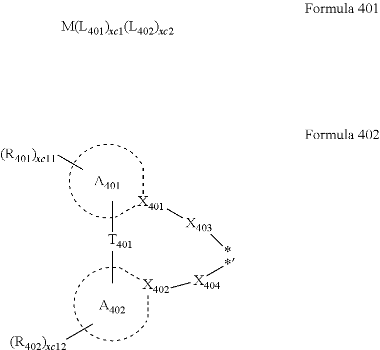

wherein, in Formulae 401 and 402,
M may be a transition metal (e.g., Ir, Pt, Pd, Os, Ti, Au, Hf, Eu, Tb, Rh, Re, or Tm),
L₄₀₁ may be a ligand represented by Formula 402, and xc1 is 1, 2, or 3, wherein, when xc1 is 2 or more, two or more of L₄₀₁ may be substantially identical to or different from each other,
L₄₀₂ may be an organic ligand, and xc2 may be 0, 1, 2, 3, or 4, wherein, when xc2 is 2 or more, two or more of L₄₀₂ may be substantially identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond (e.g., a single covalent bond), *—O—*′, *—S—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)=*′, or *═C(Q₄₁₁)=*′,
X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ are each as described in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ are each as described in connection with Q₁,
xc11 and xc12 may each independently be an integer from 0 to 10, and
* and *′ in Formula 402 each indicate a binding site to M in Formula 401.

In one or more embodiments, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 401 is 2 or more, two ring A₄₀₁ among two or more of L₄₀₁ may be optionally linked together via T₄₀₂, which is a linking group, and two ring A₄₀₂ may be optionally linked together via T₄₀₃, which is a linking group (see compounds PD1 to PD4 and PD7). T₄₀₂ and T₄₀₃ are each as described in connection with T₄₀₁.

L₄₀₂ in Formula 401 may be an organic ligand. In one or more embodiments, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, and/or the like), or any combination thereof.

The phosphorescent dopant may include, for example, one of compounds PD1 to PD39, or any combination thereof:

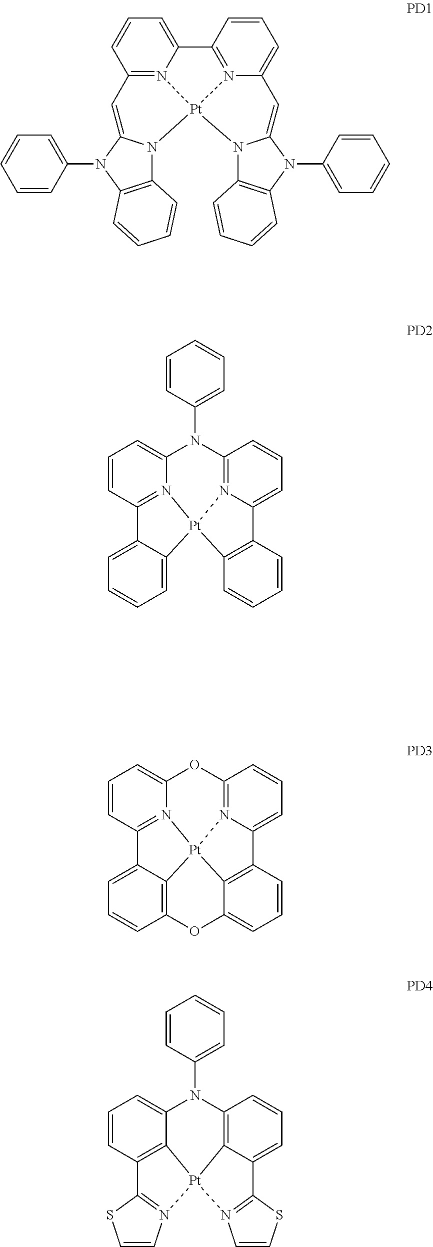

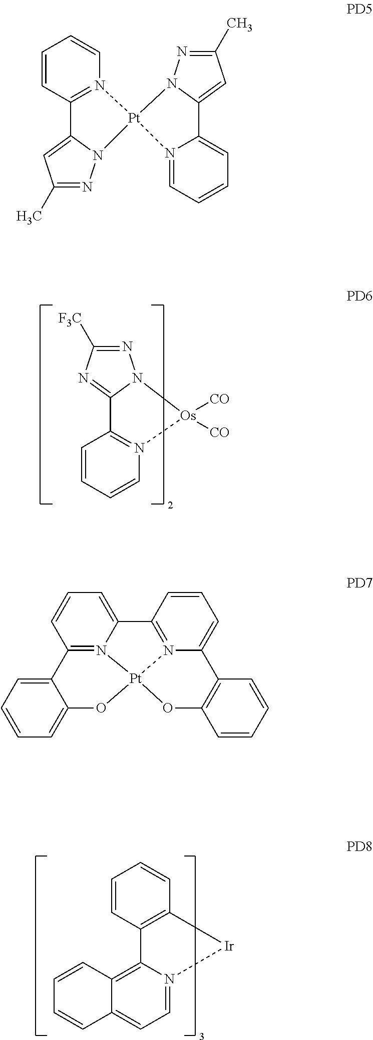

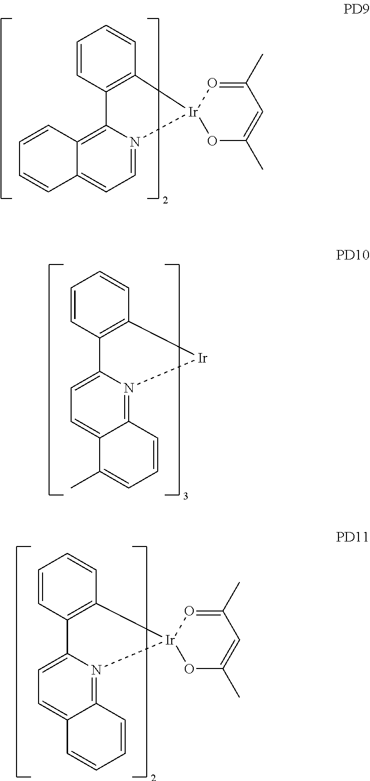

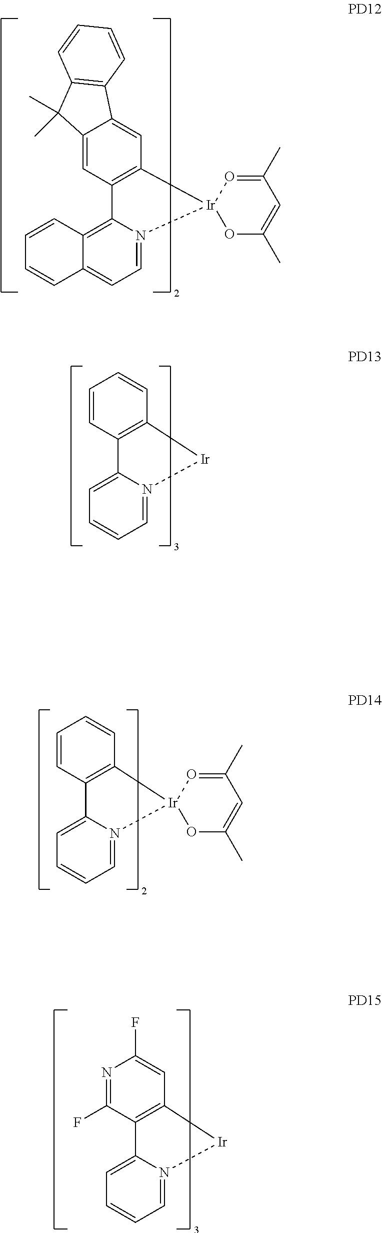

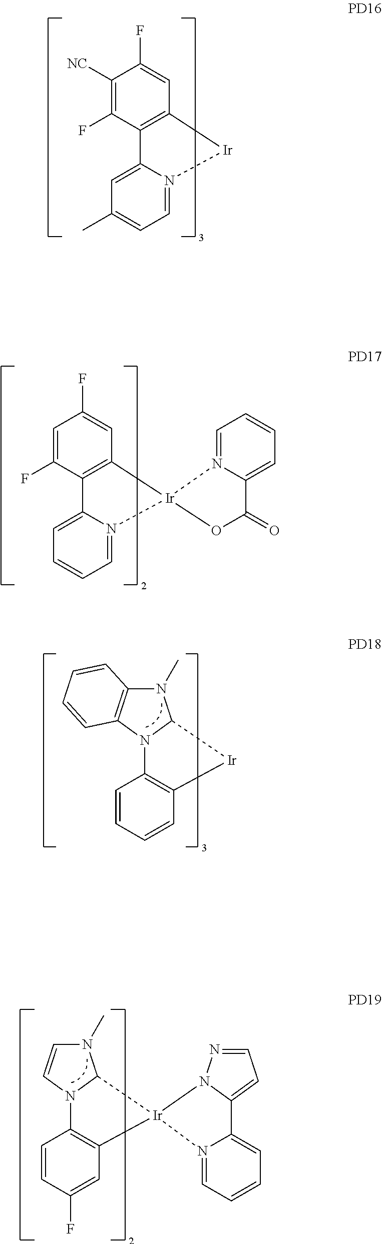

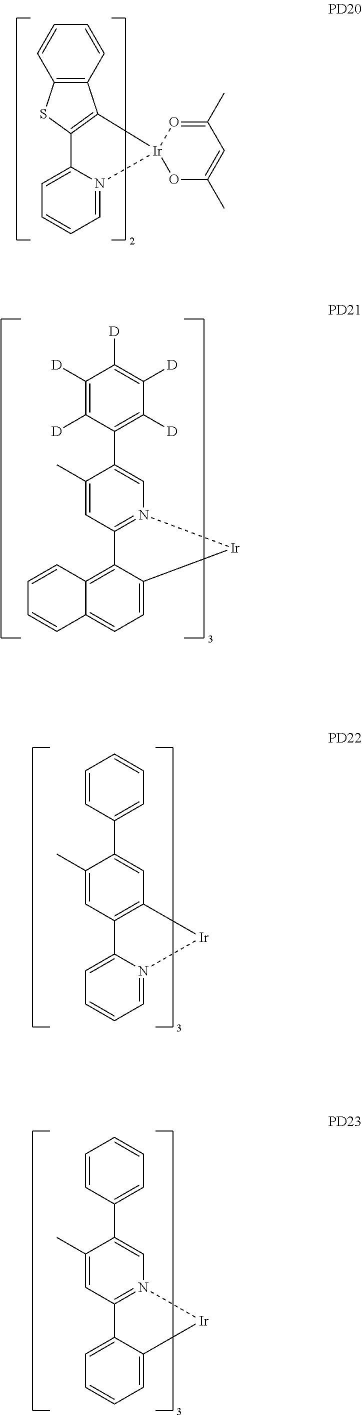

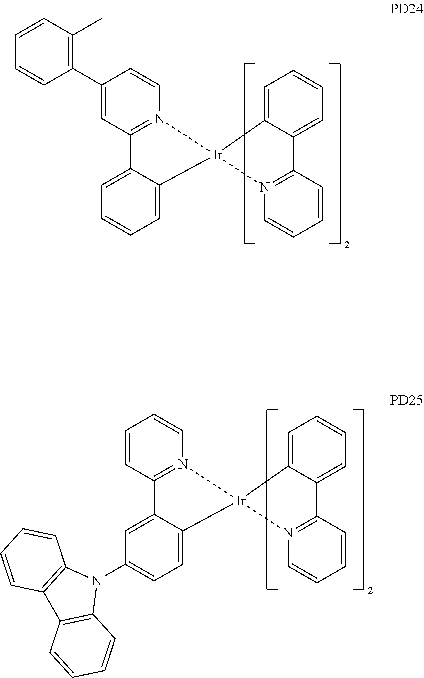

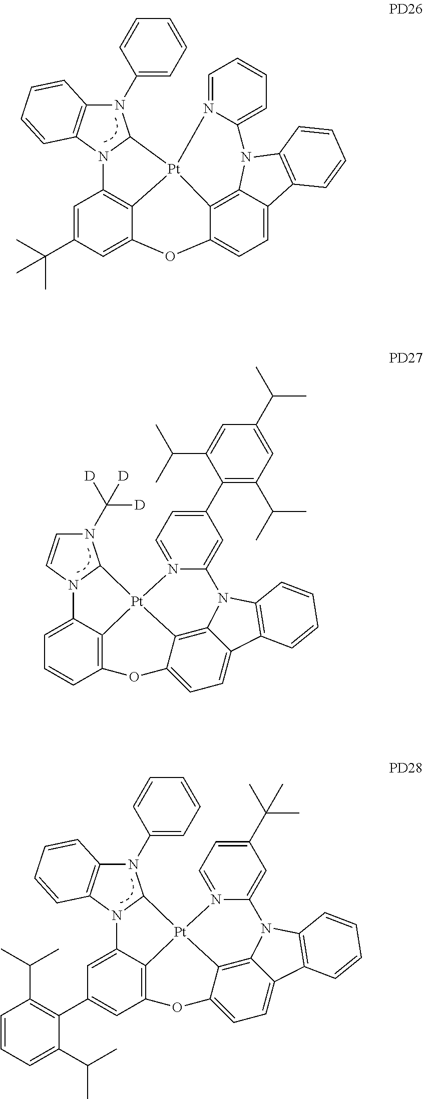

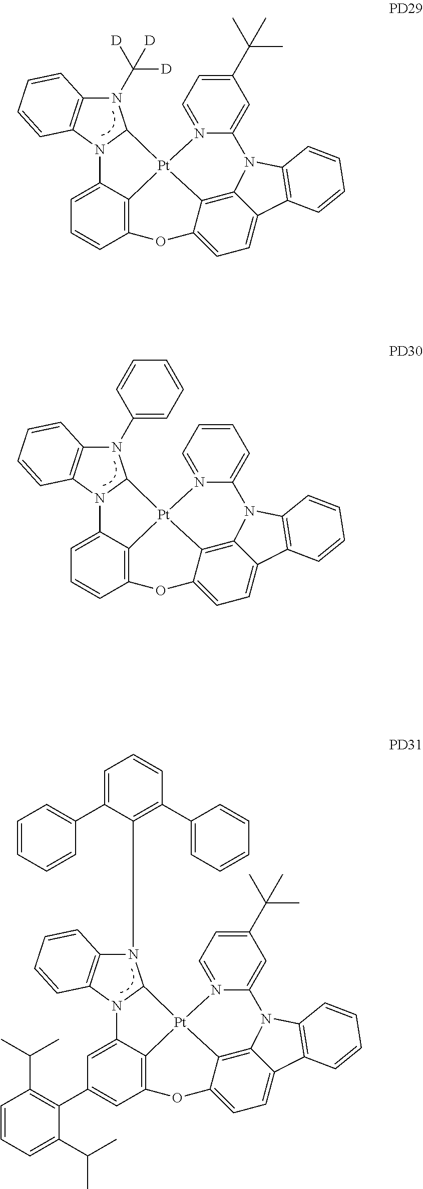

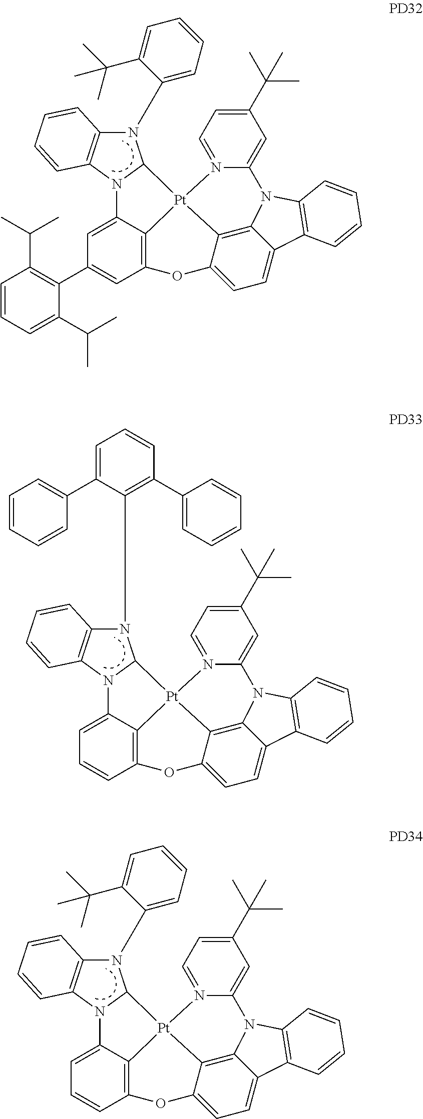

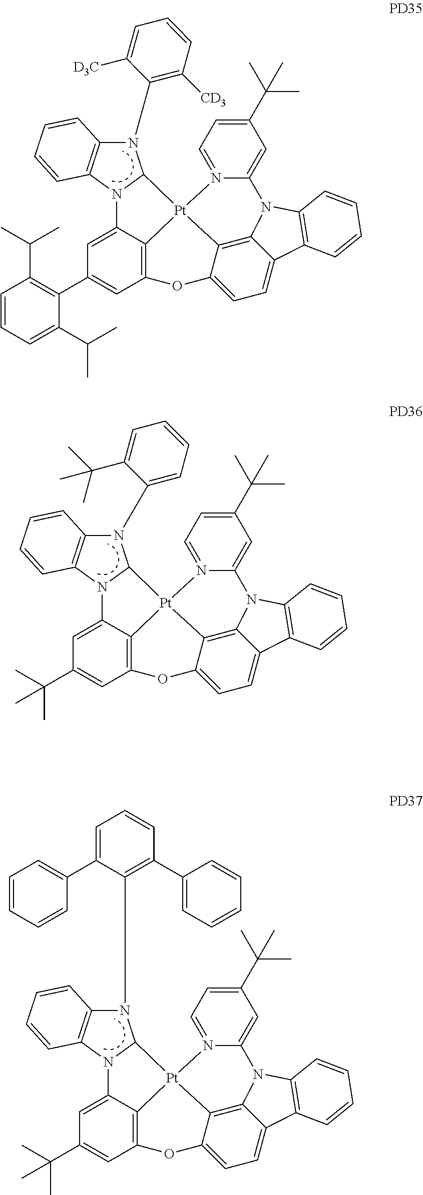

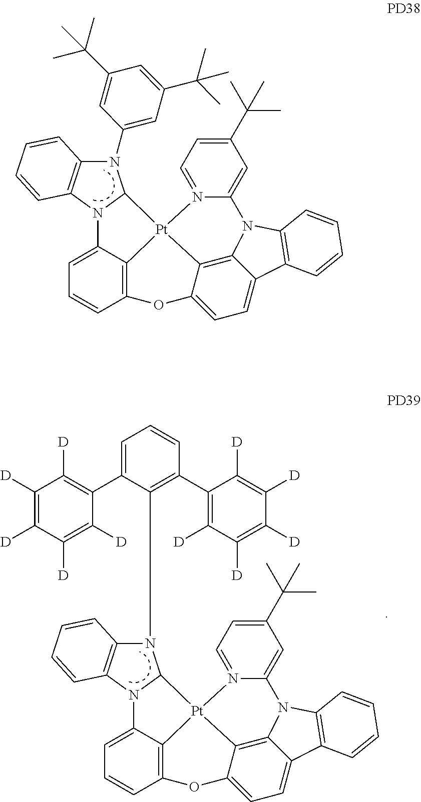

### Fluorescent Dopant

The emission layer may include a fluorescent dopant and/or an auxiliary dopant.

For example, the fluorescent dopant and/or the auxiliary dopant may each independently include a compound represented by Formula 501:

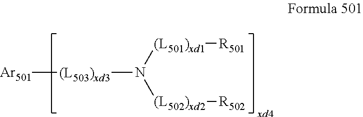

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

In one or more embodiments, Ar₅₀₁ in Formula 501 may be a condensed cyclic group (for example, an anthracene group, a chrysene group, a pyrene group, and/or the like) in which three or more monocyclic groups are condensed together.

In one or more embodiments, xd4 in Formula 501 may be 2.

In one or more embodiments, the fluorescent dopant and the auxiliary dopant may each include: at least one of compounds FD1 to FD37; DPVBi; DPAVB; or any combination thereof:

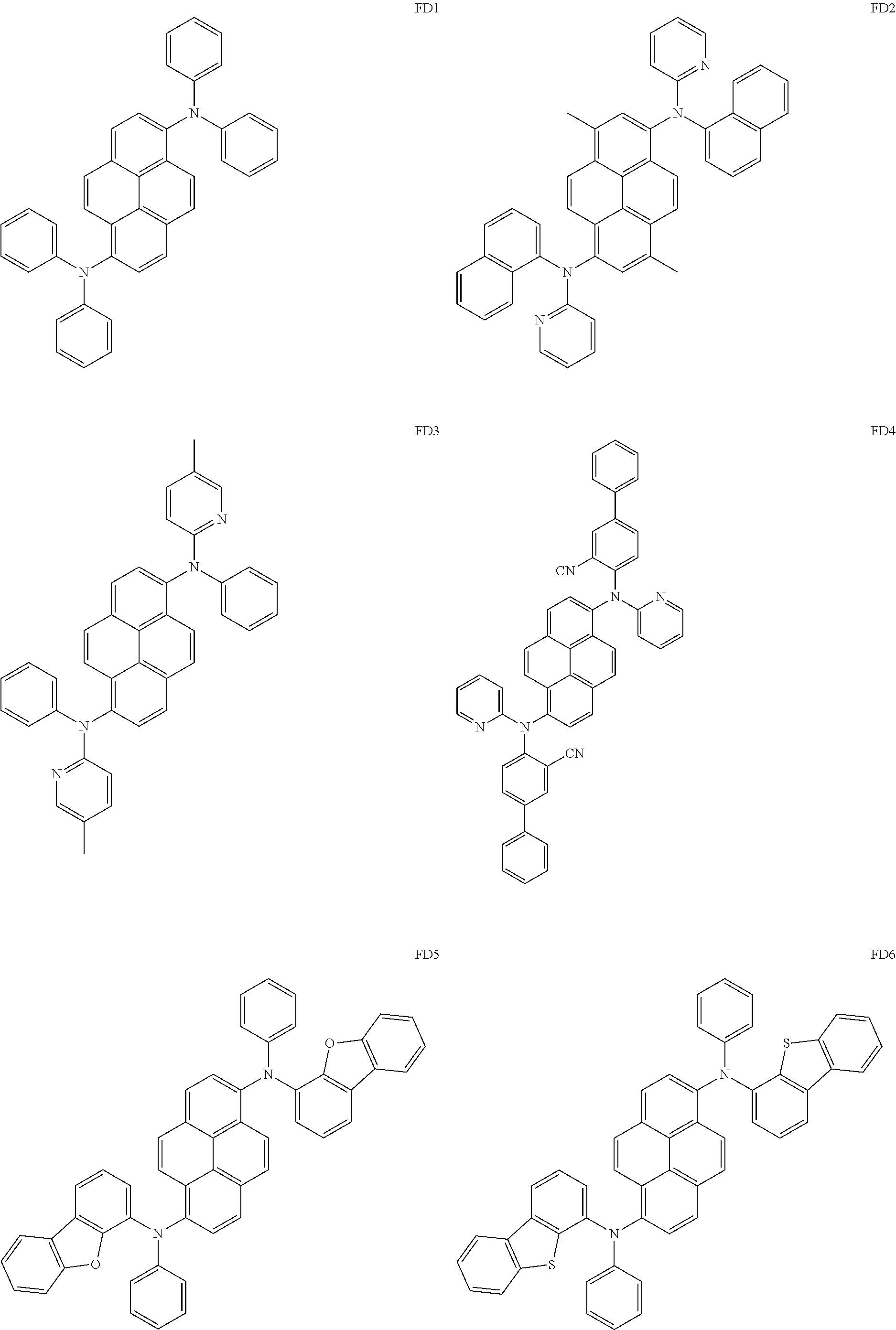

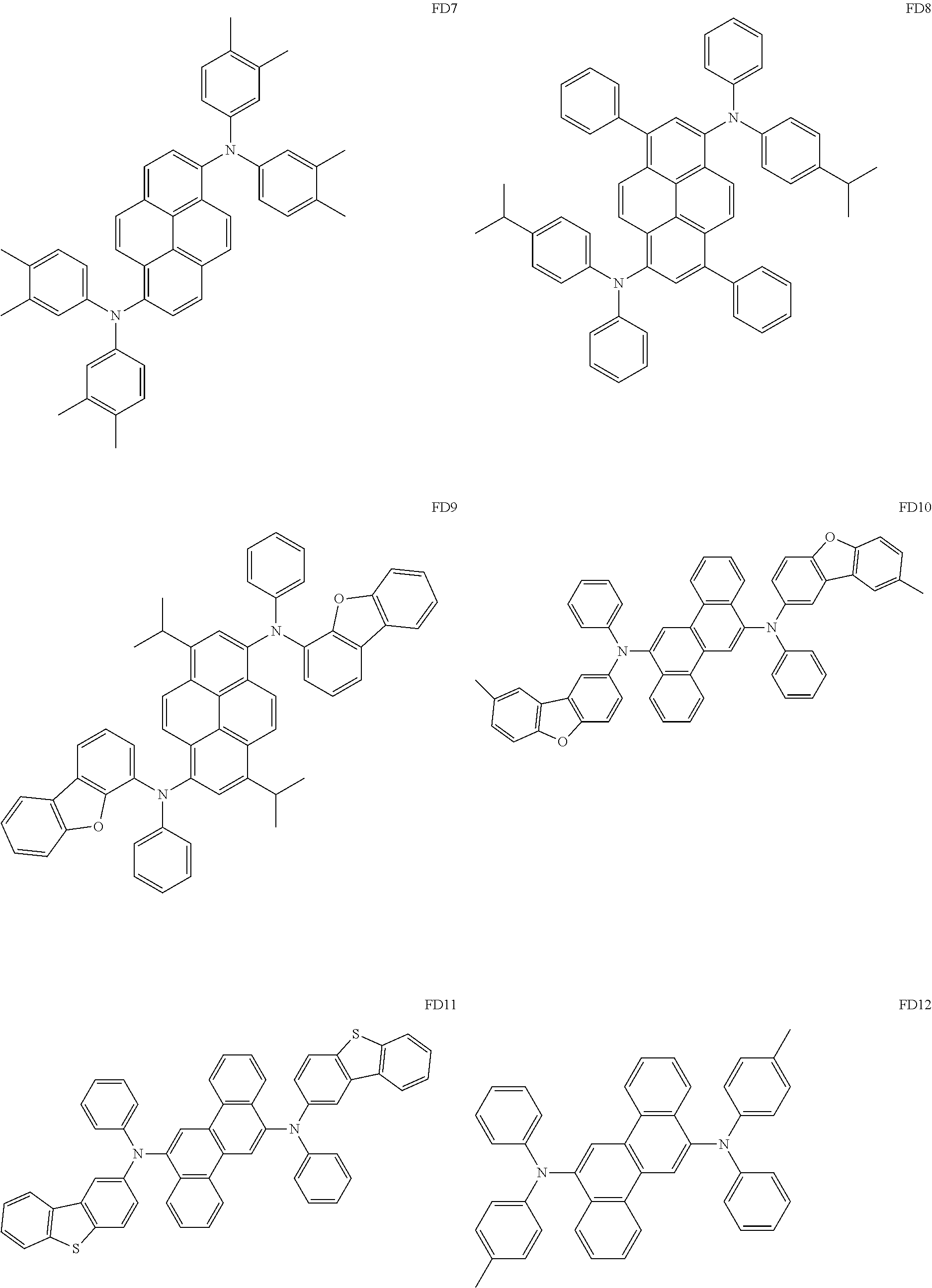

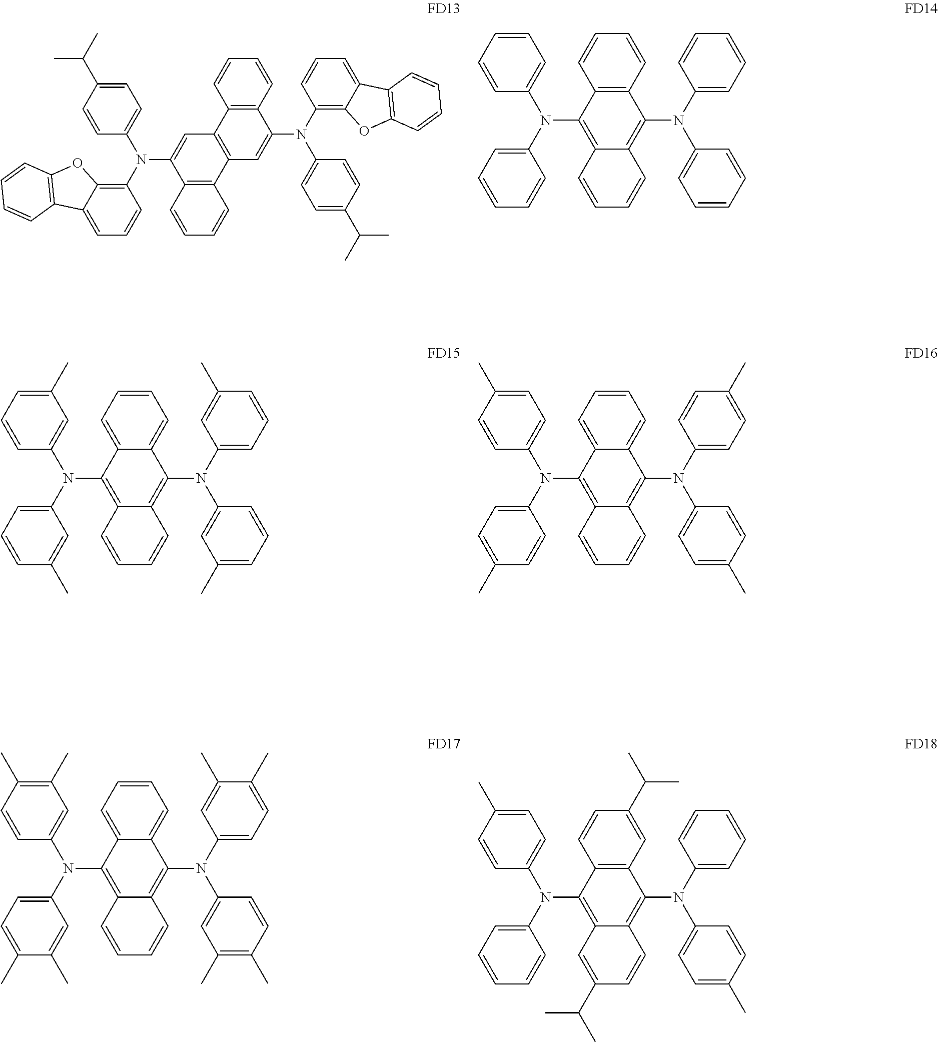

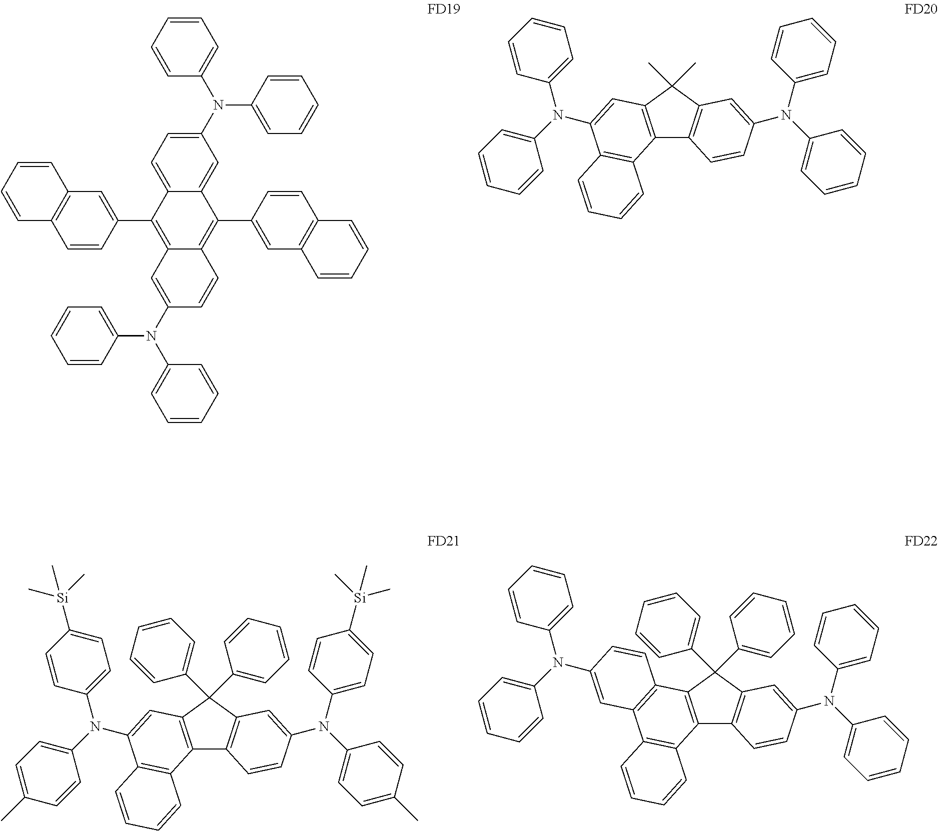

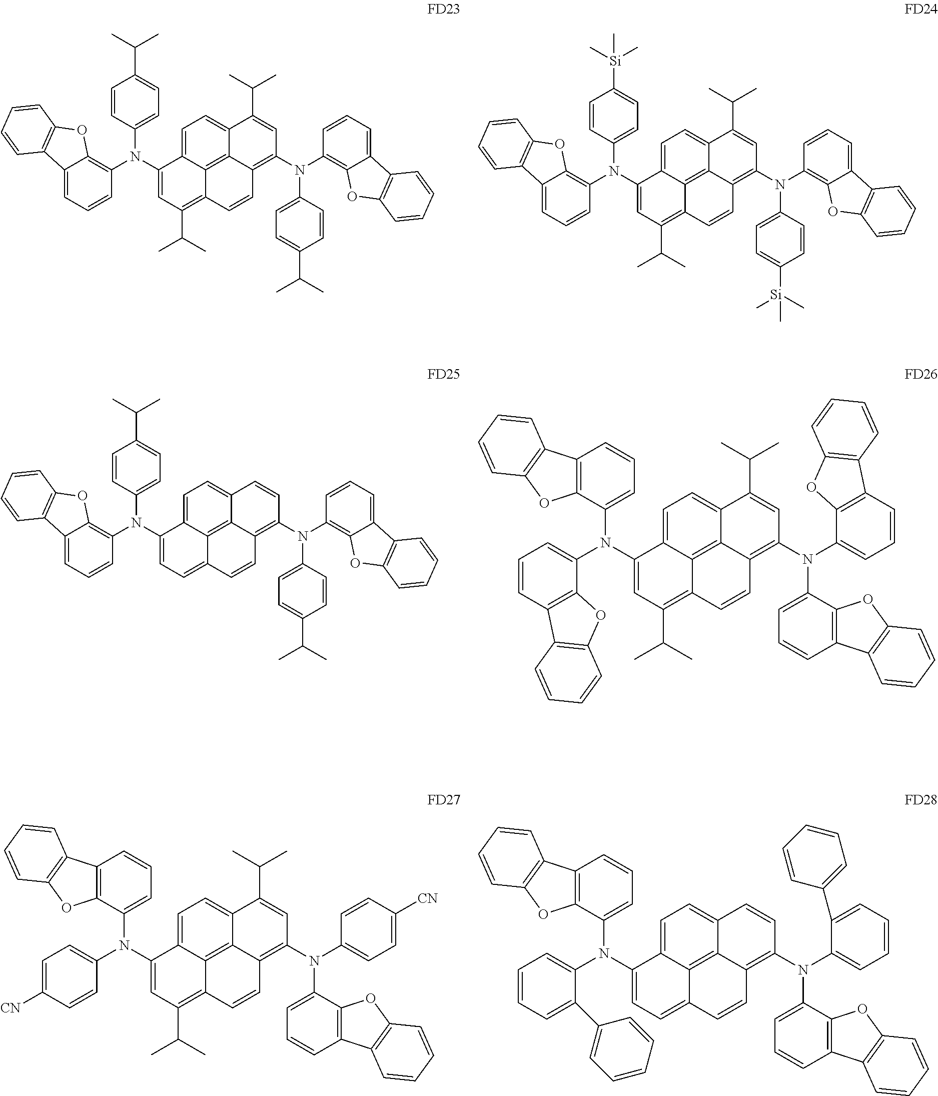

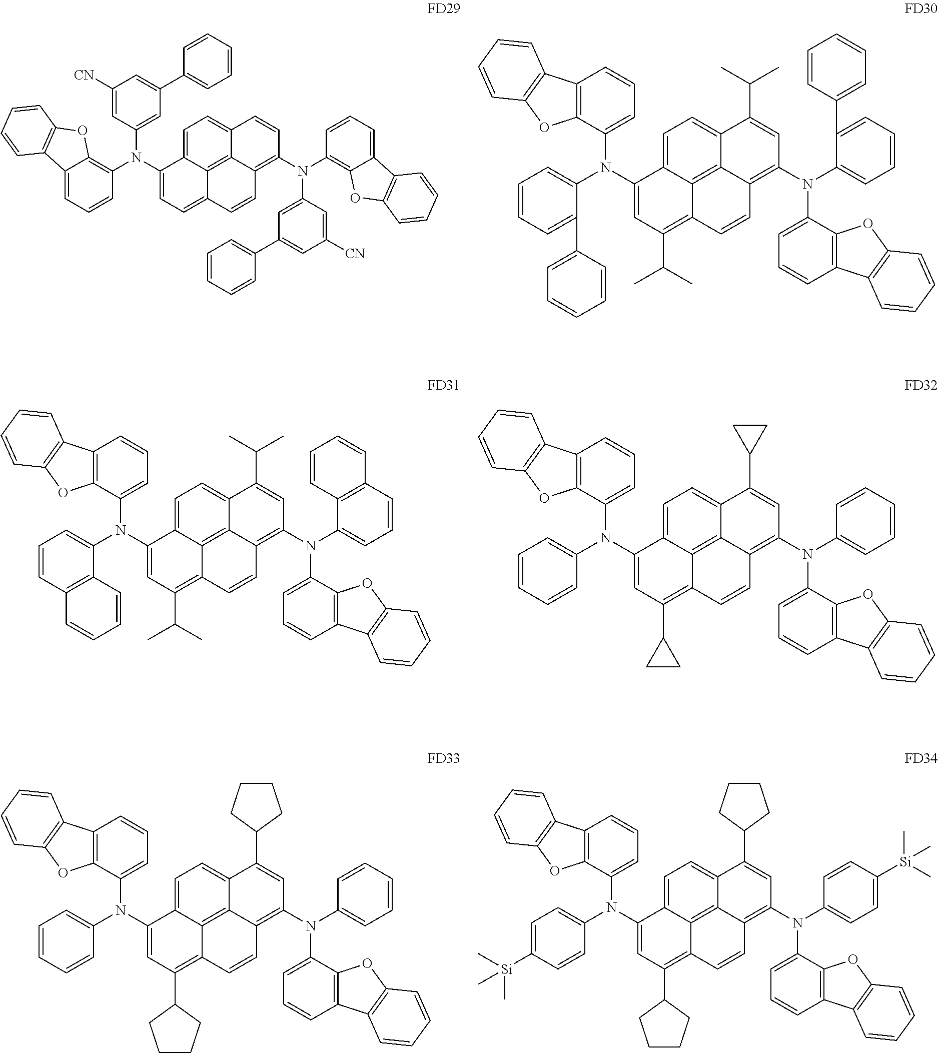

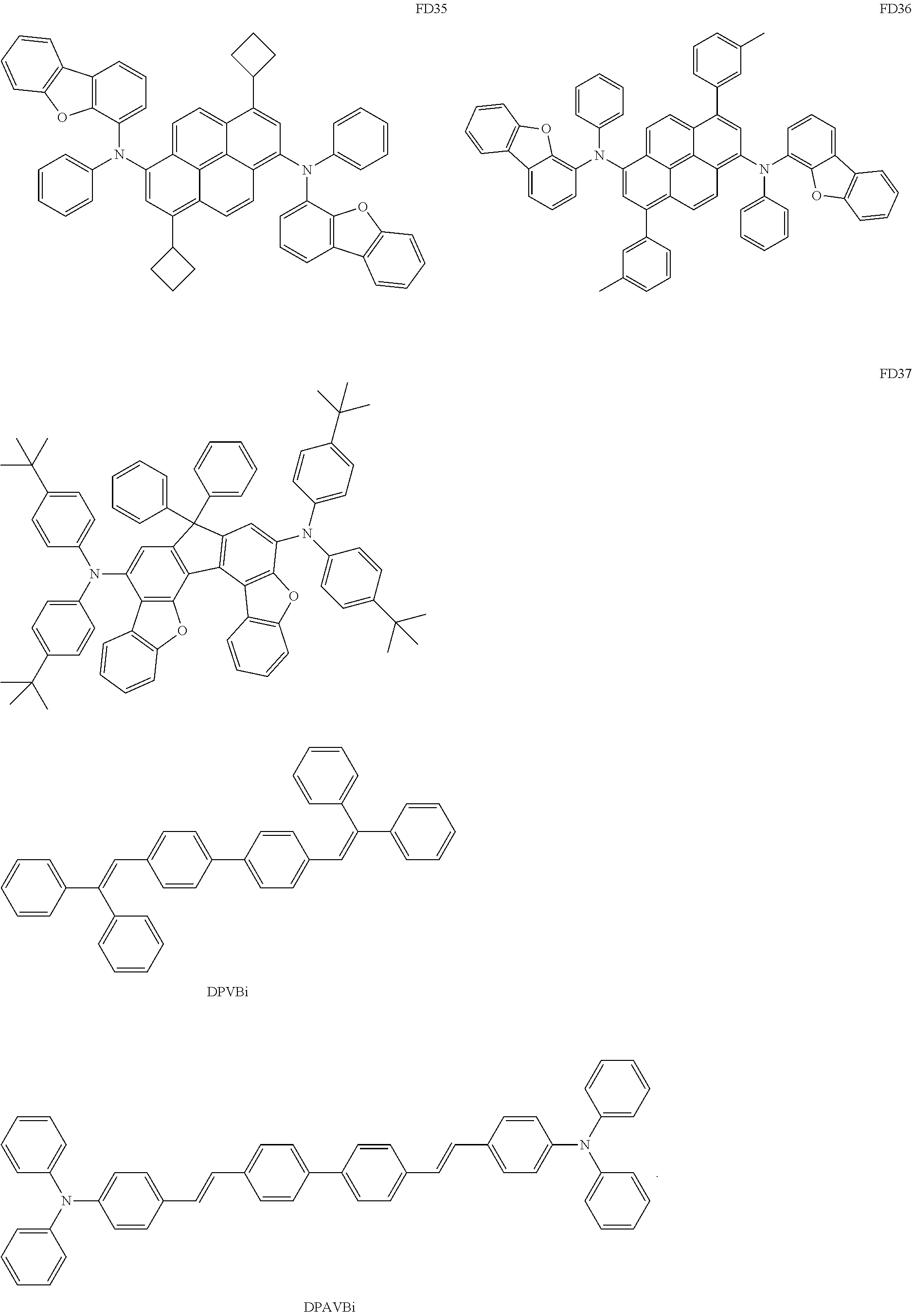

### Delayed Fluorescence Material

The emission layer may contain a delayed fluorescence material (compound 5).

Herein, the delayed fluorescence material may be selected from compounds capable of emitting delayed fluorescence based on a delayed fluorescence emission mechanism.

The delayed fluorescence material included in the emission layer may act as a host or a dopant depending on the type (kind) of other materials included in the emission layer.

In one or more embodiments, a difference between a triplet energy level (eV) of the delayed fluorescence material and the singlet energy level (eV) of the delayed fluorescence material may be at least about 0 eV and not more than about 0.5 eV. When the difference between the triplet energy level (eV) of the delayed fluorescence material and the singlet energy level (eV) of the delayed fluorescence material satisfies the herein-described range, up-conversion from the triplet state to the singlet state of the delayed fluorescence materials may effectively occur, and thus, the luminescence efficiency of the light-emitting device 10 may be improved.

In one or more embodiments, the delayed fluorescence material may include: i) a material including at least one electron donor (for example, a π electron-rich C₃-C₆₀ cyclic group such as a carbazole group) and at least one electron acceptor (for example, a sulfoxide group, a cyano group, a π electron-deficient nitrogen-containing C₁-C₆₀ cyclic group, and/or the like), ii) a material including a C₈-C₆₀ polycyclic group including at least two cyclic groups that are condensed with each other while sharing boron (B).

Examples of the delayed fluorescence material may include at least one of compounds DF1 to DF14:

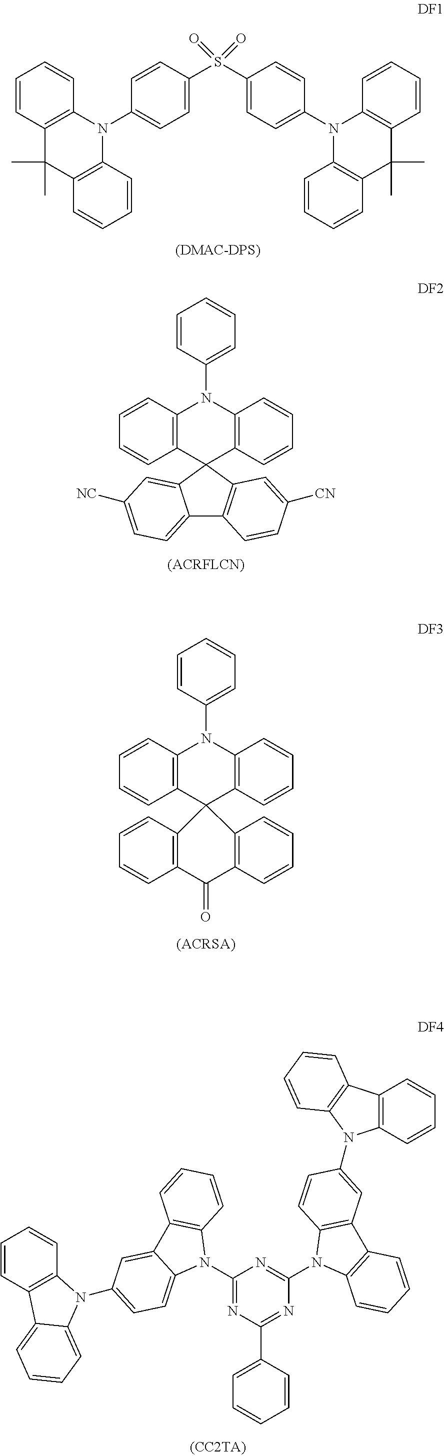

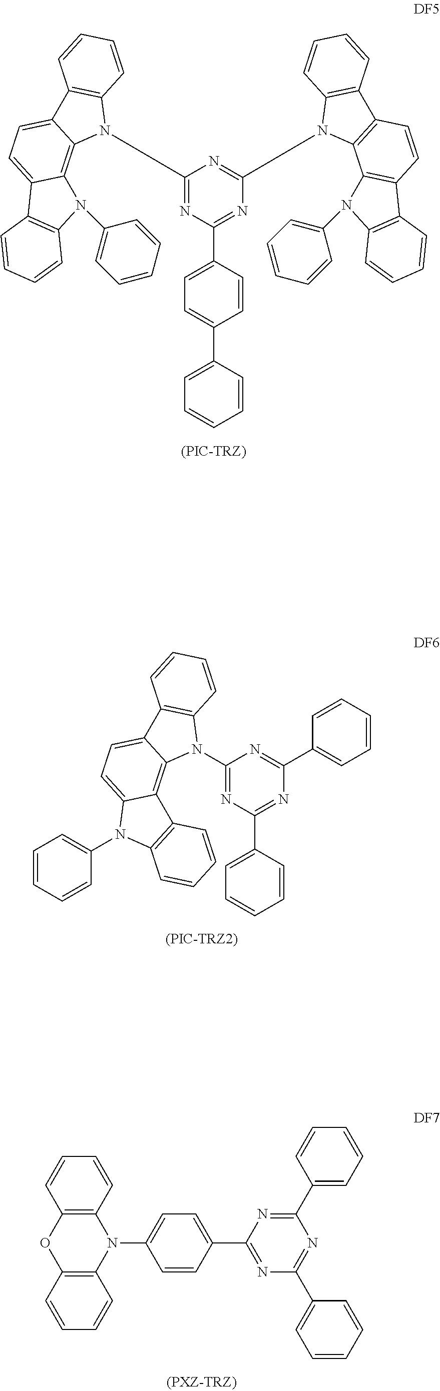 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

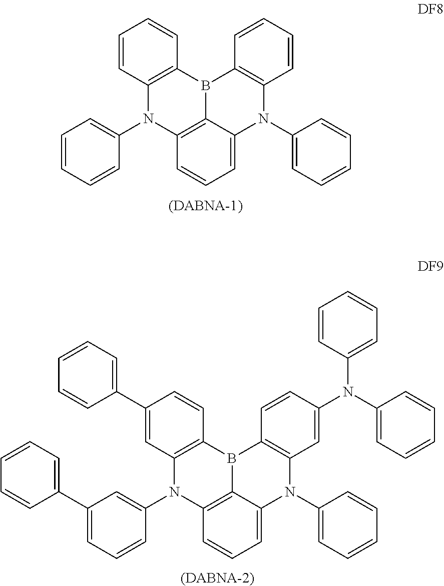

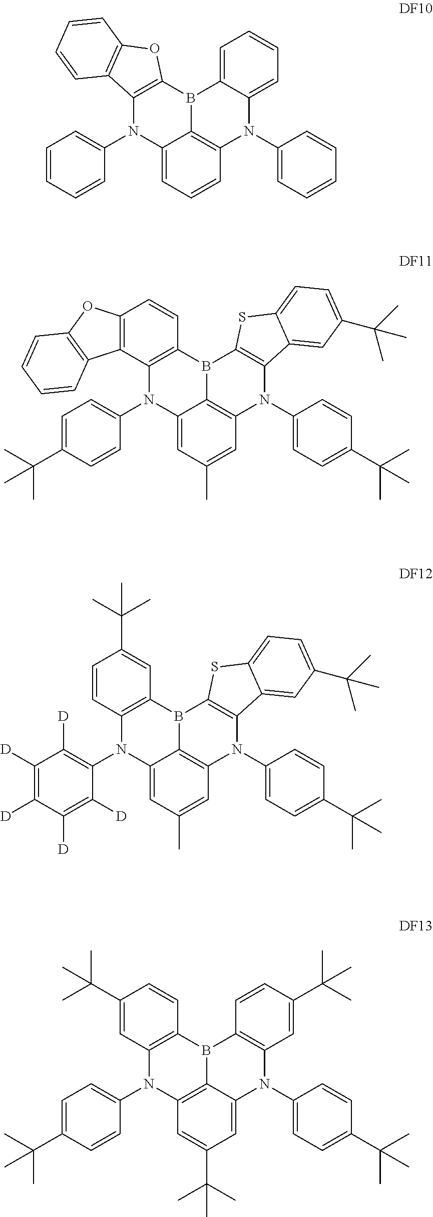

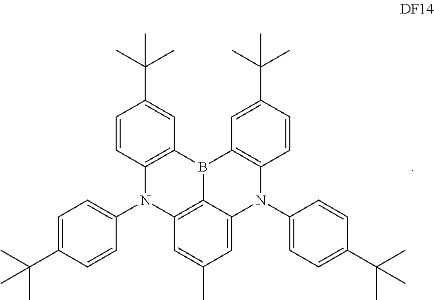 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### Quantum Dot

The emission layer may include a quantum dot.

The term “quantum dot” as used herein refers to a crystal of a semiconductor compound, and may include any material capable of emitting light of one or more suitable emission wavelengths according to the size of the crystal. Quantum dots may emit light of one or more suitable emission wavelengths by adjusting the element ratio in the quantum dot compound.

A diameter of the quantum dot may be, for example, in a range of about 1 nm to about 10 nm. In the present disclosure, when quantum dot, quantum dots, or quantum dot particles are spherical, “diameter” indicates a particle diameter or an average particle diameter, and when the particles are non-spherical, the “diameter” indicates a major axis length or an average major axis length. The diameter of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter is referred to as D50. D50 refers to the average diameter of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The quantum dot may be synthesized by a wet chemical process, a metal organic chemical vapor deposition process, a molecular beam epitaxy process, and/or any suitable process similar thereto.

The wet chemical process is a method including mixing a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled or selected through a process which costs lower, and is easier than vapor deposition methods, such as metal organic chemical vapor deposition (MOCVD) or molecular beam epitaxy (MBE).

The quantum dot may include Group II-VI semiconductor compounds, Group III-V semiconductor compounds, Group III-VI semiconductor compounds, Group I-III-VI semiconductor compounds, Group IV-VI semiconductor compounds, a Group IV element or compound, or a combination thereof.

Examples of the Group II-VI semiconductor compound are a binary compound, such as CdS, CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or a combination thereof.

Examples of the Group III-V semiconductor compound are: a binary compound, such as GaN, GaP, GaAs, GaSb, AlN, AIP, AIAs, AISb, InN, InP, InAs, InSb, and/or the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, GaAlNP, and/or the like; a quaternary compound, such as GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and/or the like; or any combination thereof. In some embodiments, the Group III-V semiconductor compound may further include a Group II element. Examples of the Group III-V semiconductor compound further including a Group II element are InZnP, InGaZnP, InAlZnP, and/or the like.

Examples of the Group III-VI semiconductor compound are: a binary compound, such as GaS, GaSe, Ga₂Se₃, GaTe, InS, InSe, In₂S₃, In₂Se₃, or InTe; a ternary compound, such as InGaS₃, or InGaSe₃; and a combination thereof.

Examples of the Group I-III-VI semiconductor compound may include: a ternary compound, such as AgInS, AgInS₂, AgInSe₂, AgGaS, AgGaS₂, AgGaSe₂, CuInS, CuInS₂, CuInSe₂, CuGaS₂, CuGaSe₂, CuGaO₂, AgGaO₂, AgAlO₂, and/or the like; a ternary compound, such as AgInGaS₂, AgInGaSe₂, and/or the like; or any combination thereof.

Examples of the Group IV-VI semiconductor compound are: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, or SnPbTe; a quaternary compound, such as SnPbSSe, SnPbSeTe, or SnPbSTe; or a combination thereof.

The Group IV element or compound may include: a single element compound, such as Si or Ge; a binary compound, such as SiC or SiGe; or a combination thereof.

Each element included in a multi-element compound such as the binary compound, the ternary compound, and the quaternary compound may be present at a uniform concentration or non-uniform concentration in a particle. The preceding formulae refer to the types (kinds) of elements included in each compound, and the element ratios in these compounds may be different from each other. For example, AgInGaS₂ may indicate AgInₓGa₁₋ₓS₂ (where x is a real number satisfying 0<x<1).

In some embodiments, the quantum dot may have a single structure in which the concentration of each element in the quantum dot is substantially uniform, or a core-shell dual structure. For example, the material included in the core and the material included in the shell may be different from each other.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases toward the center of the core.

Examples of the shell of the quantum dot may be an oxide of metal, metalloid, or non-metal, a semiconductor compound, and a combination thereof. Examples of the oxide of metal, metalloid, or non-metal are a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; and a combination thereof. Examples of the semiconductor compound are, as described herein, a Group II-VI semiconductor compound; a Group III-V semiconductor compound; a Group III-VI semiconductor compound; a Group I-III-VI semiconductor compound; a Group IV-VI semiconductor compound; and a combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or a combination thereof.

Each element included in a multi-element compound, such as the binary compound and the ternary compound, may be present at a substantially uniform concentration or substantially non-uniform concentration in a particle. The preceding formulae refer to the types (kinds) of elements included in each compound, and the element ratios in these compounds may be different from each other.

A full width at half maximum (FWHM) of the emission wavelength spectrum of the quantum dot may be about 45 nm or less, for example, about 40 nm or less, for example, about 30 nm or less, and within these ranges, color purity or color reproducibility may be increased. In some embodiments, because the light emitted through the quantum dot is emitted in all directions, the wide viewing angle may be improved.

In some embodiments, the quantum dot may be in the form of a spherical particle, a pyramidal particle, a multi-arm particle, a cubic nanoparticle, a nanotube particle, a nanowire particle, a nanofiber particle, or a nanoplate particle.

Because an energy band gap may be adjusted by controlling or selecting the size of the quantum dot, light having one or more suitable wavelength bands may be obtained from the quantum dot emission layer. Accordingly, by using quantum dots of different sizes, a light-emitting device that emits light of one or more suitable wavelengths may be implemented. In one or more embodiments, the size of the quantum dots or the ratio of elements in the quantum dot compound may be selected so that red light, green light, and/or blue light can be emitted. In one or more embodiments, the quantum dots may be configured to emit white light by combination of light of one or more suitable colors.

### Electron Transport Region in Interlayer 130

The electron transport region may include the organic compound by Formula 1 and/or nanocomposites including the organic compound.

The electron transport region may have: i) a single-layered structure including (e.g., consisting of) a single layer including (e.g., consisting of) a single material, ii) a single-layered structure including (e.g., consisting of) a single layer including multiple different materials, or iii) a multilayer structure including multiple layers including multiple different materials.

The electron transport region may include a buffer layer, a hole-blocking layer, an electron control layer, an electron transport layer, an electron injection layer, or any combination thereof.

For example, the electron transport region may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, wherein layers in each structure are sequentially stacked from the emission layer.

The electron transport region (e.g., a buffer layer, a hole-blocking layer, an electron control layer, or an electron transport layer in the electron transport region) may include a metal-free compound including at least one π electron-deficient nitrogen-containing C₁-C₆₀ heterocyclic group.

In one or more embodiments, the electron transport region may include a compound represented by Formula 601.

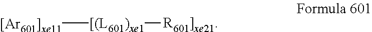

In Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ are each as described in connection with Q₁,
xe21 may be 1, 2, 3, 4, or 5, and
at least one of (e.g., selected from among) Ar₆₀₁, L₆₀₁, and R₆₀₁ may each independently be a π electron-deficient nitrogen-containing C₁-C₆₀ cyclic group that is unsubstituted or substituted with at least one R₁₀ₐ.

In one or more embodiments, when xe11 in Formula 601 is 2 or more, two or more of Ar₆₀₁ may be linked together via a single bond (e.g., a single covalent bond).

In one or more embodiments, Ar₆₀₁ in Formula 601 may be a substituted or unsubstituted anthracene group.

In one or more embodiments, the electron transport region may include a compound represented by Formula 601-1:

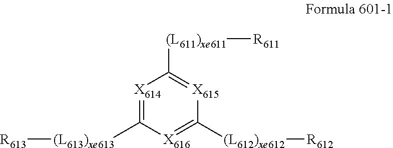

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ are each as described in connection with L₆₀₁,
xe611 to xe613 are each as described in connection with xe1,
R₆₁₁ to R₆₁₃ are each as described in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group that is unsubstituted or substituted with at least one R₁₀ₐ, or a C₁-C₆₀ heterocyclic group that is unsubstituted or substituted with at least one R₁₀ₐ.

In one or more embodiments, xe1 and xe611 to xe613 in Formulae 601 and 601-1 may each independently be 0, 1, or 2.

The electron transport region may include: at least one of compounds ET1 to ET46; 2,9-dimethyl-4,7-diphenyl-1,10-phenanthroline (BCP); 4,7-diphenyl-1,10-phenanthroline (Bphen); Alq₃; BAlq; TAZ; NTAZ; or any combination thereof:

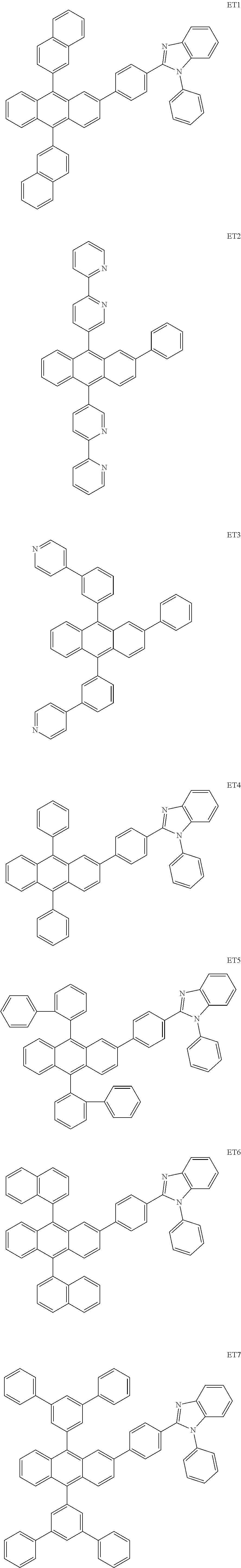

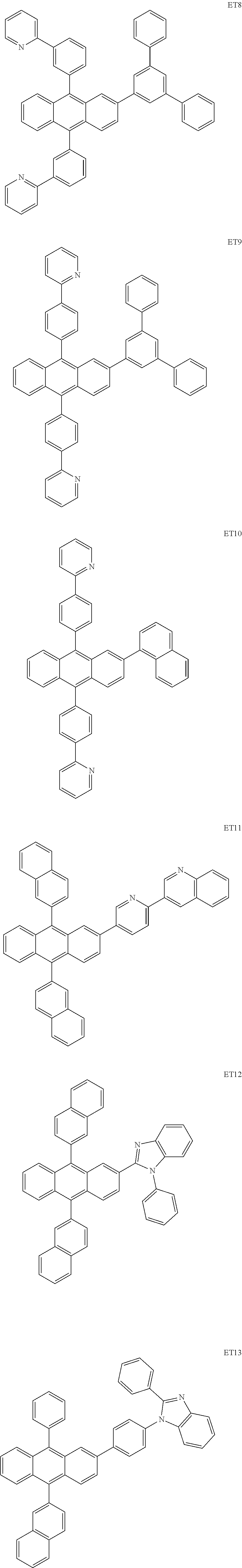

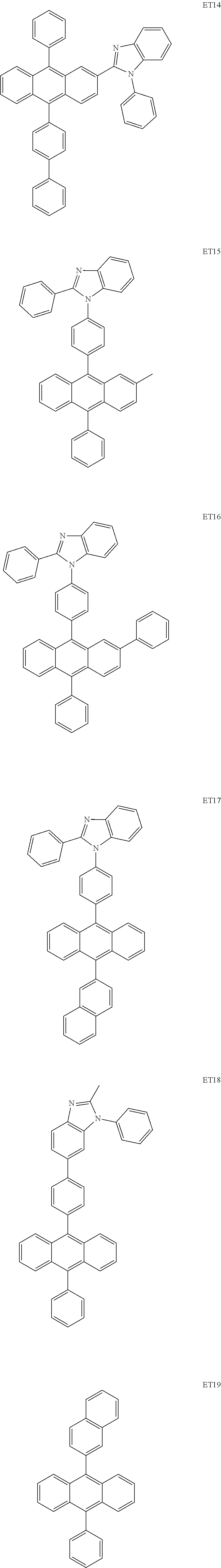

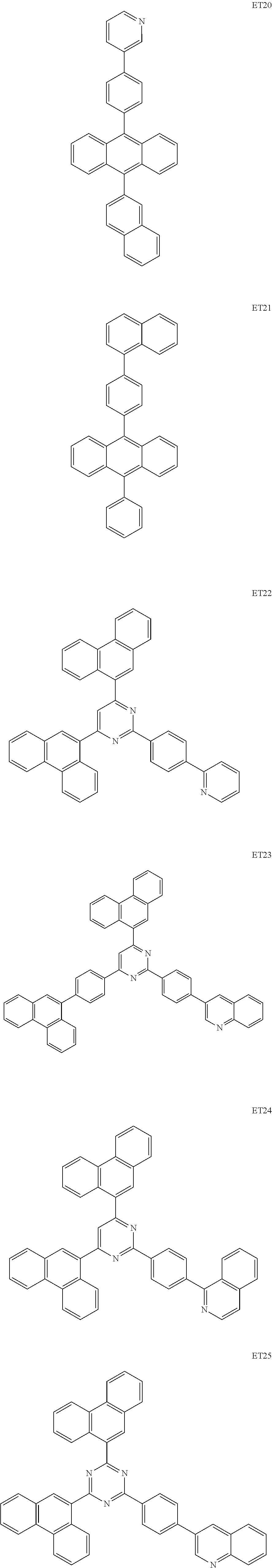

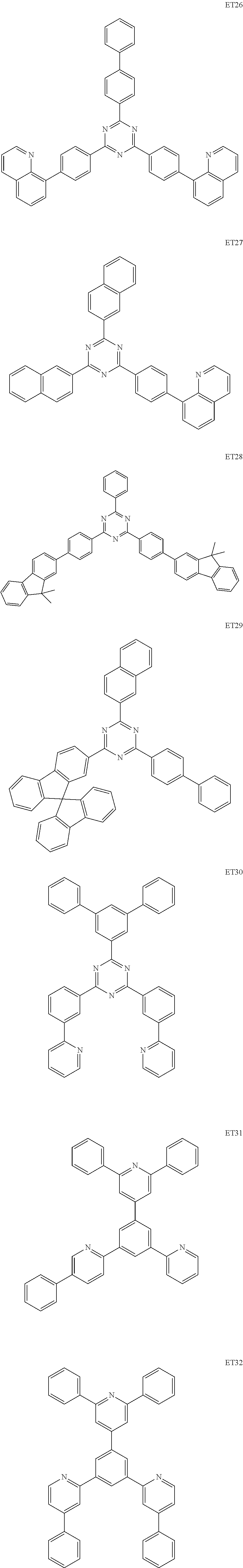

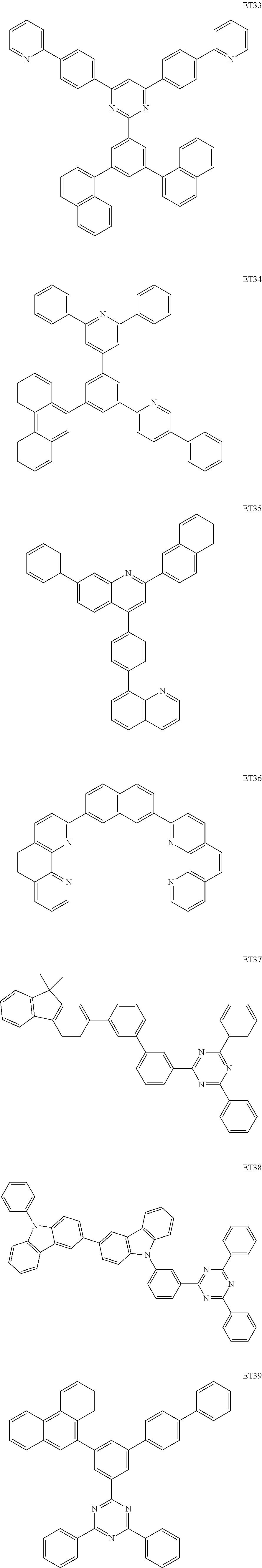

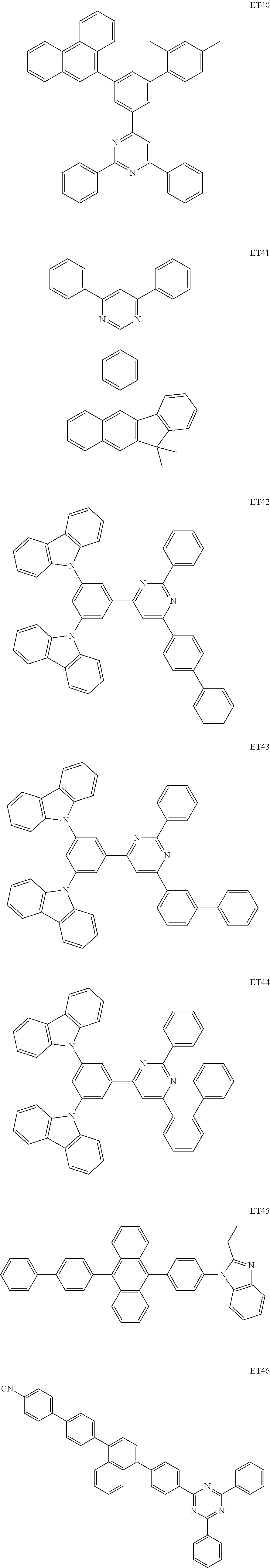

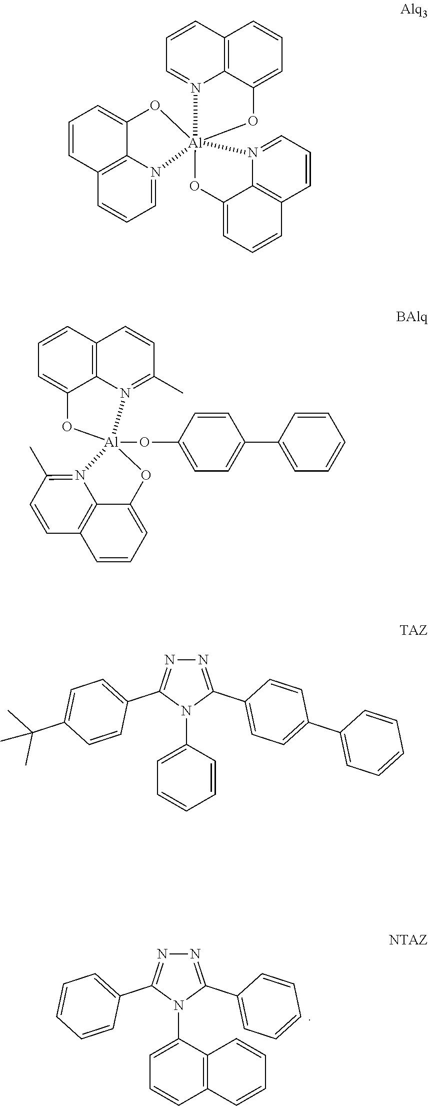

The thickness of the electron transport region may be about 100 Å to about 5,000 Å, for example, about 160 Å to about 4,000 Å. When the electron transport region includes a buffer layer, a hole-blocking layer, an electron control layer, an electron transport layer, or any combination thereof, a thickness of the buffer layer, the hole-blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole-blocking layer, the electron control layer, the electron transport layer, and/or the electron transport region are within the ranges described herein, satisfactory electron transporting characteristics may be obtained without a substantial increase in driving voltage.

The electron transport region (for example, the electron transport layer in the electron transport region) may further include, in addition to the materials described herein, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

In one or more embodiments, the metal-containing material may include a Li complex. The Li complex may include, for example, compound ET-D1 (LiQ) or ET-D2:

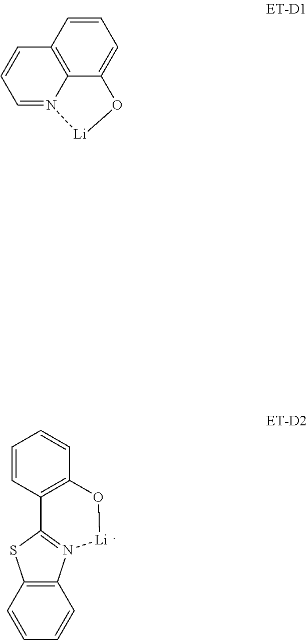

The electron transport region may include an electron injection layer that facilitates the injection of electrons from the second electrode 150. The electron injection layer may directly contact the second electrode 150.

The electron injection layer may have: i) a single-layered structure including (e.g., consisting of) a single layer including (e.g., consisting of) a single material, ii) a single-layered structure including (e.g., consisting of) a single layer including multiple different materials, or iii) a multilayer structure including multiple layers including multiple different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (for example, fluorides, chlorides, bromides, iodides, and/or the like), or tellurides of the alkali metal, the alkaline earth metal, and the rare earth metal, or any combination thereof.

The alkali metal-containing compound may include: an alkali metal oxide, such as Li₂O, Cs₂O, K₂O, and/or the like; alkali metal halides, such as LiF, NaF, CsF, KF, LiI, NaI, CsI, KI, and/or the like; or any combination thereof. The alkaline earth metal-containing compound may include an alkaline earth metal compound, such as BaO, SrO, CaO, BaₓSr₁₋ₓO (x is a real number satisfying 0<x<1), or BaₓCa₁₋ₓO (x is a real number satisfying 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of metal ions of the alkali metal, the alkaline earth metal, and the rare earth metal, and ii) as a ligand bonded to the metal ions, for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described herein. In one or more embodiments, the electron injection layer may further include an organic material (for example, a compound represented by Formula 601).

In one or more embodiments, the electron injection layer may include (e.g., consist of) i) an alkali metal-containing compound (for example, alkali metal halide), ii) a) an alkali metal-containing compound (for example, alkali metal halide); and b) an alkali metal, an alkaline earth metal, a rare earth metal, or any combination thereof. In one or more embodiments, the electron injection layer may be a KI:Yb co-deposited layer, an RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly (e.g., substantially uniformly) or non-uniformly (e.g., substantially non-uniformly) dispersed in a matrix including the organic material.

The thickness of the electron injection layer may be about 1 Å to about 100 Å, and, for example, about 3 Å to about 90 Å. When the thickness of the electron injection layer is within the range as described herein, satisfactory electron injection characteristics may be obtained without a substantial increase in driving voltage.

### Second Electrode 150

The second electrode 150 is on the interlayer 130. The second electrode 150 may be a cathode, which is an electron injection electrode, and as a material for forming the second electrode 150, a metal, an alloy, an electrically conductive compound, or any combination thereof, each having a low-work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a transflective electrode, or a reflective electrode.

The second electrode 150 may have a single-layer structure or a multilayer structure including a plurality of layers.

### Capping Layer

A first capping layer may be outside (and e.g., on) the first electrode 110, and/or a second capping layer may be outside (and e.g., on) the second electrode 150. In particular, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the emission layer of the interlayer 130 of the light-emitting device 10 may be extracted toward the outside through the first electrode 110 which is a transflective electrode or a transmissive electrode, and the first capping layer. Light generated in the emission layer of the interlayer 130 of the light-emitting device 10 may be extracted toward the outside through the second electrode 150 which is a transflective electrode or a transmissive electrode, and the second capping layer.

The first capping layer and the second capping layer may increase external emission efficiency according to the principle of constructive interference. Accordingly, the light extraction efficiency of the light-emitting device 10 is increased, such that the luminescence efficiency of the light-emitting device 10 may be increased.

Each of the first capping layer and the second capping layer may include a material having a refractive index of 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

At least one of (e.g., selected from among) the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In one or more embodiments, at least one of (e.g., selected from among) the first capping layer and the second capping layer may each independently include an amine group-containing compound.

In one or more embodiments, at least one of (e.g., selected from among) the first capping layer and/or the second capping layer may each independently include a compound represented by Formula 201, a compound represented by Formula 202, or any combination thereof.

In one or more embodiments, at least one of (e.g., selected from among) the first capping layer and/or the second capping layer may each independently include at least one of compounds HT28 to HT33, at least one of compounds CP1 to CP6, β-NPB, or any combination thereof:

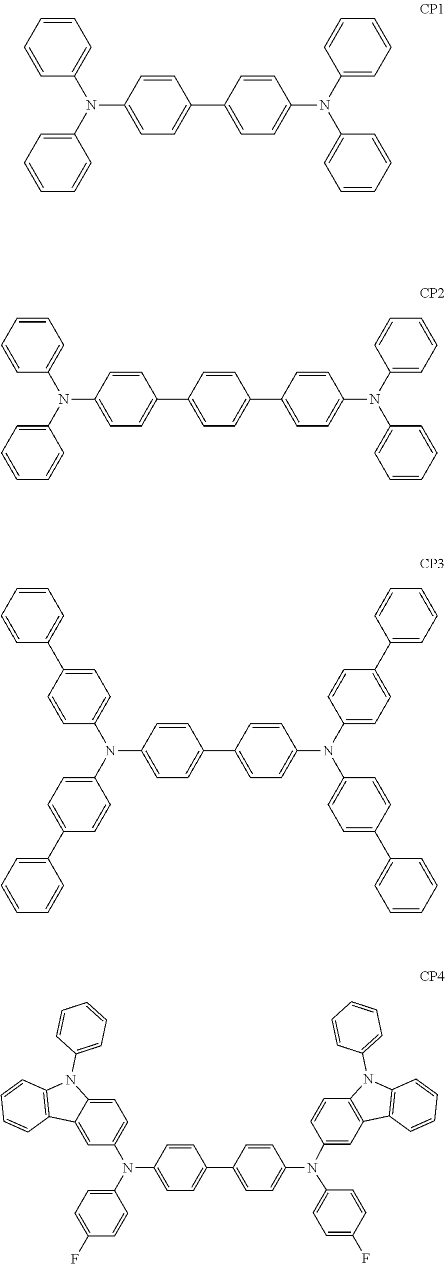 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

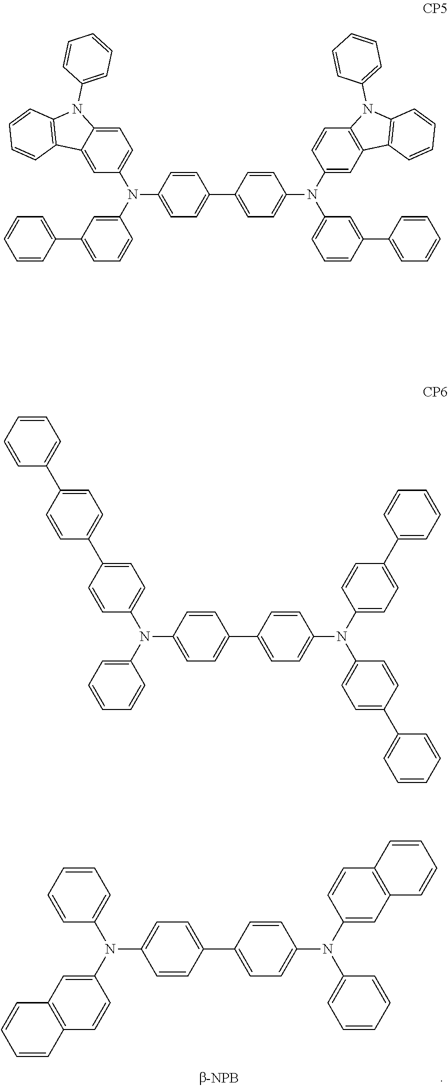 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The organic compound represented by Formula 1 may be included in one or more suitable films. Accordingly, another aspect of the disclosure provides a film including the organic compound represented by Formula 1. The film may be, for example, an optical member (or a light control component) (e.g., a color filter, a color conversion member, a capping layer, a light extraction efficiency enhancement layer, a selective light absorbing layer, a polarizing layer, a quantum dot-containing layer, and/or the like), a light-blocking member (e.g., a light reflective layer, a light absorbing layer, and/or the like), a protective member (e.g., an insulating layer, a dielectric layer, and/or the like), and/or the like.

### Electronic Apparatus

The light-emitting device may be included in one or more suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be in at least one traveling direction of light emitted from the light-emitting device. For example, light emitted from the light-emitting device may be blue light, green light, or white light. A detailed description of the light-emitting device is provided herein. In one or more embodiments, the color conversion layer may include quantum dots.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be among the subpixel areas to define each of the subpixel areas.

The color filter may further include a plurality of color filter areas and light-shielding patterns among the color filter areas, and the color conversion layer may further include a plurality of color conversion areas and light-shielding patterns among the color conversion areas.

The plurality of color filter areas (or the plurality of color conversion areas) may include a first area emitting first color light, a second area emitting second color light, and/or a third area emitting third color light, wherein the first color light, the second color light, and/or the third color light may have different maximum emission wavelengths. In one or more embodiments, the first color light may be red light, the second color light may be green light, and the third color light may be blue light. In one or more embodiments, the plurality of color filter areas (or the plurality of color conversion areas) may include quantum dots. In detail, the first area may include red quantum dots, the second area may include green quantum dots, and the third area may not include quantum dots. A detailed description of the quantum dots is provided herein. The first area, the second area, and/or the third area may each further include a scatterer.

In one or more embodiments, the light-emitting device may be to emit first light, the first area may be to absorb the first light to emit first-1 color light, the second area may be to absorb the first light to emit second-1 color light, and the third area may be to absorb the first light to emit third-1 color light. In this case, the first-1 color light, the second-1 color light, and the third-1 color light may have each different maximum emission wavelengths. In detail, the first light may be blue light, the first-1 color light may be red light, the second-1 color light may be green light, and the third-1 color light may be blue light.

The electronic apparatus may further include a thin-film transistor, in addition to the light-emitting device as described herein. The thin-film transistor may include a source electrode, a drain electrode, and an active layer, wherein any one of the source electrode and the drain electrode may be electrically connected (coupled) to any one of the first electrode and the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the light-emitting device. The sealing portion may be between the color filter and/or the color conversion layer and the light-emitting device. The sealing portion allows light from the light-emitting device to be extracted to the outside, and simultaneously prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

One or more suitable functional layers may be additionally on the sealing portion, in addition to the color filter and/or the color conversion layer, according to the use of the electronic apparatus. Examples of the functional layers may include a touch screen layer and a polarizing layer. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, and/or the like).

The authentication apparatus may further include, in addition to the light-emitting device as described herein, a biometric information collector.

The electronic apparatus may be applied to one or more suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

### Electronic Device

The light-emitting device may be included in one or more suitable electronic device(s).

In one or more embodiments, the electronic device including the light-emitting device may be at least one of (e.g., selected from among) a flat panel display, a curved display, a computer monitor, a medical monitor, a television, a billboard, an indoor light, an outdoor light, a signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a 3-dimensional (3D) display, a virtual or augmented-reality display, a vehicle, a video wall including multiple displays tiled together, a theater or stadium screen, a phototherapy device, and a signboard.

Because the light-emitting device has excellent or suitable effects in terms of luminescence efficiency long lifespan, the electronic device including the light-emitting device may have characteristics with high luminance, high resolution, and low power consumption.

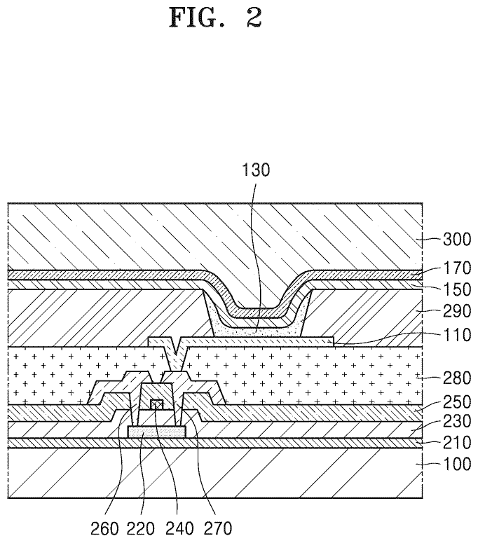

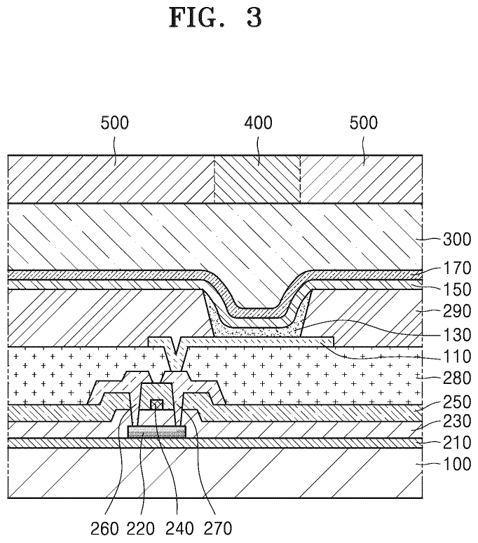

### Description of FIGS. 2 and 3

FIG. 2 is a cross-sectional view showing a light-emitting apparatus as an example of the electronic apparatus according to one or more embodiments.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be on the substrate 100. The buffer layer 210 may prevent or reduce penetration of impurities through the substrate 100 and may provide a flat surface on the substrate 100.

A TFT may be on the buffer layer 210. The TFT may include an activation layer 220, a gate electrode 240, a source electrode 260, and a drain electrode 270.

The activation layer 220 may include an inorganic semiconductor, such as silicon or polysilicon, an organic semiconductor, or an oxide semiconductor, and may include a source region, a drain region, and a channel region.

A gate insulating film 230 for insulating the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270, to insulate these electrodes from one another.

The source electrode 260 and the drain electrode 270 may be on the interlayer insulating film 250. The interlayer insulating film 250 and the gate insulating film 230 may be formed to expose the source region and the drain region of the activation layer 220, and the source electrode 260 and the drain electrode 270 may be in contact with the exposed portions of the source region and the drain region of the activation layer 220.

The TFT may be electrically connected (coupled) to a light-emitting device to drive the light-emitting device, and may be covered and protected by a passivation layer 280. The passivation layer 280 may include an inorganic insulating film, an organic insulating film, or any combination thereof. A light-emitting device may be provided on the passivation layer 280. The light-emitting device may include the first electrode 110, the interlayer 130, and the second electrode 150.

The first electrode 110 may be on the passivation layer 280. The passivation layer 280 may be to expose a portion of the drain electrode 270, not fully covering the drain electrode 270, and the first electrode 110 may be to be connected (coupled) to the exposed portion of the drain electrode 270.

A pixel-defining film 290 including an insulating material may be on the first electrode 110. The pixel-defining film 290 may expose a certain region of the first electrode 110, and the interlayer 130 may be formed in the exposed region of the first electrode 110. The pixel-defining film 290 may be a polyimide-based organic film or a polyacrylic organic film. In one or more embodiments, at least some layers of the interlayer 130 may extend beyond the upper portion of the pixel-defining film 290 to be in the form of a common layer.

The second electrode 150 may be on the interlayer 130, and a second capping layer 170 may be additionally formed on the second electrode 150. The second capping layer 170 may be formed to cover the second electrode 150.

The encapsulation portion 300 may be on the second capping layer 170. The encapsulation portion 300 may be on a light-emitting device to protect the light-emitting device from moisture or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or any combination thereof; or a combination of the inorganic film and the organic film.

FIG. 3 is a cross-sectional view of a light-emitting apparatus as an example of the electronic apparatus according to another embodiment.

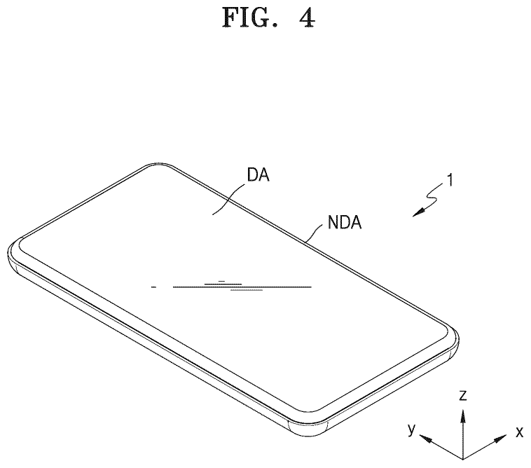

The light-emitting apparatus of FIG. 3 is the same as the light-emitting apparatus of FIG. 2, except that a light-shielding pattern 500 and a functional region 400 are additionally on the encapsulation portion 300. The functional region 400 may be i) a color filter area, ii) a color conversion area, or iii) a combination of the color filter area and the color conversion area. In one or more embodiments, a light-emitting device included in the light-emitting apparatus of FIG. 4 may be a tandem light-emitting device.

### Description of FIG. 4

FIG. 4 is a schematic perspective view of an electronic device 1 including a light-emitting device according to one or more embodiments. The electronic device 1 may be, as an apparatus that displays a moving image or a still image, portable electronic device, such as a mobile phone, a smartphone, a tablet personal computer (PC), a mobile communication terminal, an electronic notebook, an electronic book, a portable multimedia player (PMP), a navigation, or an ultra-mobile PC (UMPC), as well as one or more suitable products, such as a television, a laptop, a monitor, a billboard, or an Internet of things (loT) device. The electronic device 1 may be such a product herein or a part thereof. In some embodiments, the electronic device 1 may be a wearable device, such as a smart watch, a watch phone, a glasses-type (kind) display, or a head mounted display (HMD), or a part of the wearable device. However, embodiments are not limited thereto. In one or more embodiments, the electronic device 1 may be a dashboard of a vehicle, a center information display (CID) on a center fascia or dashboard of a vehicle, a room mirror display instead of a side-view mirror of a vehicle, an entertainment for the back seat of a vehicle, or a display on the back of the front seat of a vehicle, a head up display (HUD) installed on the front of a vehicle or projected on a front window glass, or a computer generated hologram augmented reality head up display (CGH AR HUD). FIG. 4 illustrates a case in which the electronic device 1 is a smartphone for convenience of explanation.

The electronic device 1 may include a display area DA and a non-display area NDA outside the display area DA. A display apparatus may implement an image through an array of a plurality of pixels that are two-dimensionally in the display area DA.

The non-display area NDA is an area that does not display an image, and may entirely be around (e.g., surround) the display area DA. On the non-display area NDA, a driver for providing electrical signals or power to display devices on the display area DA may be arranged. On the non-display area NDA, a pad, which is an area to which an electronic element or a printed circuit board, may be electrically connected (coupled) may be arranged.

In the electronic device 1, the length in an x-axis direction and the length in a y-axis direction may be different from each other. In one or more embodiments, as shown in FIG. 4, the length in the x-axis direction may be less than the length in the y-axis direction. In one or more embodiments, the length in the x-axis direction may be the same as the length in the y-axis direction. In one or more embodiments, the length in the x-axis direction may be greater than the length in the y-axis direction.

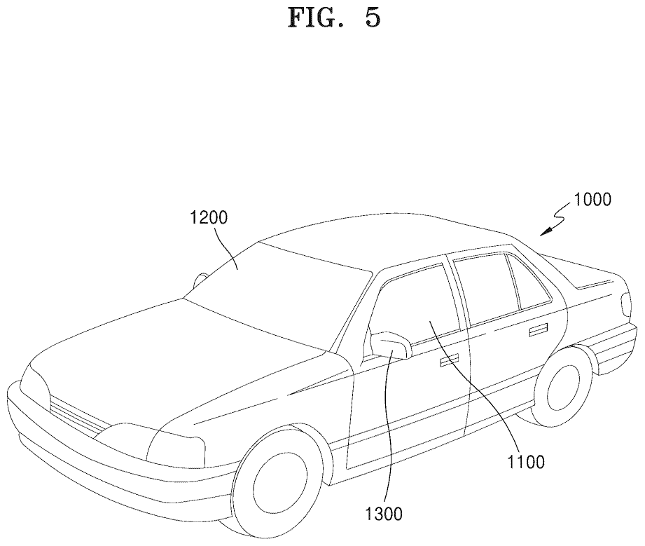

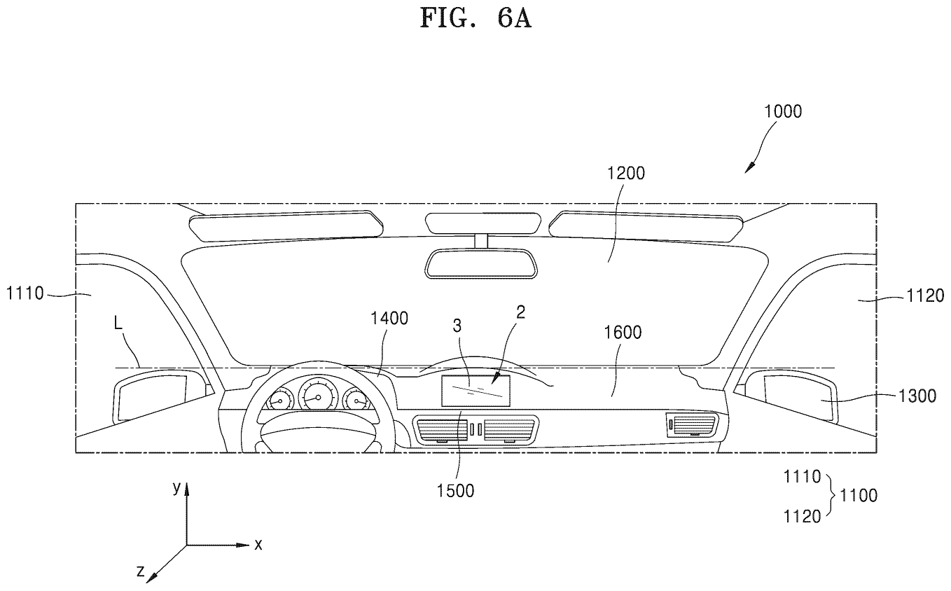

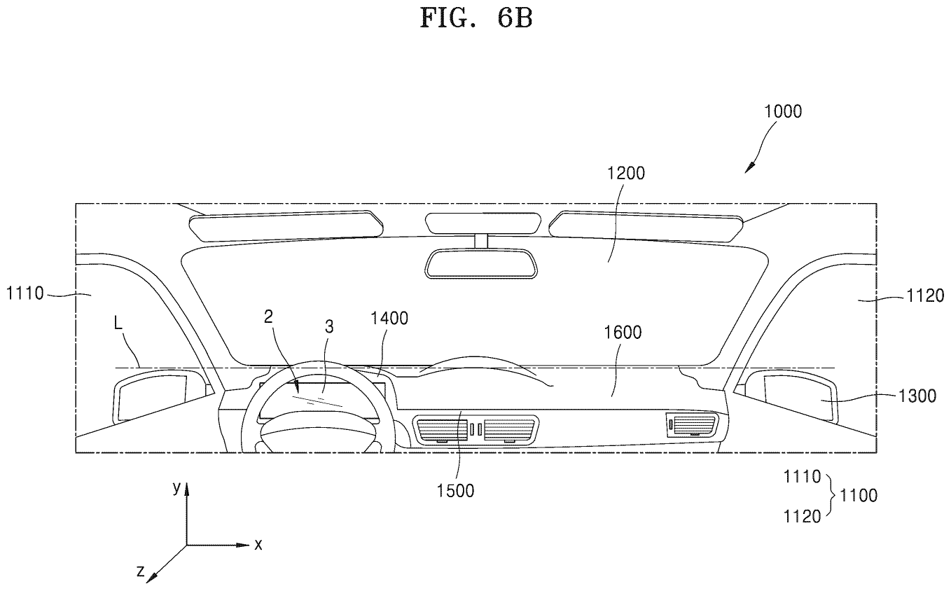

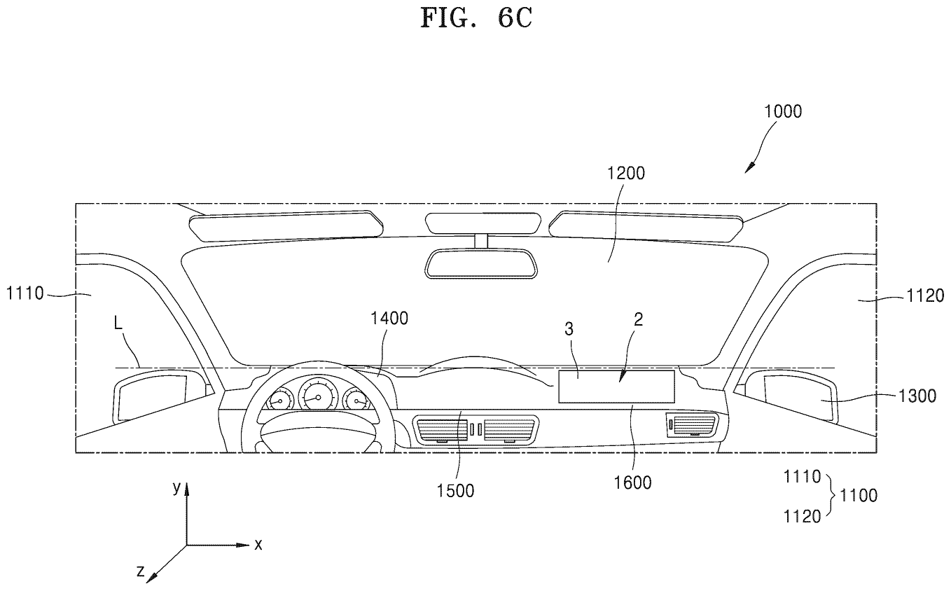

### Descriptions of FIGS. 5 and 6A to 6C

FIG. 5 is a schematic view of the exterior of a vehicle 1000 as an electronic device including a light-emitting device, according to one or more embodiments. FIGS. 6A to 6C are each a schematic view of the interior of the vehicle 1000 according to one or more embodiments.

Referring to FIGS. 5, 6A, 6B, and 6C, the vehicle 1000 may refer to one or more suitable apparatuses for moving a subject to be transported, such as a human, an object, or an animal, from a departure point to a destination point. The vehicle 1000 may include a vehicle traveling on a road or track, a vessel moving over the sea or river, an airplane flying in the sky using the action of air, and/or the like.

The vehicle 1000 may travel on a road or a track. The vehicle 1000 may move in a certain direction according to rotation of at least one wheel. In one or more embodiments, the vehicle 1000 may include a three-wheeled or four-wheeled vehicle, a construction machine, a two-wheeled vehicle, a prime mover device, a bicycle, and a train running on a track.

The vehicle 1000 may include a body having an interior and an exterior, and a chassis in which mechanical apparatuses necessary for driving are installed as other parts except for the body of the vehicle 1000. The exterior of the body of the vehicle may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side-view mirror 1300, a cluster 1400, a center fascia 1500, a passenger seat dashboard 1600, and a display apparatus 2.

The side window glass 1100 and the front window glass 1200 may be partitioned by a pillar between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be adjacent to the cluster 1400. The second side window glass 1120 may be adjacent to the passenger seat dashboard 1600.

In one or more embodiments, the side window glasses 1100 may be spaced and/or apart (e.g., spaced apart or separated) from each other in an x direction or a −x direction. In one or more embodiments, the first side window glass 1110 and the second side window glass 1120 may be spaced and/or apart (e.g., spaced apart or separated) from each other in the x direction or the−x direction. In other words, an imaginary straight line L connecting the side window glasses 1100 may extend in the x direction or the−x direction. In one or more embodiments, an imaginary straight line L connecting the first side window glass 1110 and the second side window glass 1120 to each other may extend in the x direction or the−x direction.

The front window glass 1200 may be installed in front of the vehicle 1000. The front window glass 1200 may be between the side window glasses 1100 opposite to (e.g., facing) each other.

The side-view mirror 1300 may provide a rear view of the vehicle 1000. The side-view mirror 1300 may be installed on the exterior of the body of the vehicle. In one or more embodiments, a plurality of side-view mirrors 1300 may be provided. Any one of the plurality of side-view mirrors 1300 may be outside the first side window glass 1110. Another of the plurality of side-view mirrors 1300 may be outside the second side window glass 1120.

The cluster 1400 may be in front of a steering wheel. The cluster 1400 may include a tachometer, a speedometer, a coolant thermometer, a fuel gauge, a turn signal indicator, a high beam indicator, a warning light, a seat belt warning light, an odometer, a tachograph, an automatic shift selector indicator, a door open warning light, an engine oil warning light, and/or a low fuel warning light.

The center fascia 1500 may include a control panel on which a plurality of buttons for adjusting an audio device, an air conditioning device, and a seat heater are on. The center fascia 1500 may be on one side of the cluster 1400.

The passenger seat dashboard 1600 may be spaced and/or apart (e.g., spaced apart or separated) from the cluster 1400, and the center fascia 1500 may be between the cluster 1400 and the passenger seat dashboard 1600. In one or more embodiments, the cluster 1400 may be to correspond to a driver seat, and the passenger seat dashboard 1600 may be to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be inside the vehicle 1000. In one or more embodiments, the display apparatus 2 may be between the side window glasses 1100 opposite to (e.g., facing) each other. The display apparatus 2 may be on at least one of (e.g., selected from among) the cluster 1400, the center fascia 1500, and the passenger seat dashboard 1600.

The display apparatus 2 may include an organic light-emitting display, an inorganic electroluminescent display, a quantum dot display, and/or the like. Hereinafter, as the display apparatus 2 according to one or more embodiments, an organic light-emitting display apparatus including the light-emitting device will be described as an example, but one or more suitable types (kinds) of display apparatuses as described herein may be used in embodiments.

Referring to FIG. 6A, the display apparatus 2 may be on the center fascia 1500. In one or more embodiments, the display apparatus 2 may display navigation information. In one or more embodiments, the display apparatus 2 may display information regarding audio settings, video setting, or vehicle settings.

Referring to FIG. 6B, the display apparatus 2 may be on the cluster 1400. In this case, the cluster 1400 may display driving information and/or the like through the display apparatus 2. In other words, the cluster 1400 may be implemented in a digital manner. The cluster 1400 operated in the digital manner may display vehicle information and driving information as images. In one or more embodiments, a needle and a gauge of a tachometer and one or more suitable warning light icons may be displayed by a digital signal.

Referring to FIG. 6C, the display apparatus 2 may be on the passenger seat dashboard 1600. The display apparatus 2 may be embedded in the passenger seat dashboard 1600 or on the passenger seat dashboard 1600. In one or more embodiments, the display apparatus 2 on the passenger seat dashboard 1600 may display an image related to information displayed on the cluster 1400 and/or information displayed on the center fascia 1500. In one or more embodiments, the display apparatus 2 on the passenger seat dashboard 1600 may display information different from information displayed on the cluster 1400 and/or information displayed on the center fascia 1500.

### Manufacturing Method

Layers constituting the hole transport region, the emission layer, and the layers constituting the electron transport region may be formed in a certain region by using one or more suitable methods such as vacuum deposition, spin coating, casting, Langmuir-Blodgett (LB) deposition, ink-jet printing, laser-printing, laser-induced thermal imaging, and/or the like.

When the layers constituting the hole transport region, the emission layer, and the layers constituting the electron transport region are formed by vacuum deposition, the deposition may be performed at a deposition temperature in a range of about 100° C. to about 500° C., at a vacuum degree in a range of about 10⁻⁸ torr to about 10⁻³ torr, and at a deposition speed in a range of about 0.01 angstrom per second (Å/sec) to about 100 Å/sec, depending on a material to be included in a layer to be formed and the structure of a layer to be formed.

### Definition of Terms

The term “C₃-C₆₀ carbocyclic group” as used herein refers to a cyclic group including (e.g., consisting of) carbon atoms as the only ring-forming atoms and having three to sixty carbon atoms, and the term “C₁-C₆₀ heterocyclic group” as used herein refers to a cyclic group that has one to sixty carbon atoms and further includes, in addition to a carbon atom, a heteroatom as a ring-forming atom. The C₃-C₆₀ carbocyclic group and the C₁-C₆₀ heterocyclic group may each be a monocyclic group including (e.g., consisting of) one ring or a polycyclic group in which two or more rings are condensed with each other. In one or more embodiments, the number of ring-forming atoms of the C₁-C₆₀ heterocyclic group may be 3 to 61.

The “cyclic group” as used herein may include both (e.g., simultaneously) the C₃-C₆₀ carbocyclic group and the C₁-C₆₀ heterocyclic group.

The term “π electron-rich C₃-C₆₀ cyclic group” as used herein refers to a cyclic group that has 3 to 60 carbon atoms and does not include *—N═*′ as a ring-forming moiety, and the term “π electron-deficient nitrogen-containing C₁-C₆₀ heterocyclic group” as used herein refers to a heterocyclic group that has 1 to 60 carbon atoms and includes *—N═*′ as a ring-forming moiety.

In one or more embodiments,
the C₃-C₆₀ carbocyclic group may be i) Group T1 or ii) a condensed cyclic group in which two or more of Group T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T2, ii) a condensed cyclic group in which two or more of Group T2 are condensed with each other, or iii) a condensed cyclic group in which at least one Group T2 and at least one Group T1 are condensed with each other (e.g., a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
the π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like),
the π electron-deficient nitrogen-containing C₁-C₆₀ heterocyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (e.g., a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,
Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
Group T3 may be a furan group, a thiophene group, a 1H-pyrrole group, a silole group, or a borole group, and
Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group”, “the C₃-C₆₀ carbocyclic group”, “the C₁-C₆₀ heterocyclic group”, “the π electron-rich C₃-C₆₀ cyclic group”, and “the π electron-deficient nitrogen-containing C₁-C₆₀ heterocyclic group” as used herein may each refer to a group condensed to any cyclic group, a monovalent group, or a polyvalent group (e.g., a divalent group, a trivalent group, a tetravalent group, and/or the like) according to the structure of a formula for which the corresponding term is used. In one or more embodiments, the “benzene group” may be a benzo group, a phenyl group, a phenylene group, and/or the like, which may be easily understood by those of ordinary skill in the art according to the structure of a formula including the “benzene group.”

Depending on context, a divalent group may refer or be a polyvalent group (e.g., trivalent, tetravalent, etc., and not just divalent) per, e.g., the structure of a formula in connection with which of the terms are utilized.

Examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the monovalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as used herein refers to a linear or branched aliphatic hydrocarbon monovalent group that has one to sixty carbon atoms, and examples thereof include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof include an ethynyl group and a propynyl group. The term “C₂-C₆₀ alkynylene group” as used herein refers to a divalent group having the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as used herein refers to a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and/or the like. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group that has one to ten carbon atoms and further includes, in addition to the carbon atoms, at least one heteroatom as a ring-forming atom, and examples thereof include a 1,2,3,4-oxatriazolidinyl group, a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent cyclic group that has three to ten carbon atoms and at least one carbon-carbon double bond in the ring thereof and no aromaticity, and examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group that has one to ten carbon atoms, further includes, in addition to the carbon atoms, at least one heteroatom as a ring-forming atom, and has at least one double bond in the ring thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system of six to sixty carbon atoms, and the term “C₆-C₆₀ arylene group” as used herein refers to a divalent group having a carbocyclic aromatic system of six to sixty carbon atoms. Examples of the C₆-C₆₀ aryl group include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the two or more rings may be condensed with each other.

The term “C₁-C₆₀ heteroaryl group” as used herein refers to a monovalent group having a heterocyclic aromatic system that has one to sixty carbon atoms and further includes, in addition to the carbon atoms, at least one heteroatom as a ring-forming atom. The term “C₁-C₆₀ heteroarylene group” as used herein refers to a divalent group having a heterocyclic aromatic system that has one to sixty carbon atoms and further includes, in addition to the carbon atoms, at least one heteroatom as a ring-forming atom. Examples of the C₁-C₆₀ heteroaryl group include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group having two or more rings condensed with each other, only carbon atoms (for example, eight to sixty carbon atoms) as ring-forming atoms, and no aromaticity in its molecular structure when considered as a whole. Examples of the monovalent non-aromatic condensed polycyclic group include an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group that has two or more rings condensed with each other, further includes, in addition to carbon atoms (for example, one to sixty carbon atoms), at least one heteroatom as a ring-forming atom, and has no aromaticity in its molecular structure when considered as a whole. Examples of the monovalent non-aromatic condensed heteropolycyclic group are a pyrrolyl group, a thiophenyl group, a furanyl group, an indolyl group, a benzoindolyl group, a naphtho indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and/or the like. The term “divalent non-aromatic condensed heteropolycyclic group” as used herein refers to a divalent group having the same structure as the monovalent non-aromatic condensed heteropolycyclic group.

The term “C₆-C₆₀ aryloxy group” as used herein refers to —OA₁₀₂ (wherein, A₁₀₂ is the C₆-C₆₀ aryl group), the term “C₆-C₆₀ arylthio group” as used herein refers to —SA₁₀₃ (wherein, A₁₀₃ is the C₆-C₆₀ aryl group), and the term “C₆-C₆₀ arylseleno group” as used herein refers to —SeA₁₀₈ (wherein, A₁₀₈ is the C₆-C₆₀ aryl group).

The term “C₇-C₆₀ arylalkyl group” as used herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as used herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ as used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as used herein refers to any atom other than a carbon atom. Examples of the heteroatom include O, S, N, P, Si, B, Ge, Se, or any combination thereof.

The term “third-row transition metal” used herein includes hafnium (Hf), tantalum (Ta), tungsten (W), rhenium (Re), osmium (Os), iridium (Ir), platinum (Pt), gold (Au), and/or the like.

The term “Ph” as used herein refers to a phenyl group, the term “Me” as used herein refers to a methyl group, the term “Et” as used herein refers to an ethyl group, the term “ter-Bu” or “Bu^{t}” as used herein refers to a tert-butyl group, and the term “OMe” as used herein refers to a methoxy group.

The term “biphenyl group” as used herein refers to “a phenyl group that is substituted with a phenyl group.” In other words, the “biphenyl group” may be a substituted phenyl group having a C₆-C₆₀ aryl group as a substituent.

The term “terphenyl group” as used herein refers to “a phenyl group substituted with a biphenyl group.” The “terphenyl group” is a substituted phenyl group having, as a substituent, a C₆-C₆₀ aryl group substituted with a C₆-C₆₀ aryl group.

* and *′ as used herein, unless defined otherwise, each refer to a binding site to a neighboring atom in a corresponding formula or moiety.

The terms “x-axis”, “y-axis”, and “z-axis” as used herein are not limited to three axes in an orthogonal coordinate system, and may be interpreted in a broader sense than the aforementioned three axes in an orthogonal coordinate system. For example, the x-axis, y-axis, and z-axis may describe axes that are orthogonal to each other, or may describe axes that are in different directions that are not orthogonal to each other.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light-emitting device, the electronic apparatus, the electronic device, a device of manufacturing thereof, and/or any other relevant devices or components according to embodiments of the present disclosure described herein may be implemented utilizing any suitable hardware, firmware (e.g., an application-specific integrated circuit), software, or a combination of software, firmware, and hardware. For example, the one or more suitable components of the light-emitting device and the electronic apparatus and/or device may be formed on one integrated circuit (IC) chip or on separate IC chips. Further, the one or more suitable components of the light-emitting device and the electronic apparatus and/or device may be implemented on a flexible printed circuit film, a tape carrier package (TCP), a printed circuit board (PCB), or formed on one substrate. Further, the one or more suitable components of the light-emitting device and the electronic apparatus and/or device may be a process or thread, running on one or more processors, in one or more computing devices, executing computer program instructions and interacting with other system components for performing the one or more suitable functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, and/or the like. Also, a person of skill in the art should recognize that the functionality of one or more suitable computing devices may be combined or integrated into a single computing device, or the functionality of a particular computing device may be distributed across one or more other computing devices without departing from the scope of the embodiments of the present disclosure.

Hereinafter, compounds according to embodiments and light-emitting devices according to embodiments will be described in detail with reference to the following Synthesis Examples and Examples. The wording “B was used instead of A” used in describing Synthesis Examples indicates that an identical molar equivalent of B was used in place of A. The Synthesis Examples and Examples described in more detail are each one example embodiment for enhancing understanding, and the scope of the present disclosure is not limited thereto.

### EXAMPLES

### Synthesis Examples

### Synthesis Example 1: Synthesis of Compound 1

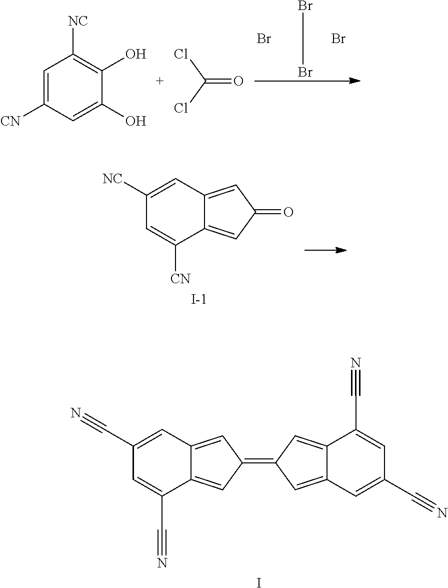

### Synthesis of Intermediate 1-1

Carbonyl dichloride (3.07 g, 0.031 mol) and carbon tetrabromide (20.56 g, 0.062 mol) were added to 3,5-diisocyanobenzene-1,2-diol (5.0 g, 0.031 mol), and the mixture was heated in acetic acid. After cooling the reaction mixture to room temperature, the resulting solid was filtered and washed with ethanol and water to obtain 4.53 g of intermediate 1-1. (Yield: 80.5%)

### Synthesis of Compound 1

Titanium tetrachloride (5.26 g, 0.028 mol) and intermediate 1-1 (4.0 g, 0.022 mol) were reacted for 24 hours in pyridine/dichloromethane as a solvent after nitrogen drying. After quenching the reaction with HCl, the mixture was washed with dichloromethane, and water was removed using anhydrous sodium sulfate. The solvent was removed under reduced pressure, and recrystallization using dichloromethane/MeOH was performed thereon to obtain 3.86 g of compound 1. (Yield: 53.0%)

### Synthesis Example 2: Synthesis of Compound 25

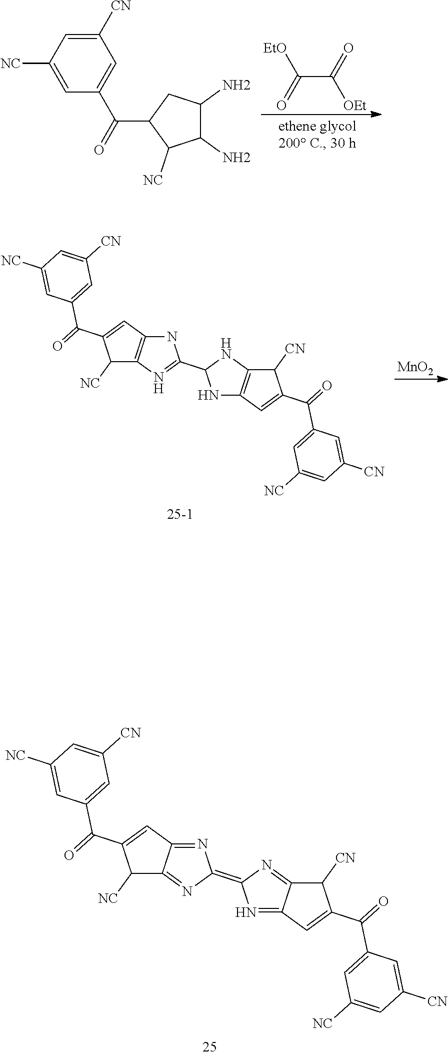

### Synthesis of Intermediate 25-1

Under argon atmosphere, 5-(3,4-diamino-2-cyanocyclopenta-2,4-diene-1-carbonyl) isophthalonitrile (10.0 g, 0.036 mol) was stirred with diethyl oxalate (1.34 g, 0.009 mol) and ethylene glycol at 200° C. for 30 hours. Distilled water was added to the reaction mixture, and the precipitated solid was filtered. The obtained solid was purified by silica gel column chromatography (chloroform:ethyl acetate=20:1) to obtain intermediate 25-1 (16.7 g, 80.6%).

### Synthesis of Compound 25

Under argon atmosphere, manganese dioxide (46.4 g, 0.53 mol) and tetrahydrofuran (300 mL) were added to intermediate 25-1 (15 g, 0.027 mol) and reacted at room temperature for 50 hours. The reaction mixture was filtered through celite and the solvent was removed under reduced pressure followed by drying. The obtained solid was purified by silica gel column chromatography (chloroform:ethyl acetate=1:1) to obtain compound 25 (0.5 g, 3.3%).

### Synthesis Example 3: Synthesis of Compound 41

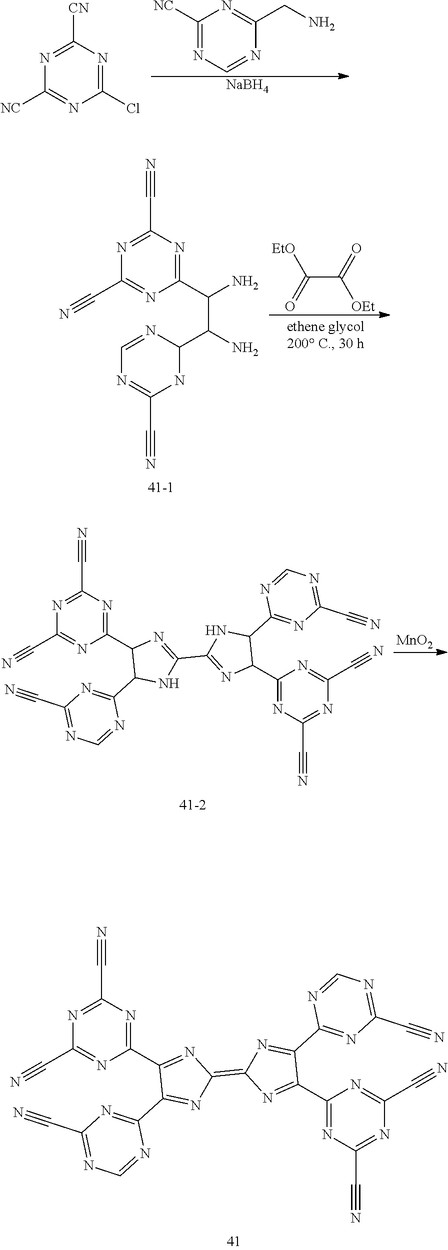

### Synthesis of Intermediate 41-1

Under nitrogen atmosphere, 6-chloro-1,3,5-triazine-2,4-dicarbonitrile (10.0 g, 0.06 mol) was stirred with tetrahydrofuran and isopropyl magnesium chloride at room temperature for 2 hours. Then, 4-(aminomethyl)-1,3,5-triazine-2-carbonitrile (8.11 g, 0.06 mol) was added thereto and stirred for 3 hours. After removal of the solvent, methanol and NaBH₄ (2.27 g, 0.06 mol) were added thereto. The mixture was continuously stirred at room temperature for 20 hours, extracted with ether, and the solvent was removed under reduced pressure to obtain intermediate 41-1 (9.44 g, 0.06 mol, yield: 53.4%).

### Synthesis of Intermediate 41-2 and Compound 41

Intermediate 41-2 and compound 41 were synthesized in the same manner as used to synthesize intermediate 25-1 and compound 25, respectively, in the synthetic process of compound 25.

### Synthesis Example 4: Synthesis of Compound 50

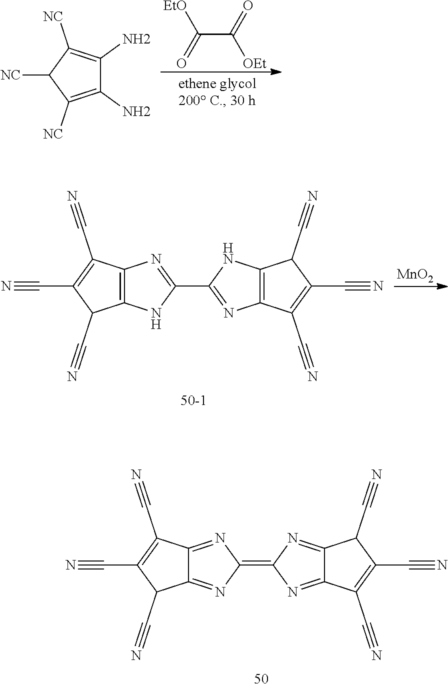

### Synthesis of Intermediate 50-1

Under argon atmosphere, 4,5-diaminocyclopenta-3,5-diene-1,2,3-tricarbonitrile (10.0 g, 0.058 mol) was stirred with diethyl oxalate (2.15 g, 0.015 mol) and ethylene glycol at 200° C. for 30 hours. After adding distilled water to the reaction mixture, the precipitated solid was purified by silica gel column chromatography (chloroform:ethyl acetate=20:1, with 1% triethylamine) to obtain intermediate 50-1 (16.74 g, 80.1%).

### Synthesis of Compound 50

Under argon atmosphere, manganese dioxide (72.4 g, 0.83 mol) and tetrahydrofuran (300 mL) were added to intermediate 50-1 (15 g, 0.042 mol) and allowed to stand at room temperature for 50 hours. The reaction mixture was filtered through celite and the solvent was removed under reduced pressure followed by drying. The obtained solid was purified by silica gel column chromatography (chloroform:ethyl acetate=1:1) to obtain compound 50 (0.38 g, 2.5%).

### Synthesis Example 5: Synthesis of Compound 53

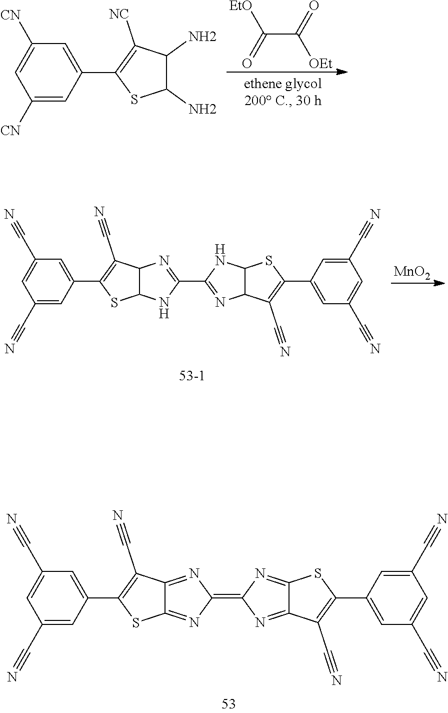

### Synthesis of Intermediate 53-1

Under argon atmosphere, 4,5-diamino-2-(3,5-diisocyanophenyl)-4,5-dihydrothiophene-3-carbonitrile (10.0 g, 0.037 mol) was stirred with diethyl oxalate (1.38 g, 0.009 mol) and ethylene glycol at 200° C. for 30 hours. After adding distilled water to the reaction mixture, the precipitated solid was filtered.

The obtained solid was purified by silica gel column chromatography (chloroform:ethyl acetate=20:1, with 1% triethylamine) to obtain intermediate 53-1 (16.23 g, yield: 79.0%).

### Synthesis of Compound 53

Under argon atmosphere, manganese dioxide (46.9 g, 0.54 mol) and tetrahydrofuran (300 mL) were added to compound 53-1 (15 g, 0.027 mol) and allowed to stand at room temperature for 50 hours. The reaction mixture was filtered through celite and the solvent was removed under reduced pressure followed by drying. The obtained solid was purified by silica gel column chromatography (chloroform:ethyl acetate=1:1) to obtain compound 53 (0.32 g, 2.2%).

TABLE 1

| Compound | | MS/FAB | |
|---|---|---|---|
| No. | ¹H-NMR(CDCl₃) | found | calc. |
| 1 | 8.04 (s, 2H), 7.19 (s, 2H), 5.62 (s, 4H) | 328.03 | 328.07 |
| 25 | 8.62 (s, 4H), 8.07 (s, 2H), 6.65(s, 2H), | 566.08 | 566.10 |
| | 3.2(s, 2H) | | |
| 41 | 10.18 (s, 2H) | 598.01 | 598.08 |
| 50 | 3.2 (s, 2H) | 357.09 | 358.05 |
| 53 | 8.11 (s, 4H), 7.67 (s, 2H) | 546.01 | 546.02 |

### Evaluation Example 1: Compound Characteristics Evaluation

The highest occupied molecular orbital (HOMO) energy level, lowest unoccupied molecular orbital (LUMO) energy level, hole mobility, electron mobility, and glass transition temperature of the compounds were confirmed. Results are shown in Table 2.

HOMO energy levels and LUMO energy levels were calculated as follows: voltage (V)-current (A) graphs of each compound were obtained using cyclic voltammetry (CV) (electrolyte: 0.1 molarity (M) Bu₄NPF₆/solvent: dimethylformamide (DMF)/electrode: 3-electrode system (working electrode: GC, reference electrode: Ag/AgCl, auxiliary electrode: Pt)), HOMO energy levels were calculated from oxidation onset of the graphs, and LUMO energy levels were calculated from reduction onset of the graphs. Hole mobility and electron mobility were evaluated using a space-charge-limited current (SCLC) method as described in “Hole mobility of N,N′-bis(naphtanlen-1-yl)-N,N′-bis(phenyl)benzidine investigated by using space-charge-limited currents,” Appl. Phys. Lett. 90, 203512 (2007) the entire content of which is herein incorporated by reference. Glass transition temperature (Tg) characteristics were analyzed using differential scanning calorimetry (DSC). Analysis conditions were as follows: sample weight of 5 milligram (mg) was heated from room temperature to 300° C. at a scan rate of 10 degrees Celsius per minute (° C./min), cooled to 25° C., and heated again to 300° C. The glass transition temperature was measured during the second heating and obtained from the inflection point on the graph.

TABLE 2

| | | | Hole | Electron | Glass transition |
|---|---|---|---|---|---|
| | HOMO | LUMO | mobility | mobility | temperature |
| Compound | (eV) | (eV) | (cm²/Vs) | (cm²/Vs) | (° C.) |
| 1 | −6.67 | −5.24 | 5.34E−02 | 4.79E−03 | 112.3 |
| 25 | −7.90 | −5.22 | 1.04E−09 | 4.48E−06 | 117.5 |
| 41 | −8.86 | −5.79 | 1.36E−06 | 2.30E−05 | 107.2 |
| 50 | −8.41 | −5.71 | 1.76E−06 | 2.39E−03 | 118.9 |
| 53 | −7.67 | −5.24 | 8.74E−05 | 9.47E−05 | 134.1 |

Referring to Table 2, it was confirmed that the compounds according to the disclosure have excellent thermal stability when employed in light-emitting devices because the compounds have high glass transition temperatures.

### LIGHT-EMITTING DEVICE EXAMPLES

### Example 1

As an anode, a glass substrate (product of Corning Inc.) with a 15 ohm per square centimeter (Q/cm²) (1,300 angstrom (A)) ITO formed thereon was cut to a size of 50 millimeter (mm)×50 mm×0.7 mm, sonicated by using isopropyl alcohol and pure water each for 5 minutes, washed by irradiation of ultraviolet rays and exposure of ozone thereto for 30 minutes, and then mounted on a vacuum deposition apparatus.

Compound 1 and compound HT3 were vacuum-deposited on the anode at a weight ratio of 3:97 to form a hole injection layer having the thickness of 100 Å, and compound HT40 was vacuum-deposited on the hole injection layer to form a hole transport layer having the thickness of 1,250 Å.

Compounds HTH57, ETH87, and PD42 were vacuum-deposited on the hole transport layer at a weight ratio of 45:45:10 to form an emission layer having the thickness of 300 Å.

Compound ET37 was vacuum-deposited on the emission layer to form a buffer layer having the thickness of 50 Å, and ET46 and LiQ were vacuum-deposited at a weight ratio of 5:5 on the buffer layer to form an electron transport layer having the thickness of 310 Å. Subsequently, Yb was vacuum-deposited on the electron transport layer to form an electron injection layer having the thickness of 15 Å, followed by vacuum deposition of Ag and Mg at a weight ratio of 5:5 to form a cathode having the thickness of 1,000 Å.

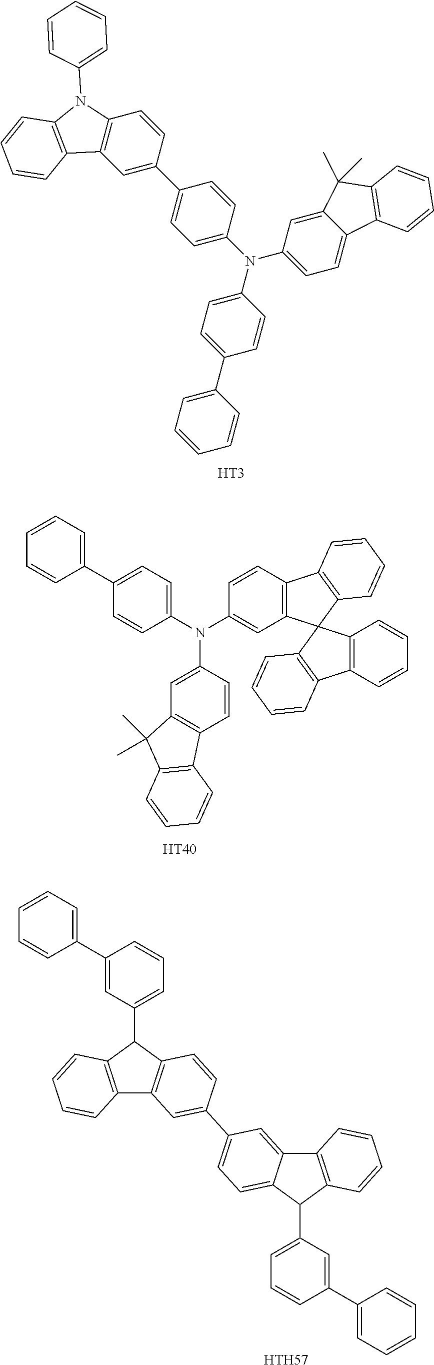

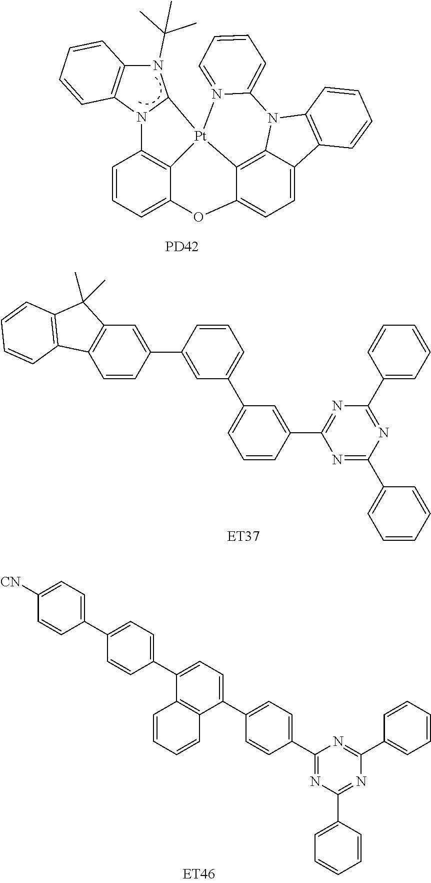

### Examples 2 to 5 and Comparative Examples 1 to 3

Light-emitting devices were manufactured in substantially the same manner as in Example 1, except that compounds shown in Table 3 were each used instead of compound 1 in forming a hole injection layer.

### Evaluation Example 2: Characterization of Light-Emitting Devices

To evaluate characteristics of the light-emitting devices manufactured according to Examples 1 to 5 and Comparative Examples 1 to 3, the driving voltage at a current density of 10 milliampere per square centimeter (mA/cm²) and current efficiency were measured. Results are shown in the Table 3. The driving voltage of each of the light-emitting devices was measured using a source meter (Keithley Instrument, 2400 series), and the current efficiency was measured using a luminance meter CS-2000 (Konica Minolta). Additionally, the device lifespan was specified as the time it takes to reach 95% of the initial luminance. All evaluation results were calculated by comparing the numerical values of Comparative Example 2 with Comparative Example 1, Comparative Example 3, and Examples 1 to 5.

TABLE 3

| | | Relative | Relative |
|---|---|---|---|
| | Compound | driving voltage | lifespan |
| Example 1 | 1 | 68% | 120% |
| Example 2 | 25 | 69% | 120% |
| Example 3 | 41 | 67% | 121% |
| Example 4 | 50 | 67% | 121% |
| Example 5 | 53 | 68% | 120% |
| Comparative | A | 83% | 114% |
| Example 1 | | | |
| Comparative | B | 100% | 100% |
| Example 2 | | | |
| Comparative | C | 112% | 90% |
| Example 3 | | | |

1 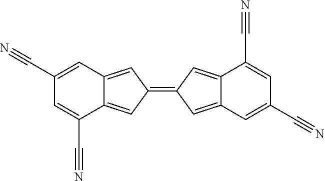 [SMILES: N#Cc1cc(C#N)c2c(c1)=C/C(=C1/C=c3cc(C#N)cc(C#N)c3=C1)C=2]
25 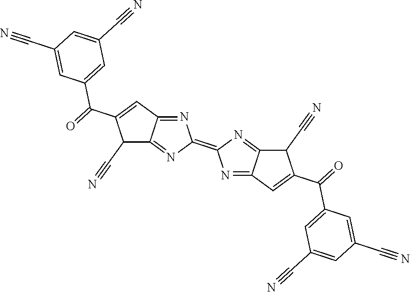 [SMILES: N#Cc1cc(C#N)cc(C(=O)C2=CC3=N/C(=C4/N=C5C=C(C(=O)c6cc(C#N)cc(C#N)c6)C(C#N)C5=N4)N=C3C2C#N)c1]
41 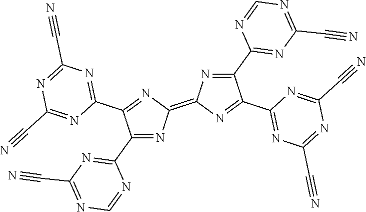 [SMILES: N#Cc1ncnc(C2=N/C(=C3/N=C(c4ncnc(C#N)n4)C(c4nc(C#N)nc(C#N)n4)=N3)N=C2c2nc(C#N)nc(C#N)n2)n1]
50 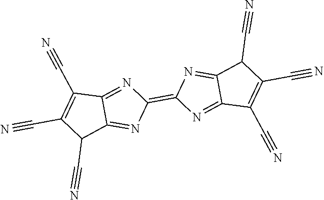 [SMILES: N#CC1=C(C#N)C(C#N)C2=N/C(=C3\N=C4C(=N3)C(C#N)C(C#N)=C4C#N)N=C21]
53 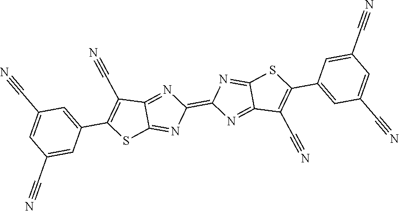 [SMILES: N#Cc1cc(C#N)cc(-c2sc3c(c2C#N)=N/C(=C2\N=c4sc(-c5cc(C#N)cc(C#N)c5)c(C#N)c4=N2)N=3)c1]
A 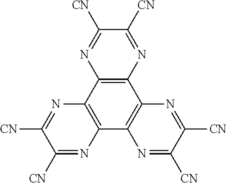
B 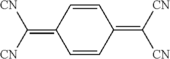
C

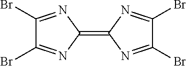 [SMILES: BrC1=NC(=C2N=C(Br)C(Br)=N2)N=C1Br]

From Table 3, it was confirmed that the light-emitting devices according to Examples 1 to 5 had superior driving voltage and lifespan characteristics to those of the light-emitting devices according to Comparative Examples 1 to 3.

The light-emitting devices have suitable or excellent low driving voltage and long lifespan characteristics by including an organic compound represented by Formula 1, and enable the manufacture of high-quality electronic apparatuses and electronic devices.

A person of ordinary skill in the art, in view of the present disclosure in its entirety, would appreciate that each suitable feature of the one or more suitable embodiments of the present disclosure may be combined or combined with each other, partially or entirely, and may be technically interlocked and operated in one or more suitable ways, and each embodiment may be implemented independently of each other or in conjunction with each other in any suitable manner unless otherwise stated or implied. However, the aspects and features of embodiments of the present disclosure are not limited to those described herein, and one or more suitable other aspects and features as would be understood by those having ordinary skill in the art may be included in the present disclosure.

In the context of the present application and unless otherwise defined, the terms “use,” “using,” and “used” may be considered synonymous with the terms “utilize,” “utilizing,” and “utilized,” respectively.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that one or more suitable changes in form and details may be made therein without departing from the spirit and scope as defined by the following claims, and equivalents thereof.

What is claimed is:

1. A light-emitting device comprising:
a first electrode;
a second electrode opposite to the first electrode;
an interlayer between the first electrode and the second electrode and comprising an emission layer; and
an organic compound, the organic compound represented by Formula 1: 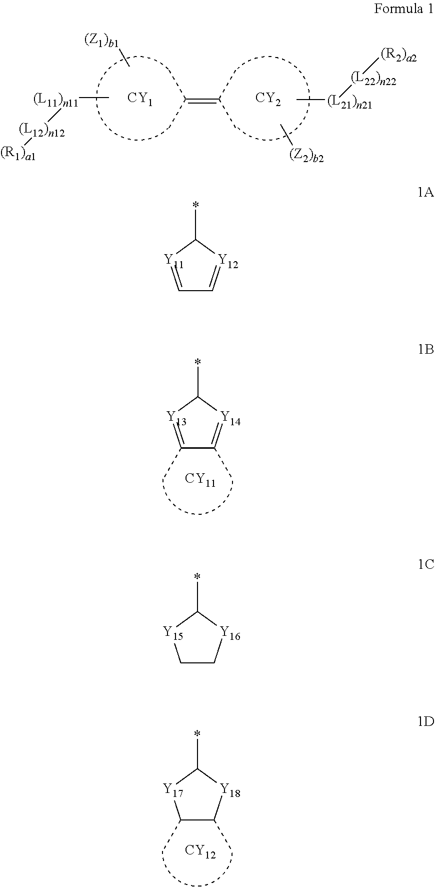 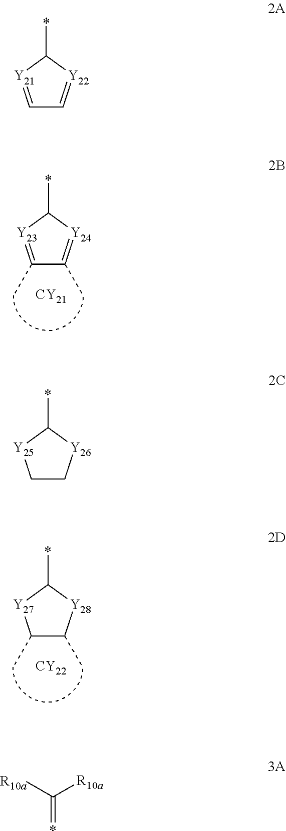
wherein, in Formulae 1, 1A to 1D, 2A to 2D, and 3A,
ring CY₁ is represented by any one of Formulae 1A to 1D,
ring CY₂ is represented by any one of Formulae 2A to 2D,
ring CY₁₁, ring CY₁₂, ring CY₂₁, and ring CY₂₂ are each independently a C₅-C₆₀ carbocyclic group or a C₁-C₆₀ heterocyclic group,
Y₁₁ is C(R₁₁), Si(R₁₁), or N,
Y₁₂ is C(R₁₂), Si(R₁₂), or N,
Y₁₃ is C(R₁₃), Si(R₁₃), or N,
Y₁₄ is C(R₁₄), Si(R₁₄), or N,
Y₁₅ is C(R₁₅)(R₁₅′), Si(R₁₅)(R₁₅′), N(R₁₅), O, S, or Se,
Y₁₆ is C(R₁₆)(R₁₆′), Si(R₁₆)(R₁₆′), N(R₁₆), O, S, or Se,
Y₁₇ is C(R₁₇)(R₁₇′), Si(R₁₇)(R₁₇′), N(R₁₇), O, S, or Se,
Y₁₈ is C(R₁₈)(R₁₈′), Si(R₁₈)(R₁₈′), N(R₁₈), O, S, or Se,
Y₂₁ is C(R₂₁), Si(R₂₁), or N,
Y₂₂ is C(R₂₂), Si(R₂₂), or N,
Y₂₃ is C(R₂₃), Si(R₂₃), or N,
Y₂₄ is C(R₂₄), Si(R₂₄), or N,
Y₂₅ is C(R₂₅)(R₂₅′), Si(R₂₅)(R₂₅′), N(R₂₅), O, S, or Se,
Y₂₆ is C(R₂₆)(R₂₆′), Si(R₂₆)(R₂₆′), N(R₂₆), O, S, or Se,
Y₂₇ is C(R₂₇)(R₂₇′), Si(R₂₇)(R₂₇′), N(R₂₇), O, S, or Se,
Y₂₈ is C(R₂₈)(R₂₈′), Si(R₂₈)(R₂₈′), N(R₂₈), O, S, or Se,
L₁₁, L₁₂, L₂₁, and L₂₂ are each independently a single bond, *—C(R₃)(R₄)—*′, *—Si(R₃)(R₄)—*′, *—Ge(R₃)(R₄)—*′, *—S—*′, *—Se—*′, *—O—*′, *—C(═O)—*′, *—S(═O)—*′, *—S(═O)₂—*′, *═C(R₃)—*′, *—C(R₃)═C(R₄)—*′, *—C(═S)—*′, *—C≡C—*′, a C₅-C₆₀ carbocyclic group unsubstituted or substituted with at least one R₁₀ₐ, or a C₁-C₆₀ heterocyclic group unsubstituted or substituted with at least one R₁₀ₐ,
n11, n12, n21, and n22 are each independently an integer from 1 to 10,
R₁ to R₄, R₁₁ to R₁₈, R₁₈′ to R₁₈′, R₂₁ to R₂₈, R₂₅′ to R₂₈′, Z₁, and Z₂ are each independently a group represented by Formula 3A, hydrogen, deuterium, —F, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group, a C₆-C₆₀ arylthio group unsubstituted or substituted with at least one R₁₀ₐ, a C₆-C₆₀ arylseleno group unsubstituted or substituted with at least one R₁₀ₐ, a C₇-C₆₀ arylalkyl group unsubstituted or substituted with at least one R₁₀ₐ, a C₂-C₆₀ heteroaryl alkyl group unsubstituted or substituted with at least one R₁₀ₐ, —Si(Q₁)(Q₂)(Q₃), —Ge(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more adjacent substituents selected from among R₁ to R₄, R₁₁ to R₁₈, R₁₅′ to R₁₈′, R₂₁ to R₂₈, R₂₅′ to R₂₈′, Z₁, and Z₂ are optionally bonded to each other to provide a C₅-C₃₀ carbocyclic group unsubstituted or substituted with at least one R₁₀ₐ, or a C₂-C₃₀ heterocyclic group unsubstituted or substituted with at least one R₁₀ₐ,
a1 is an integer from 0 to 30,
a2 is an integer from 0 to 30,
b1 is an integer from 0 to 10,
b2 is an integer from 0 to 10,
* indicates a binding site to a double bond in Formula 1,
the organic compound represented by Formula 1 may comprise at least one cyano group,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

2. The light-emitting device of claim 1, wherein
the first electrode is an anode,
the second electrode is a cathode,
the interlayer further comprises a hole transport region between the first electrode and the emission layer and an electron transport region between the emission layer and the second electrode,
the hole transport region comprises at least one selected from among a hole injection layer, a hole transport layer, a buffer layer, an emission auxiliary layer, and an electron-blocking layer, and
the electron transport region comprises at least one selected from among a hole-blocking layer, an electron transport layer, and an electron injection layer.

3. The light-emitting device of claim 2, wherein
the hole injection layer comprises the organic compound.

4. The light-emitting device of claim 1, further comprising:
a second compound comprising a group represented by Formula 20, a third compound comprising at least one π electron-deficient nitrogen-containing C₁-C₆₀ cyclic group, a fourth compound comprising a transition metal, or any combination thereof, wherein
the second compound, the third compound, and the fourth compound are different from each other: 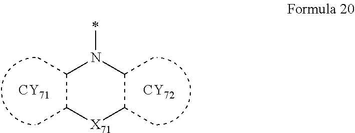
wherein ring CY₇₁ and ring CY₇₂ are each independently a π electron-rich C₃-C₆₀ cyclic group or a pyridine group,
X₇₁ is a single bond, or a linking group comprising O, S, N, B, C, Si, or any combination thereof,
* in Formula 20 is a bonding site to a neighboring atom in compound 20, and
CBP and mCBP are excluded from the second compound:

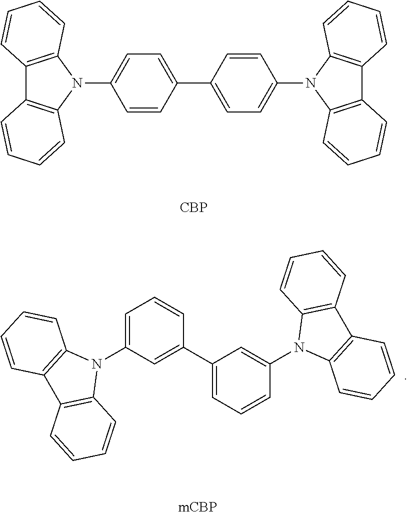 [SMILES: c1ccc2c(c1)c1ccccc1n2-c1ccc(-c2ccc(-n3c4ccccc4c4ccccc43)cc2)cc1 ; "CBP" ; c1cc(-c2cccc(-n3c4ccccc4c4ccccc43)c2)cc(-n2c3ccccc3c3ccccc32)c1]

5. The light-emitting device of claim 4, wherein the third compound comprises a pyridine group, a pyrimidine group, a pyrazine group, a pyridazine group, a triazine group, or any combination thereof.

6. An electronic apparatus comprising the light-emitting device of claim 1.

7. The electronic apparatus of claim 6, further comprising
a color filter, a color conversion layer, a touch screen layer, a polarizing layer, or any combination thereof.

8. The electronic apparatus of claim 6, further comprising a thin-film transistor, wherein
the thin-film transistor includes a source electrode and a drain electrode, and
the first electrode of the light-emitting device is electrically coupled to the source electrode or the drain electrode.

9. An electronic device comprising the light-emitting device of claim 1.

10. The electronic device of claim 9, wherein
the electronic device is at least one selected from among a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, and a signboard.

11. An organic compound represented by Formula 1: 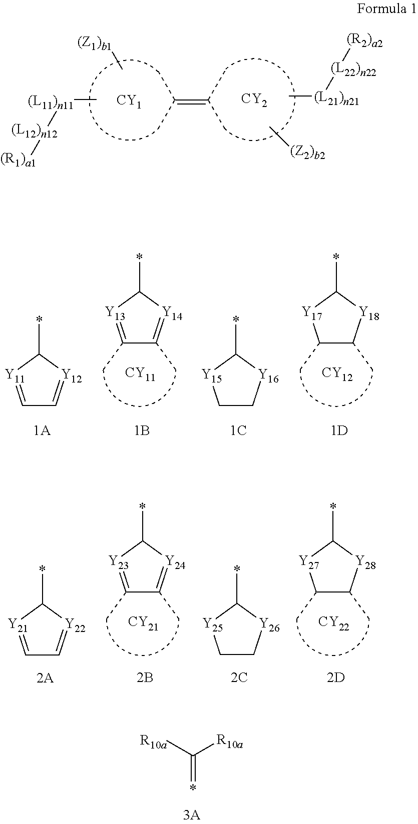
wherein, in Formulae 1, 1A to 1D, 2A to 2D, and 3A,
ring CY₁ is represented by any one of Formulae 1A to 1D,
ring CY₂ is represented by any one of Formulae 2A to 2D,
ring CY₁₁, ring CY₁₂, ring CY₂₁, and ring CY₂₂ are each independently a C₅-C₆₀ carbocyclic group or a C₁-C₆₀ heterocyclic group,
Y₁₁ is C(R₁₁), Si(R₁₁), or N,
Y₁₂ is C(R₁₂), Si(R₁₂), or N,
Y₁₃ is C(R₁₃), Si(R₁₃), or N,
Y₁₄ is C(R₁₄), Si(R₁₄), or N,
Y₁₅ is C(R₁₅)(R₁₅′), Si(R₁₅)(R₁₅′), N(R₁₅), O, S, or Se,
Y₁₆ is C(R₁₆)(R₁₆′), Si(R₁₆)(R₁₆′), N(R₁₆), O, S, or Se,
Y₁₇ is C(R₁₇)(R₁₇′), Si(R₁₇)(R₁₇′), N(R₁₇), O, S, or Se,
Y₁₈ is C(R₁₈)(R₁₈′), Si(R₁₈)(R₁₈′), N(R₁₈), O, S, or Se,
Y₂₁ is C(R₂₁), Si(R₂₁), or N,
Y₂₂ is C(R₂₂), Si(R₂₂), or N,
Y₂₃ is C(R₂₃), Si(R₂₃), or N,
Y₂₄ is C(R₂₄), Si(R₂₄), or N,
Y₂₅ is C(R₂₅)(R₂₅′), Si(R₂₅)(R₂₅′), N(R₂₅), O, S, or Se,
Y₂₆ is C(R₂₆)(R₂₆′), Si(R₂₆)(R₂₆′), N(R₂₆), O, S, or Se,
Y₂₇ is C(R₂₇)(R₂₇′), Si(R₂₇)(R₂₇′), N(R₂₇), O, S, or Se,
Y₂₈ is C(R₂₈)(R₂₈′), Si(R₂₈)(R₂₈′), N(R₂₈), O, S, or Se,
L₁₁, L₁₂, L₂₁, and L₂₂ are each independently a single bond, *—C(R₃)(R₄)—*′, *—Si(R₃)(R₄)—*′, *—Ge(R₃)(R₄)—*′, *—S—*′, *—Se—*′, *—O—*′, *—C(═O)—*′, *—S(═O)—*′, *—S(═O)₂—*′, *═C(R₃)—*′, *—C(R₃)═C(R₄)—*′, *—C(═S)—*′, *—C≡C—*′, a C₅-C₆₀ carbocyclic group unsubstituted or substituted with at least one R₁₀ₐ, or a C₁-C₆₀ heterocyclic group unsubstituted or substituted with at least one R₁₀ₐ,
n11, n12, n21, and n22 are each independently an integer from 1 to 10,
R₁ to R₄, R₁₁ to R₁₈, R₁₅′ to R₁₈′, R₂₁ to R₂₈, R₂₅′ to R₂₈′, Z₁, and Z₂ are each independently a group represented by Formula 3A, hydrogen, deuterium, —F, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group, a C₆-C₆₀ arylthio group unsubstituted or substituted with at least one R₁₀ₐ, a C₆-C₆₀ arylseleno group unsubstituted or substituted with at least one R₁₀ₐ, a C₇-C₆₀ arylalkyl group unsubstituted or substituted with at least one R₁₀ₐ, a C₂-C₆₀ heteroaryl alkyl group unsubstituted or substituted with at least one R₁₀ₐ, —Si(Q₁)(Q₂)(Q₃), —Ge(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more adjacent substituents selected from among R₁ to R₄, R₁₁ to R₁₈, R₁₅′ to R₁₈′, R₂₁ to R₂₈, R₂₅′ to R₂₈′, Z₁, and Z₂ are optionally bonded to each other to provide a C₅-C₃₀ carbocyclic group unsubstituted or substituted with at least one R₁₀ₐ, or a C₂-C₃₀ heterocyclic group unsubstituted or substituted with at least one R₁₀ₐ,
a1 is an integer from 0 to 30,
a2 is an integer from 0 to 30,
b1 is an integer from 0 to 10,
b2 is an integer from 0 to 10,
* indicates a bonding site to a double bond of Formula 1 or a bonding site with a neighboring atom,
the organic compound represented by Formula 1 may comprise at least one cyano group, and
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

12. The organic compound of claim 11, wherein
ring CY₁₁, ring CY₁₂, ring CY₂₁, and ring CY₂₂ are each independently
a benzene group, a naphthalene group, an anthracene group, a phenanthrene group, a triphenylene group, a pyrene group, a chrysene group, a cyclopentene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a dibenzooxasiline group, a dibenzothiasiline group, a dibenzodihydroazasiline group, a dibenzodihydrodihydrodisiline group, a dibenzodihydrosiline group, a dibenzodioxine group, a dibenzooxathiine group, a dibenzooxazine group, a dibenzopyran group, a dibenzodithiine group, a dibenzothiazine group, a dibenzothiopyran group, a dibenzocyclohexadiene group, a dibenzodihydropyridine group, or a dibenzodihydropyrazine group.

13. The organic compound of claim 11, wherein
ring CY₁₁, ring CY₁₂, ring CY₂₁, and ring CY₂₂ are each independently a benzene group, a cyclopentene group, a cyclopentadiene group, a furan group, or a thiophene group.

14. The organic compound of claim 11, wherein
the organic compound is represented by any one of Formulae 1-1 to 1-5: 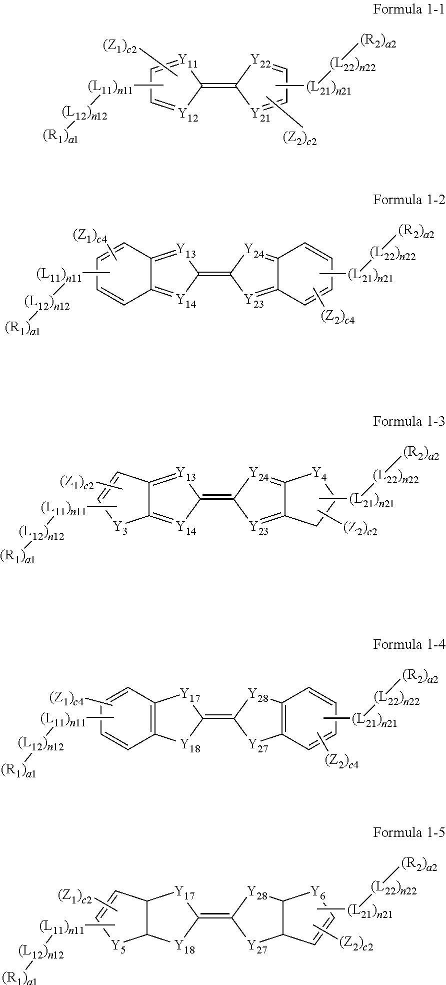
wherein, in Formulae 1-1 to 1-5,
Y₁₁, Y₁₂, Y₂₁, Y₂₂, Y₁₃, Y₁₄, Y₂₃, Y₂₄, Y₁₇, Y₁₈, Y₂₇, Y₂₈, L₁₁, L₁₂, L₂₁, L₂₁, n11, n12, n21, n22, R₁, R₂, a1, a2, Z₁, and Z₂ are as described in Formula 1,
Y₃ is C(R₃₁)(R₃₂), N(R₃₁), O, or S,
Y₃ is C(R₄₁)(R₄₂), N(R₄₁), O, or S,
Y₄ is C(R₅₁)(R₅₂), N(R₅₁), O, or S,
Y₆ is C(R₆₁)(R₆₂), N(R₆₁), O, or S,
c2 is an integer from 0 to 2,
c4 is an integer from 0 to 4, and
R₃₁, R₃₂, R₄₁, R₄₂, R₅₁, R₅₂, R₆₁, and R₆₂ are as described for R₁ in Formula 1.

15. The organic compound of claim 11, wherein
L₁₁, L₁₂, L₂₁, and L₂₂ are each independently a single bond, *—C(R₃)(R₄)—*′, *—Si(R₃)(R₄)—*′, *—Ge(R₃)(R₄)—*′, *—S—*′, *—Se—*, *—O—*′, *—C(═O)—*′, *—S(═O)—*′, *—S(═O)₂*′, *═C(R₃)—*′, *—C(R₃)═C(R₄)—*′, *—C(═S)*′, *—C≡C—*′, a benzene group, a naphthalene group, a phenanthrene group, a fluorene group, a cyclopentene group, a cyclopentadiene group, a carbazole group, a dibenzofuran group, a dibenzothiophene group, an indole group, a pyridine group, a pyrimidine group, a triazine group, an azacarbazole group, an azadibenzofuran group, or an azadibenzothiophene group, each unsubstituted or substituted with at least one R₁₀ₐ.

16. The organic compound of claim 11, wherein
L₁₁, L₁₂, L₂₁, and L₂₂ are each independently
a single bond, *—C(R₃)(R₄)*′, *—S—*′, *—C(═O)—*′, *═C(R₃)—*′, *—C(═S)—*′, or any one of Formulae 2-1 to 2-15: 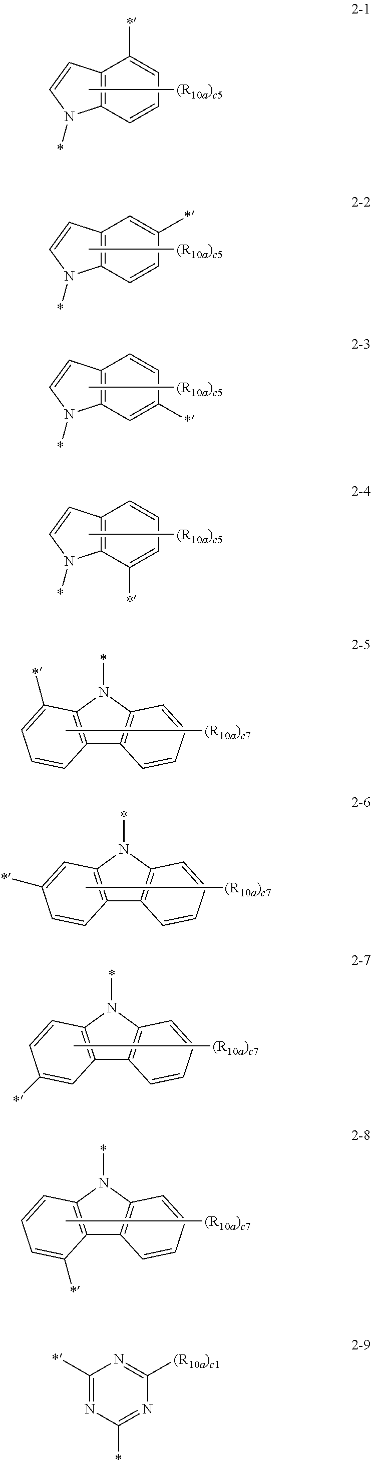 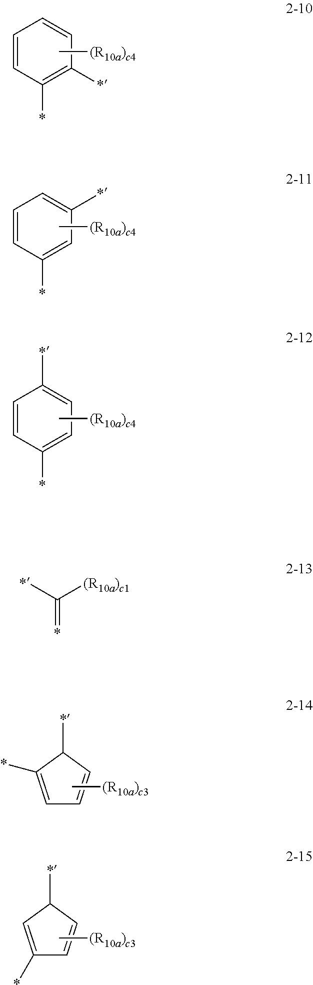
wherein, in Formulae 2-1 to 2-15,
R₁₀ₐ is as described in Formula 1,
c1 is an integer from 0 to 1,
c3 is an integer from 0 to 3,
c4 is an integer from 0 to 4,
c5 is an integer from 0 to 5,
c7 is an integer from 0 to 7, and
* and *′ each indicate a binding site to a neighboring atom.

17. The organic compound of claim 11, wherein
R₁ to R₄, R₁₁ to R₁₈, R₁₈′ to R₁₈′, R₂₁ to R₂₈, R₂₅′ to R₂₈′, Z₁, and Z₂ are each independently selected from among:
a group represented by Formula 3A, hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a phenyl group, a biphenyl group, or a terphenyl group;
a C₁-C₆₀ alkyl group, a C₁-C₆₀ cycloalkyl group, a C₁-C₆₀ heterocycloalkyl group, a phenyl group, a biphenyl group, a terphenyl group, a naphthyl group, an anthracenyl group, a phenanthrenyl group, a phenalenyl group, a triphenylenyl group, a pyrenyl group, a chrysenyl group, a pyrrolyl group, a furanyl group, a thiophenyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a dibenzofuranyl group, a benzothiophenyl group, a dibenzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a carbazolyl group, a benzocarbazolyl group, a fluorenyl group, a benzoisothiazolyl group, a benzoxazolyl group, a benzoisoxazolyl group, a phenoxazinyl group, an acridinyl group, and a xanthenyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a phenyl group, a biphenyl group, a terphenyl group, or any combination thereof; and
—Si(Q₁)(Q₂)(Q₃), —Ge(Q₁)(Q₂)(Q₃), and —N(Q₁)(Q₂).

18. The organic compound of claim 11, wherein
the organic compound is represented by any one of Formula 1-1-A to Formula 1-1-D, Formula 1-2-A to Formula 1-2-D, Formula 1-3-A to 1-3-C, Formula 1-4-A, or Formula 1-5-A: 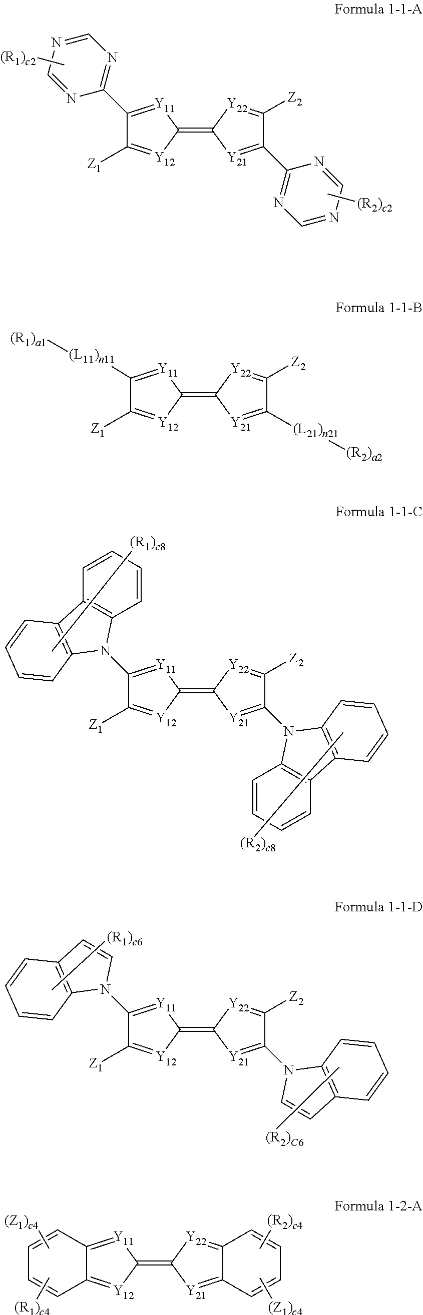 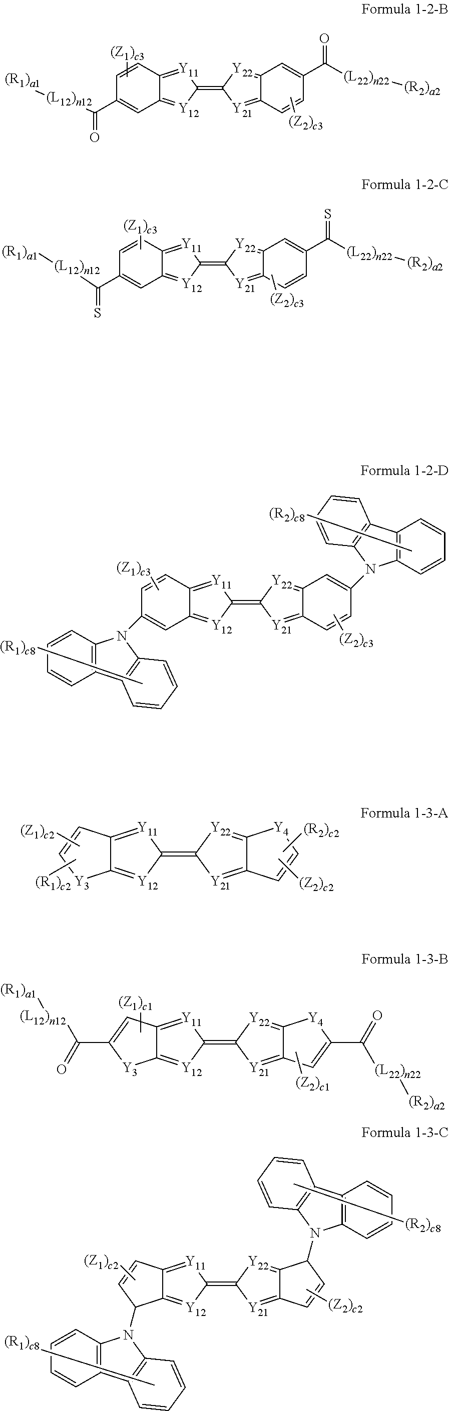 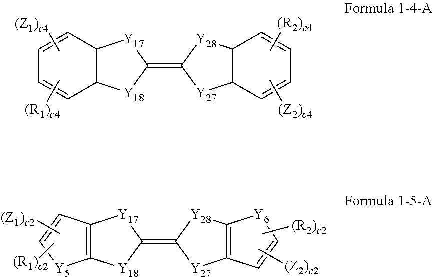
wherein, in Formula 1-1-A to Formula 1-1-D, Formula 1-2-A to Formula 1-2-D, Formula 1-3-A to 1-3-C, Formula 1-4-A, and Formula 1-5-A,
Y₁₁, Y₁₂, Y₂₁, Y₂₂, Y₁₃, Y₁₄, Y₂₃, Y₂₄, Y₁₇, Y₁₈, Y₂₇, Y₂₈, L₁₁, L₁₂, L₂₁, L₂₁, n11, n12, n21, n22, R₁, R₂, a1, a2, Z₁, and Z₂ are as described in Formula 1,
Y₃ is C(R₃₁)(R₃₂), N(R₃₁), O, or S,
Y₄ is C(R₄₁)(R₄₂), N(R₄₁), O, or S,
Y₅ is C(R₅₁)(R₅₂), N(R₅₁), O, or S,
Y₆ is C(R₆₁)(R₆₂), N(R₆₁), O, or S,
c1 is an integer from 0 to 1,
c2 is an integer from 0 to 2,
c3 is an integer from 0 to 3,
c4 is an integer from 0 to 4,
c6 is an integer from 0 to 6,
c8 is an integer from 0 to 8, and
R₃₁, R₃₂, R₄₁, R₄₂, R₅₁, R₅₂, R₆₁, and R₆₂ are as described in connection with R₁ in Formula 1.

19. The organic compound of claim 11, wherein
the organic compound has point symmetry.

20. The organic compound of claim 11, wherein
the organic compound is represented by any one of compounds 1 to 78: 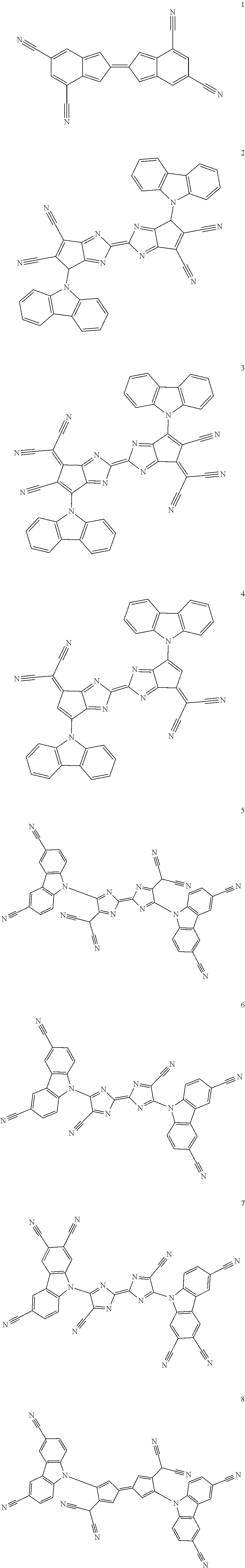 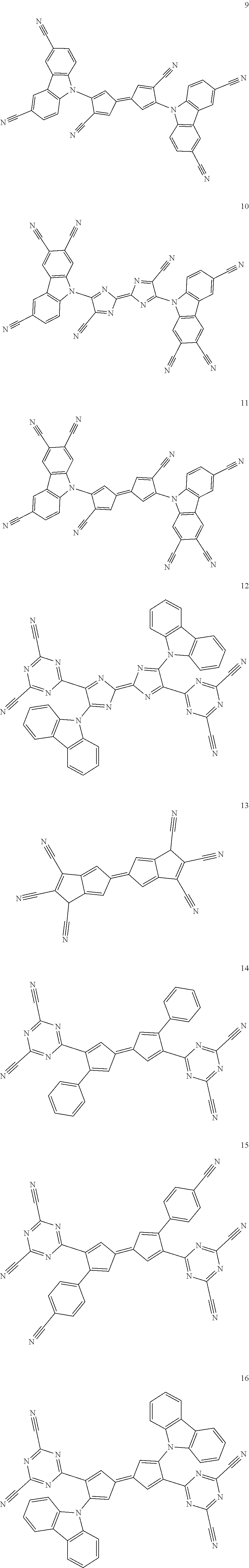 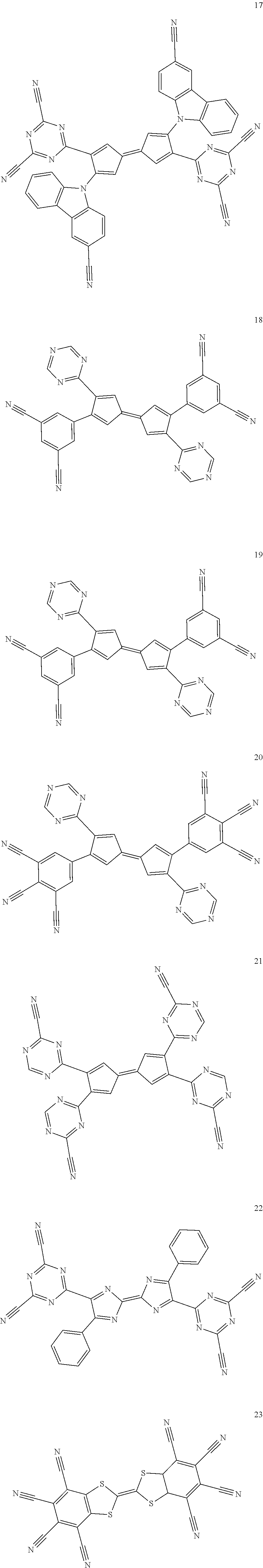 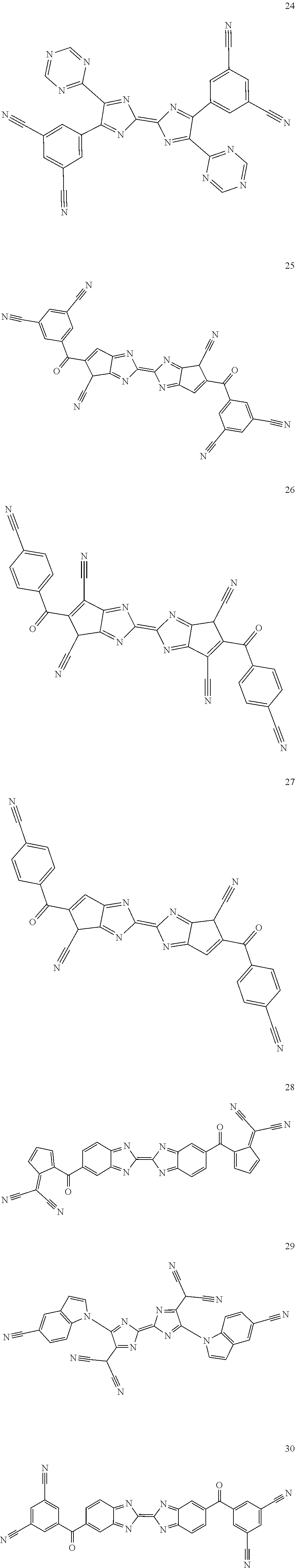 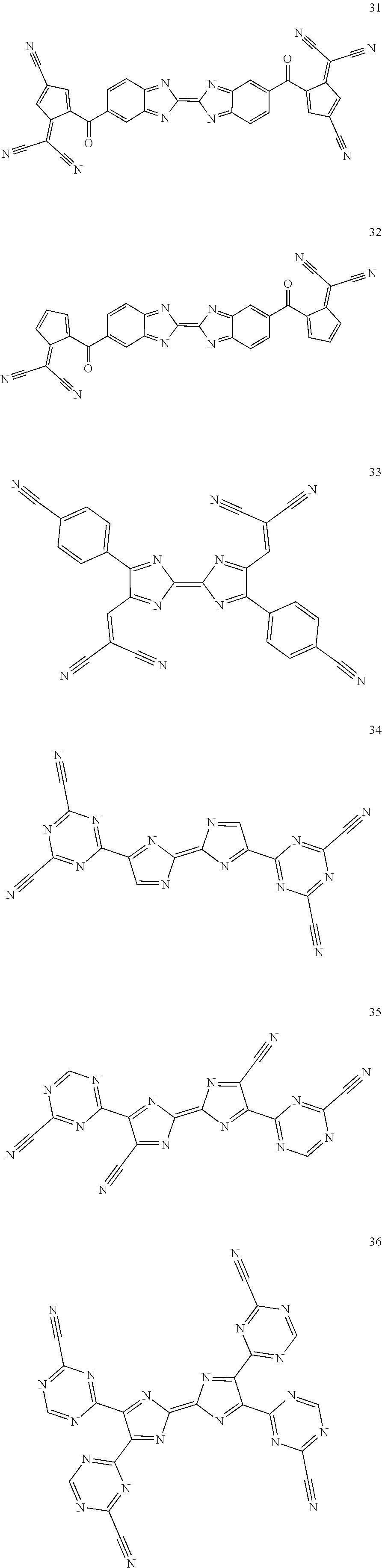 [SMILES: N#CC1=CC(=C(C#N)C#N)C(C(=O)c2ccc3c(c2)=N/C(=C2\N=c4ccc(C(=O)C5=CC(C#N)=CC5=C(C#N)C#N)cc4=N2)N=3)=C1 ; N#CC(C#N)=C1C=CC=C1C(=O)c1ccc2c(c1)=N/C(=C1\N=c3ccc(C(=O)C4=CC=CC4=C(C#N)C#N)cc3=N1)N=2 ; N#CC(C#N)=CC1=N/C(=C2/N=C(C=C(C#N)C#N)C(c3ccc(C#N)cc3)=N2)N=C1c1ccc(C#N)cc1 ; N#Cc1nc(C#N)nc(C2=N/C(=C3\N=CC(c4nc(C#N)nc(C#N)n4)=N3)N=C2)n1 ; N#CC1=N/C(=C2/N=C(C#N)C(c3ncnc(C#N)n3)=N2)N=C1c1ncnc(C#N)n1 ; N#Cc1ncnc(C2=NC(=C3N=C(c4ncnc(C#N)n4)C(c4ncnc(C#N)n4)=N3)N=C2c2ncnc(C#N)n2)n1] 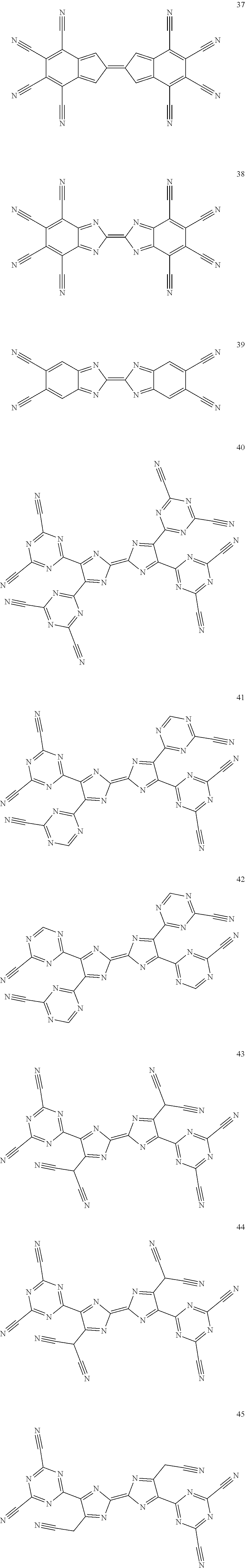 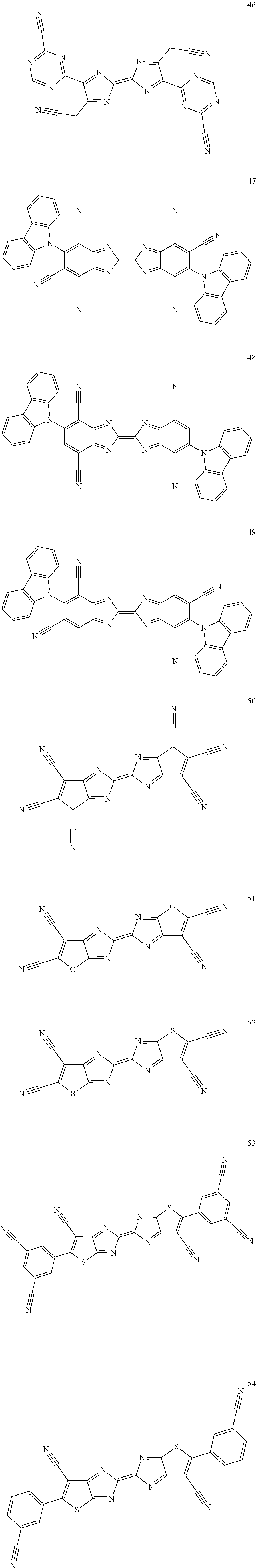 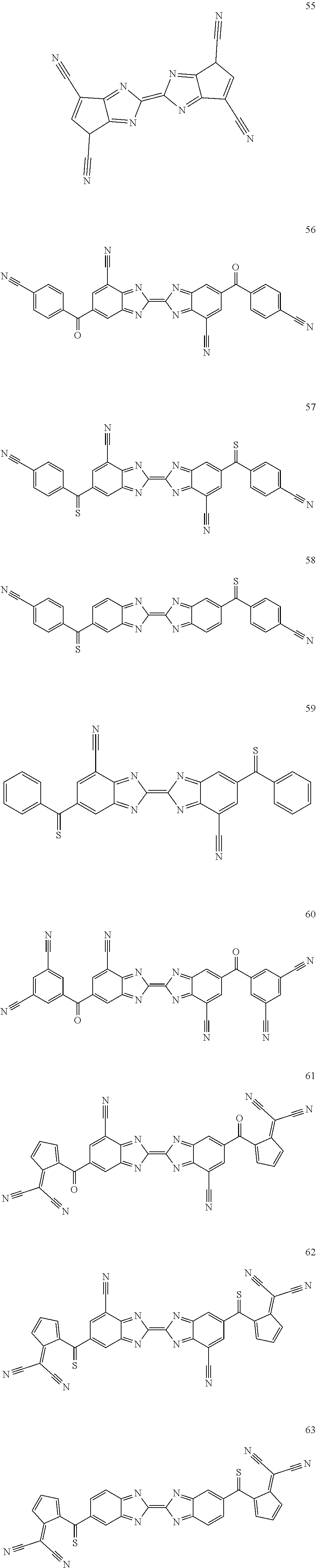 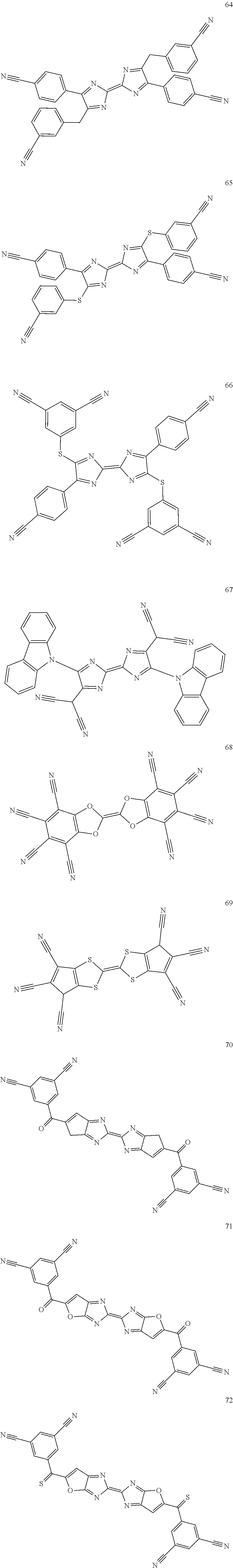 [chemical structure]
